(19) United States
(12) Patent Application Publication
(10) Pub. No.: US 2016/0375071 A1
(43) Pub. Date: Dec. 29, 2016

(54) COMPOSITIONS AND METHODS COMPRISING YEAST ORGANISMS AND LIPID EXTRACTS THEREOF

(71) Applicant: R.F. Technology Consultants, Inc., Hollywood, FL (US)

(72) Inventors: Ramon F. Figueroa, Hollywood, FL (US); Maria Corena McLeod, Jacksonville, FL (US)

(21) Appl. No.: 15/137,820

(22) Filed: Apr. 25, 2016

Related U.S. Application Data
(63) Continuation of application No. PCT/US2014/062464, filed on Oct. 27, 2014.
(60) Provisional application No. 61/895,490, filed on Oct. 25, 2013.

(51) Int. Cl. A61K 36/064; A61K 9/127; A61Q 19/10; A61K 8/97

(52) U.S. Cl. CPC A61K 36/064; A61K 8/975; A61K 9/127; A61Q 19/10; A61K 2236/30

(57) ABSTRACT

The invention provides compositions comprising whole yeast organisms and/or lipid yeast extract. Such compositions may be medical or cosmetic compositions, comprising one or more cosmetic or medical, pharmaceutical ingredients. The invention comprises methods of making such compositions. The yeast components of the compositions may be derived from yeast cultures which comprise at least 0.1% oil by dry weight.

### RELATED APPLICATION

The present application is a continuation of, and claims benefit of and priority to International Patent Application No. PCT/US2014/062464, filed Oct. 27, 2014, which claims the benefit of filing date and priority to U.S. Provisional Patent Application Ser. No. 61/895,490, filed Oct. 25, 2013, each of which is herein incorporated in its entirety. REFERENCE TO A SEQUENCE LISTING SUBMITTED AS A TEXT FILE VIA EFS-WEB The Sequence Listing submitted Sep. 9, 2016 as a text file named “31465_110625_6U2_Sequence Listing.txt,” created on Sep. 9, 2016, and having a size 3,863 bytes is hereby incorporated by reference pursuant to 37 C.F.R. §1.52(e)(5).

### FIELD OF THE INVENTION

Disclosed herein methods and compositions comprising yeast and yeast extracts that are useful for medical and cosmetic compositions and methods using such compositions.

### BACKGROUND OF THE INVENTION

Phospholipids are an important class of lipids, for example, in cell structure due to their amphiphilicity. Phospholipids are the major components of cell membranes. Their hydrophobic tail and hydrophilic head provide phospholipids to form lipid bilayers. These bilayers are made up of several compounds, including, but not limited to, phospholipids, sphingolipids, and sterols such as phophatidylcholine, sphingomylin, and cholesterol, respectively. Other phospholipid components include phophophatidylserine, phosphatidylehtanolamine, and phosphtadildylglycerol. The lipid bilayer creates a practically impermeable barrier to the interior of cells.

In living skin, lipids play a role in the formation and maintenance of both the permeability and antimicrobial barriers. A hydrophobic extracellular lipid matrix in the stratum corneum is composed primarily of lipids, such as phospholipids, sphingolipids, and cholesterol contributing to the barrier, cohesion, antimicrobial and other metabolic effects.

What is needed are cosmetic and medical compositions and methods comprising yeast or lipid yeast extract, or a combination of both yeast and lipid yeast extract, such as an extremophile yeast, that are effective in cosmetic and medical compositions and procedures comprising such compositions.

### SUMMARY OF THE INVENTION

Disclosed herein are compositions and methods comprising yeast or lipid yeast extract, or a combination of both yeast and lipid yeast extract as disclosed herein, for example, yeasts may serve as a source of lipids for cosmetic and medical compositions and methods. Disclosed herein are methods and compositions comprising yeast disclosed herein, and compounds or compositions isolated, such as extracted, from such yeast, such as lipids, for methods and compositions, including, but not limited to, personal care compositions, food and nutritional compositions, pharmaceutical compositions, incorporation of compositions disclosed herein into medical devices, methods of biotechnology and agriculture.

In an aspect, D. hansenii or other yeast disclosed herein may be used in medical and cosmetic compositions and methods. The yeast is provided in the composition in a substantially whole cell form, in that the yeast cell is not lysed, but is provided relatively intact, or the entire cell may be lysed and components of the yeast cell provided. Lysed cells' components may be homogenized.

In an aspect, compositions and methods disclosed comprise a lipid yeast extract. Compositions may comprise lipid yeast extract, for example, as liposome carriers made from the yeasts or compounds isolated from yeasts disclosed herein or liposome carriers may comprise phospholipids derived from yeast, such as D. hansenii.

In an aspect, compositions and methods disclosed comprise a combination of yeast cells and a lipid yeast extract.

Methods and compositions may comprise effective treatments for lipid replenishment, such as replenishment of skin lipids for animal skin and/or hair. Compositions disclosed herein may be used for topical administration and provide enhanced transdermal penetration and delivery. Delivery vehicles such as liposomes, for example, comprising a lipid yeast extract from D. hansenii, can be used as carriers of medicaments or actives via administration by topical routes, transdermal patch, oral routes of administration including liquids, tablets capsules, or injectable compositions including, but not limited to, intradermal, subcutaneous, intramuscular, intravenous, intraosseous, intraperitoneal, intrathecal, epidural, intracardiac, intraarticular, intracavernous, or intravitreal. Liposomes made with the lipids of yeast disclosed herein can be produced by those of skill in the art using methods of producing micropsheres, for example by sonification. Formulations comprising of emulsions that produce lamellar structures, such as liquid crystals, can be used in methods and compositions disclosed herein.

In an aspect, disclosed are cosmetic or medical compositions comprising at least 0.1% w/w whole yeast organisms yeast or lipid yeast extract, or a combination of both yeast and lipid yeast extract, and at least one other cosmetic or medical ingredient. For example, a composition may comprise at least 10% yeast by dry weight, comprise at least 20% yeast by dry weight, at least 30% yeast by dry weight, at least 40% yeast by dry weight, at least 50% yeast by dry weight, at least 60% yeast by dry weight, at least 70% yeast by dry weight, at least 80% yeast by dry weight, at least 90% yeast by dry weight, or 100% yeast by dry weight.

In an aspect, a composition may comprise at least 1% w/w yeast, which as used herein means the entire body of the yeast organism with its internal and external components, whether the body is intact (non-lysed yeast body) or not (lysed yeast body). In other aspects, a composition may comprise at least 10% w/w yeast. In an aspect, a composition may comprise at least 25% w/w yeast. In an aspect, a composition may comprise at least 50% w/w yeast. In an aspect, a cosmetic or medical composition disclosed is free of lipid other than lipid entrapped inside the yeast body.

In an aspect, disclosed herein are compositions comprising at least 0.1% w/w lipid yeast extract, and optionally, at least one other cosmetic or medical ingredient, in which the lipid yeast extract is derived from yeast disclosed herein. In an aspect, a yeast extract composition comprises 10-90% w/w lipid yeast extract by dry weight. In an aspect, a composition comprises 25-80% w/w lipid yeast extract by dry weight. In an aspect, a composition comprises 35-70% w/w lipid yeast extract by dry weight. In an aspect, a composition comprises 45-60% w/w lipid yeast extract by dry weight.

In an aspect, disclosed herein are compositions comprising at least 0.1% w/w yeast and lipid yeast extract, and optionally, at least one other cosmetic or medical ingredient, in which the lipid yeast extract is derived from yeast disclosed herein. In an aspect, a yeast and lipid yeast extract composition comprises 10-90% w/w yeast and lipid yeast extract by dry weight. In an aspect, a yeast and lipid yeast extract composition comprises 25-80% w/w yeast and lipid yeast extract by dry weight. In an aspect, a yeast and lipid yeast extract composition comprises 35-70% w/w yeast and lipid yeast extract by dry weight. In an aspect, a yeast and lipid yeast extract composition comprises 45-60% w/w yeast and lipid yeast extract by dry weight.

In an aspect, a yeast extract composition may comprise a mixture of lipids extracted from at least two distinct species of yeast. In an aspect, a composition comprises a mixture of at least two distinct species of yeast. In an aspect, at least two of the distinct species of yeast have been separately cultured. In an aspect, each distinct species has a lipid profile that is different from the other yeast used in a composition. In an aspect, a composition comprises yeast comprising a mixture of at least two different yeasts, each yeast having a lipid profile different from the other yeasts. As used herein, “yeast” means one or more individual organisms and may comprise a plurality of yeast organisms.

In an aspect, disclosed herein is a method of making a cosmetic or medical composition comprising combining yeast with optionally, at least one other cosmetic or medical ingredient, to form a cosmetic or medical composition. In an aspect, a method may comprise a method of making a cosmetic or medical composition comprising combining a lipid extract, extracted from yeast disclosed herein with at least one other cosmetic or medical ingredient to form a cosmetic or medical composition. In an aspect, a method comprises drying the yeast or extracted yeast lipids prior to combining the yeast or extracted yeast lipids, or a combination of yeast and extracted yeast lipids, with at least one other cosmetic or medical ingredient.

In an aspect, a method disclosed may comprise a method of making a cosmetic or medical composition comprising combining a lipid yeast extract with at least one other cosmetic or medical ingredient to form a cosmetic or medical composition.

In an aspect, a method may comprise a method of using a yeast and/or lipid yeast extract composition for cosmetic purposes, such as to soften and impart pliability to skin. In an aspect, a method comprises contacting the external surface of an animal, for example, human skin, with a yeast or lipid yeast extract, or a combination of both yeast and lipid yeast extract composition comprising intact yeast cells and at least 10% w/w lipid yeast extract by dry weight. In an aspect, a method comprises retaining the composition in contact with the external surface, for example, skin, for a predetermined time period such as, for example, 30 minutes, 1 hour, or longer. In an aspect, a yeast and/or lipid yeast extract composition is retained in contact with an external surface, for example, skin for at least 3 hours. In an aspect, a method of using a yeast and/or lipid yeast extract composition further comprises maintaining the composition in contact with an external surface, for example, skin, for a period of time sufficient to release at least 50% w/w of the oil from intact yeast cells, which may occur, for example, by enzymatic degradation of the yeast.

In disclosed methods of using a yeast composition to soften and impart pliability to external surface of animals, such as skin and/or hair, a composition may comprise yeast cells containing at least 15% oil by dry weight. In an aspect, a composition may comprise yeast cells containing at least 35% oil by dry weight. In an aspect, a composition may comprise yeast cells containing at least 45% oil by dry weight. In an aspect, a composition may comprise yeast cells containing 15-90% oil by dry weight. In an aspect, a composition may comprise yeast cells containing 25-80% oil by dry weight. In an aspect, a composition may comprise yeast cells containing 35-70% oil by dry weight.

In an aspect, a composition may comprise yeast cells containing 45-60% oil by dry weight. In a cosmetic or medical composition and/or method disclosed herein, a yeast cell may be one or more known yeasts. In an aspect, the yeast is an extremophile. In an aspect the yeast is Candida apicola, Candida etchellsii, Candida famata, Candida glabrata, Gandida guilliermondii, Candida lactis-condens, Candida magnolia, Candida parapsilosis, Candida tropicalis, Candida versatilis, Citeromyces matritensis, Debaryomyces hansenii, Hanseniaspora guilliermondii, Hyphopichia burtonii, Issatchenkia orientalis, Kluyveromyces thermotolerans, Pichia angusta, Pichia anomala, Pichia farinose, Pichia guilliermondii, Pichia membranaefaciens, Pichia ohmeri, Schizosaccharomyces octosporus, Schizosaccharomyces pombe, Torulaspora delbrueckii, Zygosaccharomyces Bailii, Zygosaccharomyces bisporus, Zygosaccharomyces microellipsoides, and Zygosaccharomyces roux.

In a composition and/or method disclosed herein, a cosmetic or medical ingredient may be one or more of absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts. In at least one embodiment, the other cosmetic or medical ingredient comprises a soap. In some cases, the soap comprises a saponified oil derived from yeast.

Compositions or methods disclosed herein can be combined together and are encompassed within the scope of the present disclosure.

### BRIEF DESCRIPTION OF THE DRAWINGS

FIGS. 1A and 1B show the separation of the aqueous and organic layers with supernatant from D. hansenii.

FIGS. 1C to 1H show the separation of the aqueous and organic layers with extracts from sonicated cells.

### DETAILED DESCRIPTION OF THE INVENTION

Disclosed herein are methods and compositions comprising lipids, such as yeasts comprising lipids, or extracts from yeasts, of which phospholipids are an example of lipids. Phospholipids are an important class of lipids in cell structure due to their amphiphilicity. Phospholipids are the major components of cell membranes. Their hydrophobic tail and hydrophilic head provide phospholipids to form lipid bilayers. These bilayers are made up of phospholipids, sphingolipids, and sterols such as phophatidylcholine, sphingomylin, and cholesterol respectively. Other phospholipid components of importance include phophophatidylserine, phosphatidylehtanolamine, and phosphtadildylglycerol. The lipid bilayer creates a practically impermeable barrier to the cells.

In skin, lipids play an essential role in the formation and maintenance of both the permeability and antimicrobial barriers. A hydrophobic extracellular lipid matrix in the stratum corneum is composed primarily of lipids, such as phospholipids, sphingolipids, and cholesterol contributing to the barrier, cohesion, antimicrobial and other metabolic effects.

Phospholipids such as phosphatidylcholine when combined with phospholipid surfactants such as phastidylethanolamine under high sheer have been shown to artificially produce spherical cell like membranes called vesicles named liposomes. Liposome have been shown useful as carriers for enhance permeability and delivery of nutrients and pharmaceutical drugs. Liposomes have been commercially produced for multiple applications. Liposomes may be made methods known to those of skill in the art.

An example of a yeast useful in the methods and compositions disclosed herein is Debaryomyces hansenii, though the invention is not limited to only one species of yeast, and the references herein to a particular yeast is for clarity and not to be seen as limiting. Debaryomyces hansenii is an oleaginous yeast with roughly 70% w/w lipid content. Though not wishing to be bound by any particular theory, it is thought that major phospholipids in D. hansenii are phosphatidylcholine, followed by phosphatidylinositol, phosphatidylethanolamine, phosphatidylserine, phophatidylglycerol and cardiolipin.

Yeast can be used to produce lipids economically, for example, for use in cosmetic or medical methods and compositions. A yeast disclosed herein for use in the invention is the lipid-producing yeast Debaryomyces hensenii. Disclosed herein are methods of culturing Debaryomyces hensenii as well as multiple other species of yeast to generate lipids for use in cosmetic or medical compositions. Any species of yeast that produces suitable oils and/or lipids can be used in accordance with the present disclosure, although yeast that produce high levels of suitable oils and/or lipids are effective for methods and compositions disclosed herein.

Considerations for selection of yeast for methods and compositions disclosed herein, in addition to production of suitable oils or lipids for compositions, include, but are not limited to (1) high lipid content as a percentage of cell weight; (2) ease of growth; (3) ease of propagation; (4) ease of biomass processing; (5) lipid profile and (6) lack of toxins. In an aspect, the yeast must be disrupted during the use of the cosmetic or medical composition (e.g., soaps containing whole yeast cells) in order to release the lipid components. Hence, in some compositions it is advantageous to comprise strains of yeast susceptible to disruption, such as when the yeast is to be used as whole yeast cells as an ingredient in the final cosmetic or medical composition.

In an aspect, wild-type or genetically engineered yeast comprise cells that are at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% or more, oil by dry weight. Processing considerations can include, for example, the availability of effective means for lysing the yeast cells. In an aspect, not all types of lipids are desirable for use in cosmetics or medicine or as cosmetic or medical ingredients, as the lipids may have aesthetic issues, such as smelling bad, having poor stability or providing a poor tactile sensation.

Yeasts useful in accordance with the methods disclosed herein are found in various locations and environments throughout the world. As a consequence of their isolation from other species and their resulting evolutionary divergence, the particular growth medium for optimal growth and generation of whole yeast and/or yeasts for lipid yeast extract from any particular species of yeast may be determined by those of skill in the art who can readily find appropriate media by routine testing for growing yeast. In some cases, certain strains of yeast may be unable to grow on a particular growth medium because of the presence of some inhibitory component or the absence of some essential nutritional requirement required by the particular strain of yeast. The fixed carbon source is a component of the medium for growing yeast. Suitable fixed carbon sources, include, for example, glucose, fructose, sucrose, galactose, xylose, mannose, rhamnose, arabinose, N-acetylglucosamine, glycerol, floridoside, glucuronic acid, and/or acetate.

In a steady growth state, the yeast cells may accumulate oil but do not undergo cell division. In an aspect, the growth state is maintained by continuing to provide all components of the original growth media to the cells with the exception of a particular component of the media. Cultivating yeast cells by feeding all nutrients originally provided to the cells except for a particular component, such as through feeding the cells for an extended period of time, results in a higher percentage of lipid by dry cell weight. Yeast grown using conditions described herein or otherwise known in the art can comprise at least about 20% lipid by dry weight, and often comprise 35%, 45%, 55%, 65%, and even 75% or more lipid by dry weight. Percentage of dry cell weight as lipid in yeast lipid production can therefore be improved by holding cells in a heterotrophic growth state in which they consume carbon and accumulate oil but do not undergo cell division.

High protein biomass from yeast is another material for inclusion in cosmetic or medical compositions disclosed herein. A method of growing yeast may comprise growing yeast so that the yeast comprises a biomass that is at least 30% of its dry cell weight as protein. Growth conditions can be adjusted to increase the percentage weight of yeast cells that is protein. Such methods may be known to those of skill in the art or disclosed herein.

A bioreactor or fermenter may be used to culture yeast cells through the various phases of their physiological cycle. As an example, an inoculum of lipid-producing yeast cells is introduced into a medium; there is a lag period (lag phase) before the cells begin to divide and reproduce (propagate). Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells. For obtaining protein rich biomass, a yeast culture is typically harvested during or shortly after the end of the exponential phase. For obtaining lipid rich biomass, a yeast culture is typically harvested well after the end of the exponential phase, which may be terminated early by allowing a key nutrient (other than carbon) to become depleted, forcing the cells to convert the carbon sources, present in excess, to lipid. Culture condition parameters can be manipulated to optimize total oil production, the combination of lipid species produced, and/or production of a specific lipid.

Bioreactors offer many advantages for use in growth and propagation methods. To produce biomass for use in cosmetics or medical compositions, yeast are preferably fermented in large quantities in liquid, such as in suspension cultures as an example. Bioreactors such as steel fermenters (5000 liter, 10,000 liter, 40,000 liter, and larger) can accommodate very large culture volumes. Bioreactors also typically allow for the control of culture conditions such as temperature, pH, oxygen tension, and carbon dioxide levels. For example, bioreactors are typically configurable, for example, using ports attached to tubing, to allow gaseous components, like oxygen or nitrogen, to be bubbled through a liquid culture.

Increased gas flow affects the turbidity of the culture as well. Turbulence can be achieved by placing a gas entry port below the level of the aqueous culture media so that gas entering the bioreactor bubbles to the surface of the culture. One or more gas exit ports allow gas to escape, thereby preventing pressure buildup in the bioreactor. Preferably a gas exit port leads to a “one-way” valve that prevents contaminating microorganisms from entering the bioreactor. The specific examples of bioreactors, culture conditions, and growth and propagation methods described herein can be combined in any suitable manner to improve efficiencies of microbial growth and lipid and/or protein production.

Yeast cultures generated according to methods disclosed herein yield yeast in fermentation media. To prepare the yeast for use as a cosmetic or medical composition, the yeast is concentrated, or harvested, from the fermentation medium. At the point of harvesting the yeast from the fermentation medium, the yeast comprises predominantly intact cells suspended in an aqueous culture medium. The present disclosure is not limited by the disclosed methods for concentrating yeast, as those of skill in the art are well aware of many methods to accomplish concentration of yeast. For example, to concentrate the yeast, a dewatering step may be performed. Dewatering or concentrating refers to the separation of the biomass from fermentation broth or other liquid medium and so is solid-liquid separation. Thus, during dewatering, the culture medium is removed from the yeast (for example, by draining the fermentation broth through a filter that retains the yeast), or the yeast is otherwise removed from the culture medium. Common processes for dewatering include centrifugation, filtration, and the use of mechanical pressure. These processes can be used individually or in any combination.

The concentrated yeast produced in accordance with the methods of the invention is itself a finished cosmetic or medical ingredient and may be used in cosmetics or medical compositions without further, or with only minimal, modifications or other composition components. For example, the concentrated yeast can be vacuum-packed or frozen. Alternatively, the yeast may be dried via lyophilization, a “freeze-drying” process, in which the yeast is frozen in a freeze-drying chamber to which a vacuum is applied. The application of a vacuum to the freeze-drying chamber results in sublimation (primary drying) and desorption (secondary drying) of the water from the biomass. However, the present disclosure provides a variety of yeast for finished cosmetic or medical composition wherein the yeast have enhanced properties resulting from processing methods of the invention.

Drying the yeast, either predominantly intact or after homogenizing (lysing and mixing to form a homogenate form), may be a step performed prior to further processing or for use of the yeast in methods and compositions described herein. Drying refers to the removal of free water or surface moisture/water from predominantly intact biomass or the removal of surface water from a slurry of homogenized (e.g., by micronization) biomass. Different textures and dispersion properties can be conferred to cosmetic or medical compositions depending on whether the yeast biomass is dried, and if so, the drying method. Drying the biomass generated from the cultured yeast described herein removes water that may be an undesirable component of finished cosmetic or medical compositions. In some cases, drying the biomass may facilitate a more efficient oil extraction process.

In an aspect, the concentrated yeast is drum dried to a flake form to produce flake. In an aspect, the concentrated yeast is spray or flash dried (i.e., subjected to a pneumatic drying process) to form a powder containing predominantly intact cells to produce powder. In an aspect, oil and/or lipids is extracted from the concentrated yeast to form yeast oil or lipids.

In an aspect, disclosed herein are methods of combining whole yeast organisms and/or a lipid yeast extract, as disclosed herein, with at least one other cosmetic or medical ingredient, as disclosed herein, to form a cosmetic or medical composition. In an aspect, a cosmetic or medical composition formed by the combination of yeast and/or lipid yeast extract comprises at least 1%, at least 5%, at least 10%, at least 25%, or at least 50% w/w yeast or lipid yeast extract, respectively. In an aspect, cosmetic or medical compositions formed as described herein comprise at least 2%, at least 3%, at least 4%, at least 15%, at least 20%, at least 30%, at least 35%, at least 40%, at least 45%, at least 55%, at least 60%, at least 65%, at least 70%, at least 75%, at least 80%, at least 85%, at least 90%, or at least 95% w/w yeast or lipid yeast extract.

In an aspect, a cosmetic or medical composition comprises predominantly intact yeast cells. In an aspect, a cosmetic or medical composition comprises at least 50% intact cells, or at least 60%, at least 70%, or at least 80% intact cells, w/w. In an aspect, a cosmetic or medical composition comprises yeast that has been homogenized to form a whole cell dispersion, but with no extraction of any components of the yeast from the whole cell dispersion.

In an aspect, yeast can be substituted for other components that would otherwise be conventionally included in a cosmetic or medical composition. In an aspect, a cosmetic or medical composition disclosed is free of oil other than oil contributed by the yeast cells and is entrapped therein if the yeast is in an intact cell form.

In an aspect, yeast can be substituted for all or a portion of conventional cosmetic or medical ingredients such as exfoliants, antioxidants, colorants, and the like, to the extent that the components of the yeast replace the corresponding conventional components in like kind, or adequately substitute for the conventional components to impart the desired characteristics to the cosmetic or medical composition.

In an aspect, a lipid yeast extract can be substituted for oils, lipids or fats conventionally used in cosmetic or medical compositions. As described herein, lipids produced by yeast can be tailored by culture conditions or lipid pathway engineering to comprise particular fatty acid components. Thus, lipids generated by yeast disclosed herein can be used to replace conventional cosmetic or medical ingredients such as essential oils, fragrance oils, and the like. In an aspect, a cosmetic or medical composition is free of oil or lipids other than lipids extracted from yeast. As used herein, oil and lipid means the fat compounds of yeast, and may be used interchangeably and are not limited by length of carbon backbone, hydrogenation, number of double bonds in the carbon chains, and understood by those of skill in the art to be characterized as fats, in contrast to compounds such as carbohydrates, proteins or nucleic acids.

Yeast or lipid yeast extract, or a combination of both yeast and lipid yeast extract may be combined with at least one cosmetic or medical ingredient in methods to form cosmetic or medical compositions. Cosmetic or medical ingredients can be selected from conventional cosmetic or medical ingredients suitable for use with the yeast or lipid yeast extract, or both, with regard to the intended use of the composition. Such other cosmetic or medical ingredients include, without limitation, absorbents, abrasives, anticaking agents, antifoaming agents, antibacterial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances and flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

Essential oils include allspice, amyris, angelica root, anise seed, basil, bay, bergamot, black pepper, cajeput, camphor, cananga, cardamom, carrot seed, cassia, catnip, cedarwood, chamomile, cinnamon bark, cinnamon leaf, citronella java, clary sage, clovebud, coriander, cornmint, cypress, davana, dill seed, elemi, eucalyptus, fennel, fir, frankincense, geranium bourbon, geranium roast, geranium, ginger, grapefruit pink, grapefruit, gurjum balsam, hyssop, juniper berry, lavandin, lavandula, lavender, lemon myrtle, lemon tea tree, lemon, lemongrass, lime, litsea cubeba, mandarin, marjoram, mullein, myrrh, neroli, nerolina, niaouli, nutmeg, orange, palmarosa, patchouli, peppermint, petitgrain, pine needle, ravensara, ravintsara, rosalina, rose, rosemary, rosewood, sage, sandalwood, spearmint, spikenard, star anise, tangerine, tea tree, thyme, tulsi, verbena, vetiver, ylang ylang, and zdravetz, or combinations thereof.

Fragrances and flavor oils include absolute tulip, almond, amaretto, amber, anais, apple, apple cinnamon, apple spice, apricot, apricot creme, arabian musk, asian pear, asian plum blossom, autumn woods, banana, basil, basil nectarine, bay rum, bayberry, bergamot, berries and cream, birthday cake, black cherry, black tea, blackberry tea, blackcurrent, blue nile, blueberry delight, brambleberry preserves, brown sugar, bubble gum, buttercream, butterscotch, calla lily, cantaloupe, caramel apple, carnation, carrot cake, chai tea, chamomile, china musk, china rain, chinese peony, chrysanthemum, cinnamon, coconut, coconut cream, cotton candy, cranberry, cucumber, cucumber melon, daffodil, dandelion, delphinium, dewberry, dulce de leche, earl grey tea, easter cookie, egg nog, egyptian musk, enchanted forest, english lavender, english pear, evergreen, fig, frangipani, frankincense, french vanilla, fresh apple, fresh brewed coffee, fruit punch, gardenia, geranium, ginger lily, gingerbread, grape, grapefruit, green apple, green grass, green tea, guava, guava flower, hawaiian white ginger, heliotrope, hemp, herbaceous, holiday fruitcake, hollyberry, honey ginger, honey, honeysuckle, jasmine, jasmine tea, juniper berries, kiwi, lavender, leather, lemon, lemon parsley, lilac, lime, loganberry, lotus blossom, magnolia, mandarin, mango, mango and kiwi, maple, milk chocolate, mimosa, minty lime, mulberry, myrrh, neroli, oakmoss, oatmeal, ocean rain, orange blossom, orange sherbet, orange vanilla, papaya, passion fruit, patchouli, peach, peaches and cream, pearberry, peppermint, pikaki, pina colada, pineapple, pomegranate, pumpkin pie, raisins and almonds, raspberry, roasted nuts, rosewood, sage, sandalwood, sassafras, sea moss, sesame, siberian pine, snowberry, spanish moss, spice, strawberry, sugar plum, suntan lotion, sweet clove, sweet grass, sweet pea, tangerine, that coconut, timber, tomato leaf, vanilla, watermelon, white chocolate, wild cherry, wisteria, witches brew, and ylang ylang, or combinations thereof.

Exfoliants include particles that can be used to dislodge dead skin cells, dirt, or other materials from the surface of the skin, and include without limitation, fruit seeds and fibers, grain powders, nut and seed meals, and oil or wax beads. Fruit fibers include blueberry, cranberry, grape, kiwi, raspberry, blackberry, strawberry, and the like. Grain powders include oat powder, and almond powder, or the like, milled to varying degrees of coarseness. Polymer beads, such as those made from polyethylene, or the like, can also be used. The removal of dead skin cells and/or the outer most layer of skin can provide an opportunity for bioactive agents, such as carotenoids, which can also be present in the compositions of the invention, to have greater access to deeper layers of the skin.

Cosmetic or medical ingredients may comprise extracts, including herbal extracts derived from conventional extraction procedures, or via the use of liquefied carbon dioxide. Herbs may include, but are not limited to, aloe vera leaf, alfalfa leaf, alkanet root, annatto seed, arrowroot, burdock root, calendula petals, carrot root, chamomile flower, comfrey leaf, cornsilk, dutch blue poppies, fennel seed, ginger root, ginseng, green tea leaf, jasmine flower, juniper berries, lavender buds, lemon peel, lemongrass, marshmallow root, nettles, oat straw, orange peel, paprika, parsley, peppermint leaf, rose buds, rose petals, rosehip, rosemary leaf, shavegrass, spearmint leaf, and St. John's wort, or combinations thereof.

Cosmetic or medical ingredients may comprise colorings, including, but not limited to, glitters, green #5, green #8, orange #4, red #22, red #33, violet #2, blue #1, green #3, red #40, yellow #5, yellow #6, green #6, red #17, as well as pearlescent micas and tinting herbs such as henna leaf, sandalwood, turmeric, cranberry, kiwi, raspberry, alkanet, annatto, carrot root, nettles, paprika, and parsley.

Specific examples of other cosmetic or medical ingredients are disclosed herein. Any one or more of these can be optionally combined with yeast or lipid yeast extract or combinations of both, to form a cosmetic or medical composition. The active ingredients disclosed herein are categorized by their cosmetic and/or therapeutic benefit or their postulated mode of action. However, it is to be understood that these ingredients can in some instances provide more than one cosmetic and/or therapeutic benefit or operate via more than one mode of action. Therefore, classifications herein are made for convenience and are not intended to limit the ingredient to that particular application or applications listed.

An anti-inflammatory agent can optionally be added to the compositions of the present invention, preferably from about 0.1% to about 10%, more preferably from about 0.5% to about 5%, of the composition, w/w. An anti-inflammatory agent may enhance the skin appearance, e.g., such agents contribute to a more uniform and acceptable skin tone or color. The exact amount of anti-inflammatory agent to be used in the compositions will depend on the particular anti-inflammatory agent utilized since such agents vary widely in potency, and those of skill in the art can determine such amounts depending on the desired effects of the compositions.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

A second class of anti-inflammatory agents which is useful in the compositions includes nonsteroidal anti-inflammatory agents. The variety of compounds encompassed by this group are well-known to those skilled in the art. For detailed disclosure of the chemical structure, synthesis, side effects, etc. of nonsteroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974), each incorporated herein by reference.

Specific non-steroidal anti-inflammatory agents useful in methdos and compositions include, but are not limited to: 1) the oxicams, such as piroxicam, isoxicam, tenoxicam, sudoxicam, and CP-14,304; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application.

Other anti-inflammatory agents are useful in methods and compositions disclosed herein, Such agents may suitably be obtained as an extract by suitable physical and/or chemical isolation from natural sources (e.g., plants, fungi, or by-products of microorganisms). For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, such as C₁₀-C₂₄, or C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

In an aspect, a composition may also optionally comprise a retinoid. Vitamin B₃ compounds and retinoids provide benefits in regulating skin condition, especially in therapeutically regulating signs of skin aging, more especially wrinkles, lines, and pores. Without intending to be bound or otherwise limited by theory, it is believed that the vitamin B₃ compounds increase the conversion of certain retinoids to trans-retinoic acid, which is believed to be the biologically active form of the retinoid, to provide synergistic regulation of skin condition (namely, increased conversion for retinol, retinol esters, and retinal). In addition, the vitamin B₃ compounds unexpectedly mitigate redness, inflammation, dermatitis and the like which may otherwise be associated with topical application of retinoid (often referred to, and hereinafter alternatively referred to as “retinoid dermatitis”). Furthermore, combined vitamin B₃ compounds and retinoid(s) tend to increase the amount and activity of thioredoxin, which tends to increase collagen expression levels via the protein AP-1. Compositions disclosed herein may provide reduced active levels, and therefore reduced potential for retinoid dermatitis, while retaining significant positive skin conditioning benefits. In addition, higher levels of retinoid(s) may be used to obtain greater skin conditioning efficacy, without undesirable retinoid dermatitis occurring.

As used herein, “retinoid(s)” includes all natural and/or synthetic analogs of Vitamin A or retinol-like compounds which possess the biological activity of Vitamin A in the skin as well as the geometric isomers and stereoisomers of these compounds. A retinoid may be retinol, retinol esters (e.g., C₂-C₂₂ alkyl esters of retinol, including retinyl palmitate, retinyl acetate, retinyl proprionate), retinal, and/or retinoic acid (including all-trans retinoic acid and/or 13-cis-retinoic acid). These compounds are well known in the art and are commercially available from a number of sources, e.g., Sigma Chemical Company (St. Louis, Mo.).

Cosmetic or medical compositions disclosed herein may contain an effective amount of a retinoid, such that the resultant composition is effective for regulating a skin condition, for example, for affecting visible and/or tactile discontinuities in skin, for affecting signs of skin aging, for affecting visible and/or tactile discontinuities in skin texture associated with skin aging. A compositions may comprise from about 0.005% to or about 2%, about 0.01% to about 2%, retinoid, w/w. Retinol may be used in an amount of from about 0.01% to about 0.15% w/w; retinol esters may be used in an amount of from about 0.01% to about 2% w/w (e.g., about 1%); retinoic acids may be used in an amount of from about 0.01% to about 0.25% w/w. The retinoid may be included as the substantially pure material, or as an extract obtained by suitable physical and/or chemical isolation from natural (e.g., plant) sources. The retinoid is preferably substantially pure.

In an aspect, a composition disclosed herein may comprise an antibacterial agent. As used herein, “antibacterial agent” means a compound capable of destroying bacteria cells, preventing the development of bacteria or preventing the pathogenic action of bacteria. Antibacterial agents are useful, for example, in controlling acne. An effective amount of an antibacterial agent can be added to cosmetic or medical compositions of the subject invention, for example, from about 0.001% to about 10%, from about 0.01% to about 5%, from about 0.05% to about 2% or from about 0.05% to about 1% (w/w) of the compositions. Antibacterial agents useful in the cosmetic or medical compositions include, but are not limited to, benzoyl peroxide, erythromycin, tetracycline, clindamycin, azelaic acid, and sulfur resorcinol.

In an aspect, compositions disclosed herein may comprise an anti-androgen compound. As used herein, “anti-androgen” means a compound capable of correcting androgen-related disorders by interfering with the action of androgens at their target organs. A target organ for a disclosed cosmetic or medical compositions can be animal skin, including but not limited to, mammalian skin, hair, nails or other integumentary structures. Exemplary antiandrogens include pregnenalone (and its derivatives), hops extract, oxygenated alkyl substituted bicyclo alkanes (e.g., ethoxyhexyl-bicyclo octanones such as marketed by Chantal Pharmaceutical of Los Angeles, Calif. under the trade names ETHOCYN and CYOCTOL, and 2-(5-ethoxy hept-1-yl)bicylo[3.3.0]octanone), and oleanolic acid. Suitable antiandrogens are disclosed in U.S. Pat. Nos. 4,689,345 and 4,855,322, both issued to Kasha et al. on Aug. 25, 1987 and Aug. 8, 1989, respectively, each incorporated herein by reference. Antiandrogens can optionally be added to cosmetic or medical compositions of the invention.

Exposure to ultraviolet light can result in excessive scaling and texture changes of the stratum corneum. Cosmetic or medical compositions disclosed herein may comprise a sunscreen or sunblock. Suitable sunscreens or sunblocks may be organic or inorganic. A wide variety of conventional sunscreening agents are suitable for use in cosmetic or medical compositions described herein. Sagarin, et al., at Chapter VIII, pages 189 et seq., of Cosmetics Science and Technology (1972), discloses numerous suitable agents, and is incorporated herein by reference. Specific suitable sunscreening agents include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylacetoumbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one) and 4-isopropyl-di-benzoylmethane.

Cosmetic or medical compositions may comprise sunscreens such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991, both of which are incorporated herein by reference, or those sunscreens known to those of skill in the art. The sunscreens disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. Members of this class of sunscreening agents include 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-15 ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof. Suitable inorganic sunscreens or sunblocks include metal oxides, e.g., zinc oxide and titanium dioxide.

An effective amount of the sunscreen or sunblock is used, typically from about 1% to about 20%, more typically from about 2% to about 10%, w/w. Exact amounts will vary depending upon the sunscreen chosen and the desired Sun Protection Factor (SPF).

Compositions disclosed herein may comprise an agent to improve the skin substantivity of those compositions, particularly to enhance their resistance to being washed off by water, or rubbed off. A substantivity agent which will provide this benefit is a copolymer of ethylene and acrylic acid. Compositions comprising this copolymer are disclosed in U.S. Pat. No. 4,663,157, Brock, issued May 5, 1987, which is incorporated herein by reference.

Cosmetic or medical compositions may comprise an anti-oxidant/radical scavenger as an ingredient. An anti-oxidant/radical scavenger is useful for providing protection against UV radiation which can cause increased scaling or texture changes in the stratum corneum and against other environmental agents which can cause skin damage. An effective amount of an anti-oxidant/radical scavenger may be added to the compositions disclosed herein, for example, from about 0.1% to about 10%, from about 1% to about 5%, (w/w) of the composition.

Anti-oxidants/radical scavengers include, but are not limited to, ascorbic acid (vitamin C) and its salts, ascorbyl esters of fatty acids, ascorbic acid derivatives (e.g., magnesium ascorbyl phosphate), tocopherol (vitamin E), tocopherol sorbate, other esters of tocopherol, butylated hydroxy benzoic acids and their salts, 6-hydroxy-2,5,7,8-tetramethylchroman-2-carboxylic acid (commercially available under the tradename Trolox.sup.R), gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, catalase, superoxide dismutase, lactoferrin, silymarin, tea extracts, grape skin/seed extracts, melanin, and rosemary extracts may be used.

As used herein, “chelating agent” refers to an active agent capable of removing a metal ion from a system by forming a complex so that the metal ion cannot readily participate in or catalyze chemical reactions. The inclusion of a chelating agent may be useful for providing protection against UV radiation which can contribute to excessive scaling or skin texture changes and against other environmental agents which can cause skin damage.

An effective amount of a chelating agent can optionally be added to a cosmetic or medical composition disclosed herein, from about 0.1% to about 10%, from about 1% to about 5%, (w/w) of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995; all incorporated herein by reference. For example, chelators useful in compositions are furildioxime and derivatives thereof.

Compositions of the present invention may comprise an organic hydroxy acid. Suitable hydroxy acids include C₁-C₁₈ hydroxy acids, such as C₈ or below. The hydroxyl acids can be substituted or unsubstituted, straight chain, branched chain or cyclic (preferably straight chain), and saturated or unsaturated (mono- or poly-unsaturated) (preferably saturated). Non-limiting examples of suitable hydroxy acids include salicylic acid, glycolic acid, lactic acid, 5 octanoyl salicylic acid, hydroxyoctanoic acid, hydroxycaprylic acid, and lanolin fatty acids. Concentrations of the organic hydroxy acid may range from about 0.1% to about 10%, from about 0.2% to about 5%, from about 0.5% to about 2%, w/w. Salicylic acid is an example of an organic hydroxyl acid. For example, organic hydroxy acids tend to improve the texture of the skin. Compositions disclosed herein may comprise a desquamation agent. In an aspect, desquamation agents, which may also be known as exfoliants, can comprise from about 0.1% to about 10%, from about 0.2% to about 5%, or from about 0.5% to about 4% w/w of a cosmetic or medical composition. Desquamation agents tend to improve the texture of the skin (e.g., smoothness). A variety of desquamation agents are known in the art and are suitable for use herein, including but not limited to the organic hydroxy agents described above.

Compositions disclosed herein may comprise an effective amount of a depilation agent. When used, the composition may comprise from about 0.1% to about 10%, from about 0.2% to about 5%, from about 0.5% to about 2% w/w of a depilation agent. A depilation agent may comprise a sulfhydryl compound, e.g., N-acetyl-L-cysteine.

Composition disclosed herein may comprise a skin lightening agent. A compositions may comprise from about 0.1% to about 10%, from about 0.2% to about 5%, from about 0.5% to about 2%, w/w of a skin lightening agent. Suitable skin lightening agents include those known in the art, including kojic acid, arbutin, ascorbic acid and derivatives thereof, e.g., magnesium ascorbyl phosphate.

Compositions disclosed herein may comprise a zinc salt. Zinc salts may be used when the composition contains a sulfhydryl compound, e.g., N-acetyl-L-cysteine. Without intending to be limited or bound by theory, it is believed that the zinc salt acts as a chelating agent capable of complexing with the sulfhydryl compound prior to topical application, stabilizes the sulfhydryl compound and/or controls odor associated with the sulfhydryl compound. Concentrations of the zinc salt can range from about 0.001% to about 10%, from about 0.01% to about 5%, from about 0.1% to about 0.5% by weight of the composition.

Zinc salts include, but are not limited to, zinc acetate, zinc acetate hydrates such as zinc acetate-2-water, zinc aluminum oxide complexes such as gahnite, zinc diamine, zinc antimonide, zinc bromate hydrates such as zinc bromate-6water, zinc bromide, zinc carbonates such as zincspar and smithsonite, zinc chlorate hydrates such as zinc chlorate-4-water, zinc chloride, zinc diamine dichloride, zinc citrate, zinc chromate, zinc dichromate, zinc diphosphate, zinc hexacyanofluoride ferrate (II), zinc fluoride, zinc fluoride hydrates such as zinc fluoride-4-water, zinc formate, zinc formate hydrates such as zinc formate-2-water, zinc hydroxide, zinc iodate, zinc iodate hydrates such as zinc iodate-2-water, zinc iodide, zinc iron oxide complexes, zinc nitrate hydrates such as zinc nitrate-6-water, zinc nitride, zinc oxalate hydrates such as zinc oxalate-2-water, zinc oxides such as zincite, zinc perchlorate hydrates such as zinc perchlorate-6-water, zinc permanganate hydrates such as zinc permanganate-6-water, zinc peroxide, zinc p-phenolsulfonate hydrates such as zinc p-phenosulfonate-8-water, zinc phosphate, zinc phosphate hydrates such as zinc phosphate-4-water, zinc phosphide, zinc-propionate, zinc selenate hydrates such as zinc selenate-5-water, zinc selenide, zinc silicates such as zinc silicate (2) and zinc silicate (4), zinc silicon oxide water complexes such as hemimorphite, zinc hexafluorosilicate hydrates such as zinc hexafluorosilicate-6-water, zinc stearate, zinc sulfate, zinc sulfate hydrates such as zinc sulfate-7-water, zinc sulfide, zinc sulfite hydrates such as zinc sulfite-2-water, zinc telluride, zinc thiocyanate, zinc (II) salts of N-acetyl L-cysteine, and mixtures thereof.

Compositions disclosed herein may a humectant, moisturizing agent or other skin conditioning agent. A variety of these materials can be employed and each can be present at a level of from or about 0.1% to or about 20%, from or about 1% to or about 10%, or from or about 2% to or about 5%, w/w. These materials include guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine; and mixtures thereof. Also useful are the propoxylated glycerols described in U.S. Pat. No. 4,976,953, which is incorporated herein by reference. Compositions disclosed herein may C₁-C₃₀ monoesters and polyesters of sugars and related materials. These esters are derived from a sugar or polyol moiety and one or more carboxylic acid moieties. Depending on the constituent acid and sugar, these esters can be in either liquid or solid form at room temperature. Examples of liquid esters include; glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C:18 mono- and/or di-unsaturated and behenic, in a molar ratio of unsaturates:behenic of 1:7 to 3:5. A solid sugar polyester is the octaester of sucrose in which there are about 7 behenic fatty acid moieties and about 1 oleic acid moiety in the molecule. The ester materials are further described in, U.S. Pat. Nos. 2,831,854, 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985; all of which are incorporated by reference herein in their entirety.

Compositions disclosed herein may comprise compounds that stimulate the production of collagen. Such compounds include Factor X (kinetin), Factor Z (zeatin), n-methyl taurine, dipalmitoyl hydroxyproline, palmitoyl hydroxyl wheat protein, biopeptide CL (palmitoyl glycyl-histidyl-lysine), ASC III (Amplifier of Synthesis of Collagen III, E. Merck, Germany), beta glucan, and ceramides or the like, for example, ceramide 1-6.

Compositions disclosed herein may an oil absorbent such as are known in the art, e.g. clays (e.g. bentonite) and polymeric absorbents (e.g., Polymeric derivatised starches, (e.g., from National Starch), Derivatised globulin proteins, such as BioPol OE (Arch PC), MICROSPONGES 5647 and POLYTRAP, both commercially available from Advanced Polymer Systems, Inc. of Redwood City, Calif., USA., MICROSPONGES 5647 is a polymer mixture derived from styrene, methyl methacrylate, and hydrogel acrylate/methacrylate.

Compositions disclosed herein may comprise one or more of the following: water-soluble vitamins and derivatives thereof (e.g., vitamin C); polyethyleneglycols and polypropyleneglycols; polymers for aiding the film-forming properties and substantivity of the composition (such as a copolymer of eicosene and vinyl pyrrolidone, an example of which is available from GAF Chemical Corporation as Ganex™ V-220). Also useful are crosslinked and noncrosslinked nonionic and cationic polyacrylamides (e.g., Salcare SC92 which has the CTFA designation polyquaternium 32 (and) mineral oil, and Salcare SC 95 which has the CTFA designation polyquaternium 37 (and) mineral oil (and) PPG-1 trideceth-6, and the nonionic Seppi-Gel polyacrylamides available from Seppic Corp.). Also useful are crosslinked and uncrosslinked carboxylic acid polymers and copolymers such as those containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (examples useful herein include the carbomers, which are homopolymers of acrylic acid crosslinked with allyl ethers of sucrose or pentaerytritol and which are available as the Carbopol™ 900 series from B.F. Goodrich, and copolymers of C.sub.10-30 alkyl acrylates with one or more monomers of acrylic acid, methacrylic acid, or one of their short chain (i.e., C.sub.1-4 alcohol) esters, wherein the crosslinking agent is an allyl ether of sucrose or pentaerytritol, these copolymers being known as acrylates/C10-30 alkyl acrylate crosspolymers and are commercially available as Carbopol™ 1342, Pemulen TR-1, and Pemulen TR-2, from B.F. Goodrich).

In an aspect, disclosed are cosmetic or medical compositions comprising at least 0.1% w/w yeast or lipid yeast extract, or a combination of both yeast and lipid yeast extract. In an aspect, a cosmetic or medical composition may comprise at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% w/w yeast or lipid yeast extract, or a combination of both yeast and lipid yeast extract. The remainder of a cosmetic or medical composition may comprise water or other conventional cosmetic or medical ingredients, including those identified herein.

Compositions disclosed herein may be in the form of finished cosmetic or medical products for use in skin care, bathing, and/or other applications pertaining to the maintenance or improvement of an individual's appearance or health. In an aspect, compositions disclosed herein are in the form of cosmetic or medical ingredients themselves, for use in combination with other cosmetic or medical ingredients in the production of finished cosmetic or medical products.

In an aspect, compositions disclosed herein may comprise at least 0.1% w/w yeast, or a greater percentage as disclosed herein. The yeast generally comprises at least 0.1% lipid yeast extract by dry weight, and can include greater amounts of lipid yeast extract as well as other constituents as disclosed herein. The yeast useful in the cosmetic or medical compositions of the invention can be derived from one or more species of yeast cultured and/or genetically engineered as described herein.

In an aspect, cosmetic or medical compositions comprising yeast can be formulated as decorative or care cosmetics with one or more other cosmetic or medical ingredients. Exemplary cosmetic or medical compositions include, without limitation, skin-care creams, lotions, powders, perfumes and deodorants, lipsticks, bath oils, bath scrubs and cleansing products, masks, and the like.

In an aspect, cosmetic or medical compositions disclosed herein comprise at least 0.1% w/w lipid yeast extract, or a greater percentage as disclosed herein. The lipid yeast extract is derived from cultures of yeast grown under heterotrophic conditions or those comprising at least 0.1% lipid yeast extract by dry cell weight, as described herein. In an aspect, the yeast can be genetically engineered.

In an aspect, cosmetic or medical compositions comprising lipid yeast extract can be formulated as decorative or care cosmetics with one or more other cosmetic or medical ingredients. Exemplary cosmetic or medical compositions include, without limitation, skin-care creams, lotions, beauty oils, perfumes and deodorants, lipsticks, bath oils, bath scrubs and cleansing products, masks, and the like.

In an aspect, yeast cosmetic or medical compositions in accordance with the present invention can be used in otherwise conventional finished cosmetic or medical products. In these instances, the cosmetic or medical composition comprising yeast or lipid yeast extract, or a combination of both yeast and lipid yeast extract, is combined with one or more other cosmetic or medical ingredients, as described herein, to form a cosmetic or medical composition that may be packaged as a finished cosmetic or medical product. In some cases, yeast cosmetic or medical compositions of the present invention can be packaged as a cosmetic or medical ingredient with optional instructions for combining the yeast composition with conventional cosmetic or medical ingredients to create finished cosmetic or medical products.

In an aspect, the present invention is directed to a method of preparing a finished cosmetic or medical composition, e.g., a skin-care product, comprising (i) culturing a population of yeast under conditions to generate yeast comprising at least 0.10% lipid yeast extract by dry weight, (ii) harvesting the biomass from the yeast culture, (iii) performing one or more optional processing steps, e.g., drying the yeast or extracting lipids from the yeast, (iv) combining the yeast or the lipid yeast extract with at least one other cosmetic or medical ingredient to form a cosmetic or medical composition, and (v) packaging the cosmetic or medical composition with optional instructions for its use as a finished cosmetic or medical product.

In an aspect, disclosed is a method of using a compositions comprising yeast or lipid yeast extract, or a combination of both yeast and lipid yeast extract to soften and impart pliability to skin. In an aspect, the yeast composition comprises predominantly intact yeast cells containing at least 0.1% lipid yeast extract by dry weight. The yeast lipid present in the composition may be encapsulated in cells of the yeast. The yeast composition is applied to human skin and retained in contact with the skin for a period of time sufficient to permit release of a specified percentage of the lipids from the intact yeast cells by enzymatic degradation of the yeast cells. For example, the composition can be retained in contact with the skin for a period of time sufficient to release at least 50% w/w of the lipid yeast extract from the predominantly intact cells. In some cases, this period may be from 1-4 hours.

Without intending to be bound by any particular theory, it is believed that enzymes present on human skin will slowly degrade the intact yeast cells, thereby releasing the intracellular contents, including lipid yeast extract, over a period of time. In an aspect, the yeast composition is retained in contact with the skin for at least 15 minutes, for at least 30 minutes, for at least 45 minutes, for at least 1 hour, for at least 2 hours, for at least 3 hours, or for at least 4 hours or more.

Yeast compositions useful in the method disclosed herein can also comprise cells containing at least 25%, at least 35%, or at least 45% lipids by dry weight. In other cases, the cells may contain other percentages of lipids as described herein. In some cases, mixtures of yeast cells having different lipid profiles can be combined together to form a yeast composition. In the extraction, both Phosphatidylcholine (PC) and Lysophosphatidylcholine (LPC) were abundant lipids identified. PC can be used in personal care as an emulsifier, as an epidermal barrier constituent, and essential to the creation of delivery vehicles (Liposomes), the identification of LPC leads to many other applications. For example, in topical products LPC could have application in skin cancer.

Furthermore, pharmaceutical compositions of PLC can be used in antitumor treatments. LPC selectively targets plasma membrane of tumor cells to signal apoptosis. These yeast cells have defense mechanisms that can be utilized for many applications and extracting the inherent antibiotics the cells produce can lead to many applications. A compound that could be cephalosporin was identified. Certain phospholipids may have anti-viral activity or be made into anti-viral analogs.

Since yeast cell extract also comprises amino acids or polypeptides, there may be peptides and enzymes involved in signaling.

Methods may comprise anti-tumor and anti-proliferative phospholipids. There may be references that further show the value of producing a comprehensive mixture of phospholipids for select optimization and utilization pf phospholipids for this application. It was demonstrated that extracts of the yeast are capable of forming vesicles when using sonification.

Methods for immunomodulation may comprise phospholipids disclosed herein.

Phospholipid amino acid complexes may be used in nutritional foods and beverages. Riboflavin and Pyruvates are involved in the production of ATP (Kreb's cycle). Delivery of these with phospholipids comprise performance enhancer products for nutritional supplements and functional beverages.

All references cited herein, including patents, patent applications, and publications, are hereby incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein.

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used with reference to a nucleic acid, “active in yeast” refers to a nucleic acid that is functional in yeast. For example, a promoter that has been used to drive an antibiotic resistance gene to impart antibiotic resistance to a transgenic yeast is active in yeast. Examples of promoters active in yeast are promoters endogenous to certain algae species and promoters found in plant viruses.

As used in the specification and the appended claims, the singular forms “a,” “an” and “the” include plural referents unless the context clearly dictates otherwise. Thus, for example, reference to “a methylation site,” “an array,” or “the patient” includes mixtures of two or more such methylation sites, arrays, or patients, and the like.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the terms “optional” or “optionally” mean that the subsequently described event or circumstance may or may not occur and that the description includes instances where said event or circumstance occurs and instances where it does not.

“Axenic” means a culture of an organism that is free from contamination by other living organisms.

“Bioreactor” means an enclosure or partial enclosure in which cells are cultured, optionally in suspension.

The term “co-culture”, and variants thereof such as “co-cultivate”, refer to the presence of two or more types of cells in the same bioreactor. The two or more types of cells may both be microorganisms, such as yeast, or may be a yeast cell cultured with a different cell type. The culture conditions may be those that foster growth and/or propagation of the two or more cell types or those that facilitate growth and/or proliferation of one, or a subset, of the two or more cells while maintaining cellular growth for the remainder.

As used herein, “cosmetic or medical ingredient” means an ingredient conventionally used in cosmetic or medical products that is not physically or chemically incompatible with the yeast components described herein. “Cosmetic or medical ingredients” include, without limitation, absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such “cosmetic or medical ingredients” are known in the art. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993).

The term “cultivated”, and variants thereof, refer to the intentional fostering of growth (increases in cell size, cellular contents, and/or cellular activity) and/or propagation (increases in cell numbers via mitosis) of one or more cells by use of intended culture conditions. The combination of both growth and propagation may be termed proliferation. The one or more cells may be those of a microorganism, such as yeast. Examples of intended conditions include the use of a defined medium (with known characteristics such as pH, ionic strength, and carbon source), specified temperature, oxygen tension, carbon dioxide levels, and growth in a bioreactor.

As used herein, the term “cytolysis” refers to the lysis of cells in a hypotonic environment. Cytolysis is caused by excessive osmosis, or movement of water, towards the inside of a cell (hyperhydration). The cell cannot withstand the osmotic pressure of the water inside, and so it explodes.

“Dispersion” refers to a distribution of particles more or less evenly throughout a medium, including a liquid or gas. One common form of dispersion is an emulsion made up of a mixture of two or more immiscible liquids such as oil and water.

As used herein, the terms “dry weight” or “dry cell weight” refer to weight as determined in the relative absence of water. For example, reference to a component of yeast as comprising a specified percentage by dry weight means that the percentage is calculated based on the weight of the biomass after all or substantially all water has been removed.

“Exogenously provided” describes a molecule provided to the culture media of a cell culture.

“Lipid profile” refers to the distribution of different carbon chain lengths and saturation levels of glycerolipids in a particular sample of biomass or lipids. For example, a sample could contain glycerolipids in which approximately 60% w/w of the glycerolipid is C18:1, 20% is C18:0, 15% is C16:0, and 5% is C14:0. In cases in which a carbon length is referenced generically, such as “C:18”, such reference can include any amount of saturation; for example, yeast that contains 20% w/w lipid as C:18 can include C18:0, C18:1, C18:2, and the like, in equal or varying amounts, the sum of which constitute 20% w/w of the biomass.

“Homogenate” means biomass that has been physically disrupted.

“Homogenize” means to blend a substance, for example, yeast cells into a homogenous or uniform mixture. In an aspect, a homogenate is created from lysed yeast cells or the lipid yeast extract. In an aspect, the biomass of yeast cells is predominantly intact, but homogeneously distributed throughout the mixture.

As used herein, the phrase “increase lipid yield” refers to an increase in the productivity of a yeast culture by, for example, increasing dry weight of cells per liter of culture, increasing the percentage of cells that constitute lipid, or increasing the overall amount of lipid per liter of culture volume per unit time.

The term “in situ” means “in place” or “in its original position”. For example, a culture may contain a first yeast secreting a catalyst and a second microorganism secreting a substrate, wherein the first and second cell types produce the components necessary for a particular chemical reaction to occur in situ in the co-culture without requiring further separation or processing of the materials.

“Lipids” are a class of molecules that are soluble in nonpolar solvents (such as ether and hexane) and are relatively or completely insoluble in water. Lipid molecules have these properties because they consist largely of long hydrocarbon tails which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, tocopherols, tocotrienols, sterol lipids including cholesterol and steroid hormones, prenol lipids including terpenoids, fatty alcohols, waxes, and polyketides); and complex lipid derivatives (sugar-linked lipids, or glycolipids, and protein-linked lipids). Lipid and oil may be used interchangeably herein and are generally referring to those compounds characterized as fats.

As used herein, the term “lysate” refers to a solution containing the contents of lysed cells.

As used herein, the term “lysis” refers to the breakage of the plasma membrane and optionally the cell wall of a biological organism sufficient to release at least some intracellular content, often by mechanical, viral or osmotic mechanisms that compromise its integrity.

As used herein, the term “lysing” refers to disrupting the cellular membrane and optionally the cell wall of a biological organism or cell sufficient to release at least some intracellular content.

As used herein, the term “osmotic shock” refers to the rupture of cells in a solution following a sudden reduction in osmotic pressure. Osmotic shock is sometimes induced to release cellular components of such cells into a solution.

As used herein, a “polysaccharide-degrading enzyme” refers to any enzyme capable of catalyzing the hydrolysis, or depolymerization, of any polysaccharide. For example, cellulases catalyze the hydrolysis of cellulose.

“Polysaccharides” (also called “glycans”) are carbohydrates made up of monosaccharides joined together by glycosidic linkages. Cellulose is an example of a polysaccharide that makes up certain plant cell walls. Cellulose can be depolymerized by enzymes to yield monosaccharides such as xylose and glucose, as well as larger disaccharides and oligosaccharides.

As used herein, “predominantly intact cells” refers to a population of cells which comprise more than 50%, 75%, or 90% w/w intact cells. “Intact” refers to the physical continuity of the cellular membrane enclosing the intracellular components of the cell and means that the cellular membrane has not been disrupted in any manner that would release the intracellular components of the cell to an extent that exceeds the permeability of the cellular membrane under conventional culture conditions or those culture conditions described herein.

As used herein, the term “sonication” refers to a process of disrupting biological materials, such as a cell, by use of sound wave energy.

Reference to proportions by volume, i.e., “v/v,” means the ratio of the volume of one substance or composition to the volume of a second substance or composition. For example, reference to a composition that comprises 5% v/v lipid yeast extract and at least one other cosmetic or medical ingredient means that 5% of the composition's volume is composed of lipid yeast extract; e.g., a composition having a volume of 100 mm³ would contain 5 mm³ of lipid yeast extract and 95 mm³ of other constituents.

Reference to proportions by weight, i.e., “w/w,” means the ratio of the weight of one substance or composition to the weight of a second substance or composition. For example, reference to a cosmetic or medical composition that comprises 5% w/w yeast and at least one other cosmetic or medical ingredient means that 5% of the cosmetic or medical composition is composed of yeast; e.g., a 100 mg cosmetic or medical composition would contain 5 mg of yeast and 95 mg of other constituents. One of skill in the art can determine whether percentages of components of compositions are w/w or v/v.

### EXAMPLES

### Example 1

### Introduction

Debaryomyces (Torulaspora) hansenii is a type of yeast that can tolerate and survive changes in sugar, salt and dryness. It is non-pathogenic and found in water with salt concentration of up to 24% w/w (Breuer and Harms, 2006). It is also found in the cheese and sausages industries (Fleet, 1990; Dalton et al., 1984). D. hansenii is able to eliminate competition by other yeasts due to its ability to tolerate salt and reproduce at low temperatures.

Molecular genetic studies for D. hansenii are still in their infancy. There were 46 gene entries corresponding to 28 different proteins in Genbank before release of the whole genome data. The whole genome is available at the ncbi/nlm.nih website. D. hansenii defines now one of the four clades which constitute this genus. The species contains two varieties, var. hansenii and var. fabryi, the second of them is not very often found and is poorly characterized (Kurtzman and Robnett, 1998).

The yeast D. hansennii uses glucose as a substrate at a very slow rate with typical times for culture reported as 21-28 days. The most common lipids produced are triglycerides, free fatty acids, phosphatidylserine and phosphatidylethanolamine (Merdinger and Frye, 1966). Commercial Applications

D. hansennii osmotolerance is highly advantageous for some biotechnological applications because it allows quasi-non-sterile production and high product/educt concentrations, conditions which can reduce production costs dramatically. The extreme capacity of D. hansenii to synthesize, accumulate and store lipids is advantageous for the biotechnological production of natural and artificial products. The ability to produce phospholipids can be modified by changing the amount of salt in the culture media allowing for more selective production of products. liposomes of small size can be produced by sonication of lipid fractions.

Odorless culture and production. Members of the genus Debaryomyces are characterized physiologically by their weak or nonexistent fermentation capacities. D. hansenii is able to use alkanes as a food source. This application is useful as a lot of by-products from mining and cracking are alkanes. This particular strain is able to assimilate a large number of sugar substrates such as sucrose, galactose, lactose, mannose, maltose and treehalose among others D. hansenii appears to have a very high coding capacity reflected in 79.2% of its genome, with 6,906 detected coding sequences or (CDs). This characteristic allows this yeast to be used in biotechnological applications. The most abundant solute produced by the yeast is glycerol and it has the capacity to regulate its glycerol metabolism under hyperosmolaric conditions. It can also produce xylitol.

Expressing the genes conferring salt resistance in D. hansenii in plants is an effective strategy to grow crops in arid regions and can make a substantial contribution to reducing hunger in the world. The yeast can also produce D-Arabinitol after the growth phase in batch culture, simultaneously with the excretion of riboflavin. Pyruvic acid can be widely used in the chemical, pharmaceutical and agrochemical industries and the biotechnological production of this acid is a viable alternative to the current chemical method, because it is a relatively cheap, one-step procedure.

D. hansenii also produces important enzymes with commercial applications such as 0-glucosidases and superoxide dismutase. This yeast can also produce alkali-soluble glucans that can be used as thickening agents, fat substitutes or sources of dietary fiber. Furthermore, they have antitumor activity, stimulate the immune system and can lower the serum cholesterol levels.

### Materials and Methods

### Lipid Extractions

D. hansenii (NRRL-Y-1448) (ATCC 10619) was reconstituted by breaking the outer glass vial and carefully removing the cotton plug of the inner vial. The yeast was rehydrated with 400 μl of sterile water and transferred to a sterile 15 ml conical tube (Corning) where it was left overnight at room temperature.

Aliquots of yeast were prepared for inoculation of media.

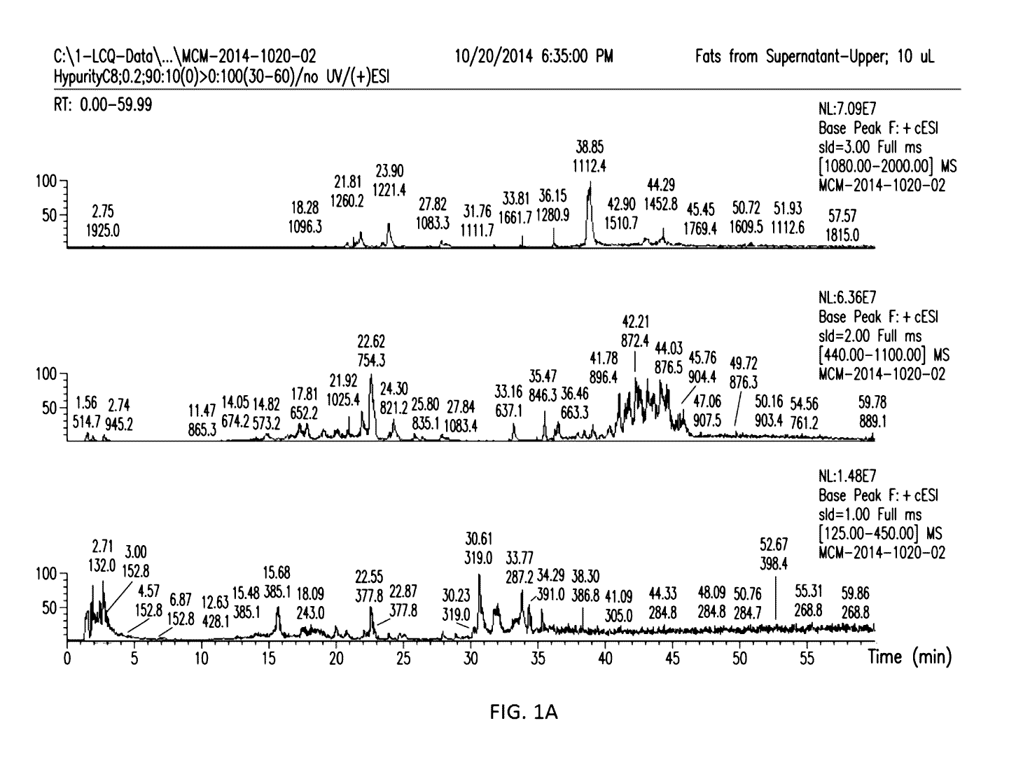

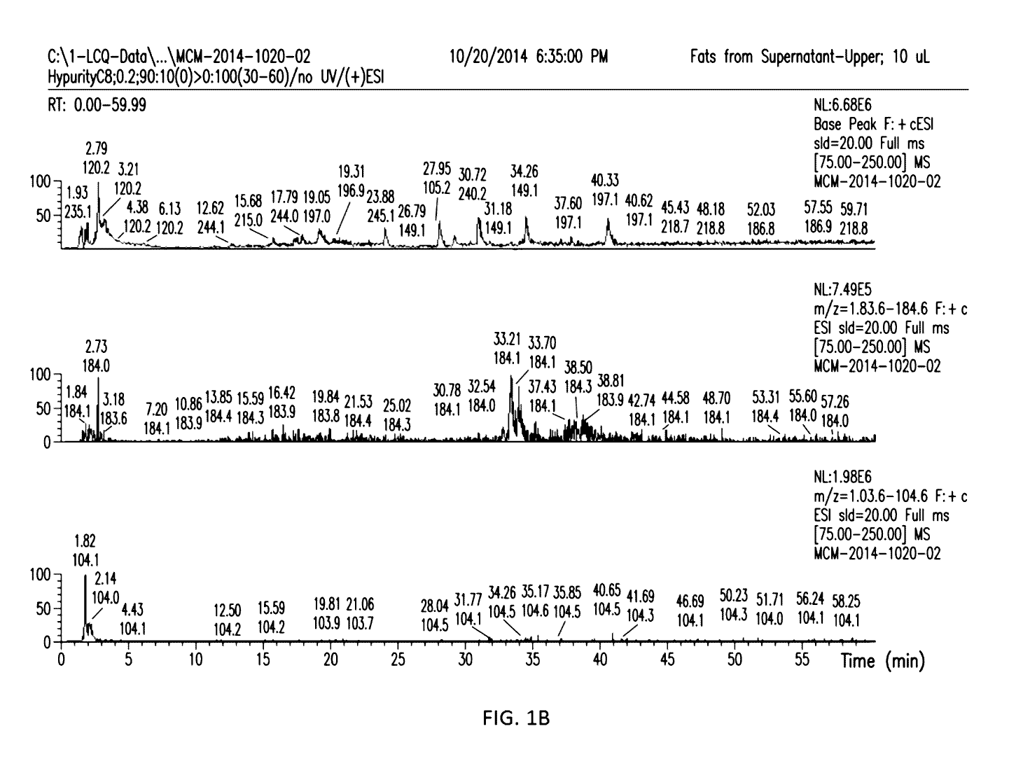

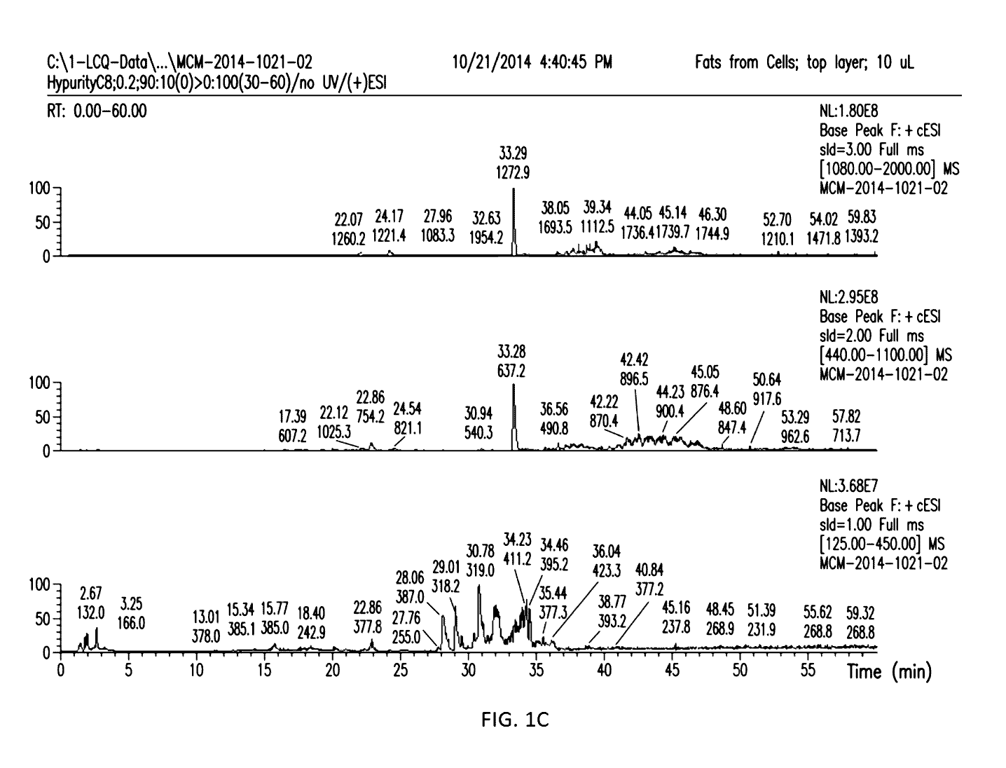

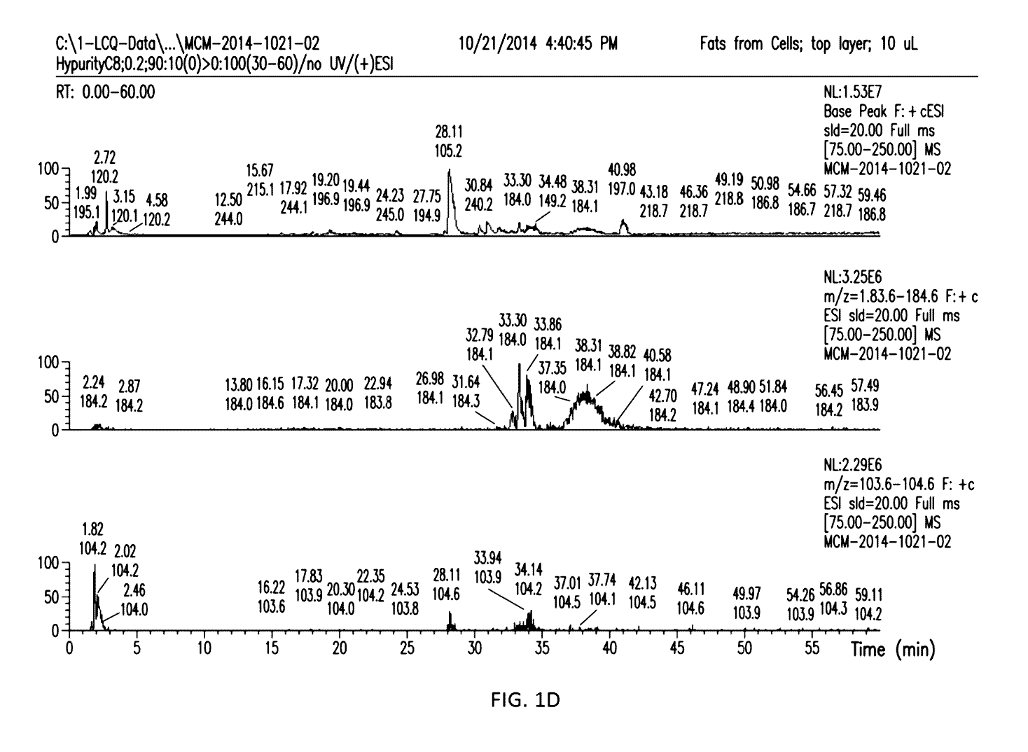

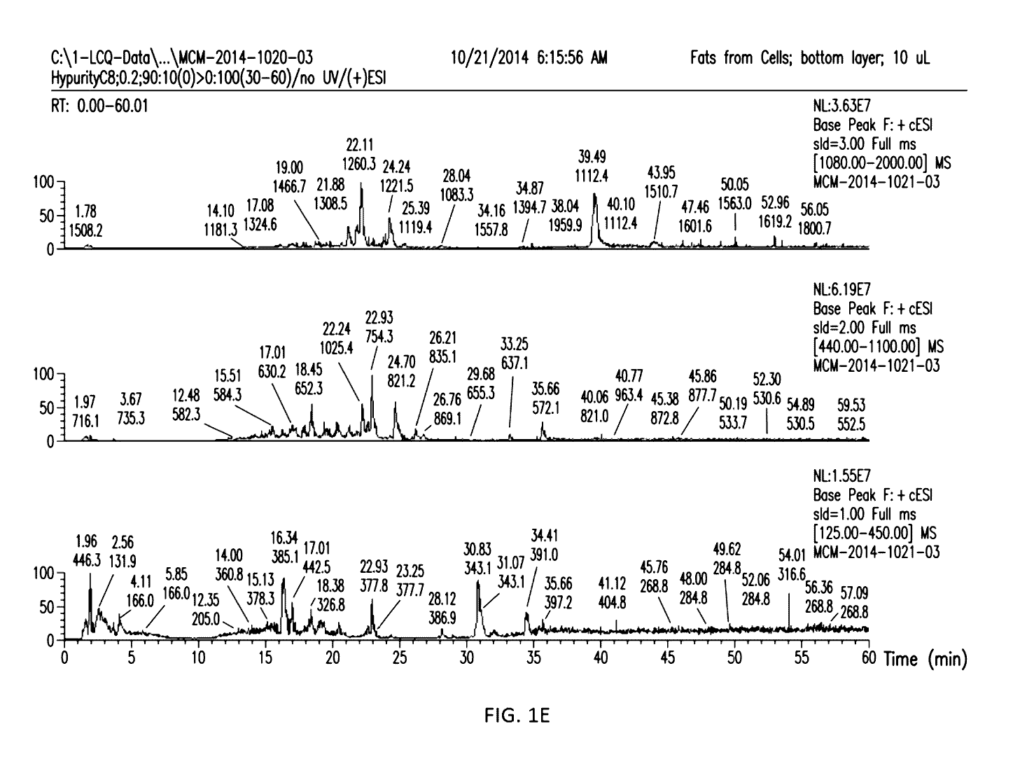

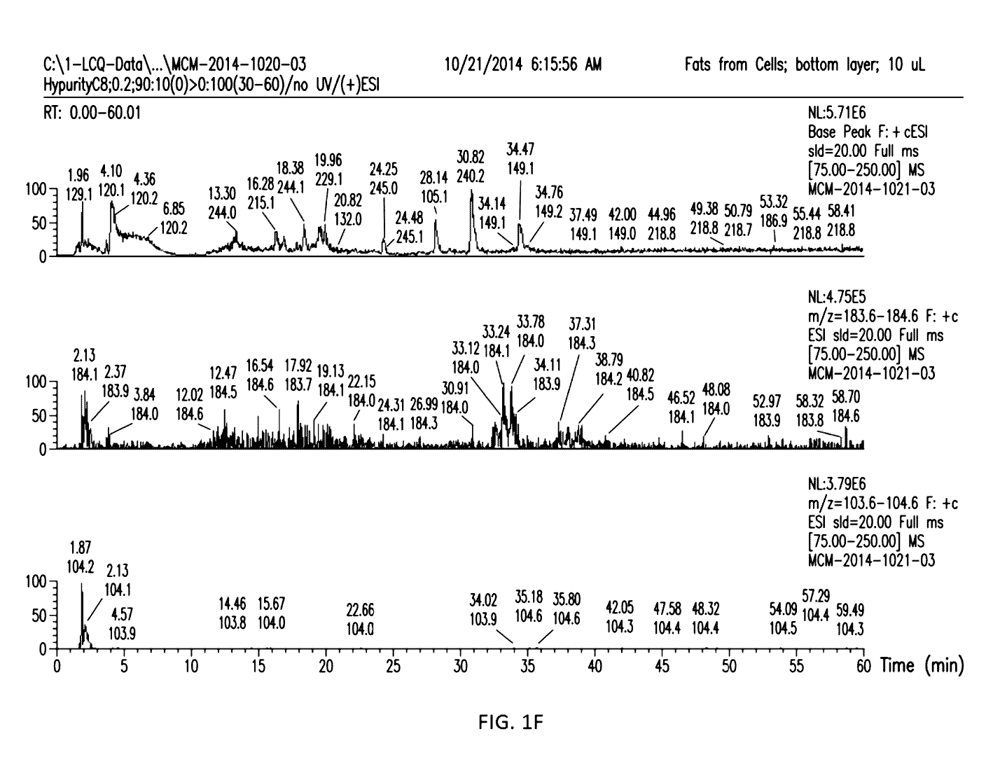

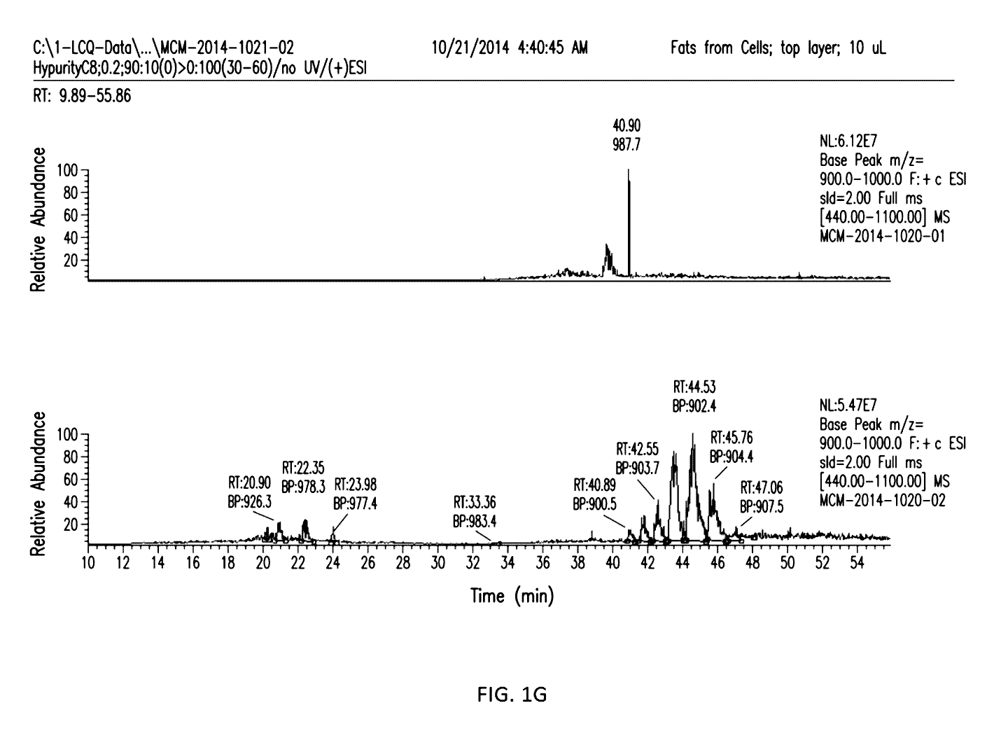

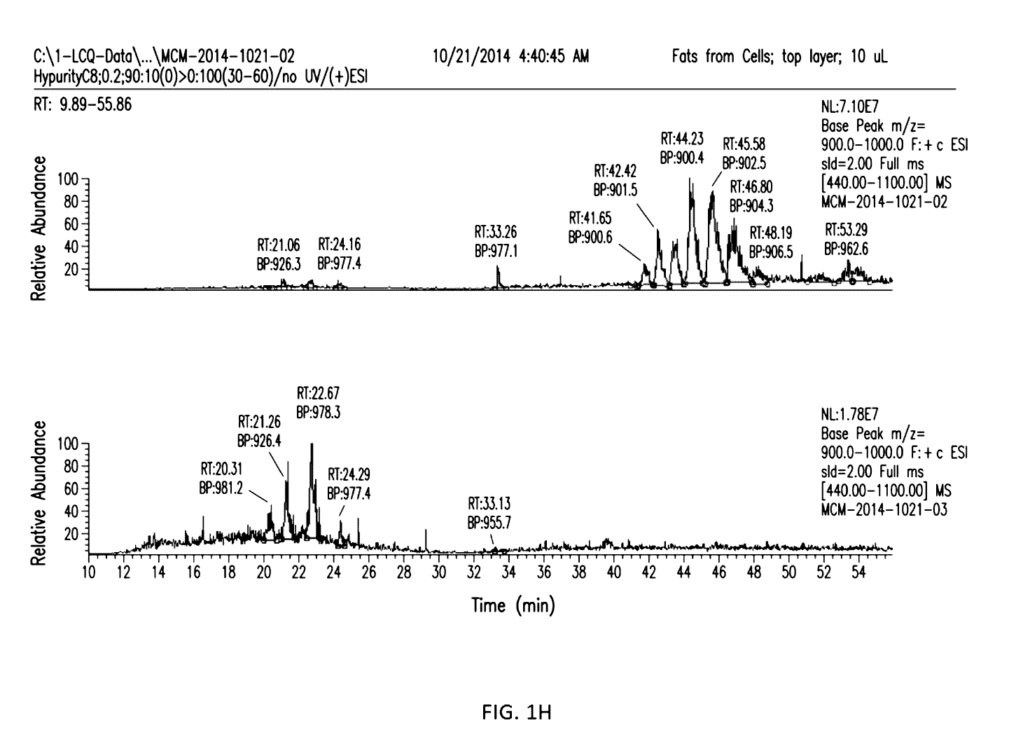

100 μl aliquots of rehydrated yeast were grown in 9 ml tubes containing Sabourad media (VWR) and at different temperatures (37° C., 24° C. and 28° C.) in a rotary shaker. Volume was added with a sterile pipette and an automated pipettor. Stocks of yeast were grown in 10 cm diameter Sabaourad agar plates (VWR) at room temperature. Optimum density was observed after 20 days. Yeast began lipid production after 24 hours as evidenced by a ring of waxy/fatty material around the top of the culture. The cultures were combined and centrifuged to isolate the yeast and the yield of the culture was calculated. 3.66 g of D. hansenii were harvested and 1.98 g were originally inoculated. % yield in culture: 3.56/1.98*100=179.80% w/w 100 ml supernatant were collected from the cells and 50 ml HPLC grade methanol were added to the supernatant and stirred overnight to dissolve the phospholipids. 100 ml chloroform were added to this supernatant and stirred for 1 hour. The phases were allowed to separate overnight in a 250 ml graduated cylinder (FIG. 1). The precipitated cells were saved at 4° C. overnight according to Turk, 2004. The fatty material was separated from the clear liquid and the supernatant (FIG. 2). Out of 100 ml of supernatant, 10 ml of fatty material were recovered. Out of 10 ml of dissolved sonicated cells, about 5 ml of fatty material were recovered with about 2.5 ml of white fat and cell debris (top layer) and about 5.0 ml of fatty material and the remaining solvent as seen in FIG. 2.

### Thin Layer Chromatography (TLC)

TLC plates (silica, Whatman LK5 equivalent with glass backing) were pre-washed to remove any UV fluorescent material by migration up to 1 cm from the top in a clean tank containing chloroform/methanol (1/1, v/v). The pre-wash step lasted 1.5 hours. The solvent level was marked on the plate with a pencil. Plates were air-dried in a fume hood for five minutes and placed in a drying rack until used. Immediately before use, plates were completely wetted using a plastic bottle to spray (VWR) with boric acid solution prepared by dissolving 2.3 g of boric acid in 100 ml ethanol. The plates were drained for 5 minutes in a fume hood and dried in a model 10 oven for 15 min at 100 C.

Lipid samples 100 μl chloroform/methanol solution (2:1 v/v) containing 20-200 μg phospholipids were rapidly deposited on plates at 1 cm parallel in the concentration zone. From left to right: T (lipids found in the top layer of cell extract), B (lipids found in the bottom layer of cell extract) and S (lipids contained in the supernatant) (FIG. 3A). The solvent was allowed to dry (a precaution used to avoid distorted spots) and the plates were rapidly placed in the chromatography tank containing chloroform/ethanol/water/triethylamine (30/35/7/35, v/v) (FIG. 3B). The migration time was 2 hours. The solvent was allowed to reach the 1 cm mark at the top.

Plates were dried in a fume hood (2-5 min max) and sprayed with primuline solution (yellow) made by dissolving 5 mg of primuline in 80/20 acetone/water. After viewing under UV light, photographs were taken and the contour of each spot was outlined. The fluorescent spots, indicating lipids, were scraped from the silica into glass tubes for further analysis by GC-MS.

As described by Vaden et al. in 2005, the neutral lipids migrated with the solvent (seen as a bright line on the top of FIG. 4). Polar lipids (phospholipids) remained at the bottom of the TLC plate. Triacylglycerols (nonpolar) migrated faster than phospholipids. A schematic of migrations is presented in FIG. 4. Actual photographs (under UV light) of the plate, revealed primuline staining (fluorescent). Although the spots are difficult to visualize due to the background fluorescence of the TLC plates, the contour of each phospholipid was outlined.

Rf values were calculated and shown in Table 1. The Rf values were in good agreement with the values reported by Leray and Pelletier, 1987.

| | | Distance | Calculated | Reported |
|---|---|---|---|---|
| | Distance | migrated | Rf | Rf |
| Spot | migrated | by solvent | value | value¹ |
| PE | 3.5 | 7.6 | 0.46 | 0.51 |
| PS | 3 | 7.6 | 0.39 | 0.38 |
| PI | 2.3 | 7.6 | 0.30 | 0.26 |
| PC | 1.5 | 7.6 | 0.20 | 0.21 |

¹Leray and Pelletier, 1987.

### Results

The inoculated yeast doubled its mass in 20 days at 28° C. and a pH of around 5.6. The yeast can also be grown at room temperature but growth is slower. Attempts to grow yeast at temperatures higher than 28° C. failed even though it has been reported in the literature that the yeast can be grown at 30 and 31° C. (Merdinger and Frye, 1966). Yield of fatty material was high compared to the actual volume of the initial cultures. Increasing the pH has been reported as useful in doubling time for these cells. The doubling time of the cells was 9.2 h at pH 4.0, 2 h at pH 6.0 and 6 h at pH 8.0 (Turk et al., 2007).

Yield of fatty material from supernatant=(10 ml/100 ml)*100=10% w/w

Yield of fatty material from the sonicated cells=(5 ml/10 ml)*100=50% w/w

TLC analysis for extracellular lipids of the cell-free supernatant was negative, consistent with the results obtained in 1966 by Merdinger and Frye. TLC analysis of the bottom layer of the sonicated cell extract was negative. TLC analysis of the top layer of the sonicated cell extract fatty material was positive. Only four distinct spots were able to be identified: The largest spot (and therefore the largest amount) corresponds to PC and LPC followed by PS and PE in a lower proportion. There was also a small spot at the base of the PS spot, which according to Vaden et al., 2005, corresponds to PI.

Sonication has been reported to induce the formation of small liposomes (Szoka F and Papahadjopoulos, 1980). These vesicles are used as drug and gene delivery vehicles.

As this protocol was adapted to separate the phospholipids of interest, neutral lipids and triacylglycerols were observed to migrate as expected with the solvent as a bright line at the top of the plate. As described by Leray and Pelletier in 1987, poor separation of PS, PE, PI and PC was observed using the normal TLC protocol. The use of boric acid improved the resolution of the spots but it can be necessary to run a two dimensional TLC in order to get better separation of the spots.

Other fluorescent compounds can interfere with the quality of images obtained under UV. Proteins and peptides, with aromatic amino acids are intrinsically fluorescent when excited with UV light. Many enzymatic cofactors, such as FMN, FAD, NAD and porphyrins, are also intrinsically fluorescent under UV light. In order to obtain better graphics, sulfuric acid or iodine can be used to visualize the spots as these methods do not require UV light to reveal spots.

The results obtained demonstrate that it is possible to culture D. hansenii obtained commercially and scale up its production in Sabaourad media with minimum requirements for the culture. For the variety used, the optimum conditions appear to be 28° C., salt concentration of 2% w/w and pH 5.6 to 6.2. Salt concentration can be varied to increase the production of the phospholipids of interest.

The cultures did not present any fermentation or sulfur odor. However, a mild odor was detected after 3 months.

As D. Hansennii is a halophile that grows at 2% w/w salt concentration, there was no contamination in the cultures. In addition, D. hansenii produces toxins that out-compete other yeasts. The use of D. hansenii in the cheese and meat industries indicates that it is safe to use in commercial applications.

The cultures are a milky tan color. There is no need to remove pigments. There was no gas detected as being produced. Moreover, there was no foam produced in the cultures.

Lipids were easily extracted using an aqueous/organic extraction procedure. It was possible to separate lipids using one dimensional TLC. However, 2D-TLC is recommended to obtain more accurate results and for quantitation. These results indicate that 50% w/w of the pelleted (wet) cells have the potential to yield phospholipids. Merdinger and Devine (1965) reported that neutral lipids comprised 67%, and phospholipids comprised 33%, w/w, of the total lipids isolated from D. hansenii.

### Example 2

### Burn Cream

The compositions of Phase A, B, and C comprised the following:

### Phase A

### Deionized Water q.s. to 100%

### Disodium EDTA—0.05%

### Aloe Barbadensis Juice—0.50%

### Phase B

### Rosehip Seed Oil—3.60%

### White Petrolatum, USP—2.80%

### Phosphotidylcholine/Phosphatidylserine Yeast Extract—4.50%

### Cetyl Alcohol NF 2.70%

### Sorbitan Monostearate—1.20%

### Glyceryl Behenate, NF—1.80%

### Emulsifying Wax, NF—1.00%

### BHT—0.05%

### Phase C

### Benzocaine Micronized—2.00%

### Diazolidinyl Urea 0.20%

### Processing Procedure:

Phase ‘A’ ingredients were added one by one with lightning mixer and heated to 75-80° C. with continued high speed mixing. In a separate vessel all Phase B ingredient were added with moderate agitation while heating to 75-80° C. The mixture was emulsified by slowly adding Phase B to Phase A with continuous vigorous mixing and the temperature was maintained for 10 minutes. The mixture was cooled to 40° C. and Phase C ingredients were added. The mixture was continuously mixed slowly. Then the mixture was cooled to 25° C. with slow mixing, completing the process.

### Example 3

### Keratolytic Cream for Seborrheic Dermatitis

The compositions of Phase A, B, C, and D comprised the following:

### Phase A

### Deionized Water—q.s. to 100%

### Disodium EDTA—0.05%

### Xanthan Gum—0.40%

### Phase B

### PEG Stearate and Glycol Stearate—2.50%

### Mineral Oil, NF—3.00%

### Emulsifying Wax, NF—1.20%

### Isopropyl Myristate, NF—4.30%

### Cetyl Alcohol, NF—1.10%

### Stearyl Alcohol, NF—0.90%

### Phosphotidylcholine Yeast Extract—3.50%

### Vitamin E, USP—0.3%

### BHT—0.10%

### Phase C

### Benzoic Acid USP, EP—0.05%

### Phase D

### Deionized Water—15.00%

### Propylene Glycol, USP—5.00%

### Poly-Pore 150 SA 50% (Salicylic Acid 50%)—4.00%

### Processing Procedure:

Phase ‘A’ ingredients were added one by one with lightning mixer to disperse Xanthan Gum. The mixture was heating to 75-80° C. with continued high speed mixing. In a separate vessel all Phase ‘B’ ingredients were added with moderate agitation while heating to 75-80° C. The mixture was emulsified by slowly adding Phase B to Phase A with continuous vigorous mixing and the temperature was maintained for 10 minutes. The batch was then cooled to 40° C. and Phase C was added. Slow mixing was continued. The mixture continued to cool to 35° C. and pre-dispersed Phase D ingredients were added. The mixture was continuously mixed slowly. The mixture was cooled to 25° C. with slow mixing, completing the process.

### Example 4

### Anti-Psoriatic Cream

The compositions of Phase A, B, C, and D comprised the following:

### Phase A

### Deionized Water q.s. to 100%

### Disodium EDTA—0.05%

### Xanthan Gum—0.40%

### Phase B

### Mineral Oil, NF—4.60%

### White Petrolatum, USP—4.80%

### Phosphotidylcholine/Phosphatidylinsoitol Yeast Extract Liposomes—4.50%

### Cetyl Alcohol, NF 1.00%

### Stearyl Alcohol, NF 1.80%

### Glyceryl Monostearate—1.80%

### Emulsifying Wax, NF—2.50%

### Polysorbate 80-1.60%

### BHT—0.05%

### Phase C

### Hydrocortisone Acetate, USP—2.00%

### Phase D

### Propylene Glycol—5.00%

### Imidazolidinyl Urea—0.20%

### Processing Procedure:

Phase ‘A’ ingredients were added one by one with lightning mixer with vigorous agitation to disperse Xanthan Gum., followed by heating to 75-80° C. with continued high speed mixing. In a separate vessel all Phase ‘B’ ingredients were added with moderate agitation while heating to 75-80° C. The mixture was emulsified by slowly adding Phase B to Phase A with continuous vigorous mixing and the temperature was maintained for 10 minutes. The batch was cooled to 40° C. and Phase C was added while continuously mixing slowly. The mixture was cooled to 35° C. and Phase D ingredients were added while continuously mixing slowly. The mixture was then cooled to room temperature 25° C. with slow mixing, completing the process.

### Example 5

### Antibiotic Anti-Acne Gel

The compositions of Phase A, B, and C comprised the following:

### Phase A

### Deionized Water—q.s. to 100%

### Disodium EDTA—0.05%

### Hydroxypropyl Cellulose—0.35%

### Phase B

### Ethoxydiglycol—3.60%

### Glycerin NF—5.00%

### Dimethyl Isosorbide—2.50%

### Phosphatidylethanolamine Yeast Extract—1.00%

### Phase C

### Clindamycin Phosphate NF—2.00%

### Processing Procedure:

Phase A ingredients were added one by one with lightning mixer with vigorous agitation to disperse HPC. The mixture was continuously mixed for 30 minutes. In a separate vessel, all Phase B ingredients were added to Phase A with moderate agitation and continued moderate mixing for 15 minutes. Phase C was added to the main batch. The mixture was continuously mixed slowly until it was uniform, completing the process.

### Example 6

### Nutritional Supplement Phospholipid—Asthaxanthin Complex

The composition comprised the following:

### Main Batch

### Extra Virgin Olive Oil—35%

### Phosphatidylcholine Yeast Extract—25%

### Phosphatidylserine Yeast Extract—20%

### Phosphatidylinositol Yeast Extract—5%

### Astaxanthin—15%

Fill of 500 mg in Vegetarian Softgel Capsule (glycerin, modified corn starch, carrageenan, sorbitol, water)

### Example 7

### Functional Beverages—Phospholipid Memory Enhancing Water

The composition comprised the following:

### Spring Mineral Enriched Water—99.2%

### Phosphatidylcholine Yeast Extract in β Cyclodextrin 0.80%

### Example 8

### Feline Phospholipid/Omega Fish Oil Supplement

The composition comprised the following:

Fish oil (mackerel)—59,80%

### Phosphatidylcholine Yeast Extract—16.5%

### Phosphatidylserine Yeast Extract—11.2%

### Phosphatidylinositol Yeast Extract—10.5%

### Tocopherol Acetate—0.50%

### Vitamin A Palmitate—1.00%

### Flavor—0.50%

### Daily Dosage: 1 ml

### Example 9

### Liposomal Saw Palmetto Hair Growth Liquid Rub

The composition comprised the following:

### Deionized Water—q.s. to 100%

### Polysorbate 80—2.30%

### Dimethyl Isosorbide—13.40%

### Ethoxydiglycol—8.00%

### Saw Palmetto Extract in Yeast Phospholipid Extract Liposome—4.50%

### Preservatives—0.50%

### Example 10

### Agricultural Grape Botrytis Bio-Control

The composition comprised the following:

### Deionized Water—q.s. to 100%

### Magnesium Aluminum Silicate—1.00%

Whole Cell Debaryomyces hansenni—8.00%

### Example 11

An antimicrobial composition may comprise:

### ETOH 62%

### Water

Ergosteroid/Pyrole Complex, isolated from D. hansenii

### Carbopol 940

### Sodium Hydroxide

Such as composition comprising one or more of ergosteroid, 7-nor-ergosterolide or 3β-hydroxyergosta-8,24(28)-dien-7-one (Ergosteroid/pyrole complex) may have cytotoxicity against eucaryotic cells, and antimicrobial activity against bacteria or yeasts such as Enterobacter aerogenes, Pseudomonas aeruginosa, and Candida albicans.

### Example 12

### Identification of Compounds

Compounds were identified by chromatography based on their m/z ratio (the number found on top of each peak—See FIGS. 1A-1H). m/z is based on the input m/z and not Exact m/z. There may be some overlap. Identification was performed for the supernatant and two fractions of yeast cells after sonication and extraction. FIGS. 1A and 1B show fats from the supernatant (lipids found in the culture media). m/z peak 1112.4 (FIG. 1A, line 1) corresponds to cyclosporin, a pentasacharide, Lyso phosphatidylcholine 20:0 or fragments derived from a peptide with the following sequence: EQGDQPAGAESGGEESAPATFQVHDGLFMTDR. (SEQ ID NO. 1) m/z peak 754.3 (FIG. 1A, line 2) corresponds to fragments derived from a peptide with the following sequence: MAMLTFLHEPAVLYNLKDR (SEQ ID NO. 2) or to the following possible lipids:

TABLE 2
Possible Lipid Structures

| | | | Input | Exact | | |
|---|---|---|---|---|---|---|
| LM_ID | Name | Systematic Name | m/z | m/z | Formula | Ion |
| LMGP03010131 | PS(14:1(9Z)/20:4(5Z,8Z,11Z,14Z)) | 1-(9Z-tetradecenoyl)-2-(5Z,8Z,11Z,14Z- | 754.3 | 754.4654 | C₄₀H₃₉NO₁₀P | M + H |
| | | eicosatetraenoyl)-glycero-3-phosphoserine | | | | |
| LMGP03010432 | PS(18:4(6Z,9Z,12Z,15Z)/16:1(9Z)) | 1-(6Z,9Z,12Z,15Z-octadecatetraenoyl)2-(9Z- | 754.3 | 754.4654 | C₄₀H₃₉NO₁₀P | M + H |
| | | hexadecenoyl)-glycero-3-phosphoserine | | | | |
| LMGP03010623 | PS(20:4(5Z,8Z,11Z,14Z)/14:1(9Z)) | 1-(5Z,8Z,11Z,14Z-eicosatetraenoyl)-2-(9Z- | 754.3 | 754.4654 | C₄₀H₃₉NO₁₀P | M + H |
| | | tetradecenoyl)-glycero-3-phosphoserine | | | | |
| LMGP03010650 | PS(20:5(5Z,8Z,11Z,14Z,17Z)/14:0) | 1-(5Z,8Z,11Z,14Z,17Z-eicosapentaenoyl)-2- | 754.3 | 754.4654 | C₄₀H₃₉NO₁₀P | M + H |
| | | tetradecanoyl-glycero-3-phosphoserine | | | | |
| LMGP03010899 | PS(16:1(9Z)/18:4(6Z,9Z,12Z,15Z)) | 1-(9Z-hexadecenoyl)-2-(6Z,9Z,12Z,15Z- | 754.3 | 754.4654 | C₄₀H₃₉NO₁₀P | M + H |
| | | octadecatetraenoyl)-glycero-3-phosphoserine | | | | |
| LMGP03010922 | PS(14:0/20:5(5Z,8Z,11Z,14Z,17Z)) | 1-tetradecenoyl-2-(5Z,8Z,11Z,14Z,17Z- | 754.3 | 754.4654 | C₄₀H₃₉NO₁₀P | M + H |
| | | eicosapentaenoyl)-glycero-3-phosphoserine | | | | |

Note:
Chains containing double bonds and/or functional groups with defined regiochemistry, geometry and stereochemistry are meant to serve as examples for structure-drawing purposes. In many cases there may be alternative isobaric structures.

TABLE 3
m/z peak 876.5 (2^{nd} row, FIG. 1A):
Possible Lipid Structures

| | | | Input | Exact | | |
|---|---|---|---|---|---|---|
| LM_ID | Name | Systematic Name | m/z | m/z | Formula | Ion |
| LMGP01010004 | PC(21:0/ | 1-heneicosanoyl-2-(4Z,7Z,10Z,13Z,16Z,19Z- | 876.5 | 876.6477 | C₅₁H₉₁NO₈P | M + H |
| | 22:6(4Z,7Z,10Z,13Z,16Z,19Z)) | docosahexaenoyl)-sn-glycero-3-phosphocholine | | | | |
| LMGP01012121 | PC(22:6(4Z,7Z,10Z,13Z,16Z,19Z)/ | 1-(4Z,7Z,10Z,13Z,16Z,19Z-docosahexaenoyl)-2- | 876.5 | 876.6477 | C₅₁H₉₁NO₈P | M + H |
| | 21:0) | heneicosanoyl-glycero-3-phosphocholine | | | | |
| LMGP03010716 | PS(22:0/21:0) | 1-docosanoyl-2-elcosanoyl-glycero-3- | 876.5 | 876.6688 | C₄₈H₉₅NO₁₀P | M + H |
| | | phosphoserine | | | | |
| LMGP03010851 | PS(21:0/21:0) | 1,2-diheneicosanoyl-sn-glycero-3-phosphoserine | 876.5 | 876.6688 | C₄₈H₉₅NO₁₀P | M + H |
| LMGP03010946 | PS(20:0/22:0) | 1-eicosanoyl-2-docosanoyl-glycero-3- | 876.5 | 876.6688 | C₄₈H₉₅NO₁₀P | M + H |
| | | phosphoserine | | | | |

Note:
Chains containing double bonds and/or functional groups with defined regiochemistry, geometry and stereochemistry are meant to serve as examples for structure-drawing purposes. In many cases there may be alternative isobaric structures.

TABLE 4
m/z 398.4 (3^{rd} row, FIG. 1A):
Possible Lipid Structures

| | | | Input | Exact | | |
|---|---|---|---|---|---|---|
| LM_ID | Name | Systematic Name | m/z | m/z | Formula | Ion |
| LMFA03010075 | PGF2alpha-EA | N-(9S,11R,15S-trihydroxy-5Z,13E-prostadienoyl)- | 398.4 | 398.2901 | C₂₂H₄₀NO₅ | M + H |
| | | ethanolamine | | | | |
| LMFA03010208 | 11beta-PGF2alpha-EA | N-(9S,11S,15S-trihydroxy-5Z,13E-prostadienoyl)- | 398.4 | 398.2901 | C₂₂H₄₀NO₅ | M + H |
| | | ethanolamine | | | | |
| LMFA03010209 | PGE1-EA | N-(9-oxo-11R,15S-dihydroxy-13E-prostenoyl)-ethanolamine | 398.4 | 398.2901 | C₂₂H₄₀NO₅ | M + H |
| LMFA03110015 | 8-iso-PGF2alpha III-EA | N-([8S,12R]9S,11R,15S-trihydroxy-5Z,13E-prostadienoyl)- | 398.4 | 398.2901 | C₂₂H₄₀NO₅ | M + H |
| | | ethanolamine | | | | |
| LMFA08040053 | Tricosanoyl-EA | N-(Tricosanoyl)-ethanolamine | 398.4 | 398.3992 | C₂₅H₅₂NO₂ | M + H |
| LMGP01050068 | PC(9:0/0:0) | 1-nonanoyl-sn-glycero-3-phosphocholine | 398.4 | 398.2302 | C₁₇H₃₇NO₇P | M + H |
| LMGP02050005 | PE(12:0/0:0) | 1-dodecanoyl-sn-glycero-3-phosphoethanolamine | 398.4 | 398.2302 | C₁₇H₃₇NO₇P | M + H |
| LMSP01050003 | Phytosphingosine 1- | (2S,3S,4R)-2-amino-3,4-dihydroxyoctadecyl dihydrogen | 398.4 | 398.2666 | C₁₈H₄₁NO₆P | M + H |
| | phosphate | phosphate | | | | |
| LMST01150006 | Verazine | (20S,25S)-22,26-iminocholesta-5,22(N)-dien-3beta-ol | 398.4 | 398.3417 | C₂₇H₄₄NO | M + H |
| LMST01150007 | Solanidine | solanid-5-en-3beta-ol | 398.4 | 398.3417 | C₂₇H₄₄NO | M + H |

Note:
Chains containing double bonds and/or functional groups with defined regiochemistry, geometry and stereochemistry are meant to serve as examples for structure-drawing purposes. In many cases there may be alternative isobaric structures.

m/z 184 (FIG. 1B, line 2) is a precursor ion for several phospholipids and corresponds to phosphocholine

TABLE 5
m/z 104.1 (FIG. 1B, line 3):
Possible Lipid Structures

| LM_ID | Name | Systematic Name | Input m/z | Exact m/z | Formula | Ion |
|---|---|---|---|---|---|---|
| LMFA01100034 | | 2S-amino-butanoic acid | 104.1 | 104.0706 | C₄H₁₀NO₂ | M + H |
| LMFA01100039 | | 4-amino-butanoic acid | 104.1 | 104.0706 | C₄H₁₀NO₂ | M + H |
| LMFA01100043 | | 2R-amino-butanoic acid | 104.1 | 104.0706 | C₄H₁₀NO₂ | M + H |
| LMFA01100049 | (R)-b-amino-isobutyric acid | 2R-methyl-3-amino-propanoic acid | 104.1 | 104.0706 | C₄H₁₀NO₂ | M + H |
| LMFA01100050 | (S)-b-amino-isobutyric acid | 2S-methyl-3-amino-propanoic acid | 104.1 | 104.0706 | C₄H₁₀NO₂ | M + H |
| LMFA01100053 | 2-amino-isobutyric acid | 2-amino-2-methyl-propanoic acid | 104.1 | 104.0706 | C₄H₁₀NO₂ | M + H |
| LMFA01100054 | 3-amino-isobutanoic acid | 3-amino-3-methyl-propionic acid | 104.1 | 104.0706 | C₄H₁₀NO₂ | M + H |

Note:
Chains containing double bonds and/or functional groups with defined regiochemistry, geometry and stereochemistry are meant to serve as examples for structure-drawing purposes. In many cases there may be alternative isobaric structures.

### Second Sample

### FATS FROM CELLS—TOP LAYER (Lipids from Less Dense Organic Phase Extracted from Sonicated D. hansenii)

### See FIG. 1C

m/z 1272.9 (Line 1, FIG. 1C) corresponds to a peptide:

(SEQ ID NO. 3)
SDWLGDQDAIHYMTEQAPASVVELENYGMPFS

M/Z 637.2 (line 2, FIG. 1C) corresponds to a peptide:

(SEQ ID NO. 4)
SPVKPGIPYKQLTVGVPK

m/z 387.0 (Line 3, FIG. 1C) corresponds to a lipid called 6-bromo-eicosa-5E,9Z-dienoic acid

TABLE 6
m/z 318.2 (Line 3, FIG. 1C) corresponds to:
Possible Lipid Structures

| LM_ID | Name | Systematic Name | Input m/z | Exact m/z | Formula | Ion |
|---|---|---|---|---|---|---|
| LMFA08020007 | N-methyl arachidonoyl amine | N-methyl-5Z,8Z,11Z,14Z-eicosatetraenoyl amine | 318.2 | 318.2791 | C₂₁H₃₆NO | M + H |
| LMSP01030001 | Phytosphingosine | 4R-hydroxysphinganine | 318.2 | 318.3003 | C₁₈H₄₀NO₃ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 7
m/z 319.0 (Line 3, FIG. 1C) corresponds to:
Possible Lipid Structures

| | | | Input | Exact | | |
|---|---|---|---|---|---|---|
| LM_ID | Name | Systematic Name | m/z | m/z | Formula | Ion |
| LMFA06000115 | 2-bromopalmitaidehyde | 2-bromo-hexadecanal | 319.0 | 319.1631 | C₁₆H₃₂O | M + H |
| LMST02020116 | 3,4,17-trihydroxy-9,10-seco- | 3,4,17beta-trihydroxy-9,10-seco-androsta- | 319.0 | 319.1904 | C₁₉H₂₇O₄ | M + H |
| | androsta-1,3,5(10)-triene-9-one | 1,3,5(10)-triene-9-one | | | | |

Note:
Chains containing double bonds and/or functional groups with defined regiochemistry, geometry and stereochemistry are meant to serve as examples for structure-drawing purposes. In many cases there may be alternative isobaric structures.

TABLE 8
m/z 411.2 (Line 3, FIG. 1C) corresponds to:
Possible Lipid Structures

| | | | Input | Exact | | |
|---|---|---|---|---|---|---|
| LM_ID | Name | Systematic Name | m/z | m/z | Formula | Ion |
| LMPR0106010005 | C30:6 Highly branched | 2,6,10,14,18-pentamethyl-13E-(3-methyl-pent- | 411.2 | 411.3985 | C₃₀H₅₁ | M + H |
| | isoprenoid A | 4-enylidene)-nonadeca-2,6E,10Z,17-tetraene | | | | |
| LMPR0106010006 | C30:6 Highly branched | 2,6,10,14,18-pentamethyl-13E-(3-methyl-pent- | 411.2 | 411.3985 | C₃₀H₅₁ | M + H |
| | isoprenoid B | 4-enylidene)-nonadeca-2,6E,10E,17-tetraene | | | | |
| LMPR0106020001 | C30:5 Monocyclic highly branched | 1-methyl-4-(prop-1-en-2-yl)-3-(3,7,11,15- | 411.2 | 411.3985 | C₃₀H₅₁ | M + H |
| | isoprenoid A | tetramethyl-hexadeca-1,10E,14-trien-7-yl)- | | | | |
| | | cyclohex-1-ene | | | | |
| LMPR0106200004 | (−)-5-Adianene | | 411.2 | 411.3985 | C₃₀H₅₁ | M + H |
| LMPR0106230002 | (−)-14-Serratene | | 411.2 | 411.3985 | C₃₀H₅₁ | M + H |
| LMPR02020055 | beta-tocotrienol | 2R,5,8-trimethyl-2-[(3E,7E)-4,8,12- | 411.2 | 411.3258 | C₂₈H₄₃O₂ | M + H |
| | | trimethyltrideca-3,7,11-trien-1-yl]-3,4-dihydro- | | | | |
| | | 2H-chromen-6-ol | | | | |
| LMPR02020057 | gamma-tocotrienol | 2R,7,8-trimethyl-2-[(3E,7E)-4,8,12- | 411.2 | 411.3258 | C₂₈H₄₃O₂ | M + H |
| | | trimethyltrideca-3,7,11-trien-1-yl]-3,4-dihydro- | | | | |
| | | 2H-chromen-6-ol | | | | |
| LMPR04000001 | Diploptene | Hop-22(29)-ene | 411.2 | 411.3985 | C₃₀H₅₁ | M + H |
| LMST01010149 | 4,4-dimethylcholesta-8,11,24- | 4,4-dimethyl-5alpha-cholesta-8,14,24-trien- | 411.2 | 411.3621 | C₂₉H₄₇O | M + H |
| | trienol | 3beta-ol | | | | |
| LMST01031020 | Delta 8,14-sterol | 4alpha-methyl-5alpha-ergosta-8,14,24(28)- | 411.2 | 411.3621 | C₂₉H₄₇O | M + H |
| | | trien-3beta-ol | | | | |
| LMST01031052 | (22E,24R)-24,26-dimethylcholesta- | 26-methylcampesta-5,22E,25(27)-trien-3beta-ol | 411.2 | 411.3621 | C₂₉H₄₇O | M + H |
| | 5,22,25(27)-trien-3beta-ol | | | | | |
| LMST01031084 | Dehydroconicasterol | 4,24-dimethylene-5alpha-cholest-8(14)-en- | 411.2 | 411.3621 | C₂₉H₄₇O | M + H |
| | | 3beta-ol | | | | |
| LMST01031114 | Nabrosteroid M | 4alpha-methyl-ergosta-6,8(14)-dien-3beta-ol | 411.2 | 411.3621 | C₂₉H₄₇O | M + H |
| LMST01040132 | | stigmasta-5,22E,25-trien-3beta-ol | 411.2 | 411.3621 | C₂₉H₄₇O | M + H |
| LMST01040138 | | stigmasta-7,22E,25-trien-3beta-ol | 411.2 | 411.3621 | C₂₉H₄₇O | M + H |
| LMST01040155 | 5-Dehydro-avenasterol | 24Z-ethylidene-cholesta-5,7-dien-3β-ol | 411.2 | 411.3621 | C₂₉H₄₇O | M + H |
| LMST01040172 | 24-altenyl-cholesterol | cholesta-5,24(28),28-trien-3beta-ol | 411.2 | 411.3621 | C₂₉H₄₇O | M + H |
| LMST01040203 | Corbisterol | Stigmasta-5,7,22E-trien-3beta-ol | 411.2 | 411.3621 | C₂₉H₄₇O | M + H |
| LMST01110001 | Catysterol | 23,28-cyclostigmasta-5,23(24)-dien-3beta-ol | 411.2 | 411.3621 | C₂₉H₄₇O | M + H |
| LMST01110004 | (23R)-isocalysterol | 23R,28-cyclostigmasta-5,24(28)-dien-3beta-ol | 411.2 | 411.3621 | C₂₉H₄₇O | M + H |
| LMST01110005 | (24S)-isocatysterol | (24S)-23,28-cyclostigmasta-5,23(28)-dien- | 411.2 | 411.3621 | C₂₉H₄₇O | M + H |
| | | 3beta-ol | | | | |
| LMST01160005 | Minabeotide-4 | 28-nor-3-oxo-witha-1,4-dienolide | 411.2 | 411.2894 | C₂₇H₃₉O₃ | M + H |
| LMST03020092 | (22E)-1alpha-hydroxy-24-oxo- | (5Z,7E,22E)-(1S,3R)-1,3-dihydroxy-26,27- | 411.2 | 411.2894 | C₂₇H₃₉O₃ | M + H |
| | 26,27-cyclo-22,23- | cyclo-9,10-seco-5,7,10(19),22-cholestatetraen- | | | | |
| | didehydrovitamin D3/(22E)- | 24-one | | | | |
| | 1alpha-hydroxy-24-oxo-26,27- | | | | | |
| | cyclo-22,23- | | | | | |
| | didehydrocholecalciferol | | | | | |
| LMST03020093 | 24,25-epoxy-1alpha-hydroxy- | (5Z,7E)-(1S,3R)-24,25-epoxy-9,10-seco- | 411.2 | 411.2894 | C₂₇H₃₉O₃ | M + H |
| | 22,22,23,23-tetradehydrovitamin | 5,7,10(19)-cholestatrien-22-yne-1,3-diol | | | | |
| | D3/24,25-epoxy-1alpha- | | | | | |
| | hydroxy-22,22,23,23- | | | | | |
| | tetradehydrocholecalciferol | | | | | |
| LMST03020094 | 24,26-epoxy-1alpha-hydroxy- | (5Z,7E)-(1S,3R)-25,26-epoxy-9,10-seco- | 411.2 | 411.2894 | C₂₇H₃₉O₃ | M + H |
| | 23,23,24,24-tetradehydrovitamin | 5,7,10(19)-cholestatrien-23-yne-1,3-diol | | | | |
| | D3/25,26-epoxy-1alpha- | | | | | |
| | hydroxy-23,23,24,24- | | | | | |
| | tetradehydrocholecalciferol | | | | | |
| LMST03020095 | 25,26-epoxy-1alpha-hydroxy- | (5Z,7E)-(1S,3R,20S)-25,26-epoxy-9,10-seco- | 411.2 | 411.2894 | C₂₇H₃₉O₃ | M + H |
| | 23,23,24,24-tetradehydro- | 5,7,10(19)-cholestatrien-23-yne-1,3-diol | | | | |
| | 20-epivitamin | | | | | |
| | D3/25,26-epoxy-1alpha-hydroxy- | | | | | |
| | 23,23,24,24-tetradehydro-20- | | | | | |
| | epicholecalciferol | | | | | |
| LMST03020096 | 1alpha,25-dihydroxy- | (5Z,7E)-(1S,3R)-9,10-seco-5,7,10(19),16- | 411.2 | 411.2894 | C₂₇H₃₉O₃ | M + H |
| | 16,17,23,23,24,24- | cholestatetraen-23-yne-1,3,25-triol | | | | |
| | hexadehydrovitamin | | | | | |
| | D3/1alpha,25-dihydroxy- | | | | | |
| | 16,17,23,23,24,24- | | | | | |
| | hexadehydrocholecalciferol | | | | | |
| LMST03020313 | callcoferol D | (22E)-(8S)-3-hydroxy-22-methyl-9,10-seco- | 411.2 | 411.3258 | C₂₈H₄₃O₂ | M + H |
| | | 1,3,5(10),22-cholestatetraen-9-one | | | | |
| LMST03050001 | Vitamin D6 | (5Z,7E,22E)-(3S)-9,10-seco-5,7,10(19),22- | 411.2 | 411.3621 | C₂₉H₄₇O | M + H |
| | | poriferastatetraen-3-ol | | | | |
| LMST03050002 | Provitamin D6 | Poriferasta-5,7,22E-trien-3beta-ol | 411.2 | 411.3621 | C₂₉H₄₇O | M + H |
| LMFA01030916 | 28:7(n-6) | 4Z,7Z,10Z,13Z,16Z,19Z,22Z- | 411.2 | 411.3258 | C₂₈H₄₃O₂ | M + H |
| | | octacosaheptaenoic acid | | | | |
| LMFA01060181 | 3-oxohexacosanoic acid | 3-oxohexacosanoic acid | 411.2 | 411.3833 | C₂₆H₅₁O₃ | M + H |
| LMFA01090040 | | 3-Iodo-octadecanoic acid | 411.2 | 411.1714 | C₁₈H₃₆O₂ | M + H |
| LMFA03010097 | PGF2alpha-11-acetate methyl ester | methyl 9S,15S-dihydroxy-11R-acetoxy- | 411.2 | 411.2741 | C₂₃H₃₉O₆ | M + H |
| | | 5Z-13E-prostadienoate | | | | |
| LMFA08020128 | N-oleoyl glutamine | N-(9Z-octadecenoyl)-glutamine | 411.2 | 411.3217 | C₂₃H₄₃N₂O₄ | M + H |
| LMGP10050006 | PA(16:0/0:0) | 1-hexadecanoyl-sn-glycero-3-phosphate | 411.2 | 411.2506 | C₁₉H₄₀O₇P | M + H |
| LMPR0103010027 | | 13-bromo-10R,11R-dichloro-7,11-dimethyl-3- | 411.2 | 411.0124 | C₁₆H₂₂O₃Cl2 | M + H |
| | | methylene-4R-hydroxy-6E,8E,12E- | | | | |
| | | tridecatrienoic acid | | | | |
| LMPR0104030004 | forskolin | (3R,4a,5S,6S,6aS,10S,10aR,10bS)-3- | 411.2 | 411.2377 | C₂₂H₃₅O₇ | M + H |
| | | ethenyl-6,10,10b-trihydroxy-3,4a,7,7,10a- | | | | |
| | | pentamethyl-1-oxododecahydro-1H- | | | | |
| | | benzo[f]chromen-5-yl acetate | | | | |
| LMPR0104030011 | (+)-subersic acid | 4-hydroxy-3-((2E)-3-methyl-5- | 411.2 | 411.2894 | C₂₇H₃₉O₃ | M + H |
| | | [(4aS,8aS)-2,5,5,8a-tetramethyl- | | | | |
| | | 3,4,4a,5,6,7,8,8a-octahydronaphthalen-1- | | | | |
| | | yl]pent-2-en-1-yl]benzoic acid | | | | |
| LMPR0104110004 | (+)-makassaric acid | 3-{[(14beta)-8,13-dimethylpodocarp-12-en-14- | 411.2 | 411.2894 | C₂₇H₃₉O₃ | M + H |
| | | yl]methyl}-4-hydroxybenzoic acid | | | | |
| LMPR0106010002 | Squalene | Squalene | 411.2 | 411.3985 | C₃₀H₅₁ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 9
m/z 105.2 (Line 1, FIG. 1D) corresponds to:
Possible Lipid Structures

| LM_ID | Name | Systematic Name | Input m/z | Exact m/z | Formula | Ion |
|---|---|---|---|---|---|---|
| LMFA01050004 | (+/−)alpha-hydroxy butyric acid | 2-hydroxy-butanoic acid | 105.2 | 105.0546 | C₄H₉O₃ | M + H |
| LMFA01050005 | D(−)-beta-hydroxy butyric acid | 3-hydroxy-butanoic acid | 105.2 | 105.0546 | C₄H₉O₃ | M + H |
| LMFA01050006 | 4-hydroxy-butyric acid | 4-hydroxy-butanoic acid | 105.2 | 105.0546 | C₄H₉O₃ | M + H |
| LMFA01050243 | | 3R-hydroxy-butanoic acid | 105.2 | 105.0546 | C₄H₉O₃ | M + H |
| LMFA01050342 | 2S-Hydroxybutanoic acid | 2S-hydroxy-butanoic acid | 105.2 | 105.0546 | C₄H₉O₃ | M + H |
| LMFA01050382 | 3R-hydroxy-isobutyric acid | 2R-methyl-3-hydroxy-propanoic acid | 105.2 | 105.0546 | C₄H₉O₃ | M + H |
| LMFA01050417 | alpha-hydroxy-isobutyric acid | 2-hydroxy-2-methyl-propanoic acid | 105.2 | 105.0546 | C₄H₉O₃ | M + H |
| LMFA01100051 | 2,3-diamino-propionic acid | 2S,3-diamino-propionic acid | 105.2 | 105.0658 | C₃H₉N₂O₂ | M + H |
| LMFA01170041 | Malonic acid | Propanedioic acid | 105.2 | 105.0182 | C₃H₅O₄ | M + H |

Note:
Chains containing double bonds and/or functional groups with defined regiochemistry, geometry and stereochemistry are meant to serve as examples for structure-drawing purposes. In many cases there may be alternative isobaric structures.

m/z 184 (Line 2, FIG. 1D) same as above.

TABLE 10
m/z 104.2 (Line 3, FIG. 1D):
Possible Lipid Structures

| LM_ID | Name | Systematic Name | Input m/z | Exact m/z | Formula | Ion |
|---|---|---|---|---|---|---|
| LMFA01100034 | | 2S-amino-butanoic acid | 104.2 | 104.0706 | C₄H₁₀NO₂ | M + H |
| LMFA01100039 | | 4-amino-butanoic acid | 104.2 | 104.0706 | C₄H₁₀NO₂ | M + H |
| LMFA01100043 | | 2R-amino-butanoic acid | 104.2 | 104.0706 | C₄H₁₀NO₂ | M + H |
| LMFA01100049 | (R)-b-amino-isobutyric acid | 2R-methyl-3-amino-propanoic acid | 104.2 | 104.0706 | C₄H₁₀NO₂ | M + H |
| LMFA01100050 | (S)-b-amino-isobutyric acid | 2S-methyl-3-amino-propanoic acid | 104.2 | 104.0706 | C₄H₁₀NO₂ | M + H |
| LMFA01100053 | 2-amino-isobutyric acid | 2-amino-2-methyl-propanoic acid | 104.2 | 104.0706 | C₄H₁₀NO₂ | M + H |
| LMFA01100054 | 3-amino-isobutanoic acid | 3-amino-3-methyl-propionic acid | 104.2 | 104.0706 | C₄H₁₀NO₂ | M + H |

Note:
Chains containing double bonds and/or functional groups with defined regiochemistry, geometry and stereochemistry are meant to serve as examples for structure-drawing purposes. In many cases there may be alternative isobaric structures.

### Third Sample

### FATS FROM CELLS—BOTTOM LAYER (Lipids from More Dense Extracted Organic Layer, from Sonicated D. hansenii) See FIG. 1E-1F

M/Z 112.4 (Line 1, FIG. 1E) same as above

TABLE 11
m/z 652.3 (Line 2, FIG. 1E):
Possible Lipid Structures

| | | | Input | Exact | | |
|---|---|---|---|---|---|---|
| LM_ID | Name | Systematic Name | m/z | m/z | Formula | Ion |
| LMGP03010931 | PS(14:0/12:0) | 1-tetradecanoyl-2-dodecanoyl-glycero-3-phosphoserine | 652.3 | 652.4184 | C₃₇H₆₃NO₁₀P | M + H |
| LMGP03010971 | PS(13:0/13:0) | 1,2-ditridecanoyl-sn-glycero-3-phosphoserine | 652.3 | 652.4184 | C₃₂H₆₃NO₁₀P | M + H |
| LMGP03010972 | PS(12:0/14:0) | 1-dodecanoyl-2-tetradecanoyl-glycero-3-phosphoserine | 652.3 | 652.4184 | C₃₂H₆₃NO₁₀P | M + H |
| LMST05010006 | 17alpha-(N-Acetyl-D- | 17-(N-Acetyl-D-glycosaminyl)-estra-1,3,5(10)-triene- | 652.3 | 652.2964 | C₃₂H₄₈NO₁₃ | M + H |
| | glucosaminyl)-estradiol 3-D- | 3,17alpha-diol 3-D-glucuronide | | | | |
| | glucuronide | | | | | |
| LMST05010027 | 17alpha-(N-Acetyl-D- | 17alpha-(2-acetamido-2-deoxy-beta-D- | 652.3 | 652.2964 | C₃₂H₄₆NO₁₃ | M + H |
| | glucosaminyl)estradiol 3- | glucopyranosyloxy)estra-1,3,5(10)-trien-3-yl beta-D- | | | | |
| | glucosiduronic acid | glucopyranosiduronic acid | | | | |

Note:
Chains containing double bonds and/or functional groups with defined regiochemistry, geometry and stereochemistry are meant to serve as examples for structure-drawing purposes. In many cases there may be alternative isobaric structures.

m/z 754.3 (Line 3, FIG. 1E) same as above

TABLE 12
m/z 131.9 (Line 1, FIG. 1E):
Possible Lipid Structures

| LM_ID | Name | Systematic Name | Input m/z | Exact m/z | Formula | Ion |
|---|---|---|---|---|---|---|
| LMFA01060169 | | 2-oxo-5-amino-pentanoic acid | 131.9 | 132.0655 | C₅H₁₀NO₃ | M + H |
| LMFA01060171 | | 2-amino-4-oxo-pentanoic acid | 131.9 | 132.0655 | C₅H₁₀NO₃ | M + H |
| LMFA01100055 | 5-amino-levulinic acid | 4-oxo-5-amino-pentanoic acid | 131.9 | 132.0655 | C₅H₁₀NO₃ | M + H |
| LMFA01170110 | Iminoaspartic acid | 2-imino-butanedioic acid | 131.9 | 132.0291 | C₄H₆NO₄ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 13
m/z 446.3 (Line 1, FIG. 1F):
Possible Lipid Structures

| | | | | Exact | | |
|---|---|---|---|---|---|---|
| LM_ID | Name | Systematic Name | Input m/z | m/z | Formula | Ion |
| LMFA07080001 | | O-hexanoyl-adenosine monophosphate | 446.3 | 446.1435 | C₁₆H₂₅N₅O₈P | M + H |
| LMFA08020099 | N-oleoyl tyrosine | N-(9Z-octadecenoyl)-tyrosine | 446.3 | 446.3265 | C₂₇H₄₄NO₄ | M + H |
| LMGP01011235 | PC(6:2)(2E,4E)/6:2(2E,4E)) | 1,2-di-(2E,4E-hexadienoyl)-sn-glycero-3- | 446.3 | 446.1938 | C₂₀H₃₃NO₈P | M + H |
| | | phosphocholine | | | | |
| LMST03010007 | 1alpha,25-dihydroxy-24-oxo-23- | (5Z,7E)-(1S,3R,24R)-23-aza-22-oxo- | 446.3 | 446.3265 | C₂₇H₄₄NO₄ | M + H |
| | azavitamin D2/1alpha,25-dihydroxy- | 9,10-seco-5,7,10(19)-ergosiatriene- | | | | |
| | 24-oxo-23-azaerocalciferol | 1,3,25-triol | | | | |

Note:
Chains containing double bonds and/or functional groups with defined regiochemistry, geometry and stereochemistry are meant to serve as examples for structure-drawing purposes. In many cases there may be alternative isobaric structures.

TABLE 14
m/z 385.1 (Line 1, FIG. 1F):
Possible Lipid Structures

| | | | Input | Exact | | |
|---|---|---|---|---|---|---|
| LM_ID | Name | Systematic Name | m/z | m/z | Formula | Ion |
| LMPR0104510001 | Rogloldiol A | | 385.1 | 385.1737 | C₂₀H₃₄O₂ | M + H |
| LMST01130003 | Sclliarenin | 3beta,14-dihydroxybuta-4,20,22-trienolide | 385.1 | 385.2373 | C₂₄H₃₃O₄ | M + H |
| LMST02010040 | Estradiol dipropionate | 3,17-dipropionyl-estra-1,3,5(10)-triene-3,17beta-diol | 385.1 | 385.2373 | C₂₄H₃₃O₄ | M + H |
| LMST02030118 | Megestrol acetate | 17alpha-hydroxy-6-methylpregna-4,6-diene-3,20- | 385.1 | 385.2373 | C₂₄H₃₃O₄ | M + H |
| | | dione acetate | | | | |
| LMSTD2030124 | Ethynodiol diacetate | 19-norpregn-4-en-20-yn-3beta,17alpha-diol, diacetate | 385.1 | 385.2373 | C₂₄H₃₃O₄ | M + H |
| LMST04010281 | | 3,12-Dioxochola-1,4-dien-24-oic Acid | 385.1 | 385.2373 | C₂₄H₃₃O₄ | M + H |
| LMST04010393 | | 12alpha-Hydroxy-3-oxochola-1,4,6-trien-24-oic Acid | 385.1 | 385.2373 | C₂₄H₃₃O₄ | M + H |
| LMST04010396 | | 3,12-Dioxochola-4,6-dien-24-oic Acid | 385.1 | 385.2373 | C₂₄H₃₃O₄ | M + H |
| LMST04010407 | | (22E)-12alpha-Hydroxy-3-oxochola-1,4;22-trien-24-oic | 385.1 | 385.2373 | C₂₄H₃₃O₄ | M + H |
| | | Acid | | | | |
| LMST05020022 | 3b,16a- | 3b,16a-Dihydroxy-5-androsten-17-one 3-sulfate | 385.1 | 385.1679 | C₁₉H₂₉O₆S | M + H |
| | Dihydroxyandrostenone | | | | | |
| | sulfate | | | | | |

Note:
Chains containing double bonds and/or functional groups with defined regiochemistry, geometry and stereochemistry are meant to serve as examples for structure-drawing purposes. In many cases there may be alternative isobaric structures.

TABLE 15
m/z 442.5 (Line 1, FIG. 1F):
Possible Lipid Structures

| LM_ID | Name | Systematic Name | Input m/z | Exact m/z | Formula | Ion |
|---|---|---|---|---|---|---|
| LMFA08020131 | N-arachidonoylhistidine | N-(5Z,8Z,11Z,14Z-eicosatetraenoyl)-histidine | 442.5 | 442.3064 | C₂₆H₄₀N₃O₃ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 16
m/z 343.1 (Line 1, FIG. 1F):
Possible Lipid Structures

| | | | Input | Exact | | |
|---|---|---|---|---|---|---|
| LM_ID | Name | Systematic Name | m/z | m/z | Formula | Ion |
| LMFA01030853 | TrHA | 5Z,8Z,11Z,14Z,17Z,20Z-tricosahexaenoic acid | 343.1 | 343.2632 | C₂₃H₃₅O₂ | M + H |
| LMFA01050149 | | methyl 9,12-dihydroxy-13-oxo-10-octadecenoate | 343.1 | 343.2479 | C₁₉H₃₅O₅ | M + H |
| LMFA01050150 | | methyl 10,13-dihydroxy-9-oxo-11-ocdadecenoate | 343.1 | 343.2479 | C₁₉H₃₅O₅ | M + H |
| LMFA01170035 | Eicosanedioic acid | Eicosanedioic acid | 343.1 | 343.2843 | C₂₀H₃₉O₄ | M + H |
| LMFA02000288 | 13-hydroxy-9-methoxy-10- | 13-hydroxy-9-melhoxy-10-oxo-11E-octadecenoic | 343.1 | 343.2479 | C₁₉H₃₅O₅ | M + H |
| | oxooctadec-11-enoic acid | acid | | | | |
| LMFA03010089 | 2,3-dinor, 6-keto-PGF1alpha | 6-oxo-9S,11R,15S-trihydroxy-2,3-dinor-13E- | 343.1 | 343.2115 | C₁₈H₃₁O₆ | M + H |
| | | prostaenoic acid | | | | |
| LMFA03010173 | PGF1a alcohol | 1,9S,11R,15S-tetrahydroxy-13E-prostaene | 343.1 | 343.2843 | C₂₀H₃₉O₄ | M + H |
| LMFA03030003 | 2,3-Dinot-TXB2 | 9S,11,5S-trihydroxy-2,3-dinor-tnromboxa- | 343.1 | 343.2115 | C₁₆H₃₁O₆ | M + H |
| | | 5Z,13E-dien-1-oic acid | | | | |
| LMFA03030012 | 2,3-Dinor-TXB1 | 9S,11,15S-trihydroxy-2,3-dinor-thrombox-13E-en- | 343.1 | 343.2479 | C₁₉H₃₅O₅ | M + H |
| | | 1-oic acid | | | | |
| LMFA04000014 | 16,17-epoxy-DHA | 16,17-epoxy-4Z,7Z,10Z,12E,14E,19Z- | 343.1 | 343.2268 | C₂₂H₃₁O₃ | M + H |
| | | docosahexaenoic acid | | | | |
| LMFA04000052 | 16,17S-DHA-epoxide | 16,17S-epoxy-4Z,7Z,10Z,12E,14E,19Z- | 343.1 | 343.2268 | C₂₂H₃₁O₃ | M + H |
| | | docosahexaenoic acid | | | | |
| LMPR0104410001 | Sinulobatin A | | 343.1 | 343.2268 | C₂₂H₃₁O₃ | M + H |
| LMST02030177 | Megestrol | 7-hydroxy-6-methylpregna-4,6-diene-3,20-dione | 343.1 | 343.2268 | C₂₂H₃₁O₃ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 17
m/z 391.0 (Line 1, FIG. 1F):
Possible Lipid Structures

| LM_ID | Name | Systematic Name | Input m/z | Exact m/z | Formula | Ion |
|---|---|---|---|---|---|---|
| LMFA02020014 | (−)-11-hydroxy-9,10- | {(1R,2R)-2-[4-(beta-D- | 391.0 | 391.1963 | C₁₈H₃₁O₉ | M + H |
| | dihydrojasmonic acid | glucopyranosyloxy)pentyl]-3-oxocyclopentyl}acetic | | | | |
| | 11-beta-D-glucoside | acid | | | | |
| LMFA02020206 | (−)-11-hydroxy-9,10- | {(1R,2R)-2-[4-(beta-D- | 391.0 | 391.1963 | C₁₈H₃₁O₉ | M + H |
| | dihydrojasmonic acid | glucopyranosyloxy)pentyl]-3-oxocyclopentyl}acetic | | | | |
| | 11-beta-D-glucoside | acid | | | | |
| LMPR0102070001 | Loganin | | 391.0 | 391.1599 | C₁₇H₂₇O₁₀ | M + H |
| LMPR0102070012 | Monotropein | | 391.0 | 391.1235 | C₁₆H₂₃O₁₁ | M + H |

Note:
Chains containing double bonds and/or functional groups with defined regiochemistry, geometry and stereochemistry are meant to serve as examples for structure-drawing purposes. In many cases there may be alternative isobaric structures.

TABLE 18
m/z 397 (Line 1, FIG. 1F):

| | | | Input | Exact | | |
|---|---|---|---|---|---|---|
| LM_ID | Name | Systematic Name | m/z | m/z | Formula | Ion |
| LMST03010001 | Vitamin D2 | (5Z,7E,22E)-(3S)-9,10-seco-5,7,10(19),22- | 397.2 | 397.3465 | C₂₈H₄₅O | M + H |
| | | ergostatetraen-3-ol | | | | |
| LMST03010014 | (5E)-vitamin D2/5.6-trans-vitamin | (5E,7E,22E)-(3S)-9,10-seco-5,7,10(19),22- | 397.2 | 397.3465 | C₂₈H₄₅O | M + H |
| | D2/(5E)-ergocaiciferol/(5E)-ercaicol | ergostatetraen-3-ol | | | | |
| LMST03010015 | previtamin D2/preergocalciferol | (6Z,24E)-(3S)-9,10-seco-5(10),6,8,22- | 397.2 | 397.3465 | C₂₈H₄₅O | M + H |
| | | ergostatetraen-3-ol | | | | |
| LMST03010016 | tachysterol2 | (6E,22E)-(3S)-9,10-seco-5(10),6,8,22- | 397.2 | 397.3465 | C₂₈H₄₅O | M + H |
| | | ergostatetraen-3-ol | | | | |
| LMST03010017 | Isotachysterol2 | (6E,22E)-(3S)-9,10-seco-5(10),6,8(14),22- | 397.2 | 397.3465 | C₂₈H₄₅O | M + H |
| | | ergostatetraen-3-ol | | | | |
| LMST03010018 | (5Z)-isovitamin D2/5,6-cis-isovitamin | (5Z,7E,22E)-(3S)-9,10-seco-1(10),5,7,22- | 397.2 | 397.3465 | C₂₈H₄₅O | M + H |
| | D2/(5Z)-isoergocalciferol/5,6-cis- | ergostatetraen-3-ol | | | | |
| | isoergocalciferol | | | | | |
| LMST03010019 | (5E)-isovitamin D2/(5E)-isoergocalciferol | (5E,7E,22E)-(3S)-9,10-seco-1(10),5,7,22- | 397.2 | 397.3465 | C₂₈H₄₅O | M + H |
| | | ergostatetraen 3-ol | | | | |
| LMST03010068 | Suprasterol II | (5E,22E)-(3S,7R,13R,14S,17S,20S)-7,19- | 397.2 | 397.3465 | C₂₈H₄₅O | M + H |
| | | cyclo-8,19-cyclo-9,10-seco-5(10),22- | | | | |
| | | ergostadien-3-ol | | | | |
| LMST03020100 | 25-hydroxy-16,17,23,24- | (5Z,7E,23E)-(3S)-9,10-seco- | 397.2 | 397.3101 | C₂₇H₄₁O₂ | M + H |
| | tetradehydrovitamin D3/25-hydroxy- | 5,7,10(19),16,23-Cholestapentaene-3,25- | | | | |
| | 16,17,23,24-tetradehydrochotecalciferol | diol | | | | |
| LMST03020101 | (23Z)-25-hydroxy-16,17,23,24- | (5Z,7E,23Z)-(3S)-9,10-seco- | 397.2 | 397.3101 | C₂₇H₄₁O₂ | M + H |
| | tetradehydrovitamin D3/(23Z)-25- | 5,7,10(19),16,23-cholestapentaene-3,25- | | | | |
| | hydroxy-16,17,23,24- | diol | | | | |
| | tetradehydrocholecalciferol | | | | | |
| LMST03020102 | 25-hydroxy-23,23,24,24-tetrahydrovitamin | (5Z,7E)-(3S)-9,10-seco-5,7,10(19)- | 397.2 | 397.3101 | C₂₇H₄₁O₂ | M + H |
| | D3/25-hydroxy-23,23,24,24- | cholestatrien-23-yne-3,25-diol | | | | |
| | tetradehydrocholecalciferol | | | | | |
| LMST03020605 | calicoferol A | (22E)-(8S)-3-hydroxy-9,10-seco- | 397.2 | 397.3101 | C₂₇H₄₁O₂ | M + H |
| | | 1,3,5(10),22-cholestatetraen-9-one | | | | |
| LMFA00000008 | (9S,10S)-10-hydroxy-9- | (9S,10S)-10-hydroxy-9- | | | | |
| | (phosphonooxy)octadecanosic acid | (phosphonooxy)octadecanoic acid | 397.2 | 397.2350 | C₁₈H₃₈O₇P | M + H |
| LMFA01040054 | | methyl 8-[3,5-epidioxy-2-(3-hydroperoxy-1- | 397.2 | 397.2585 | C₂₂H₃₇O₆ | M + H |
| | | pentenyl)-cyclopentyl]-octanoate | | | | |
| LMFA03010076 | PGF2alpha isopropyl ester | Isopropyl 9S,11R,15S-trihydroxy-5Z,13E- | 397.2 | 397.2948 | C₂₃H₃₁O₅ | M + H |
| | | prostadienoate | | | | |
| LMFA03010096 | PGF2alpha-11-acetate | 9S,15S-dihydroxy-11R-acetoxy-5Z,13E- | 397.2 | 397.2585 | C₂₂H₃₇O₆ | M + H |
| | | prostadienoic acid | | | | |
| LMFA03110168 | 10-F2-dihomo-isoP | 1a,1b-dihomo-8,12,14-trihydroxy-5Z,9E- | 397.2 | 397.2948 | C₂₃H₄₁O₅ | M + H |
| | | prostadienoic acid-cyclo[11,15] | | | | |
| LMFA03120012 | 10,11-epoxy-chlorovulone I | methyl 9-oxo-10R-chloro-10,11S-epoxy- | | | | |
| | | 12S-hydroxy-5Z,7E13Z-prostatrienoate- | | | | |
| | | cyclo[8,12] | 397.2 | 397.1776 | C₂₁H₃₀O₅Cl | M + H |
| LMFA08070137 | N-oleoyl asparagine | N-(9Z-octadecenoyl)-asparagine | 397.2 | 397.3061 | C₂₂H₄₁N₂O₄ | M + H |
| LMGP10050037 | PA(15:0/0:0) | 1-pentadecanoyl-glycero-3-phosphate | 397.2 | 397.2350 | C₁₈H₃₈O₇P | M + H |
| LMGP10060005 | PA(O-16:0/0:0) | 1-hexadecyl-glycero-3-phosphate | 397.2 | 397.2713 | C₁₉H₄₂O₆P | M + H |
| LMPR02020056 | delta-tocotrienol | 2R,6-dimethyl-2-[(3E,7E)-4,8,12- | 397.2 | 397.3101 | C₂₇H₄₁O₂ | M + H |
| | | trimethyltrideca-3,7,11-trien-1-yl]-3,4- | | | | |
| | | dihydro-2H-chromen-6-ol | | | | |
| LMST01010167 | 3-dehydro-4-methylzymosterol | 4-methyl-5alpha-cholesta-8,24-dien-3-one | 397.2 | 397.3465 | C₂₈H₄₅O | M + H |
| LMST01010237 | 3-keto-4alpha-methyl-zynosterol | 4alpha-methyl-5alpha-cholesta-8,24-dien-3- | 397.2 | 397.3465 | C₂₈H₄₅O | M + H |
| | | one | | | | |
| LMST01030093 | Ergosterol | ergosta-5,7,22E-trien-3beta-ol | 397.2 | 397.3465 | C₂₈H₄₅O | M + H |
| LMST01030135 | 5-dehydroepisterol | 24-methylene-cholesta-5,7-dien-3beta-ol | 397.2 | 397.3465 | C₂₈H₄₅O | M + H |
| LMST01031025 | | ergosta-4,7,22E-trien-3beta-ol | 397.2 | 397.3465 | C₂₈H₄₅O | M + H |
| LMST01031026 | | ergosta-5,8,22E-trien-3beta-ol | 397.2 | 397.3465 | C₂₈H₄₅O | M + H |
| LMST01031057 | | cholesta-5,24(28),25-trien-3beta-ol | 397.2 | 397.3465 | C₂₈H₄₅O | M + H |
| LMST01040156 | Balkalosterol | 24-ethyl-26-norcholesta-5,22E,25-trien- | 397.2 | 397.3465 | C₂₈H₄₅O | M + H |
| | | 3-beta-ol | | | | |
| LMST04060001 | | 24-Nor-5beta-cholane- | 397.2 | 397.2948 | C₂₃H₄₁O₅ | M + H |
| | | 3alpha,7alpha,12alpha,22,23-pentol | | | | |
| LMST05020014 | pregnenolone sulfate | 20-oxopregn-5-en-3beta-yl sulfate | 397.2 | 397.2043 | C₂₁H₃₃O₅S | M + H |

TABLE 19
m/z 129.1 (Line 2, FIG. 1F):

| LM_ID | Name | Systematic Name | Input m/z | Exact m/z | Formula | Ion |
|---|---|---|---|---|---|---|
| LMFA01020100 | | 2-methyl-2Z-hexenoic acid | 129.1 | 129.0910 | C₇H₁₃O₂ | M + H |
| LMFA01020101 | | 2-methyl-2E-hexenoic acid | 129.1 | 129.0910 | C₇H₁₃O₂ | M + H |
| LMFA01020117 | | 5-methyl-5-hexenoic acid | 129.1 | 129.0910 | C₇H₁₃O₂ | M + H |
| LMFA01020118 | 2-butyl acrylic acid | 2-butyl-2-propenoic acid | 129.1 | 129.0910 | C₇H₁₃O₂ | M + H |
| LMFA01020119 | | 4,4-dimethyl-2E-pentenoic acid | 129.1 | 129.0910 | C₇H₁₃O₂ | M + H |
| LMFA01020120 | | 4,4-dimethyl-2Z-pentenoic acid | 129.1 | 129.0910 | C₇H₁₃O₂ | M + H |
| LMFA01020121 | | 2,2-dlmethyl-4-pentenoic acid | 129.1 | 129.0910 | C₇H₁₃O₂ | M + H |
| LMFA01020122 | 2-Isopropyl trans-crotonic acid | 2-Isopropyl-2E-butenoic acid | 129.1 | 129.0910 | C₇H₁₃O₂ | M + H |
| LMFA01020123 | | 3-Isopropyl-3-butenoic acid | 129.1 | 129.0910 | C₇H₁₃O₂ | M + H |
| LMFA01030012 | alpha-heptenoic acid | 2-heptenoic acid | 129.1 | 129.0910 | C₇H₁₃O₂ | M + H |
| LMFA01030013 | beta-heptenoic acid | 3-heptenoic acid | 129.1 | 129.0910 | C₇H₁₃O₂ | M + H |
| LMFA01030014 | gamma-heptenoic acid | 4-heptenoic acid | 129.1 | 129.0910 | C₇H₁₃O₂ | M + H |
| LMFA01030015 | delta-heptenoic acid | 5-heptenoic acid | 129.1 | 129.0910 | C₇H₁₃O₂ | M + H |
| LMFA01030016 | epslion-heptenoic acid | 6-heptenoic acid | 129.1 | 129.0910 | C₇H₁₃O₂ | M + H |
| LMFA01030452 | | 4Z-heptenoic acid | 129.1 | 129.0910 | C₇H₁₃O₂ | M + H |
| LMFA01030793 | | 3-Methyl-2E-hexenoic acid | 129.1 | 129.0910 | C₇H₁₃O₂ | M + H |
| LMFA01030794 | | 3-Methyl-2Z-hexenoic acid | 129.1 | 129.0910 | C₇H₁₃O₂ | M + H |
| LMFA01050273 | | 6-hydroxy-2-hexynoic acid | 129.1 | 129.0546 | C₆H₉O₃ | M + H |
| LMFA01050367 | oct-1-en-3S-ol | oct-1-en-3S-ol | 129.1 | 129.1274 | C₈H₁₇O | M + H |
| LMFA01060174 | | 2-Oxo-4E-hexenoic acid | 129.1 | 129.0546 | C₆H₉O₃ | M + H |
| LMFA01090070 | Dichloroacetic acid | 2,2-dichloroacetic acid | 129.1 | 128.9505 | C₂H₃O₂Cl2 | M + H |
| LMFA01100037 | | 4-amino-4-cyano-butanoic acid | 129.1 | 129.0658 | C₅H₉N₂O₂ | M + H |
| LMFA01100038 | | 2-amino-4-cyano-butanoic acid | 129.1 | 129.0658 | C₅H₉N₂O₂ | M + H |
| LMFA05000090 | oct-1-en-3-ol | oct-1-en-3-ol | 129.1 | 129.1274 | C₈H₁₇O | M + H |
| LMFA05000093 | (R)-oct-1-en-3-ol | (3R)-oct-1-en-3-ol | 129.1 | 129.1274 | C₈H₁₇O | M + H |
| LMFA05000124 | | 2E-Octen-1-ol | 129.1 | 129.1274 | C₈H₁₇O | M + H |
| LMFA05000126 | | 2Z-Octen-1-ol | 129.1 | 129.1274 | C₈H₁₇O | M + H |
| LMFA05000128 | | 3Z-Octen-1-ol | 129.1 | 129.1274 | C₈H₁₇O | M + H |
| LMFA05000469 | R-sulcatol | 6-Methyl-5-hepten-2R-ol | 129.1 | 129.1274 | C₈H₁₇O | M + H |
| LMFA05000470 | S-sutcatol | 6-Methyl-5-hepten-2S-ol | 129.1 | 129.1274 | C₈H₁₇O | M + H |
| LMFA05000474 | | 4-Methyl-4E-hepten-3-ol | 129.1 | 129.1274 | C₈H₁₇O | M + H |
| LMFA05000475 | Rhynchophorol. | 6-Methyl-2E-hepten-4-ol | 129.1 | 129.1274 | C₈H₁₇O | M + H |
| LMFA05000481 | S-Rhynchophorol | 6-Methyl-2E-hepten-4S-ol | 129.1 | 129.1274 | C₈H₁₇O | M + H |
| LMFA06000026 | Pimelic dlaldehyde | heptanedial | 129.1 | 129.0910 | C₇H₁₃O₂ | M + H |
| LMFA06000028 | Caprylaldehyde | octanal | 129.1 | 129.1274 | C₈H₁₇O | M + H |
| LMFA06000050 | | 2-nonene-4,6,8-trlynal | 129.1 | 129.0335 | C₉H₅O | M + H |
| LMFA06000124 | | 3,5-Dimethylhexanal | 129.1 | 129.1274 | C₈H₁₇O | M + H |
| LMFA07040005 | | 3-Acetyldihydro-2(3H)-furanone | 129.1 | 129.0546 | C₆H₉O₃ | M + H |
| LMFA07040011 | cis-2-Methyl-5-hexanolide | 2R-Methyl-5S-hexanollde | 129.1 | 129.0910 | C₇H₁₃O₂ | M + H |
| LMFA07040012 | 4-heptanolide | Dihydro-5-propylfuran-2(3H)-one | 129.1 | 129.0910 | C₇H₁₃O₂ | M + H |
| LMFA11000333 | | 2,2,4-Trimethylhexane | 129.1 | 129.1638 | C₉H₂₁ | M + H |
| LMFA11000433 | | 4-Methyloctane | 129.1 | 129.1638 | C₉H₂₁ | M + H |
| LMFA11000579 | | Nonane | 129.1 | 129.1638 | C₉H₂₁ | M + H |
| LMFA11000603 | | 2,5-dimethyl-heptane | 129.1 | 129.1638 | C₉H₂₁ | M + H |
| LMFA11000607 | | 4-ethyl-heptane | 129.1 | 129.1638 | C₉H₂₁ | M + H |
| LMFA11000611 | | 2,3-dimethylhexane | 129.1 | 129.1638 | C₉H₂₁ | M + H |
| LMFA11000612 | | 2,4-dimethylhexane | 129.1 | 129.1638 | C₉H₂₁ | M + H |
| LMFA11000623 | | 3-methyloctane | 129.1 | 129.1638 | C₉H₂₁ | M + H |
| LMFA11000626 | | 2,2,3,3-tetramethylpentane | 129.1 | 129.1638 | C₉H₂₁ | M + H |
| LMFA11000671 | | 2,2,3,4-Tetramethylpentane | 129.1 | 129.1638 | C₉H₂₁ | M + H |
| LMFA11000699 | | 2,2-Oimethyl-3-ethylpentane | 129.1 | 129.1638 | C₉H₂₁ | M + H |
| LMFA11000701 | | 3-Ethylheptane | 129.1 | 129.1638 | C₉H₂₁ | M + H |
| LMFA12000013 | | 2,3-Heptanedione | 129.1 | 129.0910 | C₇H₁₃O₂ | M + H |
| LMFA12000025 | | 3-Methylheptan-2-one | 129.1 | 129.1274 | C₈H₁₇O | M + H |
| LMFA12000026 | | 3-Methylheptan-4-one | 129.1 | 129.1274 | C₈H₁₇O | M + H |
| LMFA12000031 | | 4-Methylheptan-3-one | 129.1 | 129.1274 | C₈H₁₇O | M + H |
| LMFA12000041 | | 6-Methylheptan-2-one | 129.1 | 129.1274 | C₈H₁₇O | M + H |
| LMFA12000042 | | 6-Methylheptan-3-one | 129.1 | 129.1274 | C₈H₁₇O | M + H |
| LMFA12000050 | | 5-Methylheptan-2-one | 129.1 | 129.1274 | C₈H₁₇O | M + H |
| LMFA12000054 | | Octan-2-one | 129.1 | 129.1274 | C₈H₁₇O | M + H |
| LMFA12000055 | | Octan-3-one | 129.1 | 129.1274 | C₈H₁₇O | M + H |
| LMFA12000091 | | 4R-Methylheptan-3-one | 129.1 | 129.1274 | C₈H₁₇O | M + H |
| LMFA12000092 | | 4S-Methylheptan-3-one | 129.1 | 129.1274 | C₈H₁₇O | M + H |
| LMFA12000100 | | 2-Methylheptan-4-one | 129.1 | 129.1274 | C₈H₁₇O | M + H |
| LMFA12000112 | | 5-Melhylheptan-3-one | 129.1 | 129.1274 | C₈H₁₇O | M + H |
| LMFA12000245 | | Heptan-2,5-dione | 129.1 | 129.0910 | C₇H₁₃O₂ | M + H |

TABLE 20
m/z 105. (Line 2, FIG. 1F):
Possible Lipid Structures

| LM_ID | Name | Systematic Name | Input m/z | Exact m/z | Formula | Ion |
|---|---|---|---|---|---|---|
| LMFA01050004 | (+/−)alpha-hydoxy-butyric acid | 2-hydroxy-butanoic acid | 105.1 | 105.0546 | C₄H₉O₃ | M + H |
| LMFA01050005 | D(−)-beta-hydroxy-butyric acid | 3-hydroxy-butanoic acid | 105.1 | 105.0546 | C₄H₉O₃ | M + H |
| LMFA01050006 | 4-hydroxy-butyric acid | 3-hydroxy-butanoic acid | 105.1 | 105.0546 | C₄H₉O₃ | M + H |
| LMFA01050243 | | 3R-hydroxy-butanoic acid | 105.1 | 105.0546 | C₄H₉O₃ | M + H |
| LMFA01050342 | 2S-Hydroxybutanoic acid | 2S-hydroxy-butanoic acid | 105.1 | 105.0546 | C₄H₉O₃ | M + H |
| LMFA01050382 | 3R-hydroxy-isobutyric acid | 2R-methyl-3-hydroxy-propanoic acid | 105.1 | 105.0546 | C₄H₉O₃ | M + H |
| LMFA01050417 | alpha-hydroxy-isobutyric acid | 2-hydroxy-2-methyl-propanoic acid | 105.1 | 105.0546 | C₄H₉O₃ | M + H |
| LMFA01100051 | 2,3-diamino-propionic acid | 2S,3-diamino-propionic acid | 105.1 | 105.0658 | C₃H₉N₂O₂ | M + H |
| LMFA01170041 | Maionic acid | Propanedioic acid | 105.1 | 105.0182 | C₃H₅O₃ | M + H |

Note:
Chains containing double bonds and/or functional groups with defined regiochemistry, geometry and stereochemistry are meant to serve as examples for struclure-drawing purposes. In many cases there may be altemative isobaric structures.

TABLE 21
m/z 149.1 (Line 2, FIG. 1F):
Possible Lipid Structures

| LM_ID | Name | Systematic Name | Input m/z | Exact m/z | Formula | Ion |
|---|---|---|---|---|---|---|
| LMFA01050352 | Mevaionic acid | 3R-methyl-3,5-dihydroxy-pentanoic acid | 149.1 | 149.0808 | C₆H₁₃O₄ | M + H |
| LMFA01060170 | | 2-oxo-4-methylthio-butanoic acid | 149.1 | 149.0267 | C₅H₉O₃S | M + H |
| LMFA01060194 | dihydroxy-fumaric acid | 2-oxo-3,4,4-trihydroxy3E-butanoic acid | 149.1 | 149.0081 | C₄H₅O₆ | M + H |
| LMFA06000061 | | 2,4,6,8-decatetraenal | 149.1 | 149.0961 | C₁₀H₁₃O | M + H |

Note:
Chains containing double bonds and/or functional groups with defined regiochemistry, geometry and stereochemistry are meant to serve as examples for struclure-drawing purposes. In many cases there may be altemative isobaric structures.

m/z 184-184.1 phosphocholine
m/z 104.2 (Line 3, FIG. 1F): same as above.
A Comparison Between the Samples was Made and the Following Probable Compounds were Found Common Between the Supernatant and the D. hansenii Extracts (Both Top and Bottom Layers):
m/z 1025.4 This m/z ratio corresponds possibly to fragments derived from a peptide with the following structure:

(SEQ ID NO. 5)
ELASQPDVDGFLVGGASLKPEFVDIINAK

m/z 1025.3 was only found in the top layer and it corresponds possibly to fragments derived from a peptide with the following structure:

(SEQ ID NO. 6)
YRIPADVDPLTITSSLSSD

m/z 1042.7 was only found it the top layer. It corresponds possibly to fragments derived from a peptide with the following structure:

(SEQ ID NO. 7)
KPVIAAVNGYAFGGGCELAMMCDIIYAGEK

On Another Range of m/z Ratios this is What was Found:
m/z 981.2, 926.4, 978.3 and 977.4 were highly abundant in the bottom layer of D. hansenni extracts. They were found in smaller amounts in the supernatant and the top layer. The identities are:

m/z 981.2 a fragment from peptide
(SEQ ID NO. 8)
SSIGTGYDLSASTFSPDGR
m/z 926.4 a fragmente from peptide
(SEQ ID NO. 9)
ASSVTTFTGEPNMCPR

m/z 978.3 could correspond to a fragment derived from this peptide:

(SEQ ID NO. 10)
FDCSNFNLTVHEAMGTGDLDLLSAFR

or to the following Coenzyme:)

TABLE 22
Possible Lipid Structures

| LM_ID | Name | Systematic Name | Input m/z | Exact m/z | Formula | Ion |
|---|---|---|---|---|---|---|
| LMFA07050008 | | Tetradecanoyl-CoA | 978.3 | 978.3209 | C₃₅H₆₃N₁₇P₃S | M + H |

Note:
Chains containing double bonds and/or functional groups with defined regiochemistry, geometry and stereochemistry are meant to serve as examples for struclure-drawing purposes. In many cases there may be altemative isobaric structures.

m/z 977.4 could correspond to a fragment derived from this peptide:

(SEQ ID NO. 11)
VEEIPEEWELYYPQK

The supernatant and top layer of D. hansenni extracts contained the following common m/z ions

TABLE 23
m/z 900.5:
Possible Lipid Structures

| LM_ID | Name | Systematic Name | Input m/z | Exact m/z | Formula | Ion |
|---|---|---|---|---|---|---|
| LMGP03010723 | PS(22:0/22:2(13Z,16Z)) | 1-docosanoly-2-(13Z,16Z-docosadienoyl)-glycero-3- | 900.5 | 900.6688 | C₅₀H₉₅NO₁₀P | M + H |
| | | phosphoserine | | | | |
| LMGP03010752 | PS(22:1(11Z)/22:1(11Z)) | 1,2-dl-(11Z-docosenoyl)-sn-glycero-3-phosphoserine | 900.5 | 900.6688 | C₅₀H₉₅NO₁₀P | M + H |
| LMGP03010782 | PS(22:2(13Z,16Z)22:0) | 1-(13Z,16Z-docosadienoyl)-2-docosanoyl-glycero-3- | 900.5 | 900.6688 | C₅₀H₉₅NO₁₀P | M + H |
| | | phosphoserine | | | | |

Note:
Chains containing double bonds and/or functional groups with defined regiochemistry, geometry and stereochemistry are meant to serve as examples for struclure-drawing purposes. In many cases there may be altemative isobaric structures.

TABLE 24
m/z 900.6:
Possible Lipid Structures

| LM_ID | Name | Systematic Name | Input m/z | Exact m/z | Formula | Ion |
|---|---|---|---|---|---|---|
| LMGP01011032 | PC(20:0/24:1(15Z)) | 1-eicosanoyl-2-(15Z-tetracosanoyl)-sn-glycero-3- | 900.6 | 900.7416 | C₅₂H₁₀₃NO₈P | M + H |
| | | phosphocholine | | | | |
| LMGP01011102 | PC(22:0/22:1(13Z)) | 1-docosanoyl-2-(13Z-docosenoyl)-sn-glycero-3- | 900.6 | 900.7416 | C₅₂H₁₀₃NO₈P | M + H |
| | | phosphocholine | | | | |
| LMGP0101115 | PC(24:0/20:1(11Z)) | 1-tetracosanoyl-2-(11Z-eicosenoyl)-sn-glycero-3- | 900.6 | 900.7416 | C₅₂H₁₀₃NO₈P | M + H |
| | | phosphocholine | | | | |
| IMGP01012000 | PC(22:0/22:1(11Z)) | 1-docosanoyl-2-(11Z-docosenoyl)-glycero-3- | 900.6 | 900.7416 | C₅₂H₁₀₃NO₈P | M + H |
| | | phosphocholine | | | | |
| LMGP01012030 | PC(22:1(11Z)/22:0) | 1-(11Z.docosenoyl)-2-docosanoyl)-glycero-3- | 900.6 | 900.7416 | C₅₂H₁₀₃NO₈P | M + H |
| | | phosphocholine | | | | |
| LMGP03010723 | PS(22:0/22:2(13Z.16Z)) | 1-docosanoyl-2-(13Z,16Z-docosadienoyl)-glycero-3- | 900.6 | 900.6688 | C₅₀H₉₅NO₁₀P | M + H |
| | | phosphocholine | | | | |
| LMGP03010752 | PS(22:1(11Z)22:1(11Z)) | 1,2-di-(11Z-docosenoyl)-sn-glycero-3-phosphoserine | 900.6 | 900.6688 | C₅₀H₉₅NO₁₀P | M + H |
| LMGP03010782 | PS(22:2(13Z,16Z)/22:0) | 1-(13Z,16Z-docosadienoyl)-2-docosanoyl-glycero-3- | 900.6 | 900.6688 | C₅₀H₉₅NO₁₀P | M + H |
| | | phosphoserine | | | | |

Note:
Chains containing double bonds and/or functional groups with defined regiochemistry, geometry and stereochemistry are meant to serve as examples for struclure-drawing purposes. In many cases there may be altemative isobaric structures.

And other m/z ions that were not easily identifiable but present in significant amounts.

On Another Range of Ratios:

Common to top, bottom layer and culture media:

m/z 821.2 corresponds to a fragment from peptide EREQIKSLNNQFASFIDKVR (SEQ ID NO. 12) and other peaks already identified such as 900.4, 900.5, etc (see chromatogram FIGS. 1G and 1H and following tables):

TABLE 25
Possible Lipid Structures

| LM_ID | Name | Systematic Name |
|---|---|---|
| LMGP03010724 | PS(22:0/22:4(7Z,10Z,13Z,16Z)) | 1-docosanoyl-2-(7Z,10Z,13Z,16Z-docosatetraenoyl)- |
| | | glycero-3-phosphoserine |
| LMGP03010784 | PS(22:2(13Z,16Z)/22:2(13Z,16Z)) | 1,2-di-(13Z,16Z-docosadienoyl)-sn-glycero-3- |
| | | phosphoserine |
| LMGP03010813 | PS(22:4(7Z,10Z,13Z,16Z)/22:0) | 1-(7Z,10Z,13Z,16Z-docosatetraenoyl)-2-docosanoyl- |
| | | glycero-3-phosphoserine |
| LMGP01011832 | PC(20:1(11Z)/22:2(13Z,16Z)) | 1-(11Z-eicosenoyl)-2-(13Z,16Z-docosadienoyl)- |
| | | glycero-3-phosphocholine |
| LMGP01011862 | PC(20:2(11Z,14Z)/22:1(11Z)) | 1-(11Z-14Z-eicosadienoyl)-2-(11Z-docosenoyl)- |
| | | glycero-3-phosphocholine |
| LMGP01011892 | PC(20:3(8Z,11Z,14Z)/22:0) | 1-(8Z,11Z,14Z-eicosatrienoyl)-2-docosanoyl-glycero-3- |
| | | phosphocholine |
| LMGP01011996 | PC(22:0/20:3(8Z,11Z,14Z)) | 1-docosanoyl-2-(8Z,11Z,14Z-eicosatrienoyl)-glycero-3- |
| | | phosphocholine |
| LMGP01012025 | PC(22:1(11Z)20:2(11Z,14Z)) | 1-(11Z-docosenoyl)-2-(11Z,14Z-eicosadienoyl)- |
| | | glycero-3-phosphocholine |
| LMGP01012055 | PC(22:2(13Z,16Z)/20:1(11Z)) | 1-(13Z,16Z-docosadienoyl)-2-(11Z-eicosenoyl)- |
| | | glycero-3-phosphocholine |
| LMGP03010527 | PS(20:0/22:4(7Z,10Z,13Z,16Z)) | 1-eicosanoyl-2-(7Z,10Z,13Z,16Z-docosatetraenoyl)- |
| | | glycero-3-phosphoserine |
| LMGP03010586 | PS(20:2(11Z,14Z)/22:2(13Z,16Z)) | 1-(11Z,14Z-eicosadienoyl-2-(13Z,16Z-docosadienoyl)- |
| | | glycero-3-phosphoserine |
| LMGP03010616 | PS(20:3(8Z,11Z,14Z)/22:1(11Z)) | 1-(8Z,11Z,14Z-eicosatrienoyl)-2-(11Z-docosenoyl)- |
| | | glycero-3-phosphoserine |
| LMGP03010644 | PS(20:4(5Z,8Z,11Z,14Z)/22:0) | 1-(5Z,8Z,11Z,14Z-eicosatetraenoyl)-2-docosanoyl)- |
| | | glycero-3-phosphoserine |
| LMGP03010719 | PS(22:0/20:4(5Z,8Z,11Z,14Z)) | 1-docosanoyl-2-(5Z,8Z,11Z,14Z-eicosatetraenoyl)- |
| | | glycero-3-phosphoserine |
| LMGP03010747 | PS(22:1(11Z)/20:3(8Z,11Z,14Z)) | 1-(11Z-docosenoyl)-2-(8Z,11Z,14Z-eicosatrienoyl)- |
| | | glycero-3-phosphoserine |
| LMGP03010777 | PS(22:2(13Z,16Z)/20:2(11Z,14Z)) | 1-(13Z,16Z-docosadienoyl)-2-(11Z,14Z-eicosadienoyl)- |
| | | glycero-3-phosphoserine |
| LMGP03010805 | PS(22:4(7Z,10Z,13Z,16Z)/20:0) | 1-(7Z,10Z,13Z,16Z-docosatetraenoyl)-2-eicosanoyl)- |
| | | glycero-3-phosphoserine |
| LMGP01010004 | PC(21:0/22:6(4Z,7Z,10Z,13Z,16Z,19Z)) | 1-henelcosanoyl-2-(4Z,7Z,10Z,13Z,16Z,19Z- |
| | | docosahexaenoyl)-sn-glycero-3-phosphocholine |
| LMGP01012121 | PC(22:6(4Z,7Z,10Z,13Z,16Z,19Z)/21:0) | 1-(4Z,7Z,10Z,13Z, 16Z,19Z-docosahexaenoyl)-2- |
| | | henelcosanoyl-glycero-3-phosphocholine |
| LMGP03010716 | PS(22:0/20:0) | 1-docosanoyl-2-elcosanoyl-glycero-3- |
| | | phosphoserine |
| LMGP03010851 | PS(21:0/21:0) | 1,2-dihenelcosanoyl-sn-glycero-3-phosphoserine |
| LMGP03010946 | PS(20:0/22:0) | 1-elcosanoyl-2-docasanoyl-glycero-3- |
| | | phosphoserine |
| LMGP01011119 | PC(22:6(4Z,7Z,10Z,13Z,16Z,19Z)/ | 1,2-di- |
| | 22:6(4Z,7Z,10Z,13Z,16Z,19Z)) | (4Z,7Z,10Z,13Z,16Z,19Z- |
| | | docosahexaenoyl)-sn- |
| | | glycero-3-phosphocholine |
| LMGP03010004 | PS(21:0/22:6(4Z,7Z,10Z,13Z,16Z,19Z)) | 1-henelcosanoyl-2- |
| | | (4Z,7Z,10Z,13Z,16Z,19Z- |
| | | docosahexaenoyl)-sn- |
| | | glycero-3-phosphoserine |
| LMGP03010841 | PS(22:6(4Z,7Z,10Z,13Z,16Z,19Z)/21:0) | 1- |
| | | (4Z,7Z,10Z,13Z,16Z,19Z- |
| | | docosahexaenoyl)-2- |
| | | henelcosanoyl-glycero-3- |
| | | phosphoserine |

| LM_ID | Input m/z | Exact m/z | Formula | Ion |
|---|---|---|---|---|
| LMGP03010724 | 896.5 | 896.6375 | C₅₀H₉₁NO₁₀P | M + H |
| LMGP03010784 | 896.5 | 896.6375 | C₅₀H₉₁NO₁₀P | M + H |
| LMGP03010813 | 896.5 | 895.6375 | C₅₀H₉₁NO₁₀P | M + H |
| LMGP01011832 | 868.5 | 868.6790 | C₅₀H₉₅NO₈P | M + H |
| LMGP01011862 | 868.5 | 868.6790 | C₅₀H₉₅NO₈P | M + H |
| LMGP01011892 | 868.5 | 868.6790 | C₅₀H₉₅NO₈P | M + H |
| LMGP01011996 | 868.5 | 868.6790 | C₅₀H₉₅NO₈P | M + H |
| LMGP01012025 | 868.5 | 868.6790 | C₅₀H₉₅NO₈P | M + H |
| LMGP01012055 | 868.5 | 868.6790 | C₅₀H₉₅NO₈P | M + H |
| LMGP03010527 | 868.5 | 868.6062 | C₁₈H₈₇NO₁₀P | M + H |
| LMGP03010586 | 868.5 | 868.6062 | C₄₈H₈₇NO₁₀P | M + H |
| LMGP03010616 | 868.5 | 868.6062 | C₄₈H₈₇NO₁₀P | M + H |
| LMGP03010644 | 868.5 | 868.6062 | C₄₈H₈₇NO₁₀P | M + H |
| LMGP03010719 | 868.5 | 868.6062 | C₄₈H₈₇NO₁₀P | M + H |
| LMGP03010747 | 868.5 | 868.6062 | C₄₈H₈₇NO₁₀P | M + H |
| LMGP03010777 | 868.5 | 868.6062 | C₄₈H₈₇NO₁₀P | M + H |
| LMGP03010805 | 868.5 | 868.6062 | C₄₈H₈₇NO₁₀P | M + H |
| LMGP01010004 | 876.5 | 876.6477 | C₅₁H₉₁NO₈P | M + H |
| LMGP01012121 | 876.5 | 876.6477 | C₅₁H₉₁NO₈P | M + H |
| LMGP03010716 | 876.5 | 876.6688 | C₁₀H₉₅NO₁₀P | M + H |
| LMGP03010851 | 876.5 | 876.6688 | C₁₀H₉₅NO₁₀P | M + H |
| LMGP03010946 | 876.5 | 876.6668 | C₄₈H₉₅NO₁₀P | M + H |
| LMGP01011119 | 878.4 | 876.5694 | C₅₂H₈₁NO₈P | M + H |
| LMGP03010004 | 878.4 | 678.5906 | C₄₉H₈₅NO₁₀P | M + H |
| LMGP03010841 | 878.4 | 878.5908 | C₄₀H₈₅NO₁₀P | M + H |

Note:
Chains containing double bonds and/or functional groups with defined regiochemistry, geometry and stereochemistry are meant to serve as examples for struclure-drawing purposes. In many cases there may be altemative isobaric structures.

### On Another Range of Ratios:

The peak common to all three samples was:
m/z 754.3

TABLE 26
Possible Lipid Structures

| | | | Input | Exact | | |
|---|---|---|---|---|---|---|
| LM_ID | Name | Systematic Name | m/z | m/z | Formula | Ion |
| LMGP03010131 | PS(14:1(9Z)/20:4(5Z,8Z,11Z,14Z)) | 1-(9Z-tetradecenoyl)-2-(5Z,8Z,11Z,14Z- | 754.3 | 754.4654 | C₄₀H₆₉NO₁₀P | M + H |
| | | elcosatetraenoyl)-glycero-3-phosphoserine | | | | |
| LMGP03010432 | PS(18:4(6Z,9Z,12Z,15Z)/16:1(9Z)) | 1-(6Z,9Z,12Z,15Z-octadecatetraenoyl)-2-(9Z- | 754.3 | 754.4654 | C₄₀H₆₉NO₁₀P | M + H |
| | | hexadecenoyl)-glycero-3-phosphoserine | | | | |
| LMGP03010623 | PS(20:4(5Z,8Z,11Z,14Z)/14:1(9Z)) | 1-(5Z,8Z,11Z,14Z-elcosatetraenoyl)-2-(9Z- | 754.3 | 754.4654 | C₄₀H₆₉NO₁₀P | M + H |
| | | tetradecenoyl)-glycero-3-phosphoserine | | | | |
| LMGP03010650 | PS(20:5(5Z,8Z,11Z,14Z,17Z)/14:0) | 1-(5Z,8Z,11Z,14Z,17Z-elcosapentaenoyl)-2- | 754.3 | 754.4654 | C₄₀H₆₉NO₁₀P | M + H |
| | | tetradecanoyl-glycero-3-phosphoserine | | | | |
| LMGP03010899 | PS(16:1(9Z)/18:4(6Z,9Z,12Z,15Z)) | 1-(9Z-hexadecenoyl)-2-(6Z,9Z,12Z,15Z- | 754.3 | 754.4654 | C₄₀H₆₉NO₁₀P | M + H |
| | | octadecatetraenoyl)-glycero-3-phosphoserine | | | | |
| LMGP03010922 | PS(14:0/20:5(5Z,8Z,11Z,14Z,17Z)) | 1-tetradecanoyl-2-(5Z,8Z,11Z,14Z,17Z- | 754.3 | 754.4654 | C₄₀H₆₉NO₁₀P | M + H |
| | | elcosapentaenoyl)-glycero-3-phosphoserine | | | | |

Note:
Chains containing double bonds and/or functional groups with defined regiochemistry, geometry and stereochemistry are meant to serve as examples for structure-drawing purposes. In many cases there may be alternative isobaric structures.

### And Common Between Supernatant and Top Layer:

TABLE 27
Possible Lipid Structures

| | | | Input | Exact | | |
|---|---|---|---|---|---|---|
| LM_ID | Name | Systematic Name | m/z | m/z | Formula | Ion |
| LMGL05010021 | MGDG(18:1(9Z)/18:1(9Z)) | 1,2 di-(9Z-octadecenoyl)-3-O-beta-D-galactosyl-sn- | 783.4 | 783.5981 | C₄₅H₈₃O₁₀ | M + H |
| | | glycerol | | | | |
| LMGL05010022 | MGDG(18:0(9Z)/ | 1-octadecanoyl-2-(9Z,12Z-octadecanoyl)-3-O-beta- | 783.4 | 783.5981 | C₄₅H₈₃O₁₀ | M + H |
| | 18:2(9Z,12Z)) | D-galactosyl-sn-glycerol | | | | |
| LMGP04010180 | PG(15:1(9Z)/ | 1-(9Z-pentadecenoyl)-2-(7Z,10Z,13Z,16Z- | 783.4 | 783.5171 | C₄₃H₇₆O₁₀P | M + H |
| | 22:4(7Z,10Z,13Z,16Z)) | docosatetraenoyl)-glycero-3-phospho-(1′-sn-glycerol) | | | | |
| LMGP04010242 | PG(17:0/ | 1-heptadecanoyl-2-(5Z,8Z,11Z,14Z,17Z- | 783.4 | 783.5171 | C₄₃H₇₆O₁₀P | M + H |
| | 20:5(5Z,8Z,11Z,14Z,17Z)) | elcosapentaenoyl)-glycero-3-phospho-(1′-sn-glycerol) | | | | |
| LMGP04010270 | PG(17:1(9Z)/ | 1-(9Z-heptadecenoyl)-2-(5Z,8Z,11Z,14Z- | 783.4 | 783.5171 | C₄₃H₇₆O₁₀P | M + H |
| | 20:4(5Z,8Z,11Z,14Z)) | elcosatetraenoyl)-glycero-3-phospho-(1′-sn-glycerol) | | | | |
| LMGP04010299 | PG(17:2(9Z,12Z)/ | 1-(9Z,12Z-heptadecadienoyl)-2-(8Z,11Z,14Z- | | | | |
| | 20:3(8Z,11Z,14Z)) | elcosatrienoyl)-glycero-3-phospho-(1′-sn-glycerol) | 783.4 | 783.5171 | C₄₃H₇₆O₁₀P | M + H |
| LMGP04010443 | PG(18:4(6Z,9Z,12Z,15Z)/ | 1-(6Z,9Z,12Z,15Z-octadecatetraenoyl)-2-(9Z- | 783.4 | 783.5171 | C₄₃H₇₆O₁₀P | M + H |
| | 19:1(9Z)) | nonadecenoyl)-glycero-3-phospho-(1′-sn-glycerol) | | | | |
| LMGP04010496 | PG(19:(9Z)/ | 1-(9Z-nonadecenoyl)-2-(6Z,9Z,12Z,15Z- | 783.4 | 783.5171 | C₄₃H₇₆O₁₀P | M + H |
| | 18:4(6Z,9Z,12Z,15Z)) | octadecatetraenoyl)-glycero-3-phospho-(1′-sn-glycerol) | | | | |
| LMGP04010599 | PG(20:3(8Z,11Z,14Z)/ | 1-(8Z,11Z,14Z-elcosatrienoyl)-2-(9Z,12Z- | 783.4 | 783.5171 | C₄₃H₇₆O₁₀P | M + H |
| | 17:2(9Z,12Z)) | heptadecadienoyl)-glycero-3-phospho-(1′-sn-glycerol) | | | | |
| LMGP04010628 | PG(20:4(5Z;8Z,11Z,14Z)/ | 1-(5Z,8Z,11Z,14Z-elcosatetraenoyl)-2-(9Z- | 783.4 | 783.5171 | C₄₃H₇₆O₁₀P | M + H |
| | 17:1(9Z)) | heptadecenoyl)-glycero-3-phospho-(1′-sn-glycerol) | | | | |
| LMGP04010656 | PG(20:5(5Z,8Z,11Z,14Z,17Z)/ | 1-(5Z,8Z,11Z,14Z,17Z-elcosapentaenoyl)-2- | 783.4 | 783.5171 | C₄₃H₇₆O₁₀P | M + H |
| | 17:0) | heptadecanoyl-glycero-3-phospho-(1′-sn-glycerol) | | | | |
| LMGP04010792 | PG(22:4(7Z,10Z,13Z,16Z)/ | 1-(7Z,10Z,13Z,16Z-docosatetraenoyl)-2-(9Z- | 783.4 | 783.5171 | C₄₃H₇₆O₁₀P | M + H |
| | 15:1(9Z)) | pentadecenoyl)-glycero-3-phospho-(1′-sn-glycerol) | | | | |
| LMGP04020085 | PG(O-18:0/ | 1-octadecyl-2-(5Z,8Z,11Z,14Z,17Z-elcosapenlaenoyl)- | 783.4 | 783.5534 | C₄₄H₈₀O₉P | M + H |
| | 20:5(5Z,8Z,11Z,14Z,17Z)) | glycero-3-phospho-(1′-sn-glycerol) | | | | |
| LMGP04030027 | PG(P-16:0/ | 1-(1Z-hexadecenyl)-2-(7Z,10Z,13Z,16Z- | 783.4 | 783.5534 | C₄₄H₈₀O₉P | M + H |
| | 22:4(7Z,10Z,13Z,16Z)) | docosatetraenoyl)-glycero-3-phospho-(1′-sn-glycerol) | | | | |
| LMGP04030072 | PG(P-20:0/ | 1-(1Z-elcosenyl)-2-(6Z,9Z,12Z,15Z- | 783.4 | 783.5534 | C₄₄H₈₀O₉P | M + H |
| | 18:4(6Z,9Z,12Z,15Z)) | octadecatetraenoyl)-glycero-3-phospho-(1′-sn-glycerol) | | | | |
| LMGP04030090 | PG(P-18:0/ | 1-(1Z-octadecenyl)-2-(5Z,8Z,11Z,14Z- | 783.4 | 783.5534 | C₄₄H₈₀O₉P | M + H |
| | 20:4(5Z,8Z,11Z,14Z)) | elcosatetraenoyl)-glycero-3-phospho-(1′-sn-glycerol) | | | | |
| LMGP06010860 | PI(18:0/12:0) | 1-octadecanoyl-2-dodecanoyl-glycero-3-phospho-(1′- | 783.4 | 783.5018 | C₃₉H₇₆O₁₃P | M + H |
| | | myo-inositol) | | | | |
| LMGP06010868 | PI(17:0/13:0) | 1-heptadecanoyl-2-tridecanoyl-glycero-3-phospho-(1′- | 763.4 | 783.5018 | C₃₉H₇₆O₁₃P | M + H |
| | | myo-inositol) | | | | |
| LMGP06010907 | PI(13:0/17:0) | 1-tridecanoyl-2-heptadecanoyl-glycero-3-phospho-(1′- | 783.4 | 783.5018 | C₃₉H₇₆O₁₃P | M + H |
| | | myo-inositol) | | | | |
| LMGP06010913 | PI(12:0/18:0) | 1-dodecanoyl-2-octadecanoyl-glycero-3-phospho-(1′- | 783.4 | 783.5018 | C₃₉H₇₆O₁₃P | M + H |
| | | myo-inositol) | | | | |
| LMGP06010945 | PI(16:0/14:0) | 1-hexadecanoyl-2-tetradecanoyl-glycero-3-phospho- | 783.4 | 783.5018 | C₃₉H₇₆O₁₃P | M + H |
| | | (1′-myo-inositol) | | | | |
| LMGP06010947 | PI(15:0/15:0) | 1,2-dipentadecanoyl-sn-glycero-3-phospho-(1′-myo- | 783.4 | 783.5018 | C₃₉H₇₆O₁₃P | M + H |
| | | inositol) | | | | |
| LMGP06010948 | PI(14:0/16:0) | 1-tetradecanoyl-2-hexadecanoyl-glycero-3-phospho- | 783.4 | 783.5018 | C₁₉H₇₆O₁₃P | M + H |
| | | (1′-myo-inositol) | | | | |
| LMGP06020006 | PI(O-16:0/15:0) | 1-hexadecyl-2-pentadecanoyl-glycero-3-phospho-(1′- | 783.4 | 783.5382 | C₄₀H₈₀O₁₂P | M + H |
| | | myo-inositol) | | | | |
| LMGP06020020 | PI(O-18:0/13:0) | 1-octadecyl-2-tridecanoyl-glcero-3-phospho-(1′-myo- | 783.4 | 783.5382 | C₄₀H₈₀O₁₂P | M + H |
| | | inositol) | | | | |
| LMGP10010553 | PA(20:1(11Z)/22:2(13Z,16Z)) | 1-(11Z,elcosenoyl)-2-(13Z,16Z-docosadienoyl)- | 783.4 | 783.5898 | C₄₅H₈₄O₈P | M + H |
| | | glycero-3-phosphate | | | | |
| LMGP10010583 | PA(20:2(11Z,14Z)/22:1(11Z)) | 1-(11Z,14Z-elcosadienoyl)-2-(11Z-docosenoyl)- | 783.4 | 783.5898 | C₄₅H₈₄O₈P | M + H |
| | | glycero-3-phosphate | | | | |
| LMGP10010613 | PA(20:3(8Z,11Z,14Z)/22:0) | 1-(8Z,11Z,14Z-elcosatrienoyl)-2-docosanoyl- | 783.4 | 783.5898 | C₄₅H₈₄O₈P | M + H |
| | | glycero-3-phosphate | | | | |
| LMGP10010717 | PA(22:0/20:3(8Z,11Z,14Z)) | 1-docosanoyl-2-(8Z,11Z,14Z-elcosaltrienoyl)- | 783.4 | 783.5898 | C₄₅H₈₄O₈P | M + H |
| | | glycero-3-phosphate | | | | |
| LMGP10010745 | PA(22:1(11Z)/20:2(11Z,14Z)) | 1-(11Z-docosenoyl)-2-(11Z,14Z-elcosadienoyl)- | 783.4 | 783.5898 | C₄₅H₈₄O₈P | M + H |
| | | glycero-3-phosphate | | | | |
| LMGP10010775 | PA(22:2(13Z,16Z)/20:1(11Z)) | 1-(13Z,16Z-docosadienoyl)-2-(11Z-elcosenoyl)- | 783.4 | 783.5898 | C₄₅H₈₄O₈P | M + H |
| | | glycero-3-phosphate | | | | |

Note:
Chains containing double bonds and/or functional groups with defined regiochemistry, geometry and stereochemistry are meant to serve as examples for structure-drawing purposes. In many cases there may be alternative isobaric structures.

### In Another Range:

m/z 652.3 was common to the supernatant and bottom layer

TABLE 28
Possible Lipid Structures

| | | | Input | Exact | | |
|---|---|---|---|---|---|---|
| LM_ID | Name | Systematic Name | m/z | m/z | Formula | Ion |
| LMGP03010931 | PS(14:0/12:0) | 1-tetradecanoyl-2-dodecanoyl-glycero-3-phosphoserine | 652.3 | 652.4184 | C₃₂H₈₃NO₁₀P | M + H |
| LMGP03010971 | PS(13.0/13:0) | 1,2-ditridecanoyl-sn-glycero-3-phosphoserine | 652.3 | 652.4184 | C₃₂H₈₃NO₁₀P | M + H |
| LMGP03010972 | PS(12:0/14:0) | 1-dodecanoyl-2-tetradecanoyl-glycero-3-phosphoserine | 652.3 | 652.4184 | C₃₂H₈₃NO₁₀P | M + H |
| LMST05010005 | 17alpha-(N-Acetyl-D- | 17-(N-Acetyl-D-glucosaminyl)-estra-1,3,5(10)-triene- | 652.3 | 652.2964 | C₃₂H₁₆NO₁₃ | M + H |
| | glucosaminyl)-estradiol 3-D- | 3,17alpha-diol 3-D-glucuronide | | | | |
| | glucuronide | | | | | |
| LMST05010027 | 17alpha-(N-acetyl-D- | 17alpha-(2-acetamido-2-deoxy-beta-D- | 652.3 | 652.2964 | C₃₂H₁₆NO₁₃ | M + H |
| | glucosaminyl)estradiol 3- | glucopyranosyloxy)estra-1,3,5(10)-trien-3-yl beta-D- | | | | |
| | glucosiduronic acid | glucopyranosiduronic acid | | | | |

Note:
Chains containing double bonds and/or functional groups with defined regiochemistry, geometry and stereochemistry are meant to serve as examples for structure-drawing purposes. In many cases there may be alternative isobaric structures.

m/z 637.1 was common to all three samples: from peptide

(SEQ ID NO. 13)
VTAAPQSVCALR

TABLE 29
LIST OF LYSO PHOSPHATIDYL CHOLINES:
Possible Lipid Structures

| | | | Input | Exact | | |
|---|---|---|---|---|---|---|
| LM_ID | Name | Systematic Name | m/z | m/z | Formula | Ion |
| LMGL02010309 | DG(14:0/0:0/14:0) (d5) | 1,3-ditetradecanoyl-sn-glycerol (d5) | 518.3 | 518.4822 | C₃₁H₅₆O₅ | M + H |
| LMGP01050038 | PC(18:3(9Z,12Z,15Z)/0.0) | 1-(9Z,12Z,15Z-octadecatrienoyl)-sn-glycero-3- | 518.3 | 518.3241 | C₂₆H₄₉NO₇P | M + H |
| | | phosphocholine | | | | |
| LMGP01050128 | PC(18:3(6Z,9Z,12Z)/0:0) | 1-(6Z,9Z,12Z-octadecatrienoyl)-glycero-3- | 518.3 | 518.3241 | C₂₆H₄₉NO₇P | M + H |
| | | phosphocholine | | | | |
| LMGP03050018 | PS(18:4(6Z,9Z,12Z,15Z)/0:0) | 1-(6Z,9Z,12Z,15Z-octadecatetraenoyl)-glycero-3- | 518.3 | 518.2513 | C₂₄H₄₁NO₉P | M + H |
| | | phosphoserine | | | | |
| LMGP01020046 | PC(O-16:0/2:0) | 1-hexadecyl-2-acetyl-sn-glycero-3-phosphocholine | 524.2 | 524.3711 | C₂₆H₅₅NO₇P | M + H |
| LMGP01050026 | PC(18:0/0:0) | 1-octadecanoyl-sn-glycero-3-phosphocholine | 524.2 | 524.3711 | C₂₆H₅₅NO₇P | M + H |
| LMGP01050076 | PC(0:0/18:0) | 2-octadecanoyl-sn-glycero-3-phosphocholine | 524.2 | 524.3711 | C₂₆H₅₅NO₇P | M + H |
| LMGP02010101 | PE(10:0/10:0) | 1,2-didecanoyl-sn-glycero-3-phosphoethanolamine | 524.2 | 524.3347 | C₂₅H₅₁NO₈P | M + H |
| LMGP02050026 | PE(21:0/0:0) | 1-heneicosanoyl-glycero-3-phosphoethanolamine | 524.2 | 524.3711 | C₂₆H₅₅NO₇P | M + H |
| LMGP03050001 | PS(18:1(9Z)/0:0) | 1-(9Z-octadecenoyl)-sn-glycero-3-phosphoserine | 524.2 | 524.2983 | C₂₄H₄₇NO₉P | M + H |
| LMGP03050017 | PS(18:3(6Z,9Z,12Z)/0:0) | 1-(6Z,9Z,12Z-octadecatrienoyl)-glycero-3-phosphoserine | 520.1 | 520.2670 | C₂₄H₄₃NO₉P | M + H |
| LMGP03050029 | PS(18:3(9Z,12Z,15Z)/0:0) | 1-(9Z,12Z,15Z-octadecatrienoyl)-glycero-3- | 520.1 | 520.2670 | C₂₄H₄₃NO₉P | M + H |
| | | phosphoserine | | | | |

Note:
Chains containing double bonds and/or functional groups with defined regiochemistry, geometry and stereochemistry are meant to serve as examples for structure-drawing purposes. In many cases there may be alternative isobaric structures.

TABLE 30
Other compounds eluting with the lysoPCs:
Possible Lipid Structures

| | | | Input | Exact | | |
|---|---|---|---|---|---|---|
| LM_ID | Name | Systematic Name | m/z | m/z | Formula | Ion |
| LMSP01050010 | Phytosphingosine-1-phospho- | 4R-hyroxyeicosasphinganine-1-phospho- | 560.2 | 560.3194 | C₂₄H₅₁NO₁₁P | M + H |
| | (1′-myo-inositol) | (1′-myo-inositol) | | | | |
| LMST03020536 | (6R)-vitamin D3 6,19-(4-phenyl-1,2,4- | (7E)—(3S,6R)-9,10-seco-5,7,10(19)- | 560.2 | 560.3847 | C₃₅H₅₀N₃O₃ | M + H |
| | triazoline-3,5-dione) adduct/(6R)- | cholestatrien-3-ol 6,19-(4-phenyl-1,2,4- | | | | |
| | cholecalciferol 6,19-(4-phenyl-1,2,4- | triazoline-3,5-dione) adduct | | | | |
| | triazoline-3,5-dione) adduct | | | | | |
| LMST03020537 | (6S)-vitamin D3 6,19-(4-phenyl- | (7E)—(3S,6S)-9,10-seco-5,7,10(19)- | 560.2 | 560.3847 | C₃₅H₅₀N₃O₃ | M + H |
| | 1,2,4-triazoline-3,5-dione) adduct/(6S)- | cholestatrien-3-ol 6,19-(4-phenyl-1,2,4- | | | | |
| | cholecalciferol 6,19-(4-phenyl-1,2,4- | triazoline-3,5-dionne) adduct | | | | |
| | triazoline-3,5-dione) adduct | | | | | |
| LMFA08020094 | N-docosahexaenoyl | N-(4Z,7Z,10Z,12E,16Z,19Z- | 476.2 | 476.3159 | C₃₁H₄₂NO₃ | M + H |
| | phenylalanine | docosahexaenoyl)-phenylalanine | | | | |
| LMGP02050017 | PE(18:3(6Z,9Z,12Z)/0:0) | 1-(6Z,9Z,12Z-octadecatrienoyl)-glycero-3- | 476.2 | 476.2772 | C₂₃H₄₃NO₇P | M + H |
| | | phosphoethanolamine | | | | |
| LMGP02050029 | PE(18:3(9Z,12Z,15Z)/0:0) | 1-(9Z,12Z,15Z-octadecatrienoyl)-glycero-3- | 476.2 | 476.2772 | C₂₃H₄₃NO₇P | M + H |
| | | phosphoethanolamine | | | | |

Note:
Chains containing double bonds and/or functional groups with defined regiochemistry, geometry and stereochemistry are meant to serve as examples for structure-drawing purposes. In many cases there may be alternative isobaric structures.

TABLE 31
LIST OF PHOSPHATIDYLCHOLINES:

| LM_ID | Name | Systematic Name | Input m/z | Exact m/z | Formula | Ion |
|---|---|---|---|---|---|---|
| LMGP01011357 | PC(13:0/20:5(5Z,8Z,11Z,14Z,17Z)) | 1-tridecanoyl-2-(5Z,8Z,11Z,14Z,17Z- | 738.4 | 738.5068 | C₄₁H₇₃NO₈P | M + H |
| | | eicosapentaenoyl)-glycero-3-phosphocholine | | | | |
| LMGP01011447 | PC(15:1(9Z)/18:4(6Z,9Z,12Z,15Z)) | 1-(9Z-pentadecenoyl)-2-(6Z,9Z,12Z,15Z- | 738.4 | 738.5068 | C₄₁H₇₃NO₈P | M + H |
| | | octadecatetraenoyl)-glycero-3-phosphocholine | | | | |
| LMGP01011705 | PC(18:4(6Z,9Z,12Z,15Z)/15:1(9Z)) | 1-(6Z,9Z,12Z,15Z-octadecatetraenoyl)-2-(9Z- | 738.4 | 738.5068 | C₄₁H₇₃NO₈P | M + H |
| | | pentadecenoyl)-glycero-3-phosphocholine | | | | |
| LMGP01011927 | PC(20:5(5Z,8Z,11Z,14Z,17Z)/13:0) | 1-(5Z,8Z,11Z,14Z,17Z-eicosapentaenoyl)- | 738.4 | 738.5068 | C₄₁H₇₃NO₈P | M + H |
| | | tridecanoyl-2-glycero-3-phosphocholine | | | | |
| LMGP01030033 | PC(P-16:0/18:4(6Z,9Z,12Z,15Z)) | 1-(1Z-hexadecenyl)-2-(6Z,9Z,12Z,15Z- | 738.4 | 738.5432 | C₄₂H₇₇NO₇P | M + H |
| | | octadecatetraenoyl)-glycero-3-phosphocholine | | | | |
| LMGP01090033 | PC(18:4(6Z,9Z,12Z,15Z)/P-16:0) | 1-(6Z,9Z,12Z,15Z-octadecatetraenoyl)-2-(1Z- | 738.4 | 738.5432 | C₄₂H₇₇NO₇P | M + H |
| | | hexadecenyl)-sn-glycero-3-phosphocholine | | | | |
| LMGP02010450 | PE(14:1(9Z)/22:4(7Z,10Z,13Z,16Z)) | 1-(9Z-tetradecenoyl)-2-(7Z,10Z,13Z,16Z- | 738.4 | 738.5068 | C₄₁H₇₃NO₈P | M + H |
| | | docosatetraenoyl)-glycero-3-phosphoethanolamine | | | | |
| LMGP02010644 | PE(18:1(9Z)/18:4(6Z,9Z,12Z,15Z)) | 1-(9Z-octadecenoyl)-2-(6Z,9Z,12Z,15Z- | 738.4 | 738.5068 | C₄₁H₇₃NO₈P | M + H |
| | | octadecatetraenoyl)-glycero-3-phosphoethanolamine | | | | |
| LMGP02010664 | PE(18:2(9Z,12Z)/18:3(6Z,9Z,12Z)) | 1-(9Z,12Z-octadecadienoyl)-2-(6Z,9Z,12Z- | 738.4 | 738.5068 | C₄₁H₇₃NO₈P | M + H |
| | | octadecatrienoyl)-glycero-3-phosphoethanolamine | | | | |
| LMGP02010665 | PE(18:2(9Z,12Z)/18:3(9Z,12Z,15Z)) | 1-(9Z,12Z-octadecadienoyl)-2-9Z,12Z,15Z- | 738.4 | 738.5068 | C₄₁H₇₃NO₈P | M + H |
| | | octadecatrienoyl)-glycero-3-phosphoethanolamine | | | | |
| LMGP02010692 | PE(18:3(6Z,9Z,12Z)/18:2(9Z,12Z)) | 1-(6Z,9Z,12Z-octadecatrienoyl)-2-(9Z,12Z,- | 738.4 | 738.5068 | C₄₁H₇₃NO₈P | M + H |
| | | octadecadienoyl)-glycero-3-phosphoethanolamine | | | | |
| LMGP02010721 | PE(18:3(9Z,12Z,15Z)/18:2(9Z,12Z)) | 1-(9Z,12Z,16Z-octadecatrienoyl)-2-(9Z,12Z- | 738.4 | 738.5068 | C₄₁H₇₃NO₈P | M + H |
| | | octadecadienoyl)-glycero-3-phosphoethanolamine | | | | |
| LMGP02010750 | PE(18:4(6Z,9Z,12Z,15Z)/18:1(9Z)) | 1-(6Z,9Z,12Z,15Z-octadecatetraenoyl)-2-(9Z- | 738.4 | 738.5068 | C₄₁H₇₃NO₈P | M + H |
| | | octadecenoyl)-glycero-3-phosphoethanolamine | | | | |
| LMGP02010939 | PE(20:4(5Z,8Z,11Z,14Z)/16:1(9Z)) | 1-(5Z,8Z,11Z,14Z-eicosatetraenoyl)-2-(9Z- | 738.4 | 738.5068 | C₄₁H₇₃NO₈P | M + H |
| | | hexadecenoyl)-glycero-3-phosphoethanolamine | | | | |
| LMGP02010968 | PE(20:5(5Z,8Z,11Z,14Z,17Z)/16:0) | 1-(5Z,8Z,11Z,14Z,17Z-eicosapentaenoyl)-2- | 738.4 | 738.5068 | C₄₁H₇₃NO₈P | M + H |
| | | hexadecanoyl)-glycero-3-phosphoethanolamine | | | | |
| LMGP02011105 | PE(22:4(7Z,10Z,13Z,16Z)/14:1(9Z)) | 1-(7Z,10Z,13Z,16Z-docosatetraenoyl)-2-(9Z- | 738.4 | 738.5068 | C₄₁H₇₃NO₈P | M + H |
| | | tetradecenoyl)-glycero-3-phosphoethanolamine | | | | |
| LMGP02011217 | PE(16:1(9Z)/20:4(5Z,8Z,11Z,14Z)) | 1-(9Z-hexadecenoyl)-2-(5Z,8Z,11Z,14Z- | 738.4 | 738.5068 | C₄₁H₇₃NO₈P | M + H |
| | | eicosatetraenoyl)-glycero-3-phosphoethanolamine | | | | |
| LMGP02011221 | PE(16:0/20:5(5Z,8Z,11Z,14Z,17Z)) | 1-hexadecanoyl-2-(5Z,8Z,11Z,14Z,17Z- | 738.4 | 738.5068 | C₄₁H₇₃NO₈P | M + H |
| | | eicosapentaenoyl)-glycero-3-phosphoethanolamine | | | | |
| LMGP01011357 | PC(13:0/20:5(5Z,8Z,11Z,14Z,17Z)) | 1-tridecanoyl-2-(5Z,8Z,11Z,14Z,17Z- | 738.6 | 738.5068 | C₄₁H₇₃NO₈P | M + H |
| | | eicosapentaenoyl)-glycero-3-phosphocholine | | | | |
| LMGP01011447 | PC(15:1(9Z)/18:4(6Z,9Z,12Z,15Z)) | 1-(9Z-pentadecenoyl)-2-(6Z,9Z,12Z,15Z- | 738.6 | 738.5068 | C₄₁H₇₃NO₈P | M + H |
| | | octadecatetraenoyl)-glycero-3-phosphocholine | | | | |
| LMGP01011705 | PC(18:4(6Z,9Z,12Z,15Z)/15:1(9Z)) | 1-(6Z,9Z,12Z,15Z-octadecatetraenoyl)-2-(9Z- | 738.6 | 738.5068 | C₄₁H₇₃NO₈P | M + H |
| | | pentadecenoyl)-glycero-3-phosphocholine | | | | |
| LMGP01011927 | PC(20:5(5Z,8Z,11Z,14Z,17Z)/13:0) | 1-(5Z,8Z,11Z,14Z,17Z-eicosapentaenoyl)-2- | 738.6 | 738.5068 | C₄₁H₇₃NO₈P | M + H |
| | | tridecanoyl-glycero-3-phosphocholine | | | | |
| LMGP01030033 | PC(P-16:0/18:4(6Z,9Z,12Z,15Z)) | 1-(1Z-hexadecenyl)-2-(6Z,9Z,12Z,15Z- | 738.6 | 738.5432 | C₄₂H₇₇NO₇P | M + H |
| | | octadecatetraenoyl)-glycero-3-phosphocholine | | | | |
| LMGP01090033 | PC(18:4(6Z,9Z,12Z,15Z)/P-16:0) | 1-(6Z,9Z,12Z,15Z-octadecatetraenoyl)-2-(1Z- | 738.6 | 738.5432 | C₄₂H₇₇NO₇P | M + H |
| | | hexadecenyl)-sn-glycero-3-phosphocholine | | | | |
| LMGP02010450 | PE(14:1(9Z)/22:4(7Z,10Z,13Z,16Z)) | 1-(9Z-tetradecenoyl)-2-(7Z,10Z,13Z,16Z- | 738.6 | 738.5068 | C₄₁H₇₃NO₈P | M + H |
| | | docosatetraenoyl)-glycero-3-phosphoethanolamine | | | | |
| LMGP02010644 | PE(18:1(9Z)/18:4(6Z,9Z,12Z,15Z)) | 1-(9Z-octadecenoyl)-2-(6Z,9Z,12Z,15Z- | 738.6 | 738.5068 | C₄₁H₇₃NO₈P | M + H |
| | | octadecatetraenoyl)-glycero-3-phosphoethanolamine | | | | |
| LMGP02010664 | PE(18:2(9Z,12Z)/18:3(6Z,9Z,12Z)) | 1-(9Z,12Z-octadecadienoyl)-2-(6Z,9Z,12Z- | 738.6 | 738.5068 | C₄₁H₇₃NO₈P | M + H |
| | | octadecatrienoyl)-glycero-3-phosphoethanolamine | | | | |
| LMGP02010665 | PE(18:2(9Z,12Z)/18:3(9Z,12Z,15Z)) | 1-(9Z,12Z-octadecadienoyl)-2-9Z,12Z,15Z- | 738.6 | 738.5068 | C₄₁H₇₃NO₈P | M + H |
| | | octadecatrienoyl)-glycero-3-phosphoethanolamine | | | | |
| LMGP02010692 | PE(18:3(6Z,9Z,12Z)/18:2(9Z,12Z)) | 1-(6Z,9Z,12Z-octadecatrienoyl)-2-(9Z,12Z,- | 738.6 | 738.5068 | C₄₁H₇₃NO₈P | M + H |
| | | octadecadienoyl)-glycero-3-phosphoethanolamine | | | | |
| LMGP02010721 | PE(18:3(9Z,12Z,15Z)/18:2(9Z,12Z)) | 1-(9Z,12Z,15Z-octadecatrienoyl)-2-(9Z,12Z- | 738.6 | 738.5068 | C₄₁H₇₃NO₈P | M + H |
| | | octadecadienoyl)-glycero-3-phosphoethanolamine | | | | |
| LMGP02010750 | PE(18:4(6Z,9Z,12Z,15Z)/18:1(9Z)) | 1-(6Z,9Z,12Z,15Z-octadecatetraenoyl)-2-(9Z- | 738.6 | 738.5068 | C₄₁H₇₃NO₈P | M + H |
| | | octadecenoyl)-glycero-3-phosphoethanolamine | | | | |
| LMGP02010939 | PE(20:4(5Z,8Z,11Z,14Z)/16:1(9Z)) | 1-(5Z,8Z,11Z,14Z-eicosatetraenoyl)-2-(9Z- | 738.6 | 738.5068 | C₄₁H₇₃NO₈P | M + H |
| | | hexadecenoyl)-glycero-3-phosphoethanolamine | | | | |
| LMGP02010968 | PE(20:5(5Z,8Z,11Z,14Z,17Z)/16:0) | 1-(5Z,8Z,11Z,14Z,17Z-eicosapentaenoyl)-2- | 738.6 | 738.5068 | C₄₁H₇₃NO₈P | M + H |
| | | hexadecanoyl)-glycero-3-phosphoethanolamine | | | | |
| LMGP02011105 | PE(22:4(7Z,10Z,13Z,16Z)/14:1(9Z)) | 1-(7Z,10Z,13Z,16Z-docosatetraenoyl)-2-(9Z- | 738.6 | 738.5068 | C₄₁H₇₃NO₈P | M + H |
| | | tetradecenoyl)-glycero-3-phosphoethanolamine | | | | |
| LMGP02011217 | PE(16:1(9Z)/20:4(5Z,8Z,11Z,14Z)) | 1-(9Z-hexadecenoyl)-2-(5Z,8Z,11Z,14Z- | 738.6 | 738.5068 | C₄₁H₇₃NO₈P | M + H |
| | | eicosatetraenoyl)-glycero-3-phosphoethanolamine | | | | |
| LMGP02011221 | PE(16:0/20:5(5Z,8Z,11Z,14Z,17Z)) | 1-hexadecanoyl-2-(5Z,8Z,11Z,14Z,17Z-eicosapentaenoyl)-glycero- | 738.6 | 738.5068 | C₄₁H₇₃NO₈P | M + H |
| | | 3-phosphoethanolamine | | | | |
| LMGP01010005 | PC(16:0/18:1(9Z)) | 1-hexadecanoyl-2-(9Z-octadecenoyl)-sn- | 760.4 | 760.5851 | C₄ [symbol] H [symbol] 3NO [symbol] P | M + H |
| | | glycero-3-phosphocholine | | | | |
| LMGP01010575 | PC(16:0/18:1(11E)) | 1-hexadecanoyl-2-(11E-octadecenoyl)-sn- | 760.4 | 760.5851 | C₄ [symbol] H [symbol] 3NO [symbol] P | M + H |
| | | glycero-3-phosphocholine | | | | |
| LMGP01010576 | PC(16:0/18:1(11Z)) | 1-hexadecanoyl-2-(11Z-octadecenoyl)-sn- | 760.4 | 760.5851 | C₄ [symbol] H [symbol] 3NO [symbol] P | M + H |
| | | glycero-3-phosphocholine | | | | |
| LMGP01010578 | PC(16:0/18:1(6E)) | 1-hexadecanoyl-2-(6E-octadecenoyl)-sn- | 760.4 | 760.5851 | C₄₂H [symbol] 3NO [symbol] P | M + H |
| | | glycero-3-phosphocholine | | | | |
| LMGP01010579 | PC(16:0/18:1(6Z)) | 1-hexadecanoyl-2-(6Z-octadecenoyl)-sn- | 760.4 | 760.5851 | C₄₂H [symbol] 3NO [symbol] P | M + H |
| | | glycero-3-phosphocholine | | | | |
| LMGP01010581 | PC(16:0/18:1(9E)) | 1-hexadecanoyl-2-(9E-octadecenoyl)-sn- | 760.4 | 760.5851 | C₄ [symbol] H [symbol] 3NO [symbol] P | M + H |
| | | glycero-3-phosphocholine | | | | |
| LMGP01010744 | PC(18:0/16:1(9Z)) | 1-octadecanoyl-2-(9Z-hexadecenoyl)-sn- | 760.4 | 760.5851 | C₄₂H₅₃NO [symbol] P | M + H |
| | | glycero-3-phosphocholine | | | | |
| LMGP01010884 | PC(18:1/(9Z)/16:0) | 1-(9Z-octadecenoyl)-2-hexadecanoyl-sn- | 760.4 | 760.5851 | C₄₂H₅₃NO [symbol] P | M + H |
| | | glycero-3-phosphocholine | | | | |
| LMGP01011335 | PC(12:0/22:1(11Z)) | 1-dodecanoyl-2-(11Z-docosenoyl)- | 760.4 | 760.5851 | C₄₂H [symbol] 3NO [symbol] P | M + H |
| | | glycero-3-phosphocholine | | | | |
| LMGP01011372 | PC(14:0/20:1(11Z)) | 1-tetradecanoyl-2-(11Z-eicosenoyl)- | 760.4 | 760.5851 | C₄₂H [symbol] NO [symbol] P | M + H |
| | | glycero-3-phosphocholine | | | | |
| LMGP01011397 | PC(14:1(9Z)/20:0) | 1-(9Z-tetradecenoyl)-2-eicosanoyl- | 760.4 | 760.5851 | C₄₂H [symbol] 3NO [symbol] P | M + H |
| | | glycero-3-phosphocholine | | | | |
| LMGP01011419 | PC(15:0/19:1(9Z)) | 1-pentadecanoyl-2-(9Z-nonadecenoyl)- | 760.4 | 760.5851 | C₄₂H [symbol] 3NO [symbol] P | M + H |
| | | glycero-3-phosphocholine | | | | |
| LMGP01011448 | PC(15:1(9Z)/19:0) | 1-(9Z-pentadecenoyl)-2-nonadecanoyl- | 760.4 | 760.5851 | C₄₂H [symbol] 3NO [symbol] P | M + H |
| | | glycero-3-phosphocholine | | | | |
| LMGP01011483 | PC(16:1(9Z)/18:0) | 1-(9Z-hexadecenoyl)-2-octadecanoyl- | 760.4 | 760.5851 | C₄₂H [symbol] 3NO [symbol] P | M + H |
| | | glycero-3-phosphocholine | | | | |
| LMGP01011503 | PC(17:0/17:1(9Z)) | 1-heptadecanoyl-2-(9Z-heptadecenoyl)- | 760.4 | 760.5851 | C₄ [symbol] H [symbol] 3NO [symbol] P | M + H |
| | | glycero-3-phosphocholine | | | | |
| LMGP01011529 | PC(17:1(9Z)/17:0) | 1-(9Z-heptadecenoyl)-2-heptadecanoyl- | 760.4 | 760.5851 | C₄₂H [symbol] 3NO [symbol] P | M + H |
| | | glycero-3-phosphocholine | | | | |
| LMGP01011732 | PC(19:0/15:1(9Z)) | 1-nonadecanoyl-2-(9Z-pentadecenoyl)- | 760.4 | 760.5851 | C₄₂H [symbol] 3NO [symbol] P | M + H |
| | | glycero-3-phosphocholine | | | | |
| LMGP01011760 | PC(19:1(9Z)/15:0) | 1-(9Z-nonadecenoyl)-2-pentadecanoyl- | 760.4 | 760.5851 | C₄₂H [symbol] 3NO [symbol] P | M + H |
| | | glycero-3-phosphocholine | | | | |
| LMGP01011767 | PC(20:0/14:1(9Z)) | 1-eicosanoyl-2-(9Z-tetradecenoyl)- | 760.4 | 760.5851 | C₄₂H [symbol] 3NO [symbol] P | M + H |
| | | glycero-3-phosphocholine | | | | |
| LMGP01011807 | PC(20:1(11Z)/14:0) | 1-(11Z-eicosenoyl)-2-tetradecanoyl- | 760.4 | 760.5851 | C₄₂H₅₃NO [symbol] P | M + H |
| | | glycero-3-phosphocholine | | | | |
| LMGP01012004 | PC(22:1(11Z)/12:0) | 1-(11Z-docosenoyl)-2-dodecanoyl- | 760.4 | 760.5851 | C₄₂H₅₃NO [symbol] P | M + H |
| | | glycero-3-phosphocholine | | | | |
| LMGP01012146 | PC(18:1(11Z)/16:0) | 1-(11Z-octadecenoyl)-2-hexadecanoyl-sn | 760.4 | 760.5851 | C₄₂H [symbol] 3NO [symbol] P | M + H |
| | | glycero-3-phosphocholine | | | | |
| LMGP02010470 | PE(15:0/22:1(11Z)) | 1-pentadecanoyl-2-(11Z-docosenoyl)- | 760.4 | 760.5851 | C₄₂H [symbol] 3NO [symbol] P | M + H |
| | | glycero-3-phosphoethanolamine | | | | |
| LMGP02010500 | PE(15:1(9Z)/22:0) | 1-(9Z-pentadecenoyl)-2-docosanoyl- | 760.4 | 760.5851 | C₄₂H [symbol] 3NO [symbol] P | M + H |
| | | glycero-3-phosphoethanolamine | | | | |
| LMGP02010534 | PE(16:1(9Z)/21:0) | 1-(9Z-hexadecenoyl)-2-heneicosanoyl- | 760.4 | 760.5851 | C₄₂H [symbol] 3NO [symbol] P | M + H |
| | | glycero-3-phosphoethanolamine | | | | |
| LMGP02010552 | PE(17:0/20:1(11Z)) | 1-heptadecanoyl-2-(11Z-eicosenoyl)- | 760.4 | 760.5851 | C₄₂H [symbol] 3NO [symbol] P | M + H |
| | | glycero-3-phosphoethanolamine | | | | |
| LMGP02010579 | PE(17:1(9Z)/20:0) | 1-(9Z-heptadecenoyl)-2-eicosanoyl- | 760.4 | 760.5851 | C₄₂H₅₃NO [symbol] P | M + H |
| | | glycero-3-phosphoethanolamine | | | | |
| LMGP02010531 | PE(18:0/19:1(9Z)) | 1-octadecanoyl-2-(9Z-nonadecenoyl)- | 760.4 | 760.5851 | C₄₂H [symbol] NO [symbol] P | M + H |
| | | glycero-3-phosphoethanolamine | | | | |
| LMGP02010645 | PE(18:1(9Z)/19:0) | 1-(9Z-octadecanoyl)-2-nonadecanoyl- | 760.4 | 760.5851 | C₄₂H₅₃NO [symbol] P | M + H |
| | | glycero-3-phosphoethanolamine | | | | |
| LMGP02010703 | PE(18:3(6Z,9Z,12Z)/20:5(5Z,8Z,11Z,14Z,17Z)) | 1-(6Z,9Z,12Z-octadecatrienoyl)-2- | 760.4 | 760.4912 | C₄₂H₇₁NO [symbol] P | M + H |
| | | (5Z,8Z,11Z,14Z,17Z-eicosapentaenoyl)- | | | | |
| | | glycero-3-phosphoethanolamine | | | | |
| LMGP02010731 | PE(18:3(9Z,12Z,15Z)/20:5(5Z,8Z,11Z,14Z,17Z)) | 1-(9Z,12Z,15Z-octadecatrienoyl)-2- | 760.4 | 760.4912 | C₄ [symbol] H₇₁NO [symbol] P | M + H |
| | | (5Z,8Z,11Z,14Z,17Z-eicosapentaenoyl)- | | | | |
| | | glycero-3-phosphoethanolamine | | | | |
| LMGP02010761 | PE(18:4(6Z,9Z,12Z)/20:4(5Z,8Z,11Z,14Z,14Z)) | 1-(6Z,9Z,12Z,15Z-octadecatetraenoyl)-2- | 760.4 | 760.4912 | C₄ [symbol] H₇₁NO [symbol] P | M + H |
| | | (5Z,8Z,11Z,14Z-eicosatetraenoyl)- | | | | |
| | | glycero-3-phosphoethanolamine | | | | |
| LMGP02010775 | PE(19:0/18:1(9Z)) | 1-nonadecanoyl-2-(9Z-octadecenoyl)- | 760.4 | 760.5851 | C₄₂H [symbol] 3NO [symbol] P | M + H |
| | | glycero-3-phosphoethanolamine | | | | |
| LMGP02010804 | PE(19:1(9Z)/18:0) | 1-(9Z-nonadecenoyl)-2-octadecanoyl- | 760.4 | 760.5851 | C₄₂H₅₃NO [symbol] P | M + H |
| | | glycero-3-phosphoethanolamine | | | | |
| LMGP02010829 | PE(20:0/17:1(9Z)) | 1-eicosanoyl-2-(9Z-heptadecenoyl) | 760.4 | 760.5851 | C₄₂H [symbol] NO [symbol] P | M + H |
| | | glycero-3-phosphoethanolamine | | | | |
| LMGP02010849 | PE(20:1(11Z)/17:0) | 1-(11Z-eicosenoyl)-2-heptadecanoyl | 760.4 | 760.5851 | C₄ [symbol] H [symbol] NO [symbol] P | M + H |
| | | glycero-3-phosphoethanolamine | | | | |
| LMGP02010948 | PE(20:4(5Z,8Z,11Z,14Z)/18:4(6Z,9Z,12Z,15Z,)) | 1-(5Z,8Z,11Z,14Z-eicosatetraenoyl)-2- | 760.4 | 760.4912 | C₄₂H₇₁NO [symbol] P | M + H |
| | | (6Z,9Z,12Z,15Z-octadecatetraenoyl)- | | | | |
| | | glycero-3-phosphoethanolamine | | | | |
| LMGP02010975 | PE(20:5(5Z,8Z,11Z,14Z,17Z)/18:3(6Z,9Z,12Z)) | 1-(5Z,8Z,11Z,14Z,17Z- | 760.4 | 760.4912 | C₄₂H₇₁NO [symbol] P | M + H |
| | | eicosapentaenoyl)-2-(6Z,9Z,12Z- | | | | |
| | | octadecatrienoyl)-glycero-3- | | | | |
| | | phosphoethanolamine | | | | |
| LMGP02010976 | PE(20:5(5Z,8Z,11Z,14Z,17Z)/18:3(9Z,12Z,15Z)) | 1-(5Z,8Z,11Z,14Z,17Z- | 760.4 | 760.4912 | C₄₃H₇₁NO [symbol] P | M + H |
| | | eicosapentaenoyl)-2-(9Z,12Z,15Z- | | | | |
| | | octadecatrienoyl)-glycero-3- | | | | |
| | | phosphoethanolamine | | | | |
| LMGP02010995 | PE(21:0/16:1(9Z)) | 1-heneicosanoyl-2-(9Z-hexadecenoyl)- | 760.4 | 760.5851 | C₄₂H₅₃NO [symbol] P | M + H |
| | | glycero-3-phosphoethanolamine | | | | |
| LMGP02011018 | PE(22:0/15:1(9Z)) | 1-docosanoyl-2-(9Z-pentadecenoyl)- | 760.4 | 760.5851 | C₄₂H₃₃NO [symbol] P | M + H |
| | | glycero-3-phosphoethanolamine | | | | |
| LMGP02011044 | PE(22:1(11Z)/15:0) | 1-(11Z-docosenoyl)-2-pentadecanoyl- | 760.4 | 760.5851 | C₄₂H [symbol] 3NO [symbol] P | M + H |
| | | glycero-3-phosphoethanolamine | | | | |
| LMGP02040017 | | 1-(8-(3)-ladderane-octanyl)-2-(8-(3)- | 760.4 | 760.5639 | C₄ [symbol] H [symbol] NO [symbol] P | M + H |
| | | ladderane-octanyl)-sn- | | | | |
| | | glycerophosphoethanolamine | | | | |
| LMGP03010066 | PS(12:0/22:2(13Z,16Z)) | 1-dodecanoyl-2-(13Z,16Z-docosadienoyl)- | 760.4 | 760.5123 | C₄ [symbol] H₇ [symbol] NO₁₀P | M + H |
| | | glycero-3-phosphoserine | | | | |
| LMGP03010103 | PS(14:0/20:2(11Z,14Z)) | 1-tetradecanoyl-2-(11Z,14Z- | 760.4 | 760.5123 | C₄ [symbol] H₇ [symbol] NO₁₀P | M + H |
| | | eicosadienoyl)-glycero-3-phosphoserine | | | | |
| LMGP03010128 | PS(14:1(9Z)/20:1(11Z)) | 1-(9Z-tetradecanoyl)-2-(11Z-eicosenoyl)- | 760.4 | 760.5123 | C₄ [symbol] H₇ [symbol] NO₁₀P | M + H |
| | | glycero-3-phosphoserine | | | | |
| LMGP03010179 | PS(15:1(9Z)/19:1(9Z)) | 1-(9Z-pentadecenoyl)-2-(9Z- | 760.4 | 760.5123 | C₄ [symbol] H₇ [symbol] NO₁ [symbol] P | M + H |
| | | nonadecenoyl)-glycero-3-phosphoserine | | | | |
| LMGP03010232 | PS(17:0/17:2(9Z,12Z)) | 1-heptadecanoyl-2-(9Z,12Z- | 760.4 | 760.5123 | C₄ [symbol] H₇ [symbol] NO₁ [symbol] P | M + H |
| | | heptadecadienoyl)-glycero-3- | | | | |
| | | phosphoserine | | | | |
| LMGP03010286 | PS(17:2/(9Z,12Z)/17:0) | 1-(9Z,12Z-heptadecadienoyl)-2- | 760.4 | 760.5123 | C₄ [symbol] H₇ [symbol] NO₁ [symbol] P | M + H |
| | | heptadecanoyl-glycero-3-phosphoserine | | | | |
| LMGP03010485 | PS(19:1/(9Z)/15:1(9Z)) | 1-(9Z-nonadecenoyl)-2-(9Z- | 760.4 | 760.5123 | C₄ [symbol] H₇ [symbol] NO₁ [symbol] P | M + H |
| | | pentadecenoyl)-glycero-3-phosphoserine | | | | |
| LMGP03010531 | PS(20:1(11Z)/14:1(9Z)) | 1-(11Z-eicosenoyl)-2-(9Z-tetradecenoyl)- | 760.4 | 760.5123 | C₄ [symbol] H₇ [symbol] NO₁ [symbol] P | M + H |
| | | glycero-3-phosphoserine | | | | |
| [symbol] MGP03010560 | PS(20:2(11Z,14Z)/14:0) | 1-(11Z,14Z-eicosadienoyl)-2- | 760.4 | 760.5123 | C₄ [symbol] H₇ [symbol] NO₁ [symbol] P | M + H |
| | | tetradecanoyl-glycero-3-phosphoserine | | | | |
| [symbol] MGP03010756 | PS(22:2(13Z,16Z)/12:0) | 1-(13Z,16Z-docosadienoyl)-2-dodecanoyl- | 760.4 | 760.5123 | C₄ [symbol] H₇ [symbol] NO₁₀P | M + H |
| | | glycero-3-phosphoserine | | | | |
| [symbol] MGP03010877 | PS(18:2(9Z,12Z)/16:0) | 1-(9Z,12Z-octadecadienoyl)-2- | 760.4 | 760.5123 | C₄ [symbol] H₇ [symbol] NO₁₀P | M + H |
| | | hexadecanoyl-glycero-3-phosphoserine | | | | |
| [symbol] MGP03010881 | PS(18:1(9Z)16:1(9Z)) | 1-(9Z-octadecenoyl)-2-(9Z- | 760.4 | 760.5123 | C₄ [symbol] H₇ [symbol] NO₁₀P | M + H |
| | | hexadecenoyl)-glycero-3-phosphoserine | | | | |
| [symbol] MGP03010891 | PS(17:1(9Z)17:1(9Z)) | 1,2-di-(9Z-heptadecenoyl)-sn-glycero-3- | 760.4 | 760.5123 | C₄₀H₇ [symbol] NO₁ [symbol] P | M + H |
| | | phosphoserine | | | | |
| [symbol] MGP03010901 | PS(16:1(9Z)/18:1(9Z)) | 1-(9Z-hexadecenoyl)-2-(9Z- | 760.4 | 760.5123 | C₄ [symbol] H₇ [symbol] NO₁ [symbol] P | M + H |
| | | octadecenoyl)-glycero-3-phosphoserine | | | | |
| [symbol] MGP03010976 | PS(16:0/18:2(9Z,12Z)) | 1-hexadecanoyl-2-(9Z-12Z- | 760.4 | 760.5123 | C₄ [symbol] H₇ [symbol] NO₁₀P | M + H |
| | | octadecadienoyl)-glycero-3-phosphoserine | | | | |
| [symbol] MGP03020026 | PS(O-18:0/17:2(9Z,12Z)) | 1-octadecyl-2-(9Z,12Z-heptadecadienoyl)-2 | 760.4 | 760.5487 | C₄₁H₇ [symbol] NO₁ [symbol] P | M + H |
| | | glycero-3-phosphoserine | | | | |
| [symbol] MGP03030017 | PS(P-16:0/19:1(9Z)) | 1-(1Z-hexadecenyl)-2-(9Z- | 760.4 | 760.5487 | C₄₁H₇ [symbol] NO₁ [symbol] P | M + H |
| | | nonadecenoyl)-glycero-3-phosphoserine | | | | |
| [symbol] MGP03030037 | PS(P-18:0/17:1(9Z)) | 1-(1Z-octadecenyl)-2-(9Z- | 760.4 | 760.5487 | C₄₁H₇ [symbol] NO₁ [symbol] P | M + H |
| | | heptadecenoyl)-glycero-3-phosphoserine | | | | |
| [symbol] MGP03030061 | PS(P-20:0/15:1(9Z)) | 1-(1Z-eicosenyl)-2-(9Z-pentadecenoyl)- | 760.4 | 760.5487 | C₄₁H₇ [symbol] NO₁ [symbol] P | M + H |
| | | glycero-3-phosphoserine | | | | |
| LMGP01010007 | PC(16:0/20:4(5Z,8Z,11Z,14Z)) | 1-hexadecanoyl-2-(5Z,8Z,11Z,14Z-eicosatetraenoyl)-sn- | 782.5 | 782.5874 | C [symbol] H [symbol] NO [symbol] P | M + H |
| | | glycero-3-phosphocholine | | | | |
| LMGP01010329 | PC(16:0/20:4(5E,8E,11E,14E)) | 1-hexadecanoyl-2-(5E,8E,11E,14E-eicosatetraenoyl)-sn- | 782.5 | 782.5874 | C [symbol] H [symbol] NO [symbol] P | M + H |
| | | glycero-3-phosphocholine | | | | |
| LMGP01010773 | PC(18:0/18:4(6Z,9Z,12Z,15Z)) | 1-octadecanoyl-2-(6Z,9Z,12Z,15Z-octadecatetraenoyl)-sn- | 782.5 | 782.5874 | C [symbol] H [symbol] NO [symbol] P | M + H |
| | | glycero-3-phosphocholine | | | | |
| LMGP01010774 | PC(18:0/18:4(9E,11E,13E,15E)) | 1-octadecanoyl-2-(9E,11E,13E,15E-octadecatetraenoyl)- | 782.5 | 782.5874 | C [symbol] H [symbol] NO [symbol] P | M + H |
| | | sn-glycero-3-phosphocholine | | | | |
| LMGP01010933 | PC(19:1(9Z)/18:3(9Z,12Z,15Z)) | 1-(9Z-octadecenoyl)-2-(9Z,12Z,15Z-octadecatraenoyl)-sn- | 782.5 | 782.5874 | C [symbol] H [symbol] NO [symbol] P | M + H |
| | | glycero-3-phosphocholine | | | | |
| LMGP01010721 | PC(18:2(2E,4E)/18:2(2E,4E)) | 1,2-di-(2E-4E-octadecadienoyl)-sn-glycero-3- | 782.5 | 782.5874 | C [symbol] H [symbol] NO [symbol] P | M + H |
| | | phosphocholine | | | | |
| LMGP01010924 | PC(18:2(2Z,4Z)/18:2(2Z,4Z)) | 1,2-di-(2Z-4Z-octadecadienoyl)-sn-glycero-3- | 782.5 | 782.5874 | C [symbol] H [symbol] NO [symbol] P | M + H |
| | | phosphocholine | | | | |
| LMGP01010927 | PC(18:2(6Z,9Z)/18:2(6Z,9Z)) | 1,2-di-(6Z-9Z-octadecadienoyl)-sn-glycero-3- | 782.5 | 782.5874 | C [symbol] H [symbol] NO [symbol] P | M + H |
| | | phosphocholine | | | | |
| LMGP01010930 | PC(19:2(9Z,11Z)/18:2(9Z,11Z)) | 1,2-di-(9Z-11Z-octadecadienoyl)-sn-glycero-3- | 782.5 | 782.5874 | C [symbol] H [symbol] NO [symbol] P | M + H |
| | | phosphocholine | | | | |
| LMGP01010937 | PC(18:2(9Z,12Z)/19:2(9Z,12Z)) | 1,2-di-(9Z-12Z-octadecadienoyl)-sn-glycero-3- | 782.5 | 782.5874 | C [symbol] H [symbol] NO [symbol] P | M + H |
| | | phosphocholine | | | | |
| LMGP01010955 | PC(18:3(9Z,12Z,15Z)/18:1(9Z)) | 1-(9Z,12Z,15Z-octadecatrienoyl)-2-(9Z-octadecenoyl)-sn- | 782.5 | 782.5874 | C [symbol] H [symbol] NO [symbol] P | M + H |
| | | glycero-3-phosphocholine | | | | |
| LMGP01011049 | PC(20:4(5Z,8Z,11Z,14Z)/18:0) | 1-(5Z,8Z,11Z,14Z-eicosatetraenoyl)-2-hexadecanoyl-sn- | 782.5 | 782.5874 | C [symbol] H [symbol] NO [symbol] P | M + H |
| | | glycero-3-phosphocholine | | | | |
| LMGP01011058 | PC(20:4(8E,11E,14E,17E)/18:0) | 1-(8E,11E,14E,17E-eicosatetraenoyl)-2-hexadecanoyl-sn- | 782.5 | 782.5874 | C [symbol] H [symbol] NO [symbol] P | M + H |
| | | glycero-3-phosphocholine | | | | |
| LMGP01011378 | PC(14:0/22:4(7Z,10Z,13Z,16Z)) | 1-tetradecanoyl-2-(7Z,10Z,13Z,18Z-docosatetraenoyl)- | 782.5 | 782.5874 | C [symbol] H [symbol] NO [symbol] P | M + H |
| | | glycero-3-phosphocholine | | | | |
| LMGP01011491 | PC(16:1(9Z)/20:3(8Z,11Z,14Z)) | 1-(9Z-hexadecenoyl)-2-(8Z,11Z,14Z-eicosatrienoyl)- | 782.5 | 782.5874 | C [symbol] H [symbol] NO [symbol] P | M + H |
| | | glycero-3-phosphocholine | | | | |
| LMGP01011603 | PC(18:1(9Z)/18:3(6Z,9Z,12Z)) | 1-(9Z-octadecenoyl)-2-(6Z,9Z,12Z-octadecatrienoyl)- | 782.5 | 782.5874 | C [symbol] H [symbol] NO [symbol] P | M + H |
| | | glycero-3-phosphocholine | | | | |
| LMGP01011852 | PC(18:3(6Z,9Z,12Z)/18:1(9Z)) | 1-(6Z,9Z,12Z-octadecatrienoyl)-2-(9Z-octadecenoyl)- | 782.5 | 782.5874 | C [symbol] H [symbol] NO [symbol] P | M + H |
| | | glycero-3-phosphocholine | | | | |
| LMGP01011711 | PC(18:4(6Z,9Z,12Z,15Z)/18:0) | 1-(8Z,9Z,12Z,15Z-octadecatetraenoyl)-2-octadecanoyl- | 782.5 | 782.5874 | C [symbol] H [symbol] NO [symbol] P | M + H |
| | | glycero-3-phosphocholine | | | | |
| LMGP01011873 | PC(20:3/(8Z,11Z,14Z)/18:1(9Z)) | 1-(8Z,11Z,14Z-eicosatrienoyl)-2-(9Z-hexadecenoyl)- | 782.5 | 782.5874 | C [symbol] H [symbol] NO [symbol] P | M + H |
| | | glycero-3-phosphocholine | | | | |
| LMGP01012038 | PC(22:4(7Z,10Z,13Z,15Z)/14:0) | 1-(7Z,10Z,13Z,18Z-docosatetraenoyl)-2-tetradecanoyl- | 782.5 | 782.5874 | C [symbol] H [symbol] NO [symbol] P | M + H |
| | | glycero-3-phosphocholine | | | | |
| LMGP01012138 | PC(16:0/20:4(8Z,11Z,14Z,17Z)) | 1-hexadecanoyl-2-(8Z,11Z,14Z,17Z-eicosatetraenoyl)-sn- | 782.5 | 782.5874 | C [symbol] H [symbol] NO [symbol] P | M + H |
| | | glycero-3-phosphocholine | | | | |
| LMGP01012139 | PC(18:1(9Z)/20:3(5Z,9Z,11Z)) | 1-(9Z-hexadecenoyl)-2-(5Z,8Z,11Z-eicosatrienoyl)-sn- | 782.5 | 782.5874 | C [symbol] H [symbol] NO [symbol] P | M + H |
| | | glycero-3-phosphocholine | | | | |
| LMGP01012150 | PC(18:1(11Z)/18:3(6Z,9Z,12Z)) | 1-(11Z-octadecenoyl)-2-(6Z,9Z,12Z-octadecatrienoyl)-sn- | 782.5 | 782.5874 | C [symbol] H [symbol] NO [symbol] P | M + H |
| | | glycero-3-phosphocholine | | | | |
| LMGP01012151 | PC(18:1(11Z)/18:3(6Z,9Z,15Z)) | 1-(11Z-octadecenoyl)-2-(9Z,12Z,15Z-octadecatrienoyl)-sn- | 782.5 | 782.5874 | C [symbol] H [symbol] NO [symbol] P | M + H |
| | | glycero-3-phosphocholine | | | | |
| LMGP01012174 | PC(18:3(6Z,9Z,12Z)/19:1(11Z)) | 1-(6Z,9Z,12Z-octadecatrienoyl)-2-(11Z-octadecenoyl)-sn- | 782.5 | 782.5874 | C [symbol] H [symbol] NO [symbol] P | M + H |
| | | glycero-3-phosphocholine | | | | |
| LMGP01012178 | PC(18:3(9Z,12Z,15Z)/19:1(11Z)) | 1-(9Z,12Z,15Z-octadecatrienoyl)-2-(11Z-octadecenoyl)-sn- | 782.5 | 782.5874 | C [symbol] H [symbol] NO [symbol] P | M + H |
| | | glycero-3-phosphocholine | | | | |
| LMGP01012195 | PC(20:3(5Z,8Z,11Z)/19:1(9Z)) | 1-(5Z,8Z,11Z-eicosatrienoyl)-2-(9Z-hexadecenoyl)-sn- | 782.5 | 782.5874 | C [symbol] H [symbol] NO [symbol] P | M + H |
| | | glycero-3-phosphocholine | | | | |
| LMGP01020081 | PC(O-17:0/20:4(5Z,8Z,11Z,14Z)) | 1-heptodecyl-2-(5Z,8Z,11Z,14Z-eicosatetraenoyl)-sn- | 782.5 | 782.8059 | C [symbol] H [symbol] NO [symbol] P | M + H |
| | | glycero-3-phosphocholine | | | | |
| LMGP02010318 | PE(17:2(9Z,12Z)/22:2(13Z,18Z)) | 1-(9Z,12Z-heptadecadenoyl)-2-(13Z,1dZ-docosenoyl)- | 782.5 | 782.5894 | C₄ [symbol] H₃₁NO [symbol] P | M + H |
| | | glycero-3-phosphocholine | | | | |
| LMGP02010783 | PE(19:4(8Z,9Z,12Z,15Z)/21:0) | 1-(8Z,9Z,12Z,15Z-octadecstetraenoyl)-2-hereicosanoyl- | 782.5 | 782.5894 | C₄ [symbol] H₃₁NO [symbol] P | M + H |
| | | glycero-3-phosphoethanolamine | | | | |
| LMGP02010785 | PE(19:0/20:4(5Z,8Z,11Z,14Z)) | 1-nonedecanoyl-2-(5Z,8Z,11Z,14Z-eicosetetranoyl)- | 782.5 | 782.5894 | C₄ [symbol] H₃₁NO [symbol] P | M + H |
| | | glycero-3-phosphoethanolamine | | | | |
| LMGP02010915 | PE(19:1(9Z)20:3(8Z,11Z,14Z)) | 1-(9Z-nonedecanoyl-2-(8Z,11Z,14Z-eicosatrienoyl)- | 782.5 | 782.5894 | C₄ [symbol] H₃₁NO [symbol] P | M + H |
| | | glycero-3-phosphoethanolamine | | | | |
| LMGP02010220 | PE(20:3(8Z,11Z,14Z)/19:1(9Z)) | 1-(8Z,11Z,14Z-eicosatrienoyl)-2-(9Z-nonedecanoyl- | 782.5 | 782.5894 | C₄ [symbol] H₃₁NO [symbol] P | M + H |
| | | glycero-3-phosphoethanolamine | | | | |
| LMGP02010749 | PE(20:4(5Z,8Z,11Z,14Z)/19:0) | 1-(5Z,8Z,11Z,14Z-eicosatrienoyl)-2-nonedecanoyl- | 782.5 | 782.5894 | C₄ [symbol] H₃₁NO [symbol] P | M + H |
| | | glycero-3-phosphoethanolamine | | | | |
| LMGP02011033 | PE(21:0/18:4(8Z,9Z,12Z,15Z)) | 1-heneicosanoyl-2-(8Z,9Z,12Z,15Z-octadecstetraenoyl)- | 782.5 | 782.5894 | C₄ [symbol] H₃₁NO [symbol] P | M + H |
| | | glycero-3-phosphoethanolamine | | | | |
| LMGP32011081 | PE(22:2(13Z,18Z)/17:2(8Z,12Z)) | 1-(13Z,18Z-docosadenoyl)-2-(9Z,12Z-heptadecdiencyl)- | 782.5 | 782.5894 | C₄ [symbol] H₃₁NO [symbol] P | M + H |
| | | glycero-3-phosphoethanolamine | | | | |
| LMGP02011110 | PE(22:4(7Z,10Z,13Z,18Z)/17:0) | 1-(7Z,10Z,13Z,18Z-docosatetraenoyl)-2-heptadecenoyl) | 782.5 | 782.5894 | C₄ [symbol] H₃₁NO [symbol] P | M + H |
| | | glycero-3-phosphoethanolamine | | | | |
| LMGP02020030 | PE(O-18:0/22:4(7Z,10Z,13Z,18Z)) | 1-octsdecyl-2-(7Z,10Z,13Z,18Z-docosatetraenoyl)- | 782.5 | 782.8358 | C₁₅H₃₅NO₇P | M + H |
| | | glycero-3-phosphoethanolamine | | | | |
| LMGP02020033 | PE(O-20:0/20:4(5Z,8Z,11Z,14Z)) | 1-eicosyl-2-(5Z,8Z,11Z,14Z-eicosatetraenoyl)glycero-3- | 782.5 | 782.8358 | C₁₅H₃₅NO₇P | M + H |
| | | phosphoethanolamine | | | | |
| LMGP02030034 | PE(P-30:0/20:3(8Z,11Z,14Z)) | 1-(1Z-eicosenyl)-2-(8Z,11Z,14Z-eicosatrienoyl)glycero-3- | 782.5 | 782.8358 | C₁₅H₃₅NO₇P | M + H |
| | | phosphoethanolamine | | | | |
| LMGP03310137 | PS(14:1(19Z)/22:4(7Z,10Z,13Z,18Z)) | 1-(9Z-tetradecenoyl)-2-(7Z,10Z,13Z,18Z- | 782.5 | 782.4987 | C [symbol] H₇₃NO₁₀P | M + H |
| | | docosatetraenoyl)glycero-3-phosphoserine | | | | |
| LMGP03010331 | PS(19:1(9Z)/18:4(6Z,9Z,12Z,15Z)) | 1-(9Z-octsdecenoyl)-2-(8Z,9Z,12Z,15Z- | 782.5 | 782.4987 | C [symbol] H₇₃NO₁₀P | M + H |
| | | octadecstetraenoyl)glycero-3-phosphoserine | | | | |
| LMGP03010351 | PS(19:2(9Z,12Z)/18:3(8Z,9Z,12Z)) | 1-(9Z,12Z-octedecstenocyl)-2-(8Z,9Z,12Z- | 782.5 | 782.4987 | C [symbol] H₇₃NO₁₀P | M + H |
| | | octadecstrienoyl)glycero-3-phosphoserine | | | | |
| LMGP03010352 | PS(19:2(9Z,12Z)/18:3(9Z,12Z,15Z)) | 1-(9Z,12Z-octedecstenocyl)-2-(9Z,12Z-15Z- | 782.5 | 782.4987 | C [symbol] H₇₃NO₁₀P | M + H |
| | | octadecatrienoyl)glycero-3-phosphoserine | | | | |
| LMGP03010379 | PS(18:3(8Z,9Z,12Z)/18:2(9Z,12Z)) | 1-(8Z,9Z,12Z-octedecstrienoyl)-2-(9Z,12Z- | 782.5 | 782.4987 | C [symbol] H₇₃NO₁₀P | M + H |
| | | octadecatrienoyl)glycero-3-phosphoserine | | | | |
| LMGP03010438 | PS(19:3(9Z,12Z,15Z)/18:2(9Z,12Z)) | 1-(9Z,12Z,15Z-octedecstrienoyl)-2-(9Z,12Z- | 782.5 | 782.4987 | C [symbol] H₇₃NO₁₀P | M + H |
| | | octadecadenoyl)glycero-3-phosphoserine | | | | |
| LMGP03010437 | PS(19:4(8Z,9Z,12Z,15Z)/18:1(8Z)) | 1-(8Z,9Z,12Z,15Z-octedecstetraenoyl)-2-(9Z- | 782.5 | 782.4987 | C [symbol] H₇₃NO₁₀P | M + H |
| | | octadecenoyl)glycero-3-phosphoserine | | | | |
| LMGP03010828 | PS(20:4(5Z,8Z,11Z,14Z)/18:1(8Z)) | 1-(5Z,9Z,11Z,14Z-eicosatetreanoyl)-2-(9Z-hexadecenoyl)- | 782.5 | 782.4987 | C [symbol] H₇₃NO₁₀P | M + H |
| | | glycero-3-phosphoserine | | | | |
| LMGP03010854 | PS(22:5(5Z,8Z,11Z,14Z,17Z)/18:0) | 1-(5Z,8Z,11Z,14Z,17Z-eicosapentsenoyl)-2-hexadecenoyl- | 782.5 | 782.4987 | C [symbol] H₇₃NO₁₀P | M + H |
| | | glycero-3-phosphoserine | | | | |
| LMGP03010790 | PS(22:4(7Z,10Z,13Z,18Z)/14:1(9Z)) | 1-(7Z,10Z,13Z,18Z-docosatetreanoyl)-3-(9Z- | 782.5 | 782.4987 | C [symbol] H₇₃NO₁₀P | M + H |
| | | tetratecenoyl)-glycero-3-phosphoserine | | | | |
| LMGP03310999 | PS(18:1(9Z)/20:4(5Z,8Z,11Z,14Z)) | 1-(8Z-hexadecenoyl)-2-(5Z,8Z,11Z,14Z-eicosatetraenoyl)- | 782.5 | 782.4987 | C [symbol] H₇₃NO₁₀P | M + H |
| | | glycero-3-phosphoserine | | | | |
| LMGP03010902 | PS(18:0/20:5(5Z,8Z,11Z,14Z,17Z)) | 1-hexadecanoyl-2-(5Z,8Z,11Z,14Z,17Z-eicosspentraenoyl)- | 782.5 | 782.4987 | C [symbol] H₇₃NO₁₀P | M + H |
| | | glycero-3-phosphoserine | | | | |
| LMSP03033003 | PI-Cer(d19:0/15:0) | N-(hexadecanoyl)-sphingaine-1-phospho-(1′-myo-inositol) | 782.5 | 782.5542 | C₄₅H₃₁NO₁₁P | M + H |
| LMSP0501AA37 | GlcCer(d18:2/22:0) | N-(docosanoyl)-1beta-glucosyl-1E,14Z-sphingaderine | 782.5 | 782.6504 | C₄₅H₅₃NO₃ | M + H |
| LMSP0501AA84 | GlcCer(d18:2(4E,8E)/24:0) | N-(tetrscosanoyl)-1beta-glucosyl-1E,8E- | 782.5 | 782.6504 | C₄₅H₅₃NO₃ | M + H |
| | | hexadecesphingadierine | | | | |
| LMSP0501AC22 | GlcCer(d18:2/22:0) | N-(docosanoyl)-1beta-galactosyl-1E,14Z-sphingaderine | 782.5 | 782.6504 | C₄₅H₅₃NO₃ | M + H |

Possible Lipid Structures

| | | | | | | |
|---|---|---|---|---|---|---|
| LMGP00000048 | PT(18:0/18:1(9Z) | 1-octadecanoyl-2-(9Z-octadecanoyl)-sn-glycero- | 834.4 | 834.5749 | C₂₃H₃₃NO₁₀P | M + H |
| | | 3-phosphothreonine | | | | |
| LMGP01010693 | PC(18:1(9Z)/22:8(4Z,7Z,10Z,13Z,18Z,19Z)) | 1-(9Z-hexadecanoyl)-2- | 834.4 | 834.5538 | C₂₅H₇₉NO₅P | M + H |
| | | (4Z,7Z,10Z,13Z,18Z,19Z-docosahexsenoyl)-sn- | | | | |
| | | glycero-3-phosphocholine | | | | |
| LMGP01011308 | PC(18:1(7Z)/22:8(4Z,7Z,10Z,13Z,18Z,19Z)) | 1-(7Z-hexadecanoyl)-2- | 834.4 | 834.5538 | C₂₅H₇₅NO₅P | M + H |
| | | (4Z,7Z,10Z,13Z,18Z,19Z-docosahexsenoyl)-sn- | | | | |
| | | glycero-3-phosphocholine | | | | |
| LMGP01011834 | PC(18:2(9Z,12Z)/20:5(5Z,8Z,11Z,14Z,17Z)) | 1-(9Z,12Z-octadecadienoyl)-2- | 834.4 | 834.5538 | C₂₅H₇₉NO₅P | M + H |
| | | (5Z,8Z,11Z,14Z,17Z,-eicosapentaenoyl)- | | | | |
| | | glycero-3-phosphocholine | | | | |
| LMGP01011883 | PC(18:3(8Z,9Z,12Z)/20:4(5Z,8Z,11Z,14Z)) | 1-(8Z,9Z,12Z-octadecatrienoyl)-2- | 834.4 | 834.5538 | C₂₅H₇₉NO₅P | M + H |
| | | (5Z,8Z,11Z,14Z-eicosatetraenoyl)-glycero-3- | | | | |
| | | phosphocholine | | | | |
| LMGP01011892 | PC(19:3(9Z,12Z,15Z)/20:4(5Z,8Z,11Z,14Z)) | 1-(9Z,12Z,15Z-octadecatrienoyl)-2- | 834.4 | 834.5538 | C₂₈H₇₉NO₅P | M + H |
| | | (5Z,8Z,11Z,14Z-eicosatetraenoyl)-glycero-3- | | | | |
| | | phosphocholine | | | | |
| LMGP01011722 | PC(19:4(6Z,9Z,12Z,15Z)/20:3(8Z,11Z,14Z)) | 1-(6Z,9Z,12Z,15Z-octadecatetraenoyl)-2- | 834.4 | 834.5538 | C₂₅H₇₉NO₅P | M + H |
| | | (8Z,11Z,14Z-eicosatrienoyl)-glycero-3- | | | | |
| | | phosphocholine | | | | |
| LMGP01011892 | PC(20:3(8Z,11Z,14Z)/19:4(8Z,9Z,12Z,15Z)) | 1-(8Z,11Z,14Z-eicosatrienoyl)-2- | 834.4 | 834.5538 | C₂₅H₇₉NO₅P | M + H |
| | | (8Z,9Z,12Z,15Z-octadecatetraenoyl)-glycero-3- | | | | |
| | | phosphocholine | | | | |
| LMGP01011910 | PC(20:4(5Z,8Z,11Z,14Z)/19:3(8Z,9Z,12Z)) | 1-(5Z,8Z,11Z,14Z-eicosatetraenoyl)-2- | 834.4 | 834.5538 | C₂₅H₇₉NO₅P | M + H |
| | | (8Z,9Z,12Z-octadecatrienoyl)-glycero-3- | | | | |
| | | phosphocholine | | | | |
| LMGP01011911 | PC(20:4(5Z,8Z,11Z,14Z)/19:3(9Z,12Z,15Z)) | 1-(5Z,8Z,11Z,14Z-eicosatetraenoyl)-2- | 834.4 | 834.5538 | C₂₅H₇₉NO₅P | M + H |
| | | (9Z,12Z,15Z-octadecatrienoyl)-glycero-3- | | | | |
| | | phosphocholine | | | | |
| LMGP01011939 | PC(20:5(5Z,8Z,11Z,14Z,17Z)/19:2(9Z,12Z)) | 1-(5Z,8Z,11Z,14Z,17Z-eicosapentaenoyl)-2- | 834.4 | 834.5538 | C₂₅H₇₉NO₅P | M + H |
| | | (9Z,12Z-octadecadienoyl)-glycero-3- | | | | |
| | | phosphocholine | | | | |
| LMGP01012103 | PC(22:8(4Z,7Z,10Z,13Z,18Z,19Z)/18:1(9Z)) | 1-(4Z,7Z,10Z,13Z,18Z,19Z- | 834.4 | 834.5538 | C₂₅H₇₉NO₅P | M + H |
| | | docosahexsenoyl)-2-(9Z-hexadecenoyl)- | | | | |
| | | glycero-3-phosphocholine | | | | |
| LMGP02010923 | PE(19:1(8Z)/22:6(4Z,7Z,10Z,13Z,18Z,19Z)) | 1-(9Z-nonadecenoyl)-2- | 834.4 | 834.5538 | C₂₅H₇₉NO₅P | M + H |
| | | (4Z,7Z,10Z-13Z,18Z,19Z-docosahexsenoyl)- | | | | |
| | | glycero-3-phosphoethanolamine | | | | |
| LMGP02011149 | PE(22:8(4Z,7Z,10Z,13Z,18Z,19Z)/19:1(9Z)) | 1-(4Z,7Z,10Z,13Z,18Z,19Z- | 834.4 | 834.5538 | C₂₅H₇₉NO₅P | M + H |
| | | docosahexsenoyl)-2-(9Z-nonadecenoyl)- | | | | |
| | | glycero-3-phosphoethanolamine | | | | |
| LMGP02030092 | PE(20:0/22:6(4Z,7Z,10Z,13Z,18Z,19Z)) | 1-(1Z-eicosenyl)-2-(4Z,7Z,10Z,13Z,18Z,19Z- | 834.4 | 834.5902 | C₂₇H₃₃NO₇P | M + H |
| | | docosahexsenoyl)-glycero-3- | | | | |
| | | phosphoethanolamine | | | | |
| LMGP03010157 | PS(15:0)/22:1(11Z) | 1-pentadecanoyl-2-(11Z-docosenoyl)-glycero-3- | 834.4 | 834.5749 | C₂₃H₃₃NO₁₀P | M + H |
| | | phosphoserine | | | | |
| LMGP03010187 | PS(15:1(9Z)/22:0) | 1-(9Z-pentadecanoyl)-2-docosanoyl-glycero-3- | 834.4 | 834.5749 | C₂₃H₃₃NO₁₀P | M + H |
| | | phosphoserine | | | | |
| LMGP03010221 | PS(16:1(9Z)/22:0) | 1-(9Z-hexadecanoyl)-2-heneicosanoyl-glycero-3- | 834.4 | 834.5749 | C₂₃H₃₃NO₁₀P | M + H |
| | | phosphoserine | | | | |
| LMGP03010239 | PS(17:0/20:1(11Z)) | 1-heptadecanoyl-2-(11Z-eicosenoyl)-glycero-3- | 834.4 | 834.5749 | C₂₃H₃₃NO₁₀P | M + H |
| | | phosphoserine | | | | |
| LMGP03010266 | PS(17:1(9Z)/20:0) | 1-(9Z-heptadecenoyl)-2-eicosenoyl-glycero-3- | 834.4 | 834.5749 | C₂₃H₃₃NO₁₀P | M + H |
| | | phosphoserine | | | | |
| LMGP03010318 | PS(18:0/19:1(9Z)) | 1-octadecanoyl-2-(8Z-nonadecenoyl)-glycero-3- | 834.4 | 834.5749 | C₂₃H₃₃NO₁₀P | M + H |
| | | phosphoserine | | | | |
| LMGP03010332 | PS(19:1(9Z)/19:0) | 1-(9Z-octadecanoyl)-2-nonadecanoyl)-glycero-3- | 834.4 | 834.5749 | C₂₃H₃₃NO₁₀P | M + H |
| | | phosphoserine | | | | |
| LMGP03310390 | PS(19:3(8Z,9Z,12Z)/20:5(5Z,8Z,11Z,14Z,17Z)) | 1-(8Z,9Z,12Z-octadecatrienoyl)-2- | 834.4 | 834.4810 | C₄₃H₇₁NO₁₀P | M + H |
| | | (5Z,8Z,11Z,14Z,17Z-eicosapentaenoyl)- | | | | |
| | | glycero-3-phosphoserine | | | | |
| LMGP03310418 | PS(18:3(9Z,12Z,15Z)/20:5(5Z,8Z,11Z,14Z,17Z)) | 1-(9Z,12Z,15Z-octadecatrienoyl)-2- | 834.4 | 834.4810 | C₄₃H₇₁NO₁₀P | M + H |
| | | (5Z,8Z,11Z,14Z,17Z-eicosapentaenoyl)- | | | | |
| | | glycero-3-phosphoserine | | | | |
| LMGP03310449 | PS(19:4(6Z,9Z,12Z,15Z)/20:4(5Z,8Z,11Z,14Z)) | 1-(6Z,9Z,12Z,15Z-octadecatetraenoyl)-2- | 834.4 | 834.4810 | C₄₃H₇₁NO₁₀P | M + H |
| | | (5Z,8Z,11Z,14Z-eicostetraenoyl)-glycero-3- | | | | |
| | | phosphoserine | | | | |
| LMGP03010482 | PS(18:0/19:1(9Z)) | 1-nonadecanoyl-2-(9Z-octadecenoyl)-glycero-3- | 834.4 | 834.5749 | C₄₃H₃₃NO₁₀P | M + H |
| | | phosphoserine | | | | |
| LMGP03010491 | PS(19:1(9Z)/19:0) | 1-(9Z-nonadecenoyl)-2-octadecenoyl-glycero-3- | 834.4 | 834.5749 | C₄₃H₃₃NO₁₀P | M + H |
| | | phosphoserine | | | | |
| LMGP03010516 | PS(20:0/17:1(9Z)) | 1-eicosanoyl-2-(9Z-heptadecenoyl)-glycero-3- | 834.4 | 834.5749 | C₄₃H₃₃NO₁₀P | M + H |
| | | phosphoserine | | | | |
| LMGP03010538 | PS(20:1(11Z)/17:0) | 1-(11Z-eicosanoyl)-2-heptadecanoyl-glycero-3- | 834.4 | 834.5749 | C₄₃H₃₃NO₁₀P | M + H |
| | | phosphoserine | | | | |
| LMGP03310835 | PS(20:4(5Z,8Z,11Z,14Z)/18:4(8Z,11Z,12Z,15Z)) | 1-(5Z,8Z,11Z,14Z-eicosatetraenoyl)-2- | 834.4 | 834.4810 | C₄₃H₇₁NO₁₀P | M + H |
| | | (8Z,9Z,12Z,15Z-octadecatetraenoyl)-glycero-3- | | | | |
| | | phosphoserine | | | | |
| LMGP03310881 | PS(20:5(5Z,8Z,11Z,14Z,17Z)/19:3(8Z,9Z,12Z)) | 1-(5Z,8Z,11Z,14Z,17Z-eicosapentaenoyl)-2- | 834.4 | 834.4810 | C₄₃H₇₁NO₁₀P | M + H |
| | | (8Z,9Z,12Z-octadecatrienoyl)-glycero-3- | | | | |
| | | phosphoserine | | | | |
| LMGP03310882 | PS(20:5(5Z,8Z,11Z,14Z,17Z)/18:3(9Z,12Z,15Z)) | 1-(5Z,8Z,11Z,14Z,17Z-eicosapentaenoyl)-2- | 834.4 | 834.4810 | C₄₃H₇₁NO₁₀P | M + H |
| | | (9Z,12Z,15Z-octadecatrienoyl)-glycero-3- | | | | |
| | | phosphoserine | | | | |
| LMGP03310881 | PS(21:0/18:1(9Z)) | 1-heneicosanoyl-2-(9Z-hexadecenoyl)-glycero- | 834.4 | 834.5749 | C₄₃H₃₃NO₁₀P | M + H |
| | | 3-phosphoserine | | | | |
| LMGP03310704 | PS(22:0/15:1(9Z)) | 1-docosanoyl-2-(9Z-pentadecenoyl)-glycero-3- | 834.4 | 834.5749 | C₄₃H₃₃NO₁₀P | M + H |
| | | 3-phosphoserine | | | | |
| LMGP03310729 | PS(22:1(11Z)/15:0) | 1-(11Z-docosanoyl-2-pentadecanoyl)-glycero-3- | 834.4 | 834.5749 | C₄₃H₃₃NO₁₀P | M + H |
| | | 3-phosphoserine | | | | |
| LMGP01010512 | PC(14:0/22:8(4Z,7Z,10Z,13Z,18Z,19Z)) | 1-tetradecanoyl-2-(4Z,7Z,10Z,13Z,18Z,19Z- | 778.6 | 778.5381 | C₄₃H₇₇NO₃P | M + H |
| | | docosahexenoyl)-sn-glycero-3-phosphocholine | | | | |
| LMGP01010952 | PC(19:3(9E,11E,13E)/19:3(9E,11E,13E)) | 1,2-di-(9E,11E,13E-octadecatrienoyl)-sn-glycero-3- | 778.6 | 778.5381 | C₄₃H₇₇NO₃P | M + H |
| | | phosphocholine | | | | |
| LMGP01010954 | PC(18:3(9Z,11E,13E)/19:3(9Z,11E,13E)) | 1,2-di-(9Z,11E,13E-octadecatrienoyl)-sn-glycero-3- | 778.6 | 778.5381 | C₄₃H₇₇NO₃P | M + H |
| | | phosphocholine | | | | |
| LMGP01010258 | PC(18:3(9Z,12Z,15Z)/19:3(9Z,12Z,15Z)) | 1,2-di-(9Z,12Z,15Z-octadecatrienoyl)-sn-glycero-3- | 778.6 | 778.5381 | C₄₃H₇₇NO₃P | M + H |
| | | phosphocholine | | | | |
| LMGP01010260 | PC(19:4(9E,11E,13E,15E)/18:2(9Z,12Z)) | 1-(9E,11E,13E,15E-octadecatetraenoyl)-2(9Z,12Z- | 778.6 | 778.5381 | C₄₃H₇₇NO₃P | M + H |
| | | octadecadienoyl)-sn-glycero-3-phosphocholine | | | | |
| LMGP01011492 | PC(18:1(9Z)/20:5(5Z,8Z,11Z,14Z,17Z)) | 1-(9Z-hexadecenoyl)-2-(5Z,8Z,11Z,14Z,17Z- | 778.6 | 778.5381 | C₄₃H₇₇NO₃P | M + H |
| | | eicosapentaenoyl)-glycero-3-phosphocholine | | | | |
| LMGP01011827 | PC(18:2(9Z,12Z)/19:4(8Z,9Z,12Z,15Z)) | 1-(9Z,12Z-octadecadienoyl)-2-(8Z,9Z,12Z,15Z- | 778.6 | 778.5381 | C₄₃H₇₇NO₃P | M + H |
| | | octadecatetraenoyl)-glycero-3-phosphocholine | | | | |
| LMGP01011654 | PC(18:3(6Z,9Z,12Z)/18:3(8Z,9Z,12Z)) | 1,2-di-(6Z,9Z,12Z-octadecatrienoyl)-sn-glycero-3- | 778.6 | 778.5381 | C₄₃H₇₇NO₃P | M + H |
| | | phosphocholine | | | | |
| LMGP01011655 | PC(19:3(6Z,9Z,12Z)/18:3(9Z,12Z,15Z)) | 1-(6Z,9Z,12Z-octadecatrienoyl)-2-(9Z,12Z,15Z- | 778.6 | 778.5381 | C₄₃H₇₇NO₃P | M + H |
| | | octadecatrienoyl)-glycero-3-phosphocholine | | | | |
| LMGP01011694 | PC(19:3(9Z,12Z,15Z)/15:3(8Z,9Z,12Z)) | 1-(9Z,12Z,15Z-octadecatrienoyl)-2-(9Z,8Z,12Z- | 778.6 | 778.5381 | C₄₃H₇₇NO₃P | M + H |
| | | octadecatrienoyl)-glycero-3-phosphocholine | | | | |
| LMGP01011713 | PC(19:4(6Z,9Z,12Z,15Z)/18:2(9Z,12Z)) | 1-(6Z,9Z,12Z,15Z-octadecatrienoyl)-2-(9Z,12Z- | 778.6 | 778.5381 | C₄₃H₇₇NO₃P | M + H |
| | | octadecatrienoyl)-glycero-3-phosphocholine | | | | |
| LMGP01011933 | PC(20:5(5Z,8Z,11Z,14Z,17Z)/18:1(9Z)) | 1-(5Z,8Z,11Z,14Z,17Z-eicosapentaenoyl)-2-(9Z- | 778.6 | 778.5381 | C₄₃H₇₇NO₃P | M + H |
| | | hexadecenoyl)-glycero-3-phosphocholine | | | | |
| LMGP01012099 | PC(20:8(4Z,7Z,10Z,13Z,18Z,19Z)/14:0) | 1-(4Z,7Z,10Z,13Z,18Z,19Z-docosahexaenoyl)-2- | 778.6 | 778.5381 | C₄₃H₇₇NO₃P | M + H |
| | | tetradecanoyl)-glycero-3-phosphocholine | | | | |
| LMGP01012134 | PC(14:1(9Z)/22:5(4Z,7Z,10Z,13Z,18Z)) | 1-(9Z-tetradecenoyl)-2-(4Z,7Z,10Z,13Z,18Z | 778.6 | 778.5381 | C₄₃H₇₇NO₃P | M + H |
| | | docosapentaenoyl)-sn-glycero-3-phosphocholine | | | | |
| LMGP01012135 | PC(14:1(9Z)/22:5(7Z,10Z,13Z,18Z,19Z)) | 1-(9Z-tetradecenoyl)-2-(7Z,10Z,13Z,18Z,19Z- | 778.6 | 778.5381 | C₄₃H₇₇NO₃P | M + H |
| | | docosapentaenoyl)-sn-glycero-3-phosphocholine | | | | |
| LMGP01012233 | PC(22:5(4Z,7Z,10Z,13Z,18Z)/14:1(9Z)) | 1-(4Z,7Z,10Z,13Z,18Z-docosapentaenoyl)-2-(9Z- | 778.6 | 778.5381 | C₄₃H₇₇NO₃P | M + H |
| | | tetradecanoyl)-glycero-3-phosphocholine | | | | |
| LMGP01012235 | PC(22:5(7Z,10Z,13Z,18Z,19Z)/14:1(9Z)) | 1-(7Z,10Z,13Z,18Z,19Z-docosapentaenoyl)-2-(9Z- | 778.6 | 778.5381 | C₄₃H₇₇NO₃P | M + H |
| | | tetradecanoyl)-glycero-3-phosphocholine | | | | |
| LMGP02010819 | PE(17:2(9Z,12Z)/22:4(7Z,10Z,13Z,18Z)) | 1-(9Z,12Z-heptadecadienoyl)-2-(7Z,10Z,13Z,18Z- | 778.6 | 778.5381 | C₄₃H₇₇NO₃P | M + H |
| | | docosatetraenoyl)-glycero-3-phosphoethanolamine | | | | |
| LMGP02010917 | PE(19:1(9Z)/20:5(5Z,8Z,11Z,14Z,17Z)) | 1-(9Z-nonadecenoyl)-2-(5Z,8Z,11Z,14Z,17Z- | 778.6 | 778.5381 | C₄₃H₇₇NO₃P | M + H |
| | | eicosapentaenoyl)-glycero-3-phosphoethanolamine | | | | |
| LMGP02010979 | PE(20:5(5Z,8Z,11Z,14Z,17Z)/19:1(9Z)) | 1-(5Z,8Z,11Z,14Z,17Z-picosapentaenoyl)-2-(9Z- | 778.6 | 778.5381 | C₄₃H₇₇NO₃P | M + H |
| | | nonadecenoyl)-glycero-3-phosphoethanolamine | | | | |
| LMGP02011112 | PE(22:4(7Z,10Z,13Z,18Z)/17:2(9Z,12Z)) | 1-(7Z,10Z,13Z,18Z-docosatetraenoyl)-2-(9Z,12Z- | 778.6 | 778.5381 | C₄₃H₇₇NO₃P | M + H |
| | | heptadecadienoyl)-glycero-3-phosphoethanolamine | | | | |
| LMGP02011139 | PE(22:8(4Z,7Z,10Z,13Z,18Z,19Z)/17:0) | 1-(4Z,7Z,10Z,13Z,18Z,19Z-docosahexaenoyl)-2- | 778.6 | 778.5381 | C₄₃H₇₇NO₃P | M + H |
| | | heptadecanoyl-glycero-3-phosphoethanolamine | | | | |
| LMGP02011210 | PE(17:0/22:8(4Z,7Z,10Z,13Z,18Z,19Z)) | 1-heptadecanoyl-2-(4Z,7Z,10Z,13Z,18Z,19Z- | 778.6 | 778.5381 | C₄₃H₇₇NO₃P | M + H |
| | | docosahexsenoyl)-glycero-3-phosphoethanolamine | | | | |
| LMGP02020104 | PE(O-18:0/22:8(4Z,7Z,10Z,13Z,18Z,19Z)) | 1-octadeceyl-2-(4Z,7Z,10Z,13Z,18Z,19Z- | 778.6 | 778.5745 | C₂₅H₅₁NO₇P | M + H |
| | | docosahexsenoyl)-sn-glycero-3- | | | | |
| | | phosphoethanolamine | | | | |
| LMGP02030038 | PE(P-20:0/20:5(5Z,8Z,11Z,14Z,17Z)) | 1-(1Z-eicosenyl)-2-(5Z,8Z,11Z,14Z,17Z- | 778.6 | 778.5745 | C₂₅H₅₁NO₇P | M + H |
| | | eicosapentaenoyl)-glycero-3-phosphoethanolamine | | | | |
| LMGP03010033 | PS(13:0/22:0) | 1-tridecanoyl-2-docosanoyl-glycero-3-phosphoserine | 778.6 | 778.5583 | C₄₁H₈₁NO₁₀P | M + H |
| LMGP03010153 | PS(15:0/20:0) | 1-pentadecanoyl-2-eicosanoyl-glycero-3- | 778.6 | 778.5583 | C₄₁H₈₁NO₁₀P | M + H |
| | | phosphoserine | | | | |
| LMGP03010312 | PS(18:0/17:0) | 1-octadecanoyl-2-heptadecanoyl-glycero-3- | 778.6 | 778.5583 | C₄₁H₈₁NO₁₀P | M + H |
| | | phosphoserine | | | | |
| LMGP03010332 | PS(18:3(6Z,9Z,12Z)/18:4(6Z,9Z,12Z,15Z)) | 1-(8Z,9Z,12Z-octadecatrienoyl)-2-(8Z,9Z,12Z,15Z- | 778.6 | 778.4854 | C₄₂H₅₃NO₁₀P | M + H |
| | | octadecatetraenoyl)-glycero-3-phosphoserine | | | | |
| LMGP03010410 | PS(19:3(9Z,12Z,15Z)/18:4(8Z,9Z,12Z,15Z)) | 1-(9Z,12Z,15Z-octadecatrienoyl)-2-(8Z,9Z,12Z,15Z- | 778.6 | 778.4854 | C₄₂H₅₃NO₁₀P | M + H |
| | | octadecatetraenoyl)-glycero-3-phosphoserine | | | | |
| LMGP03010439 | PS(19:4(6Z,9Z,12Z,15Z)/19:3(6Z,9Z,12Z)) | 1-(6Z,9Z,12Z,15Z-octadecatetraenoyl)-2- | 778.6 | 778.4854 | C₄₂H₅₃NO₁₀P | M + H |
| | | (6Z,9Z,12Z-octadecatrienoyl)-glycero-3- | | | | |
| | | phosphoserine | | | | |
| LMGP03010440 | PS(19:4(6Z,9Z,12Z,15Z)/19:3(6Z,9Z,12Z)) | 1-(6Z,9Z,12Z,15Z-octadecatetraenoyl)-2- | 778.6 | 778.4854 | C₄₂H₅₃NO₁₀P | M + H |
| | | (9Z,12Z,15Z-octadecatrienoyl)-glycero-3- | | | | |
| | | phosphoserine | | | | |
| LMGP03010512 | PS(20:0/15:0) | 1-eicosanoyl-2-pentadecanoyl-glycero-3- | 778.6 | 778.5583 | C₄₁H₈₁NO₁₀P | M + H |
| | | phosphoserine | | | | |
| LMGP03010920 | PS(22:6(4Z,7Z,10Z,13Z,16Z,19Z)/14:1(9Z)) | 1-(4Z,7Z,10Z,13Z,16Z,19Z-docosahexaenoyl-2- | 778.6 | 778.4854 | C₄₂H₅₃NO₁₀P | M + H |
| | | (9Z-tetradecenoyl)-glycero-3-phosphoserine | | | | |
| LMGP03010849 | PS(22:0/13:0) | 1-docosanoyl-2-tridecanoyl-glycero-3-phosphoserine | 778.6 | 778.5583 | C₄₁H₈₁NO₁₀P | M + H |
| LMGP03010853 | PS(21:0/14:0) | 1-heneicosanoyl-2-tetradecanoyl-glycero-3- | 778.6 | 778.5583 | C₄₁H₈₁NO₁₀P | M + H |
| | | phosphoserine | | | | |
| LMGP03010894 | PS(17:0/18:0) | 1-heptadecanoyl-2-octadecanoyl-glycero-3- | 778.6 | 778.5583 | C₄₁H₈₁NO₁₀P | M + H |
| | | phosphoserine | | | | |
| LMGP03010953 | PS(19:0/18:0) | 1-nonadecanoyl-2-hexadecanoyl-glycero-3- | 778.6 | 778.5583 | C₄₁H₈₁NO₁₀P | M + H |
| | | phosphoserine | | | | |
| LMGP03010968 | PS(18:0/19:0) | 1-hexadecanoyl-2-nonadecanoyl-glycero-3- | 778.6 | 778.5583 | C₄₁H₈₁NO₁₀P | M + H |
| | | phosphoserine | | | | |
| LMGP03020070 | PS(O-20:0/18:0) | 1-eicosyl-2-hexadecanoyl-glycero-3-phosphoserine | 778.6 | 778.5958 | C₄₂H₅₅NO₉P | M + H |
| LMGP03020097 | PS(O-19:0/18:0) | 1-octadecyl-2-octadecanoyl-glycero-3- | 778.6 | 778.5958 | C₄₂H₅₅NO₉P | M + H |
| | | phosphoserine | | | | |
| LMGP03020091 | PS(O-18:0/20:0) | 1-hexadecyl-2-eicosanoyl-glycero-3-phosphoserine | 778.6 | 778.5958 | C₄₂H₅₅NO₉P | M + H |
| LMGP01010633 | PC(18:0/20:5(5Z,8Z,11Z,14Z,17Z)) | 1-hexadecanoyl-2-(5Z,8Z,11Z,14Z,17Z- | 780.4 | 780.5533 | C₄₄H₇₉NO₃P | M + H |
| | | eicosapentraenoyl)-sn-glycero-3-phosphocholine | | | | |
| LMGP01010895 | PC(18:1(9Z)/20:4(5Z,8Z,11Z,14Z)) | 1-(9Z-hexadecanoyl-2-(5Z,8Z,11Z,14Z- | 780.4 | 780.5533 | C₄₄H₇₉NO₃P | M + H |
| | | eicostetraenoyl)-sn-glycero-3-phosphocholine | | | | |
| LMGP01011305 | PC(18:1(7Z)/20:4(5Z,8Z,11Z,14Z)) | 1-(7Z-hexadecanoyl-2-(5Z,8Z,11Z,14Z,- | 780.4 | 780.5533 | C₄₄H₇₉NO₃P | M + H |
| | | eicostetraenoyl)-sn-glycero-3-phosphocholine | | | | |
| LMGP01011407 | PC(14:1(9Z)/22:4(7Z,10Z,13Z,18Z)) | 1-(9Z-tetradecenoyl)-2-(7Z,10Z,13Z,16Z,- | 780.4 | 780.5533 | C₄₄H₇₉NO₃P | M + H |
| | | docosatetraenoyl)-glycero-3-phosphocholine | | | | |
| LMGP01011834 | PC(18:1(9Z)/18:4(8Z,9Z,12Z,15Z)) | 1-(9Z-octadecenoyl)-2-(8Z,9Z,12Z,15Z,- | 780.4 | 780.5533 | C₄₄H₇₉NO₃P | M + H |
| | | octadecstetraenoyl)-glycero-3-phosphocholine | | | | |
| LMGP01011825 | PC(18:2(9Z,12Z)/18:3(8Z,9Z,12Z)) | 1-(9Z,12Z-octadecenoyl)-2-(8Z,9Z,12Z- | 780.4 | 780.5533 | C₄₄H₇₉NO₃P | M + H |
| | | octadecatrienoyl)-glycero-3-phosphocholine | | | | |
| LMGP01011628 | PC(18:2(9Z,12Z)/18:3(9Z,12Z,15Z)) | 1-(9Z,12Z-octadecenoyl)-2-(9Z,12Z,15Z- | 780.4 | 780.5533 | C₄₄H₇₉NO₃P | M + H |
| | | octadeostnienoyl)-glycero-3-phosphocholine | | | | |
| LMGP01011653 | PC(19:3(8Z,9Z,12Z)/13:2(9Z,12Z)) | 1-(8Z,9Z,12Z-octadecenoyl)-2-(9Z,12Z- | 780.4 | 780.5533 | C₄₄H₇₉NO₃P | M + H |
| | | octadecatenoyl)-glycero-3-phosphocholine | | | | |
| LMGP01011833 | PC(18:3(9Z,12Z,15Z)/18:2(9Z,12Z)) | 1-(9Z,12Z,15Z-octadecenoyl)-2-(9Z,12Z- | 780.4 | 780.5533 | C₄₄H₇₉NO₃P | M + H |
| | | octadecatenoyl)-glycero-3-phosphocholine | | | | |
| LMGP01011712 | PC(18:4(6Z,9Z,12Z,15Z)/18:1(9Z)) | 1-(8Z,9Z,12Z,15Z-octadecstetraenoyl)-2-(9Z- | 780.4 | 780.5533 | C₄₄H₇₉NO₃P | M + H |
| | | octadecenoyl)-glycero-3-phosphocholine | | | | |
| LMGP01011933 | PC(20:4(5Z,8Z,11Z,14Z)/18:1(9Z)) | 1-(5Z,8Z,11Z,14Z-eicosatetraenoyl)-2-(9Z- | 780.4 | 780.5533 | C₄₄H₇₉NO₃P | M + H |
| | | hexadecenoyl)-glycero-3-phosphocholine | | | | |
| LMGP01011932 | PC(20:5(5Z,8Z,11Z,14Z,17Z)/18:0) | 1-(5Z,8Z,11Z,14Z,17Z-eicosapentoenoyl)-2- | 780.4 | 780.5533 | C₄₄H₇₉NO₃P | M + H |
| | | hexadecenoyl)-glycero-3-phosphocholine | | | | |
| LMGP01012059 | PC(22:4(7Z,10Z,13Z,12Z)/14:1(9Z)) | 1-(7Z,10Z,13Z,18Z-docosetetranoyl-)-2-(9Z- | 780.4 | 780.5533 | C₄₄H₇₉NO₃P | M + H |
| | | tetradeoenoyl)-glycero-3-phosphocholine | | | | |
| LMGP01012130 | PC(14:0/22:5(4Z,7Z,10Z,13Z,12Z)) | 1-tetradecanoyl-2-(4Z,7Z,10Z,13Z,18Z- | 780.4 | 780.5533 | C₄₄H₇₉NO₃P | M + H |
| | | docosapartaenoyl)-sn-glycero-3-phosphocholine | | | | |
| LMGP01012131 | PC((14:0/22:5(7Z,10Z,13Z,18Z,19Z)) | 1-tetradecanoyl-2-(7Z,10Z,13Z,18Z,19Z- | 780.4 | 780.5533 | C₄₄H₇₉NO₃P | M + H |
| | | docosapartaenoyl)-sn-glycero-3-phosphocholine | | | | |
| LMGP01012132 | PC(18:1(9Z)/20:4(7Z,11Z,14Z,17Z)) | 1-(9Z-hexodecanoyl)-2-(8Z,11Z,14Z,17Z- | 780.4 | 780.5533 | C₄₄H₇₉NO₃P | M + H |
| | | eicosstetraenoyl)-sn-glycero-3-phosphocholine | | | | |
| LMGP01012152 | PC(18:1(11Z)/18:4(6Z,9Z,12Z,15Z)) | 1-(11Z-octadeoanoyl)-2-(8Z,9Z,12Z,15Z- | 780.4 | 780.5533 | C₄₄H₇₉NO₃P | M + H |
| | | octadecatetrsenoyl)-sn-glycero-3-phosphocholine | | | | |
| LMGP01012182 | PC(19:4(6Z,9Z12Z,15Z)/18:1(11Z)) | 1-(8Z,9Z,12Z,15Z-octadeostetraenoyl)-2-(11Z- | 780.4 | 780.5533 | C₄₄H₇₉NO₃P | M + H |
| | | octadecenoyl)-sn-glycero-3-phosphocholine | | | | |
| LMGP01012210 | PC(20:4(8Z,11Z,14Z,17Z)/18:1(9Z)) | 1-(8Z,11Z,14Z,17Z-eicosatetraenoyl)-2-(9Z- | 780.4 | 780.5533 | C₄₄H₇₉NO₃P | M + H |
| | | hexadecenoyl)-sn-glycero-3-phosphocholine | | | | |
| LMGP01012232 | PC(22:5(4Z,7Z,10Z,13Z)18Z)/14:0) | 1-(4Z,7Z,10Z,13Z,18Z-docosaperisonoyl)-2- | 780.4 | 780.5533 | C₄₄H₇₉NO₃P | M + H |
| | | tetradecanoyl-sn-glycero-3-phosphocholine | | | | |
| LMGP01012234 | PC(22:5(7Z,10Z,13Z,18Z,19Z)/14:0) | 1-(7Z,10Z,13Z,18Z,19Z-docosapentsenoyl)-2- | 780.4 | 780.5533 | C₄₄H₇₉NO₃P | M + H |
| | | tetradecanoyl-sn-glycero-3-phosphocholine | | | | |
| LMGP03010599 | PE(17:1)9Z)/22:4(7Z,10Z,13Z,18Z)) | 1-(9Z-heptadecenoyl)-2-(7Z,10Z,13Z,18Z- | 780.4 | 780.5533 | C₄₄H₇₉NO₃P | M + H |
| | | dososatetrasenoyl)-glycero-3-phosphoethanoamine | | | | |
| LMGP02010788 | PE(19:0)/20:5(5Z,8Z,11Z,14Z,17Z)) | 1-nonadecanoyl-2-(5Z,8Z,11Z,14Z,17Z- | 780.4 | 780.5533 | C₄₄H₇₉NO₃P | M + H |
| | | eicosatetraenoyl)-glycero-3-phosphoethanoamine | | | | |
| LMGP02010318 | PE(19:1(2Z)/20:4(5Z,8Z,11Z,14Z,)) | 1-(9Z-nonadecenoyl)-2-(5Z,8Z,11Z,14Z, | 780.4 | 780.5533 | C₄₄H₇₉NO₃P | M + H |
| | | eicosatetraenoyl)-glycero-3-phosphoethanoamine | | | | |
| LMGP02010950 | PE(20:4(5Z,8Z,11Z,14Z)/12:1(9Z)) | 1-(5Z,8Z,11Z,14Z-eicosatetraenoyl)-2-(9Z- | 780.4 | 780.5533 | C₄₄H₇₉NO₃P | M + H |
| | | nonadecenoyl)-glycero-3-phosphoethanoamine | | | | |
| LMGP02010278 | PE(20:5(5Z,8Z,11Z,14Z)17Z)/19:0) | 1-(5Z,8Z-11Z,14Z,17Z-eicosaperteanoyl)-2- | 780.4 | 780.5533 | C₄₄H₇₉NO₃P | M + H |
| | | nonadecanoyl)-glycero-3-phosphoethanoamine | | | | |
| LMGP02011111 | PE(22:4(7Z,10Z,13Z,16Z)/17:1(9Z)) | 1-(7Z,10Z,13Z,18Z-docosatetreanoyl)-2-(9Z- | 780.4 | 780.5533 | C₄₄H₇₉NO₃P | M + H |
| | | heptadecenoyl)-glycero-3-phosphoethanoamine | | | | |
| LMGP03230084 | PE(O-20:0/20:5(5Z,8Z,11Z,14Z,17Z)) | 1-eisosyl-2-(5Z,8Z,11Z,14Z,17Z-eicosapentaenoyl)- | 780.4 | 780.5932 | C₄₅H₃₃NO₇P | M + H |
| | | glycero-3-phosphoethanoamine | | | | |
| LMGP02030031 | PE(P-18:0/22:4(7Z,10Z,13Z,18Z)) | 1-(1Z-octadecenyl)-2-(7Z,10Z,13Z,18Z- | 780.4 | 780.5932 | C₄₅H₃₃NO₇P | M + H |
| | | docosatetroenoyl)-glycero-3-phosphoethanoamine | | | | |
| LMGP03030095 | PE(P-20:0/20:4(5Z,8Z,11Z,14Z)) | 1-(1Z-eicosenyl)-2-(5Z,8Z,11ZZ,14Z-eicosatetreanoyl)- | 780.4 | 780.5932 | C₄₅H₃₃NO₇P | M + H |
| | | glycero-3-phosphoethanoamine | | | | |
| LMGP03010220 | PS(16:1(9Z)/20:5(5Z,8Z,11Z,14Z,17Z)) | 1-(9Z-hexadecenoyl)-2-(5Z,8Z,11Z,14Z,17Z- | 780.4 | 780.4810 | C [symbol] H₇₁NO₁₀P | M + H |
| | | eicosapenteanoyl)-glycero-3-phosphoserine | | | | |
| LMGP03010353 | PS(18:2(9Z,12Z)/18:4(6Z,9Z,12Z,15Z)) | 1-(9Z,12Z-octadecatrienoyl)-2-(6Z,9Z,12Z,15Z- | 780.4 | 780.4910 | C [symbol] H₇₁NO₁₀P | M + H |
| | | octadecatrienoyl)-glycero-3-phosphoserine | | | | |
| LMGP03010380 | PS(18:3(6Z,9Z,12Z)/19:3(6Z,9Z,12Z)) | 1,2-di-(6Z,9Z,12Z-octadecatrienoyl)-sn-glycero-3- | 780.4 | 780.4810 | C [symbol] H₇₁NO₁₀P | M + H |
| | | phosphoserine | | | | |
| LMGP03010381 | PS(18:3(6Z,9Z,12Z)/18:3(9Z,12Z,15Z)) | 1-(6Z,9Z,12Z-octadecatrienoyl)-2-(9Z,12Z,15Z- | 780.4 | 780.4810 | C [symbol] H₇₁NO₁₀P | M + H |
| | | octadecatrienoyl)-glycero-3-phosphoserine | | | | |
| LMGP03010400 | PS(18:3(9Z,12Z,15Z)/18:3(6Z,9Z,12Z)) | 1-(9Z,12Z,15Z-octadecatrienoyl)-2-(6Z,9Z,12Z- | 780.4 | 780.4910 | C [symbol] H₇₁NO₁₀P | M + H |
| | | octadecatrienoyl)-glycero-3-phosphoserine | | | | |
| LMGP03010438 | PS(18:4(6Z,9Z,12Z,15Z)/19:2(9Z,12Z)) | 1-(6Z,9Z,12Z,15Z-octadecatetraenoyl)-2-(9Z,12Z- | 780.4 | 780.4910 | C [symbol] H₇₁NO₁₀P | M + H |
| | | octadecadienoyl)-glycero-3-phosphoserine | | | | |
| LMGP03010655 | PS(20:5(5Z,8Z,11Z,14Z,17Z)/16:1(9Z)) | 1-(5Z,8Z,11Z,14Z,17Z-eicosapentaenoyl)-2-(9Z- | 780.4 | 780.4910 | C [symbol] H₇₁NO₁₀P | M + H |
| | | hexadecenoyl)-glycero-3-phosphoserine | | | | |
| LMGP03010919 | PS(22:6(4Z,7Z,10Z,13Z,18Z,19Z)/14:0) | 1-(4Z,7Z,10Z,13Z,16Z,19Z-docosahexaenoyl-2- | 780.4 | 780.4910 | C [symbol] H₇₁NO₁₀P | M + H |
| | | tetradecanoyl)-glycero-3-phosphoserine | | | | |
| LMGP03010920 | PS(14:0/22.6(4Z,7Z,10Z,13Z,16Z,19Z)) | 1-tetradecanoyl-2-(4Z,7Z,10Z,13Z,16Z,19Z- | 780.4 | 780.4910 | C [symbol] H₇₁NO₁₀P | M + H |
| | | docosahexaenoyl)-glycero-3-phosphoserine | | | | |
| LMGP03010974 | PS(18:3(9Z,12Z,15Z)/18:3(9Z,12Z,15Z)) | 1,2-di-(9Z,12Z,15Z-octadecatrienoyl)-sn-glycero-3- | 780.4 | 780.4910 | C [symbol] H₇₁NO₁₀P | M + H |
| | | phosphoserine | | | | |
| LMGP20020003 | PE(16:0/22:6(54Z,7Z,10Z,12E,16Z,19Z) | 1-hexadecanoyl-2-(14-hydroxy- | 780.4 | 780.5174 | C [symbol] H₇₅NO₃P | M + H |
| | (14CH)) | 4Z,7Z,10Z,12E,16Z,19Z-docosahexaenoyl)-sn-glycreo- | | | | |
| | | 3-phosphoserine | | | | |
| LMGP06020002 | C16 Sulfatide | (3′-sulfo)Galbeta-Cer(d18:1/16:0) | 780.4 | 780.5293 | C [symbol] H₇₅NO₁₁P | M + H |
| LMGP01010322 | PC(16:0/20:3(5Z,8Z,11Z)) | 1-hexadecanoyl-2-(5Z,8Z,11Z- | 784.4 | 784.5351 | C [symbol] H₃₃NO₃P | M + H |
| | | eicosatrienoyl)-sn-glycero-3- | | | | |
| | | phosphocholine | | | | |
| LMGP01010324 | PC(16:0/20:3(8E,11E,14E)) | 1-hexadecanoyl-2-(8E,11E,14E- | 784.4 | 784.5351 | C [symbol] H₃₃NO₃P | M + H |
| | | eicosatrienoyl)-sn-glycero-3- | | | | |
| | | phosphocholine | | | | |
| LMGP01010327 | PC(16:0/20:3(8Z,11Z,14Z)) | 1-hexadecanoyl-2-(8Z,11Z,14Z- | 784.4 | 784.5351 | C [symbol] H₃₃NO₃P | M + H |
| | | eicosatrienoyl)-sn-glycero-3- | | | | |
| | | phosphocholine | | | | |
| LMGP01010393 | PC(19:1(9Z)/19:2(6Z,9Z)) | 1-(2Z-octadecenoyl)-2-(8Z,9Z- | 784.4 | 784.5351 | C [symbol] H₃₃NO₃P | M + H |
| | | octadecadienoyl)-sn-glycero-3- | | | | |
| | | phosphocholine | | | | |
| LMGP01010395 | PC(19:1(9Z)/19:2(9Z,12Z)) | 1-(9Z-octadecenoyl)-2-(9Z,12Z- | 784.4 | 784.5351 | C [symbol] H₃₃NO₃P | M + H |
| | | octadecadienoyl)-sn-glycero-3- | | | | |
| | | phosphocholine | | | | |
| LMGP01011406 | PC(14:1(9Z)/22:2(13Z,18Z)) | 1-(9Z-tetradecenoyl)-2-(13Z,16Z- | 784.4 | 784.5351 | C [symbol] H₃₃NO₃P | M + H |
| | | decosadienoyl)-glycero-3- | | | | |
| | | phosphocholine | | | | |
| LMGP01011490 | PC(16:1(9Z)/20:2(11Z,14Z)) | 1-(9Z-hexadecanoyl)-2-(11Z,14Z- | 784.4 | 784.5551 | C [symbol] H₃₃NO₃P | M + H |
| | | eicosadienoyl)-glycero-3- | | | | |
| | | phosphocholine | | | | |
| LMGP01011588 | PC(17:2(9Z,12Z)/19:1(9Z)) | 1-(9Z,12Z-heptadecadienoyl)-2-(9Z- | 784.4 | 784.5551 | C [symbol] H₃₃NO₃P | M + H |
| | | nonadecenoyl)-glycero-3- | | | | |
| | | phosphocholine | | | | |
| LMGP01011589 | PC(18:0/19:3(6Z,9Z,12Z)) | 1-octadecanoyl-2-(6Z,9Z,12Z- | 784.4 | 784.5551 | C [symbol] H₃₃NO₃P | M + H |
| | | octadecatrienoyl)-glycero-3- | | | | |
| | | phosphocholine | | | | |
| LMGP01011592 | PC(18:0/19:3(9Z,12Z,15Z)) | 1-octadecanoyl-2-(9Z,12Z,15Z- | 784.4 | 784.5551 | C [symbol] H₃₃NO₃P | M + H |
| | | octadecatrienoyl)-glycero-3- | | | | |
| | | phosphocholine | | | | |
| LMGP01011624 | PC(18:2(9Z,12Z)/18:1(9Z)) | 1-(2Z,12Z-octadecadienoyl)-2-(9Z- | 784.4 | 784.5551 | C [symbol] H₃₃NO₃P | M + H |
| | | octadecenoyl)-glycero-3- | | | | |
| | | phosphocholine | | | | |
| LMGP01011651 | PC(18:3(6Z,9Z,12Z)/18:0) | 1-(6Z,9Z,12Z-octadecatrienoyl)-2- | 784.4 | 784.5551 | C [symbol] H₃₃NO₃P | M + H |
| | | octadecanoyl-glycero-3-phosphocholine | | | | |
| LMGP01011682 | PC(18:3(9Z,12Z,15Z)/19:0) | 1-(9Z,12Z,15Z-octadecatrienoyl)-2- | 784.4 | 784.5951 | C [symbol] H₃₃NO₃P | M + H |
| | | octadecanoyl-glycero-3-phosphocholine | | | | |
| LMGP01011766 | PC(19:1(9Z)/17:2(9Z,12Z)) | 1-(9Z-nonadecanoyl)-2-(9Z,12Z- | 784.4 | 784.5551 | C [symbol] H₃₃NO₃P | M + H |
| | | heptadecadienoyl)-glycero-3- | | | | |
| | | phosphocholine | | | | |
| LMGP01011842 | PC(20:2(11Z,14Z)/16:1(9Z)) | 1-(11Z,14Z-eicosadienoyl)-2-(9Z- | 784.4 | 784.5951 | C [symbol] H₃₃NO₃P | M + H |
| | | hexadecenoyl)-glycero-3- | | | | |
| | | phosphocholine | | | | |
| LMGP01011872 | PC(20:3(9Z,11Z,14Z)/16:0) | 1-(9Z,11Z,14Z-eicosatrienoyl)-2- | 784.4 | 784.5951 | C [symbol] H₃₃NO₃P | M + H |
| | | hexadecanoyl-glycero-3-phosphocholine | | | | |
| LMGP01012339 | PC(22:2(13Z,16Z)/14:1(9Z)) | 1-(13Z,16Z-docosadienoyl)-2-(9Z- | 784.4 | 784.5951 | C [symbol] H₃₃NO₃P | M + H |
| | | tetradecenoyl)-glycero-3- | | | | |
| | | phosphocholine | | | | |
| LMGP01012149 | PC(18:1(11Z)/19:2(9Z,12Z)) | 1-(11Z-octadecenoyl)-2-(9Z,12Z- | 784.4 | 784.5351 | C [symbol] H₃₃NO₃P | M + H |
| | | octadecadienoyl)-sn-glycero-3- | | | | |
| | | phosphocholine | | | | |
| LMGP01012169 | PC(19:2(9Z,12Z)/19:1(11Z)) | 1-(9Z,12Z-octadecadienoyl)-2-(11Z- | 784.4 | 784.5551 | C [symbol] H₃₃NO₃P | M + H |
| | | octadecenoyl)-sn-glycero-3- | | | | |
| | | phosphocholine | | | | |
| LMGP01012194 | PC(20:3(5Z,8Z,11Z)/16:0) | 1-(5Z,8Z,11Z-eicosatrienoyl)-2- | 784.4 | 784.5851 | C [symbol] H₃₃NO₃P | M + H |
| | | hexadecanoyl-sn-glycero-3- | | | | |
| | | phosphocholine | | | | |
| LMGP02010588 | PE(17:1(9Z)/22:2(13Z,16Z)) | 1-(9Z-heptadecenoyl)-2-(13Z,16Z- | 784.4 | 784.5951 | C [symbol] H₃₃NO₃P | M + H |
| | | docosadienoyl)-glycero-3- | | | | |
| | | phosphoethanolamine | | | | |
| LMGP02010617 | PE(17:2(9Z,12Z)/22:1(11Z)) | 1-(9Z,12Z-heptadecadienoyl)-2-(11Z- | 784.4 | 784.5951 | C [symbol] H₃₃NO₃P | M + H |
| | | docosenoyl)-glycero-3- | | | | |
| | | phosphoethanolamine | | | | |
| LMGP02010704 | PE(18:3(6Z,9Z,12Z)/21:0) | 1-(6Z,9Z,12Z-octadecatrienoyl)-2- | 784.4 | 784.5951 | C [symbol] H₃₃NO₃P | M + H |
| | | heneicosanoyl-glycero-3- | | | | |
| | | phosphoethanolamine | | | | |
| LMGP02010732 | PE(18:3(9Z,12Z,15Z)/21:0) | 1-(9Z,12Z,15Z-octadecatrienoyl)-2- | 784.4 | 784.5951 | C [symbol] H₃₃NO₃P | M + H |
| | | heneicosanoyl-glycero-3- | | | | |
| | | phosphoethanolamine | | | | |
| LMGP02010768 | PE(18:4(6Z,9Z,12Z,15Z)/22:6(4Z,7Z,10Z,13Z,16Z,19Z)) | 1-(6Z,9Z,12Z,15Z- | 784.4 | 784.4921 | C [symbol] H₇₁NO₃P | M + H |
| | | octadecatetraenoyl)-2- | | | | |
| | | (4Z,7Z,10Z,13Z,16Z,19Z- | | | | |
| | | docosahexaenoyl)-glycero-3- | | | | |
| | | phosphoethanolamine | | | | |
| LMGP02010784 | PE(19:0/20:3(8Z,11Z,14Z)) | 1-nonadecanoyl-2-(8Z,11Z,14Z- | 784.4 | 784.5951 | C [symbol] H₃₃NO₃P | M + H |
| | | eicosatrienoyl)-glycero-3- | | | | |
| | | phosphoethanolamine | | | | |
| LMGP02010914 | PE(19:1(9Z)/20:2(11Z,14Z)) | 1-(2Z-nonadecenoyl)-2-(11Z,14Z- | 784.4 | 784.5951 | C [symbol] H₃₃NO₃P | M + H |
| | | eicosadienoyl)-glycero-3- | | | | |
| | | phosphoethanolamine | | | | |
| LMGP02010889 | PE(20:2(11Z,14Z)/19:1(9Z)) | 1-(11Z,14Z-eicosadienoyl)-2-(9Z- | 784.4 | 784.5851 | C [symbol] H₃₃NO₃P | M + H |
| | | nonadecenoyl)-glycero-3- | | | | |
| | | phosphoethanolamine | | | | |
| LMGP02010919 | PE(20:3(8Z,11Z,14Z)/19:0) | 1-(8Z,11Z,14Z-eicosatrienoyl)-2-(9Z- | 784.4 | 784.5351 | C [symbol] H₃₃NO₃P | M + H |
| | | nonadecanoyl)-glycero-3- | | | | |
| | | phosphoethanolamine | | | | |
| LMGP02010985 | PE(20:5(5Z,8Z,11Z,14Z,17Z)/20:5(5Z,8Z,11Z,14Z,17Z)) | 1,2-di-(5Z,8Z,11Z,14Z,17Z- | 784.4 | 784.4912 | C [symbol] H₇₁NO₃P | M + H |
| | | eicosapentsenoyl)-sn-glycero-3- | | | | |
| | | phosphoethanolamine | | | | |
| LMGP02011001 | PE(21:0/18:3(6Z,9Z,12Z)) | 1-heneicosanoyl-2-(8Z,9Z,12Z- | 784.4 | 784.5951 | C [symbol] H₃₃NO₃P | M + H |
| | | octadecatrienoyl)-glycero-3- | | | | |
| | | phosphoethanolamine | | | | |
| LMGP02011002 | PE(21:0/18:3(9Z,12Z,15Z)) | 1-heneicosanoyl-2-(9Z,12Z,15Z- | 784.4 | 784.5951 | C [symbol] H₃₃NO₃P | M + H |
| | | octadecatrienoyl)-glycero-3- | | | | |
| | | phosphoethanolamine | | | | |
| LMGP02011050 | PE(22:1(11Z)/17:2(9Z,12Z)) | 1-(11Z-docosenoyl)-2-(9Z,12Z- | 784.4 | 784.5851 | C [symbol] H₃₃NO₃P | M + H |
| | | heptadecadienoyl)-glycero-3- | | | | |
| | | phosphoethanolamine | | | | |
| LMGP02011080 | PE(22:2(13Z,18Z)/17:1(9Z,)) | 1-(13Z,16Z-docosadienoyl)-2-(9Z- | 784.4 | 784.5851 | C [symbol] H₃₃NO₃P | M + H |
| | | heptadecanoyl)-glycero-3- | | | | |
| | | phosphoethanolamine | | | | |
| LMGP02011147 | PE(22:8(4Z,7Z,10Z,13Z,16Z,19Z)/18:4(6Z,9Z,12Z,15Z)) | 1-(4Z,7Z,10Z,13Z,16Z,19Z- | 784.4 | 784.4912 | C [symbol] H₇₁NO₃P | M + H |
| | | docosahexsanoyl)-2-(6Z,9Z,12Z,15Z- | | | | |
| | | octadecstetraenoyl)-glycero-3- | | | | |
| | | phosphoethanolamine | | | | |
| LMGP03010023 | PS(19:2(9Z,12Z)/18:2(9Z,12Z)) | 1,2-di-(9Z,12Z-octadecadienoyl)-sn- | 784.4 | 784.5123 | C [symbol] H₇₅NO₁₀P | M + H |
| | | glycero-3-phosphoserine | | | | |
| LMGP03010038 | PS(18:0/20:4(5Z,8Z,11Z,14Z)) | 1-hexadecanoyl-2-(5Z,8Z,11Z,14Z- | 784.4 | 784.5123 | C [symbol] H₇₅NO₁₀P | M + H |
| | | eicosatetreanoyl)-sn-glycero-3- | | | | |
| | | phosphoserine | | | | |
| LMGP03010109 | PS(14:0/22:4(7Z,10Z,13Z,16Z)) | 1-tetradecanoyl-2-(7Z,10Z,13Z,16Z- | 784.4 | 784.5123 | C [symbol] H₇₅NO₁₀P | M + H |
| | | docosatetraenoyl)-glycero-3- | | | | |
| | | phosphoserine | | | | |
| LMGP03010219 | PS(16:1(9Z)/20:3(8Z,11Z,14Z)) | 1-(9Z-hexadecenoyl)-2-(8Z,11Z,14Z- | 784.4 | 784.5123 | C [symbol] H₇₅NO₁₀P | M + H |
| | | eicosatrienoyl)-glycero-3-phosphoserine | | | | |
| LMGP03010330 | PS(18:1(9Z)/19:3(6Z,9Z,12Z)) | 1-(9Z-octadecenoyl)-2-(6Z,9Z,12Z- | 784.4 | 784.5123 | C [symbol] H₇₅NO₁₀P | M + H |
| | | octadecatrienoyl)-glycero-3-phosphoserine | | | | |
| LMGP03010378 | PS(18:3(8Z,9Z,12Z)/18:1(9Z)) | 1-(8Z,9Z,12Z-octadecatrienoyl-2-(9Z- | 784.4 | 784.5123 | C [symbol] H₇₅NO₁₀P | M + H |
| | | octadecanoyl)-glycero-3-phosphoserine | | | | |
| LMGP03010436 | PS(18:4(6Z,9Z,12Z,15Z)/18:0) | 1-(6Z,9Z,12Z,15Z- | 784.4 | 784.5123 | C [symbol] H₇₅NO₁₀P | M + H |
| | | octadecatetraenoyl-2-octadecanoyl)- | | | | |
| | | glycero-3-phosphoserine | | | | |
| LMGP03010596 | PS(20:3(8Z,11Z,14Z)/16:1(9Z) | 1-(8Z,11Z,14Z-eicosatrienoyl)-2-9Z- | 784.4 | 784.5123 | C [symbol] H₇₅NO₁₀P | M + H |
| | | hexadecenoyl)-glycero-3-phosphoserine | | | | |
| LMGP03010789 | PS(22:4(7Z,10Z,13Z,16Z)/14:0) | 1-(7Z,10Z,13Z,16Z- | 784.4 | 784.5123 | C [symbol] H₇₅NO₁₀P | M + H |
| | | docosatetraenoyl)-2-tetradecanoyl- | | | | |
| | | glycero-3-phosphoserine | | | | |
| LMGP03010859 | PS(20:4(5Z,8Z,11Z,14Z)/16:0) | 1-(5Z,8Z,11Z,14Z-eicosatetraenoyl)-2- | 784.4 | 784.5123 | C [symbol] H₇₅NO₁₀P | M + H |
| | | hexadecanoyl)-glycero-3-phosphoserine | | | | |
| LMGP03010874 | PS(18:3(9Z,12Z,15Z)/19:1(9Z) | 1-(9Z,12Z,15Z-octadecatrienoyl)-2-9Z- | 784.4 | 784.5123 | C [symbol] H₇₅NO₁₀P | M + H |
| | | octadecenoyl)-glycero-3-phosphoserine | | | | |
| LMGP03010880 | PS(19:1(9Z)/18:3(9Z,12Z,15Z)) | 1-(9Z-octadecenoyl)-2-(9Z,12Z,15Z- | 784.4 | 784.5123 | C [symbol] H₇₅NO₁₀P | M + H |
| | | octadecatrienoyl)-glycero-3-phosphoserine | | | | |
| LMGP03010998 | PS(18:0/18:4(6Z,9Z,12Z,15Z)) | 1-octadecanoyl-2-(6Z,9Z,12Z,15Z- | 784.4 | 784.5123 | C [symbol] H₇₅NO₁₀P | M + H |
| | | octadecatetranoyl)-glycero-3-phosphoserine | | | | |
| LMGP20020001 | PE(18:0/20:4(5Z,8Z,10E,14Z)(12CH[S])) | 1-octadecanoyl-2-(12S-hydroxy- | 784.4 | 784.5497 | C [symbol] H₇₅NO₃P | M + H |
| | | 5Z,8Z,10E,14Z-eicosatetraenoyl)-sn- | | | | |
| | | glycero-3-phosphoethanolamine | | | | |
| LMGP20020002 | PE(18:0/20:4(5Z,8Z,11Z,13E)(15CH[S])) | 1-octadecanoyl-2-(15S-hydroxy- | 784.4 | 784.5497 | C [symbol] H₇₅NO₃P | M + H |
| | | 5Z,8Z,11Z,13E-eicosatetraenoyl)-sn- | | | | |
| | | glycero-3-phosphoethanolamine | | | | |

Note:
Chains containing double bonds and/or functional groups with defined regiochemistry, geometry and stereochemistry are meant to serve as examples for structure-drawing purposes. In many cases there may be alternative isobaric structures. [symbol]
indicates data missing or illegible when filed

TABLE 32
M/Z 786.4

| | | | Input | Exact | | |
|---|---|---|---|---|---|---|
| LM_ID | Name | Systematic Name | m/z | m/z | Formula | Ion |
| LMPG01093031 | | 1-(8-[5]-ladderane-hexanoyl)-2-(8-[3]- | 783.4 | 786.5432 | C [symbol] H₇₇NO₇P | M + H |
| | | ladderane-octantyl)-sn- | | | | |
| | | glycerophosphocholine | | | | |
| LMPG02010709 | PE(19:3(8Z,9Z,12Z)/22:6(4Z,7Z, | 1-(8Z,9Z,10Z-octadecatrienoyl)-2- | 788.4 | 786.5089 | C [symbol] H₇₃NO₃P | M + H |
| | 10Z,13Z,18Z,19Z)) | (4Z,7Z,10Z,13Z,18Z,19Z- | | | | |
| | | docosahexaenoyl)-glycero-3- | | | | |
| | | phosphoethanolamine | | | | |
| LMPG02010737 | PE(18:3(9Z,12Z,15Z)/22:6(4Z,7Z, | 1-(9Z,12Z,15Z-octadecatrienoyl)-2- | 788.4 | 786.5089 | C [symbol] H₇₃NO₃P | M + H |
| | 10Z,13Z,18Z,19Z)) | (4Z,7Z,10Z,13Z,18Z,19Z- | | | | |
| | | docosahexaenoyl)-glycero-3- | | | | |
| | | phosphoethanolamine | | | | |
| LMPG02010955 | PE(20:4(5Z,8Z,11Z,14Z)/20:5(5Z, | 1-(5Z,8Z,11Z,14Z-elcosatetraenoyl)-2- | 788.4 | 786.5089 | C [symbol] H₇₃NO₃P | M + H |
| | 8Z,11Z,14Z,17Z)) | (5Z,8Z,11Z,14Z,17Z-elcosatetraenoyl)- | | | | |
| | | glycero-3-phosphoethanolamine | | | | |
| LMPG02010984 | PE(20:5(5Z,8Z,11Z,14Z,17Z)/20:4(5Z, | 1-(5Z,8Z,11Z,14Z,17Z- | 788.4 | 786.5089 | C [symbol] H₇₃NO₃P | M + H |
| | 8Z,11Z,14Z)) | elcosapentaenoyl)-2-(5Z,8Z,11Z,14Z- | | | | |
| | | elcosatetraenoyl)-glycero-3- | | | | |
| | | phosphoethanolamine | | | | |
| LMPG2011145 | PE(22:8(4Z,7Z,10Z,13Z,18Z,19Z)/ | 1-(4Z,7Z,10Z,13Z,18Z,19Z- | 788.4 | 786.5089 | C [symbol] H₇₃NO₃P | M + H |
| | 18:3(8Z,9Z,12Z)) | docosahexaenoyl)-2-(8Z,9Z,12Z- | | | | |
| | | octadecatrienoyl)-glycero-3- | | | | |
| | | phosphoethanolamine | | | | |
| LMPG2011148 | PE(22:8(4Z,7Z,10Z,13Z,18Z,19Z)/ | 1-(4Z,7Z,10Z,13Z,18Z,19Z- | 788.4 | 786.5089 | C [symbol] H₇₃NO₃P | M + H |
| | 18:3(8Z,12Z,15Z)) | docosahexaenoyl)-2-(8Z,12Z,15Z- | | | | |
| | | octadecatrienoyl)-glycero-3- | | | | |
| | | phosphoethanolamine | | | | |
| LMPG03310138 | PS(14:1(9Z)/22:2(13Z,18Z) | 1-(9Z-tetradecanoyl)-2-(13Z,18Z- | 788.4 | 786.5290 | C [symbol] H₇₇NO₁₀P | M + H |
| | | docosadienoyl)-glycero-3-phophoserine | | | | |
| LMPG03310218 | PS(18:1(9Z)/20:2(11Z,14Z) | 1-(9Z-hexadecenoyl)-2-(11Z,14Z- | 788.4 | 786.5290 | C [symbol] H₇₇NO₁₀P | M + H |
| | | eicosadienoyl)-glycero-3-phophoserine | | | | |
| LMPG03010295 | PS(17:2(9Z,12Z)/19:1(9Z)) | 1-(9Z,12Z-heptodicadienoyl)-2-(9Z- | 788.4 | 786.5290 | C [symbol] H₇₇NO₁₀P | M + H |
| | | nondicenoyl)-glycero-3-phophoserine | | | | |
| LMPG03310315 | PS(18:0/18:3(6Z,9Z,12Z)) | 1-octadecanoyl)-2-(6Z,9Z,12Z- | 788.4 | 786.5290 | C [symbol] H₇₇NO₁₀P | M + H |
| | | octadecatrienoyl)-glycero-3-phophoserine | | | | |
| LMPG03310316 | PS(18:0/18:3(9Z,12Z,15Z)) | 1-octadecanoyl)-2-(9Z,12Z,15Z- | 788.4 | 786.5290 | C [symbol] H₇₇NO₁₀P | M + H |
| | | octadecatrienoyl)-glycero-3-phophoserine | | | | |
| LMPG03310350 | PS(18:2(8Z,12Z)/19:1(9Z)) | 1-(9Z,12Z-octadecatrienoyl)-2-(9Z- | 788.4 | 786.5290 | C [symbol] H₇₇NO₁₀P | M + H |
| | | octadecenoyl)-glycero-3-phophoserine | | | | |
| LMPG03310377 | PS(18:3(8Z,9Z,12Z)/18:0) | 1-(8Z,9Z,12Z-octadecatrienoyl)-2- | 788.4 | 786.5290 | C [symbol] H₇₇NO₁₀P | M + H |
| | | octadecenoyl)-glycero-3-phophoserine | | | | |
| LMPG03310407 | PS(18:3(9Z,12Z,15Z)/19:0) | 1-(9Z,12Z,15Z-octadecatrienoyl)-2- | 788.4 | 786.5290 | C [symbol] H₇₇NO₁₀P | M + H |
| | | octadecenoyl)-glycero-3-phophoserine | | | | |
| LMPG03310490 | PS(19:1(9Z)/17:2(9Z,12Z)) | 1-(9Z-nonadecenoyl)-2-(9Z,12Z- | 788.4 | 786.5290 | C [symbol] H₇₇NO₁₀P | M + H |
| | | heptadecadienoyl)-glycero-3-phophoserine | | | | |
| LMPG03310585 | PS(30:2(11Z,14Z)/18:1(9Z) | 1-(11Z,14Z-eicosadienoyl)-2-(9Z- | 788.4 | 786.5290 | C [symbol] H₇₇NO₁₀P | M + H |
| | | hexadecanoyl)-glycero-3-phophoserine | | | | |
| LMPG03310595 | PS(20:3(8Z,11Z,14Z)/18:0) | 1-(8Z,11Z,14Z-eicosadienoyl)-2- | 788.4 | 786.5290 | C [symbol] H₇₇NO₁₀P | M + H |
| | | hexadecanoyl)-glycero-3-phophoserine | | | | |
| LMPG03310759 | PS(22:2(13Z,18Z)/14:1(9Z)) | 1-(13Z,18Z-docosadienoyl)-2-(9Z- | 788.4 | 786.5290 | C [symbol] H₇₇NO₁₀P | M + H |
| | | tetradecenoyl)-glycero-3-phophoserine | | | | |
| LMPG03310933 | PS(18:0/20:3(8Z,11Z,14Z)) | 1-hexadecanoyl-2-(8Z,11Z,14Z- | 788.4 | 786.5290 | C [symbol] H₇₇NO₁₀P | M + H |
| | | eicosatrienoyl)-glycero-3-phophoserine | | | | |
| LMPG03310959 | PS(18:1(9Z)/18:2(9Z,12Z)) | 1-(9Z-octadecanoyl)-2-(9Z,12Z- | 788.4 | 786.5290 | C [symbol] H₇₇NO₁₀P | M + H |
| | | octadecantrienoyl)-glycero-3-phophoserine | | | | |
| LMPG03333065 | PS(P-20:0/17:2(9Z,12Z)) | 1-(1Z-elcosenyl)-2-(9Z,12Z- | 788.4 | 786.5290 | C [symbol] H [symbol] NO₉P | M + H |
| | | heptodecotrienoyl)-glycero-3-phophoserine | | | | |
| LMGP01010750 | PC(18:0/18:1(11Z)) | 1-octadecanoyl-2-(11Z-octadecenoyl)-sn- | 788.5 | 788.6164 | C₄₄H₃₇NO₃P | M + H |
| | | glycero-3-phosphocholine | | | | |
| LMGP01010751 | PC(18:0/18:1(12Z)) | 1-octadecanoyl-2-(12Z-octadecenoyl)-sn- | 788.5 | 788.6164 | C₄₄H₃₇NO₃P | M + H |
| | | glycero-3-phosphocholine | | | | |
| LMGP01010753 | PC(18:0/18:1(13Z)) | 1-octadecanoyl-2-(13Z-octadecenoyl)-sn- | 788.5 | 788.6164 | C₄₄H₃₇NO₃P | M + H |
| | | glycero-3-phosphocholine | | | | |
| LMGP01010754 | PC(18:0/18:1(16Z)) | 1-octadecanoyl-2-(16Z-octadecenoyl)-sn- | 788.5 | 788.6164 | C₄₄H₃₇NO₃P | M + H |
| | | glycerol-3-phosphocholine | | | | |
| LMGP01010756 | PC(18:0/18:1(6Z)) | 1-octadecanoyl-2-(6Z-octadecenoyl)-sn- | 788.5 | 788.6164 | C₄₄H₃₇NO₃P | M + H |
| | | glycero-3-phosphocholine | | | | |
| LMGP01010758 | PC(18:0/18:1(7Z)) | 1-octadecanoyl-2-(2Z-octadecenoyl)-sn- | 788.5 | 788.6164 | C₄₄H₃₇NO₃P | M + H |
| | | glycero-3-phosphocholine | | | | |
| LMGP01010759 | PC(18:0/18:1(9Z)) | 1-octadecanoyl-2-(9Z-octadecenoyl)-sn- | 788.5 | 788.6164 | C₄₄H₃₇NO₃P | M + H |
| | | glycero-3-phosphocholine | | | | |
| LMGP01010761 | PC(18:0/18:1(9Z)) | 1-octadecanoyl-2-(9Z-octadecenoyl)-sn- | 788.5 | 788.6164 | C₄₄H₃₇NO₃P | M + H |
| | | glycero-3-phosphocholine | | | | |
| LMGP01010840 | PC(18:1(11Z)/18:0) | 1-(11Z-octadecenoyl)-2-octadecanoyl-sn- | 788.5 | 788.6164 | C₄₄H₃₇NO₃P | M + H |
| | | glycero-3-phosphocholine | | | | |
| LMGP01010888 | PC(18:1(9Z)/18:0) | 1-(9Z-octadecenoyl)-2-octadecanoyl-sn- | 788.5 | 788.6164 | C₄₄H₃₇NO₃P | M + H |
| | | glycero-3-phosphocholine | | | | |
| LMGP01011376 | PC(14:0/22:1(11Z)) | 1-tetradecanoyl-2-(11Z-docosenoyl)- | 788.5 | 788.6164 | C₄₄H₃₇NO₃P | M + H |
| | | glycero-3-phosphocholine | | | | |
| LMGP01011404 | PC(14:1(9Z)/22:0) | 1-(9Z-tetradecenoyl)-2-docosanoyl- | 788.5 | 788.6164 | C₄₄H₃₇NO₃P | M + H |
| | | glycero-3-phosphocholine | | | | |
| LMGP01011456 | PC(18:1(9Z)/21:0) | 1-(9Z-pentadecenoyl)-2-heneicosanoyl- | 788.5 | 788.6164 | C₄₄H₃₇NO₃P | M + H |
| | | glycero-3-phosphocholine | | | | |
| LMGP01011468 | PC(16:0/20:0(11Z)) | 1-hexadecanoyl-2-(11Z-eicosenoyl)- | 788.5 | 788.6164 | C₄₄H₃₇NO₃P | M + H |
| | | glycero-3-phosphocholine | | | | |
| LMGP01011488 | PC(16:1(9Z)/20:0) | 1-(9Z-hexadecanoyl)-2-eicosanoyl- | 788.5 | 788.6164 | C₄₄H₃₇NO₃P | M + H |
| | | glycero-3-phosphocholine | | | | |
| LMGP01011510 | PC(17:0/19:1(9Z)) | 1-heptadecanoyl-2-(9Z-nonadecenoyl)- | 788.5 | 788.6164 | C₄₄H₃₇NO₃P | M + H |
| | | glycero-3-phosphocholine | | | | |
| LMGP01011537 | PC(17:1(9Z)/19:0) | 1-(9Z-heptadecenoyl)-2-nonadecanoyl- | 788.5 | 788.6164 | C₄₄H₃₇NO₃P | M + H |
| | | glycero-3-phosphocholine | | | | |
| LMGP01011736 | PC(19:0/17:1(9Z)) | 1-nonadecanoyl-2-(9Z-heptadecenoyl)- | 788.5 | 788.6164 | C₄₄H₃₇NO₃P | M + H |
| | | glycero-3-phosphocholine | | | | |
| LMGP01011764 | PC(19:1(9Z)/17:0) | 1-(9Z-nonadecenoyl)-2-heptadecanoyl- | 788.5 | 788.6164 | C₄₄H₃₇NO₃P | M + H |
| | | glycero-3-phosphocholine | | | | |
| LMGP01011790 | PC(20:0/16:1(9Z)) | 1-eicosanoyl-2-(9Z-hexadecenoyl)- | 788.5 | 788.6164 | C₄₄H₃₇NO₃P | M + H |
| | | glycero-3-phosphocholine | | | | |
| LMGP01011811 | PC(20:1(11Z)/16:0) | 1-(11Z-eicosenoyl)-2-hexadecanoyl- | 788.5 | 788.6164 | C₄₄H₃₇NO₃P | M + H |
| | | glycero-3-phosphocholine | | | | |
| LMGP01011957 | PC(21:0/15:1(9Z)) | 1-heneicosanoyl-2-(9Z-pentadecenoyl)- | 788.5 | 788.6164 | C₄₄H₃₇NO₃P | M + H |
| | | glycero-3-phosphocholine | | | | |
| LMGP01011980 | PC(22:0/14:1(9Z)) | 1-docosanoyl-2-(9Z-tetradecenoyl)- | 788.5 | 788.6164 | C₄₄H₃₇NO₃P | M + H |
| | | glycero-3-phosphocholine | | | | |
| LMGP01012006 | PC(22:1(11Z)/14:0) | 1-(11Z-docosenoyl)-2-tetradecanoyl- | 788.5 | 788.6164 | C₄₄H₃₇NO₃P | M + H |
| | | glycero-3-phosphocholine | | | | |
| LMGP01012129 | PC(14:0/228:1(13Z)) | 1-tetradecanoyl-2-(13Z-docosenoyl)-sn- | 788.5 | 788.6164 | C₄₄H₃₇NO₃P | M + H |
| | | glycero-3-phosphocholine | | | | |
| LMGP01012218 | PC(22:1(13Z)/14:0) | 1-(13Z-docosenoyl)-2-tetradecanoyl-sn- | 788.5 | 788.6164 | C₄₄H₃₇NO₃P | M + H |
| | | glycero-3-phosphocholine | | | | |

[symbol]

indicates data missing or illegible when filed

TABLE 33
LIST OF TRIACYLGLYCERIDES:

| | | | Input | Exact | | |
|---|---|---|---|---|---|---|
| LM_ID | Name | Systematic Name | m/z | m/z | Formula | Ion |
| LMGL03012681 | TG(12:0/20:4(5Z,8Z,11Z,14Z)/20:4(5Z,8Z,11Z,14Z)) | 1-dodecanoyl-2,3-di-(5Z,8Z,11Z,14Z-eicosatetraenoyl)-sn-glycerol | 847.5 | 847.6810 | C₄₄H₃₇O₃ | M + H |
| | [iso3] | | | | | |
| LMGL03012953 | TG(15:1(9Z)/15:1(9Z)/22:6(4Z,7Z,10Z,13Z,16Z,19Z)) | 1,2-di-(9Z-pentadecenoyl)-3-(4Z,7Z,10Z,13Z,16Z,19Z-docosahexaenoyl)-sn- | 847.5 | 847.6810 | C₄₄H₃₇O₃ | M + H |
| | [iso3] | glycerol | | | | |
| LMGL03012986 | TG(16:0/18:4(6Z,9Z,12Z,15Z)/18:4(6Z,9Z,12Z,15Z)) | 1-hexadecanoyl-2,3-di-(6Z.9Z,12Z,15Z-octadecatetraenoyl)-sn-glycerol | 847.5 | 847.6810 | C₄₄H₃₇O₃ | M + H |
| | [iso3] | | | | | |
| LMGL03013014 | TG(17:2(9Z,12Z)/17:2(9Z.12Z)/18:4(6Z,9Z,12Z,15Z)) | 1,2-di-(9Z,12Z-heptadecadienoyl)-3-(6Z,9Z,12Z,15Z-octadecatetraenoyl)-sn- | 847.5 | 847.6810 | C₄₄H₃₇O₃ | M + H |
| | [iso3] | glycerol | | | | |
| LMGL03013500 | TG(12:0/18:2(9Z.12Z)/22:6(4Z,7Z,10Z,13Z,16Z,19Z)) | 1-dodecanoyl-2-(9Z,9Z,12Z-octadecadienoyl)-3-(7Z,10Z,13Z,16Z,19Z- | 847.5 | 847.6810 | C₄₄H₃₇O₃ | M + H |
| | [iso6] | docosapentaenoyl)-sn-glycerol | | | | |
| LMGL03013517 | TG(12:0/18:3(6Z,9Z,12Z)/22:5(7Z,10Z,13Z,16Z,19Z)) | 1-dodecanoyl-2-(6Z,9Z,12Z-octadecatrinoyl)-3-(7Z,10Z,13Z,16Z,19Z- | 847.5 | 847.6810 | C₄₄H₃₇O₃ | M + H |
| | [iso6] | docosapentaenoyl)-sn-glycerol | | | | |
| LMGL03013534 | TG(12:0/18:3(9Z,12Z,15Z)/22:5(7Z,10Z,13Z,16Z,19Z)) | 1-dodecanoyl-2-(9Z,12Z,15Z-octadecatrienoyl)-3-(7Z,10Z,13Z,16Z,19Z- | 847.5 | 847.6810 | C₄₄H₃₇O₃ | M + H |
| | [iso6] | docosapentaenoyl)-sn-glycerol | | | | |
| LMGL03013549 | TG(12:0/18:4(6Z,9Z,12Z,15Z)/22:4(7Z,10Z,13Z,16Z)) | 1-dodecanoyl-2-(6Z,9Z,12Z,15Z-octadecatetraenoyl)-3-(7Z,10Z,13Z,16Z- | 847.5 | 847.6810 | C₄₄H₃₇O₃ | M + H |
| | [iso6] | docosatetraenoyl)-sn-glycerol | | | | |
| LMGL03013618 | TG(12:0/20:3(8Z,11Z,14Z)/20:5(5Z,8Z,11Z,14Z,17Z)) | 1-dodecanoyl-2-(8Z,11Z,14Z-eicosatrienoyl)-3-(5Z,8Z,11Z,14Z,17Z- | 847.5 | 847.6810 | C₄₄H₃₇O₃ | M + H |
| | [iso6] | eicosapentaenoyl)-sn-glycerol | | | | |
| LMGL03013905 | TG(13:0/17:2(9Z,12Z)/22:6(4Z,7Z,10Z,13Z,16Z,19Z)) | 1-tridecanoyl-2-(9Z,12Z-heptadecadienoyl)-3-(4Z,7Z,10Z,13Z,16Z,19Z- | 847.5 | 847.6810 | C₄₄H₃₇O₃ | M + H |
| | [iso6] | docosahexaenoyl)-sn-glycerol | | | | |
| LMGL03014410 | TG(14:0/18:3(6Z,9Z,12Z)/20:5(5Z,8Z,11Z,14Z,17Z)) | 1-tetradecanoyl-2-(6Z,9Z,12Z-octadecatrienoyl)-3-(5Z,8Z,11Z,14Z,17Z- | 847.5 | 847.6810 | C₄₄H₃₇O₃ | M + H |
| | [iso6] | eicosapentaenoyl)-sn-glycerol | | | | |
| LMGL03014427 | TG(14:0/18:3(9Z,12Z,15Z)/20:5(5Z,8Z,11Z,14Z,17Z)) | 1-tetradecanoyl-2-(9Z,12Z,15Z-octadecatrienoyl)-3-(5Z,8Z,11Z,14Z,17Z- | 847.5 | 847.6810 | C₄₄H₃₇O₃ | M + H |
| | [iso6] | eicosapentaenoyl)-sn-glycerol | | | | |
| LMGL03014442 | TG(14:0/18:4(6Z,9Z,12Z,15Z)/20:4(5Z,8Z,11Z,14Z)) | 1-tetradecanoyl-2-(6Z,9Z,12Z,15Z-octadecatetraenoyl)-3-(5Z,8Z,11Z,14Z- | 847.5 | 847.6810 | C₄₄H₃₇O₃ | M + H |
| | [iso6] | eicosatetraenoyl)-sn-glycerol | | | | |
| LMGL03014677 | TG(14:1(9Z)/16:1(9Z)/22:6(4Z,7Z,10Z,13Z,16Z,19Z)) | 1-(9Z-tetradecenoyl)-2-(9Z-hexadecenoyl)-3-(4Z,7Z,10Z,13Z,16Z,19Z- | 847.5 | 847.6810 | C₄₄H₃₇O₃ | M + H |
| | [iso6] | docosahexaenoyl)-sn-glycerol | | | | |
| LMGL03014798 | TG(14:1(9Z)/18:2(9Z,12Z)/20:5(5Z,8Z,11Z,14Z,17Z)) | 1-(9Z-tetradecenoyl)-2-(9Z,12Z-octadecadienoyl)-3-(5Z,8Z,11Z,14Z,17Z,- | 847.5 | 847.6810 | C₄₄H₃₇O₃ | M + H |
| | [iso6] | eicosapentaenoyl)-sn-glycerol | | | | |
| LMGL03014815 | TG(14:1(9Z)/18:3(6Z,9Z,12Z)/20:4(5Z,8Z,11Z,14Z)) | 1-(9Z-tetradecenoyl)-2-(6Z,9Z,12Z-octadecatrienoyl)-3-(5Z,8Z,11Z,14Z- | 847.5 | 847.6810 | C₄₄H₃₇O₃ | M + H |
| | [iso6] | eicosatetraenoyl)-sn-glycerol | | | | |
| LMGL03014832 | TG(14:1(9Z)/18:3(9Z,12Z,15Z)/20:4(5Z,8Z,11Z,14Z)) | 1-(9Z-tetradecenoyl)-2-(9Z,12Z,15Z-octadecatrienoyl)-3-(5Z,8Z,11Z,14Z- | 847.5 | 847.6810 | C₄₄H₃₇O₃ | M + H |
| | [iso6] | eicosatetraenoyl)-sn-glycerol | | | | |
| LMGL03014847 | TG(14:1(9Z)/18:4(6Z,9Z,12Z,15Z)/20:3(8Z,11Z,14Z)) | 1-(9Z-tetradecenoyl)-2-(6Z,9Z,12Z,15Z-octadecatetraenoyl)-3-(6Z,11Z,14Z- | 847.5 | 847.6810 | C₄₄H₃₇O₃ | M + H |
| | [iso6] | eicosatrienoyl)-sn-glycerol | | | | |
| LMGL03015467 | TG(15:1(9Z)/17:2(9Z,12Z)/20:5(5Z,8Z,11Z,14Z,17Z)) | 1-(9Z-pentadecenoyl)-2-(9Z,12Z-heptadecadienoyl)-3-(5Z,8Z,11Z,14Z,17Z- | 847.5 | 847.6810 | C₄₄H₃₇O₃ | M + H |
| | [iso6] | eicosapentaenoyl)-sn-glycerol | | | | |
| LMGL03015827 | TG(16:1(9Z)/18:3(6Z,9Z,12Z)/18:4(6Z,9Z,12Z,15Z)) | 1-(9Z-hexadiecenoyl)-2-(6Z,9Z,12Z-octadecatrienoyl)-3-(6Z,9Z,12Z,15Z- | 847.5 | 847.6810 | C₄₄H₃₇O₃ | M + H |
| | [iso6] | octadecatetraenoyl)-sn-glycerol | | | | |
| LMGL03015844 | TG(16:1(9Z)/18:3(9Z,12Z,15Z)/18:4(6Z,9Z,12Z,15Z)) | 1-(9Z-hexadecenoyl)-2-(9Z,12Z,15Z-octadecatrienoyl)-3-(6Z,9Z,12Z,15Z- | 847.5 | 847.6810 | C₄₄H₃₇O₃ | M + H |
| | [iso6] | octadecatetraenoyl)-sn-glycerol | | | | |
| LMGP04010476 | PG(19:0/22:1(11Z)) | 1-nonadecanoyl-2-(11Z-docosenoyl)-glycero-3-phospho-(1′-sn-glycerol) | 847.5 | 847.6423 | C₄₇H₃₇O₁₆P | M + H |
| LMGP04010506 | PG(19:1(9Z)/22:0) | 1-(9Z-nonadecenoyl)-2-docosanoyl-glycero-3-phospho-(1′-sn-glycerol) | 847.5 | 847.6423 | C₄₇H₃₇O₁₆P | M + H |
| LMGP04010552 | PG(20:1(11Z)/21:0) | 1-(11Z-eicosenoyl)-2-heneicosanoyl-glycero-3-phospho-(1′-sn-glycerol) | 847.5 | 847.6423 | C₄₇H₃₇O₁₆P | M + H |
| LMGP04010588 | PG(20:2(11Z,14Z)/22:6(4Z,7Z,10Z,13Z,16Z,19Z)) | 1-(11Z,14Z-eicosadienoyl)-2-(4Z,7Z,10Z,13Z,16Z,19Z-docosahexaenoyl)- | 847.5 | 847.5484 | C₄₇H₃₇O₁₆P | M + H |
| | | glycerol-3-phospho-(1′-sn-glycerol) | | | | |
| LMGP04010647 | PG(20:4(5Z,8Z,11Z,14Z)/22:4(7Z,10Z,13Z,16Z)) | 1-(5Z,8Z,11Z,14Z-eicosatetraenoyl)-2-(7Z,10Z,13Z,16Z-docosatetraenoyl)- | 847.5 | 847.5484 | C₄₇H₃₇O₁₆P | M + H |
| | | glycerol-3-phospho-(1′-sn-glycerol) | | | | |
| LMGP04010693 | PG(21:0/20:1(11Z)) | 1-heneicosanoyl-2-(11Z-eicosenoyl)-glycero-3-phospho-(1′-sn-glycerol) | 847.5 | 847.6423 | C₄₇H₃₇O₁₆P | M + H |
| LMGP04010715 | PG(22:0/19:1(9Z)) | 1-docosanoyl-2-(9Z-nonadecenoyl)-glycero-3-phospho-(1′-sn-glycerol) | 847.5 | 847.6823 | C₄₇H₃₇O₁₆P | M + H |
| LMGP04010742 | PG(22:1(11Z)/19:0) | 1-(11Z-docosenoyl)-2-nonadecanoyl-glycero-3-phospho-(1′-sn-glycerol) | 847.5 | 847.6823 | C₄₇H₃₇O₁₆P | M + H |
| LMGP04010810 | PG(22:4(7Z,10Z,13Z,16Z)/20:4(5Z,8Z,11Z,14Z)) | 1-(7Z.10Z,13Z,16Z-docosatetraenoyl)-2-(5Z,8Z,11Z,14Z-eicosatetraenoyl)- | 847.5 | 847.5484 | C₄₇H₃₇O₁₆P | M + H |
| | | glycero-3-phospho-(1′-sn-glycerol) | | | | |
| LMGP04010837 | PG(22:6(4Z,7Z,10Z,13Z,16Z.19Z)/20:2(11Z,14Z) | 1-(4Z.7Z,10Z,13Z,16Z,19Z-docosahexaenoyl)-2-(11Z,14Z-eicosadienoyl)- | 847.5 | 847.5484 | C₄₇H₃₇O₁₆P | M + H |
| | | glycero-3-phospho-(1′-sn-glycerol) | | | | |
| LMGP04020066 | PG(O-20:0/22:1(11Z)) | 1-eicosyl-2-(11Z-docosanoyl)-glycero-3-phospho-(1′-sn-glycerol) | 847.5 | 847.6786 | C₄₃H₃₇O₃P | M + H |
| LMGP04030082 | PG(P-20:0/22:0) | 1-(1Z-eicosenyl)-2-docosanoyl-glycero-3-phospho-(1′-sn-glycerol) | 847.5 | 847.6786 | C₄₃H₃₇O₃P | M + H |
| LMGP06010121 | PI(15:0/20:3(8Z,11Z,14Z)) | 1-pentadecanoyl-2-(8Z,11Z,14Z-eicosatrienoyl)-glycero-3-phospho-(1′-myo- | 847.5 | 847.5331 | C₄₄H₃₇O₁₃P | M + H |
| | | inositol) | | | | |
| LMGP06010150 | PI(15:1(9Z)/20:2(11Z,14Z)) | 1-(9Z-pentadecenoyl)-2-(11Z,14Z-eicosadienoyl)-glycero-3-phospho-(1′-myo- | 847.5 | 847.5331 | C₄₄H₃₇O₁₃P | M + H |
| | | inositol) | | | | |
| LMGP06010202 | PI(17:0/18:3(6Z,9Z,12Z) | 1-heptadecanoyl-2-(6Z,9Z,12Z-octadecatrienoyl)-glycero-3-phospho-(1′-myo- | 847.5 | 847.5331 | C₄₄H₃₇O₁₃P | M + H |
| | | inositol) | | | | |
| LMGP06010203 | PI(17:0/18:3(9Z,12Z,15Z)) | 1-heptadecanoyl-2-(9Z,12Z,15Z-octadecatrienoyl)-glycero-3-phospho-(1′- | 847.5 | 847.5331 | C₄₄H₃₇O₁₃P | M + H |
| | | myo-inositol) | | | | |
| LMGP06010228 | PI(17:1(9Z,)18:2(9Z,12Z)) | 1-(9Z-heptadacenoyl)-2-(9Z,12Z-octadecadienoyl)-glycero-3-phospho-(1′- | 847.5 | 847.5331 | C₄₄H₃₇O₁₃P | M + H |
| | | myo-inositol) | | | | |
| LMGP06010257 | PI(17:2(9Z,12Z)/18:1(9Z)) | 1-(9Z,12Z-heptadecadienoyl)-2-(9Z-octadecenoyl)-glycero-3-phospho-(1′- | 847.5 | 847.5331 | C₄₄H₃₇O₁₃P | M + H |
| | | myo-inositol) | | | | |
| LMGP06010297 | PI(18:1(9Z)/17:2(9Z,12Z)) | 1-(9Z-octadecenoyl)-2-(9Z,12Z-heptadecadienoyl)-glycero-3-phospho-(1′- | 847.5 | 847.5331 | C₄₄H₃₇O₁₃P | M + H |
| | | myo-inositol) | | | | |
| LMGP06010316 | PI(18:2(9Z,12Z)/17:1(9Z)) | 1-(9Z,12Z-octadecadienoyl)-2-(9Z-heptadecenoyl)-glycero-3-phospho-(1′- | 847.5 | 847.5331 | C₄₄H₃₇O₁₃P | M + H |
| | | myo-inositol) | | | | |
| LMGP06010342 | PI(18:3(6Z,12Z)/17:0) | 1-(6Z,9Z,12Z-octadecatrienoyl)-2-heptadecanoyl-glycero-3-phospho-(1′-myo- | 847.5 | 847.5331 | C₄₄H₃₇O₁₃P | M + H |
| | | inositol) | | | | |
| LMGP06010372 | PI(18:3(9Z,12Z,5Z)/17:0) | 1-(9Z,12Z,15Z-octadecatrienoyl)-2-heptadecanoyl-glycero-3-phospho-(1′- | 847.5 | 847.5331 | C₄₄H₃₇O₁₃P | M + H |
| | | myo-inositol) | | | | |
| LMGP06010531 | PI(20:2(11Z,14Z)/15:1(9Z)) | 1-(11Z,14Z-eicosadienoyl)-2-(9Z-heptadecenoyl)-glycero-3-phospho-(1′-myo- | 847.5 | 847.5331 | C₄₄H₃₇O₁₃P | M + H |
| | | inositol) | | | | |
| LMGP06010561 | PI(20:3(8Z,11Z,14Z)/15:0) | 1-(8Z,11Z,14Z-eicosatrienoyl)-2-pentadecenoyl-glycero-3-phospho-(1′-myo- | 847.5 | 847.5331 | C₄₄H₃₇O₁₃P | M + H |
| | | inositol) | | | | |
| LMGP06020030 | PI(O-18:0/18:3(6Z,9Z,12Z)) | 1-octadecyl-2-(6Z,9Z,12Z-octadecatrienoyl)-glycero-3-phospho-(1′-myo- | 847.5 | 847.5695 | C₄₄H₃₇O₁₃P | M + H |
| | | inositol) | | | | |
| LMGP05020931 | PI(O-18:0/18:3(9Z,12Z,15Z)) | 1-octadetyl-2-(9Z,12Z,15Z-octadecatrienoyl)-glycero-3-phospho-(1′-myo- | 847.5 | 847.5695 | C₄₄H₃₇O₁₃P | M + H |
| | | inositol) | | | | |
| LMGP06020076 | PI(O-16:0/20:3(8Z,11Z,14Z)) | 1-hexadecyl-2-(8Z,11Z,14Z-eicosatrienoyl)-glycero-3-phospho-(1′-myo- | 847.5 | 847.5695 | C₄₄H₃₇O₁₃P | M + H |
| | | inositol) | | | | |
| LMGP06030021 | PI(P-16:0/20:2(11Z,14Z)) | 1-(1Z-hexadecenyl)-2-(11Z,14Z-eicosadienoyl)-glycero-3-phospho-(1′-myo- | 847.5 | 847.5695 | C₄₄H₃₇O₁₃P | M + H |
| | | inositol) | | | | |
| LMGP06030041 | PI(P-18:0/18:2(9Z,12Z)) | 1-(1Z-octadecenoyl)-2-(9Z,12Z-octadecadienoyl)-glycero-3-phospho-(1′-myo- | 847.5 | 847.5695 | C₄₄H₃₇O₁₃P | M + H |
| | | inositol) | | | | |
| LMPR03020001 | undecaprenyl phosphate | 3,7,11,15,19,23,27,31,35,39,43-undecamethytetratetraconta- | 847.5 | 847.6728 | C₄₄H_{14 O}_{4L P} | M + H |
| | | 2Z,8Z,10Z,14Z,18Z,22Z,26Z,30Z,34E,38E,42-undecaen-1-yl phosphate | | | | |
| LMPR03020005 | all-trans-Undecaprenyl phosphate | (2E,6E,10E,14E,18E,22E,26E,30E,34E,38E)-3,7,11,15,19,23,27,31,35,39,43- | 847.5 | 847.6728 | C₄₄H_{14 O}_{4L P} | M + H |
| | | undecamethytetratetraconta-2,6,10,14,18,22,26,30,34,38,42-undecaen-1-yl | | | | |
| | | dihydrogen phosphate | | | | |

Possible Lipid Structures

| | | | | | | |
|---|---|---|---|---|---|---|
| LMGP03010510 | PS(19:1(9Z)/22:6(4Z,7Z,10Z,13Z,16Z,19Z)) | 1-(9Z-nonadecenoyl)-2- | 848.4 | 848.5436 | C₄₇H₇₉NO₁₆P | M + H |
| | | (4Z,7Z,10Z,13Z,16Z,19Z-docosahexaenoyl)- | | | | |
| | | glycero-3-phosphoserine | | | | |
| LMGP03010834 | PS(22:6(4Z,7Z,10Z,13Z,16Z,19Z)/19:1(9Z)) | 1-(4Z,7Z,10Z,13Z,16Z,19Z- | 848.4 | 848.5436 | C₄₇H₇₉NO₁₆P | M + H |
| | | docosahexaenoyl)-2-(9Z-nenadecenoyl)- | | | | |
| | | glycero-3-phosphoserine | | | | |
| LMGP03030093 | PS(P-20:0/22:6(4Z,7Z,10Z,13Z,16Z,19Z)) | 1-(1Z-eicosenyl)-2-(4Z,7Z,10Z,13Z,16Z,19Z- | 848.4 | 848.5800 | C₄₅H₅₃NO₅P | M + H |
| | | docosahexaenoyl)-glycero-3-phosphoserine | | | | |
| LMGP01011785 | PC(19:1(9Z)/22:4(7Z,10Z,13Z,16Z)) | 1-(9Z-nonadecenoyl)-2-(7Z,10Z,13Z,16Z- | 850.5 | 850.6320 | C₄₉H₅₉NO₃P | M + H |
| | | docosaletraenoyl)-glycero-3-phosphocholine | | | | |
| LMGP01011950 | PC(20:5(5Z,8Z,11Z,14Z,17Z)/21:0) | 1-(5Z,8Z,11Z,14Z,17Z-eicosapentaenoyl)-2- | 850.5 | 850.6320 | C₄₉H₅₉NO₃P | M + H |
| | | heneicosanoyl-glycero-3-phosphocholine | | | | |
| LMGP01011975 | PC(21:0/20:5(5Z,8Z,11Z,14Z,17Z)) | 1-heneicosanoyl-2-(5Z,8Z,11Z,14Z,17Z- | 850.5 | 850.6320 | C₄₉H₅₉NO₃P | M + H |
| | | eicosapentaenoyl)-glycero-3-phosphocholine | | | | |
| LMGP01012084 | PC(22:4(7Z,10Z,13Z,16Z)/19:1(9Z)) | 1-(7Z,10Z,13Z,16Z-docosatetraenoyl)-2-(9Z- | 850.5 | 850.6320 | C₄₉H₅₉NO₃P | M + H |
| | | nonadecenoyl)-glycero-2-phosphocholine | | | | |
| LMGP01030103 | PC(P-20:0/22:4(7Z,10Z,13Z,16Z)) | 1-(1Z-eicosenyl)-2-(7Z,10Z,13Z,16Z- | 850.5 | 850.6684 | C₅₀H₉₃NO₇P | M + H |
| | | docosatetraenoyl)-glycero-3-phosphocholine | | | | |
| LMGP02011069 | PE(22:1(11Z)/22:4(7Z,10Z,13Z,16Z)) | 1-(11Z-docosenoyl)-2-(7Z,10Z,13Z,16Z- | 850.5 | 850.6320 | C₄₉H₅₉NO₃P | M + H |
| | | docosatetraenoyl)-glycero-3-phosphoethanolamine | | | | |
| LMGP02011129 | PE(22:4(7Z,10Z,13Z,16Z)/22:1(11Z)) | 1-(7Z,10Z,13Z,16Z-docosatetraenoyl)-2-(11Z- | 850.5 | 850.6320 | C₄₉H₅₉NO₃P | M + H |
| | | docosenoyl)-glycero-3-phosphoethanolamine | | | | |
| LMGP03010479 | PS(19:0/22:6(4Z,7Z,10Z,13Z,16Z,19Z)) | 1-nonadecanoyl-2-(4Z,7Z,10Z,13Z,16Z,19Z- | 850.5 | 850.5593 | C₄₇H₅₁NO₁₀P | M + H |
| | | docosahexaenoyl)-glycero-3-phosphoserine | | | | |
| LMGP03010833 | PS(22:6(4Z,7Z,10Z,13Z,16Z,19Z)/19:0) | 1-(4Z,7Z,10Z,13Z,16Z,19Z-docosahexaenoyl)-2- | 850.5 | 850.5593 | C₄₇H₅₁NO₁₀P | M + H |
| | | nonadecanoyl-glycero-3-phosphoserine | | | | |
| LMGP03020093 | PS(O- | 1-eicosyl-2-(4Z,7Z,10Z,13Z,16Z,19Z- | 850.5 | 850.5956 | C₄₅H₅₅NO₉P | M + H |
| | 20:0/22:6(4Z,7Z,10Z,13Z,16Z,19Z)) | docosahexaenoyl)-glycero-3-phosphoserine | | | | |
| LMFA07050001 | | R-hexanoyl CoA | 866.2 | 866.1957 | C₄₇H₄₇N₇O₁₇P₃S | M + H |
| LMGP01011119 | PC(22:6(4Z,7Z,10Z,13Z,16Z,19Z)/22:6(4Z,7Z,10Z,13Z,16Z,19Z)) | 1,2-di | 878.5 | 876.5694 | C₅₂H₈₁NO₈P | M + H |
| | | (4Z,7Z,10Z,13Z,16Z,19Z- | | | | |
| | | docosahexaenoyl)-sn- | | | | |
| | | glycero-3-phosphocholine | | | | |
| LMGP01030127 | PC(O-22:2(13Z,16Z)/22:3(10Z,13Z,16Z)) | 1-(13Z,16Z-docosenyl)-2- | 878.5 | 878.6997 | C₅₇H₂₇NO₇P | M + H |
| | | (10Z,13Z,16Z- | | | | |
| | | docosatrienoyl)-sn- | | | | |
| | | glycero-3-phosphocholine | | | | |
| LMGP03010004 | PS(21:0/22:6(4Z,7Z,10Z,13Z,16Z,19Z)) | 1-heneicosanoyl-2- | 878.5 | 878.5906 | C₄₉H₈₅NO₁₀P | M + H |
| | | (4Z,7Z,10Z,13Z,16Z,19Z- | | | | |
| | | docosahexaenoyl)-sn- | | | | |
| | | glycero-3-phosphoserine | | | | |
| LMGP03010841 | PS(22:6(4Z,7Z,10Z,13Z,16Z,19Z)/21:0) | 1- | 878.5 | 878.5906 | C₄₉H₅₅NO₁₀P | M + H |
| | | (4Z,7Z,10Z,13Z,16Z,19Z- | | | | |
| | | docosahexaenoyl)-2- | | | | |
| | | heneicosanoyl-glycero-3- | | | | |
| | | phosphoserine | | | | |
| LMFA07050024 | | 2E-octenoyl CoA | 892.4 | 892.2113 | C₂₉H₄₉N₇O₁₇P₃S | M + H |
| LMGP03010724 | PS(22:0/22:4(7Z,10Z,13Z,16Z)) | 1-docosanoyl-2-(7Z,10Z,13Z,16Z-docosatetraenoyl)- | 896.5 | 896.6375 | C₅₀H₉₁NO₁₀P | M + H |
| | | glycero-3-phosphoserine | | | | |
| LMGP03010784 | PS(22:2(13Z,16Z)/22:2(13Z,16Z)) | 1,2-di-(13Z,16Z-docosadienoyl)-sn-glycero-3- | 896.5 | 896.6375 | C₅₀H₉₁NO₁₀P | M + H |
| | | phosphoserine | | | | |
| LMGP03010813 | PS(22:4(7Z,10Z,13Z,16Z)/22:0) | 1-(7Z,10Z,13Z,16Z-docosatetraenoyl)-2-docosanoyl- | 896.5 | 896.6375 | C₅₀H₉₁NO₁₀P | M + H |
| | | glycero-3-phosphoserine | | | | |
| LMGP03010722 | PS(22:0/22:1(11Z)) | 1-docosanoyl-2-(11Z-docosenoyl)-glycero-3-phosphoserine | 902.5 | 902.6845 | C₄₈H₉₇NO₁₀P | M + H |
| LMGP03010751 | PS(22:1/(11Z)/22:0) | 1-(11Z-docosenoyl)-2-docosanoyl-glycero-3-phosphoserine | 902.5 | 902.6845 | C₄₈H₉₇NO₁₀P | M + H |
| LMGP06010213 | PI(17:0/22:1(11Z)) | 1-heptadecanoyl-2-(11Z-docosenoyl)- | 907.5 | 907.6270 | C₄₈H₂₂O₁₃P | M + H |
| | | glycero-3-phospho-(1′-myo-inositol) | | | | |
| LMGP06010241 | PI(17:1(9Z)/22:0) | 1-(9Z-heptadecenoyl)-2-docosanoyl-glycero- | 907.5 | 907.6270 | C₄₈H₂₂O₁₃P | M + H |
| | | 3-phospho-(1′-myo-inositol) | | | | |
| LMGP06010304 | PI(18:1(9Z)/21:0) | 1-(9Z-octadecenoyl)-2-heneicosanoyl- | 907.5 | 907.6270 | C₄₈H₂₂O₁₃P | M + H |
| | | glycero-3-phospho-(1′-myo-inositol) | | | | |
| LMGP06010422 | PI(18:4(6Z,9Z,12Z,15Z)/22:4(7Z,10Z,13Z,16Z)) | 1-(6Z,9Z,12Z,15Z-octadecateiraenoyl)-2- | 907.5 | 907.5331 | C₄₈H₅₀O₁₂P | M + H |
| | | (7Z,10Z,13Z,16Z-docosatetraenoyl)-glycero- | | | | |
| | | 3-phospho-(1′-myo-inositol) | | | | |
| LMGP06010437 | PI(19:0/20:1(11Z)) | 1-nonadecanoyl-2-(11Z-eicosenoyl)-glycero- | 907.5 | 907.6270 | C₄₈H₂₂O₁₃P | M + H |
| | | 3-phospho-(1′-myo-inositol) | | | | |
| LMGP06010467 | PI(19:1(9Z)/20:0) | 1-(9Z-nonadecanoyl)-2-eicosenoyl-glycero- | 907.5 | 907.6270 | C₄₈H₂₂O₁₃P | M + H |
| | | 3-phospho-(1′-myo-inositol) | | | | |
| LMGP06010489 | PI(20:0/19:1(9Z)) | 1-eicosanoyl-2-(9Z-nonadecenoyl)-glycero- | 907.5 | 907.6270 | C₄₈H₂₂O₁₃P | M + H |
| | | 3-phospho-(1′-myo-inositol) | | | | |
| LMGP06010513 | PI(20:1(11Z)/19:0) | 1-(11Z-eicosenoyl)-2-nonadecanoyl-glycero- | 907.5 | 907.6270 | C₄₈H₂₂O₁₃P | M + H |
| | | 3-phospho-(1′-myo-inositol) | | | | |
| LMGP06010581 | PI(20:3(8Z,11Z,14Z)/20:5(5Z,8Z,11Z,14Z,17Z)) | 1-(6Z,11Z,14Z-eicosatrienoyl)-2- | 907.5 | 907.5331 | C₄₈H₅₀O₁₂P | M + H |
| | | (5Z,8Z,11Z,14Z,17Z-eicosapentaenoyl)- | | | | |
| | | glycero-3-phospho-(1′-myo-inositol) | | | | |
| LMGP06010638 | PI(20:5(5Z,8Z,11Z,14Z,17Z)/20:3(8Z,11Z,14Z)) | 1-(5Z,8Z,11Z,14Z,17Z-eicosapentaenoyl)-2- | 907.5 | 907.5331 | C₄₈H₅₀O₁₂P | M + H |
| | | (8Z,11Z,14Z,-eicosatrienoyl)-glycero-3- | | | | |
| | | phospho-(1′-myo-inositol) | | | | |
| LMGP06010654 | PI(21:0/18:1(9Z)) | 1-heneicosenoyl-2-(9Z-octadecenoyl)- | 907.5 | 907.6270 | C₄₈H₂₂O₁₃P | M + H |
| | | glycero-3-phospho-(1′-myo-inositol) | | | | |
| LMGP06010677 | PI(22:0/17:1(9Z)) | 1-dicosanoyl-2-(9Z-heptadecenoyl)-glycero- | 907.5 | 907.6270 | C₄₈H₂₂O₁₃P | M + H |
| | | 3-phospho-(1′-myo-inositol) | | | | |
| LMGP06010702 | PI(22:1(11Z)/17:0) | 1-(11Z-docosenoyl)-2-heptadecanoyl- | 907.5 | 907.6270 | C₄₈H₂₂O₁₃P | M + H |
| | | glycero-3-phospho-(1′-myo-inositol) | | | | |
| LMGP06010772 | PI(22:4(7Z,10Z,13Z,16Z)/18:4(6Z,9Z,12Z,15Z)) | 1-(7Z,10Z,13Z,16Z-docosatetraenoyl)-2- | 907.5 | 907.5331 | C₄₈H₅₀O₁₂P | M + H |
| | | (6Z,9Z,12Z,15Z-octadecatetraenoyl)- | | | | |
| | | glycero-3-phospho-(1′-myo-inositol) | | | | |
| LMGP06010798 | PI(22:6(4Z,7Z,10Z,13Z,16Z,19Z)/18:2(9Z,12Z)) | 1-(4Z,7Z,10Z,13Z,16Z,19Z- | 907.5 | 907.5331 | C₄₈H₅₀O₁₂P | M + H |
| | | docosahexaenoyl)-2-(9Z,19Z- | | | | |
| | | octadecadienoyl)-glycero-3-phospho-(1′- | | | | |
| | | myo-inositol) | | | | |
| LMGP06010827 | PI(20:4(5Z,8Z,11Z,14Z)/20:4(5Z,8Z,11Z,14Z)) | 1,2-di-(5Z,8Z,11Z,14Z-eicosatetraenoyl)-sn- | 907.5 | 907.5331 | C₄₈H₅₀O₁₂P | M + H |
| | | glycero-3-phospho-(1′-myo-inositol) | | | | |
| LMGP06010844 | PI(18:2(9Z,12Z)/22:6(4Z,7Z,10Z,13Z,16Z,19Z)) | 1-(9Z,12Z-octadecadienoyl)-2- | 907.5 | 907.5331 | C₄₈H₅₀O₁₂P | M + H |
| | | (4Z,7Z,10Z,13Z,16Z,19Z- | | | | |
| | | docosahexaenoyl)-glycero-3-phospho-(1′- | | | | |
| | | myo-inositol) | | | | |
| LMGP06020039 | PI(O-18:0/22:1(11Z)) | 1-octadecyl-2-(11Z-docosenoyl)-glycero-3- | 907.5 | 907.6634 | C₄₈H₅₀O₁₂P | M + H |
| | | phospho-(1′-myo-inositol) | | | | |
| LMGP06020061 | PI(O-20:0/20:1(11Z)) | 1-eicosyl-2-(11Z-eicosenoyl)-glycero-3- | 907.5 | 907.6634 | C₄₈H₅₀O₁₂P | M + H |
| | | phospho-(1′-myo-inositol) | | | | |
| LMGP06030053 | PI(P-18:0/22:0) | 1-(1Z-octadecenyl)-2-docosenoyl-glycero-3- | 907.5 | 907.6634 | C₄₈H₅₀O₁₂P | M + H |
| | | phospho-(1′-myo-inositol) | | | | |
| LMGP06030076 | PI(P-20:0/20:0) | 1-(1Z-eicosenyl)-2-eicosanoyl-glycero-3- | 907.5 | 907.6634 | C₄₈H₅₀O₁₂P | M + H |
| | | phospho-(1′-myo-inositol) | | | | |
| LMST5050019 | Kuriensoside I | 3beta-(2-O-methyl-beta-D- | 907.5 | 907.4897 | C₄₄H₇₅O₁₂P | M + H |
| | | xylopyranosyloxy)-24R-[2-O-methyl-beta-D- | | | | |
| | | xylopyranosyl-(1-5)-alpha-L- | | | | |
| | | arabinofuranosyloxy]-5alpha-cholest-22E-en- | | | | |
| | | 4beta-6alpha,7alpha,8,15beta-pentol | | | | |
| LMGL03010653 | TG(18:3(9Z,12Z,15Z)/18:3(9Z,12Z,15Z)/20:0) | 1,2-di-(9Z,12Z,15Z-octadecatrienoyl)-3- | 907.6 | 907.7749 | C₅₉H₁₀₃O₈ | M + H |
| | [iso3] | eicosanoyl-sn-glycerol | | | | |
| LMGL03010657 | TG(18:2(9Z,12Z)/18:3(9Z,12Z,15Z)/20:1(11Z)) | 1-(9Z,12Z,-octadecadienoyl)-2- | 907.6 | 907.7749 | C₅₉H₁₀₃O₈ | M + H |
| | [iso6] | (9Z,12Z,15Z-octadecatrienoyl)-3-(11Z- | | | | |
| | | eicosenoyl)-sn-glycerol | | | | |
| LMGL03010682 | TG(18:1(9Z)/18:3(9Z,12Z,15Z)/20:2(11Z,14Z)) | 1-(9Z-octadecenoyl)-2-(9Z,12Z,15Z- | 907.6 | 907.7749 | C₅₉H₁₀₃O₈ | M + H |
| | [iso6] | octadecatrienoyl)-3-(11Z,14Z- | | | | |
| | | eicosadienoyl)-sn-glycerol | | | | |
| LMGL03010663 | TG(18:2(9Z,12Z)/18:2(9Z,12Z)/20:2(11Z,14Z)) | 1,2-di-(9Z,12Z-octadecadienoyl)-3- | 907.6 | 907.7749 | C₅₉H₁₀₃O₈ | M + H |
| | [iso3] | (11Z,14Z-eicosadienoyl)-sn-glycerol | | | | |
| LMGL03010664 | TG(16:0/20:3(8Z,11Z,14Z)/20:3(8Z,11Z,14Z)) | 1-hexadecanoyl-2,3-di-(8Z,11Z,14Z- | 907.6 | 907.7749 | C₅₉H₁₀₃O₈ | M + H |
| | [iso3] | eicosatrienoyl)-sn-glycerol | | | | |
| LMGL03010665 | TG(16:1(9Z)/20:2(11Z,14Z)/20:3(8Z,11Z,14Z)) | 1-(9Z-hexadecanoyl)-2-(11Z,14Z- | 907.6 | 907.7749 | C₅₉H₁₀₃O₈ | M + H |
| | [iso6] | eicosadienoyl)-3-(8Z,11Z,14Z- | | | | |
| | | eicosatrienoyl)-sn-glycerol | | | | |
| LMGL03010669 | TG(18:0/18:3(9Z,12Z,15Z)/20:3(8Z,11Z,14Z)) | 1-octadecanoyl-2-(9Z,12Z,15Z- | 907.6 | 907.7749 | C₅₉H₁₀₃O₈ | M + H |
| | [iso6] | octadecatrienoyl)-3-(8Z,11Z,14Z- | | | | |
| | | eicosatrienoyl)-sn-glycerol | | | | |
| LMGL03010670 | TG(18:1(9Z)/18:2(9Z,12Z)/20:3(8Z,11Z,14Z)) | 1-(9Z-octadecenoyl)-2-(9Z,12Z- | 907.6 | 907.7749 | C₅₉H₁₀₃O₈ | M + H |
| | [iso6] | octadecadienoyl)-3-(8Z,11Z,14Z- | | | | |
| | | eicosatrienoyl)-sn-glycerol | | | | |
| LMGL03010671 | TG(16:0/20:2(11Z,14Z)/20:4(5Z,8Z,11Z,14Z)) | 1-hexadecanoyl)-2-(11Z,14Z- | 907.6 | 907.7749 | C₅₉H₁₀₃O₈ | M + H |
| | [iso6] | eicosadienoyl)-3-(5Z,8Z,11Z,14Z- | | | | |
| | | eicosatetraenoyl)-sn-glycerol | | | | |
| LMGL03010672 | TG(16:1(9Z)/20:1(11Z)/20:4(5Z,8Z,11Z,14Z)) | 1-(9Z-hexadecanoyl)-2-(11Z-eicosadienoyl)-3- | 907.6 | 907.7749 | C₅₉H₁₀₃O₈ | M + H |
| | [iso6] | (5Z,8Z,11Z,14Z-eicosatetraenoyl)-sn- | | | | |
| | | glycerol | | | | |
| LMGL03010676 | TG(18:0/18:2(9Z,12Z)/20:4(5Z,8Z,11Z,14Z)) | 1-octadecenoyl-2-(9Z,12Z- | 907.6 | 907.7749 | C₅₉H₁₀₃O₈ | M + H |
| | [iso6] | octadecadienoyl)-3-(8Z,8Z,11Z,14Z- | | | | |
| | | eicosatetranoyl)-sn-glycerol | | | | |
| LMGL03010677 | TG(18:1(9Z)/18:1(9Z)/20:4(5Z,8Z,11Z,14Z)) | 1,2-di-(9Z-octadecenoyl)-3- | 907.6 | 907.7749 | C₅₉H₁₀₃O₈ | M + H |
| | [iso3] | (5Z,8Z,11Z,14Z-eicosatetraenoyl)-sn- | | | | |
| | | glycerol | | | | |
| LMGL03010678 | TG(16:0/20:1(11Z)/20:5(5Z,8Z,11Z,14Z,17Z)) | 1-hexadecanoyl-2-(11Z-eicosenoyl)-3- | 907.6 | 907.7749 | C₅₉H₁₀₃O₈ | M + H |
| | [iso6] | (5Z,8Z,11Z,14Z,17Z-eicosapentaenoyl)-sn- | | | | |
| | | glycerol | | | | |
| LMGL03010679 | TG(16:1(9Z)/20:0/20.5(5Z,8Z,11Z,14Z,17Z)) | 1-(9Z-hexadecanoyl)-2-eicosanoyl-3- | 907.6 | 907.7749 | C₅₉H₁₀₃O₈ | M + H |
| | [iso6] | (5Z,8Z,11Z,14Z,17Z-eicosapentaenoyl)-sn- | | | | |
| | | glycerol | | | | |
| LMGL03010683 | TG(18:0/18:1(9Z)/20:5(5Z,8Z,11Z,14Z,17Z)) | 1-octadecanoyl-2-(9Z-octadecenoyl)-3- | 907.6 | 907.7749 | C₅₉H₁₀₃O₈ | M + H |
| | [iso6] | (5Z,8Z,11Z,14Z,17Z-eicosapentaenoyl)-sn- | | | | |
| | | glycerol | | | | |
| LMGL03010703 | TG(17:1(9Z)/17:2(9Z,12Z)/22:3(10Z,13Z,16Z)) | 1-(9Z-heptadecenoyl-2-(6Z,12Z- | 907.6 | 907.7749 | C₅₉H₁₀₃O₈ | M + H |
| | [iso6] | heptadecadienoyl)-3-(10Z,13Z,16Z- | | | | |
| | | docosatrienoyl)-sn-glycerol | | | | |
| LMGL03010739 | TG(17:2(9Z,12Z)/19:0/20:4(5Z,8Z,11Z,14Z)) | 1-(9Z,12Z-heptadecadienoyl)-2- | 907.6 | 907.7749 | C₅₉H₁₀₃O₈ | M + H |
| | [iso6] | nonadecanoyl-3-(5Z,8Z,11Z,14Z- | | | | |
| | | eicosatetraenoyl)-sn-glycerol | | | | |
| LMGL03010745 | TG(17:1(9Z)/19:0/20:5(5Z,8Z,11Z,14Z,17Z)) | 1-(9Z-heptadecenoyl)-2-nonadecanoyl-3- | 907.6 | 907.7749 | C₅₉H₁₀₃O₈ | M + H |
| | [iso6] | (5Z,8Z,11Z,14Z,17Z-eicosapentaenoyl)-sn- | | | | |
| | | glycerol | | | | |
| LMGL03010766 | TG(16:0/18:3(9Z,12Z,15Z)/22:3(10Z,13Z,16Z)) | 1-hexadecanoyl-2-(9Z,12Z,15Z- | 907.6 | 907.7749 | C₅₉H₁₀₃O₈ | M + H |
| | [iso6] | octadecatrienoyl)-3-(10Z,13Z,16Z- | | | | |
| | | docosatrienoyl)-sn-glycerol | | | | |
| LMGL03010767 | TG(16:1(9Z)/18:2(9Z,12Z)/22:3(10Z,13Z,16Z)) | 1-(9Z-hexadecenoyl)-2-(9Z,12Z- | 907.6 | 907.7749 | C₅₉H₁₀₃O₈ | M + H |
| | [iso6] | octadecadienoyl)-3-(10Z,13Z,16Z- | | | | |
| | | docosatrienoyl)-sn-glycerol | | | | |
| LMGL03010777 | TG(17:0/17:2(9Z,12Z)/22:4(7Z,10Z,13Z,16Z)) | 1-heptadecanoyl-2-(9Z,12Z- | 907.6 | 907.7749 | C₅₉H₁₀₃O₈ | M + H |
| | [iso6] | heptadecadienoyl)-3-(7Z,10Z,13Z,16Z- | | | | |
| | | docosatetraenoyl)-sn-glycerol | | | | |
| LMGL03010778 | TG(17:1(9Z)/17:1(9Z)/22:4(7Z,10Z,13Z,16Z)) | 1,2-di-(9Z-heptadecanoyl)-3- | 907.6 | 907.7749 | C₅₉H₁₀₃O₈ | M + H |
| | [iso3] | (7Z,10Z,13Z,16Z-docosatetraenoyl)-sn- | | | | |
| | | glycerol | | | | |
| LMGL03010781 | TG(17:0/17:1(9Z)/22:5(7Z,10Z,13Z,16Z,19Z)) | 1-heptadecanoyl)-2-(9Z-heptadecanoyl)-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03010784 | TG(17:0/17:0/22:6(4Z,7Z,10Z,13Z,16Z,19Z)) | 1,2-diheptadecanoyl)-3- | 907.6 | 907.7749 | C₅₉H₁₀₃O₈ | M + H |
| | [iso3] | (7Z,10Z,13Z,16Z,19Z-docosapentaenoyl)- | | | | |
| | | sn-glycerol | | | | |
| LMGL03010844 | TG(17:2(9Z,12Z)/17:2(9Z,12Z)/22:2(13Z,16Z)) | 1,2-di-(9Z,12Z-heptadecadienoyl)-3- | 907.6 | 907.7749 | C₅₉H₁₀₃O₈ | M + H |
| | [iso3] | (13Z,16Z-docosadienoyl)-sn-glycerol | | | | |
| LMGL03010845 | TG(16:0/18:2(9Z,12Z)/22:4(7Z,10Z,13Z,16Z)) | 1-hexadecanoyl-2-(9Z,12Z- | 907.6 | 907.7749 | C₅₉H₁₀₃O₈ | M + H |
| | [iso6] | octadecadienoyl-3-(7Z,10Z,13Z,16Z- | | | | |
| | | docosatetraenoyl)-sn-glycerol | | | | |
| LMGL03010846 | TG(16:1(9Z)/18:1(9Z)/22:4(7Z,10Z,13Z,16Z)) | 1-(9Z-hexadecenoyl)-2-(9Z, | 907.6 | 907.7749 | C₅₉H₁₀₃O₈ | M + H |
| | [iso6] | octadecenoyl)-3-(7Z,10Z,13Z,16Z- | | | | |
| | | docosatetraenoyl)-sn-glycerol | | | | |
| LMGL03010849 | TG(16:0/18:1(9Z)/22:5(7Z,10Z,13Z,16Z,19Z)) | 1-hexadecanoyl)-2-(9Z-octadecanoyl)-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03010850 | TG(16:1(9Z)/18:0/22:5(7Z,10Z,13Z,16Z,19Z)) | 1-(9Z-hexadecenoyl)-2-octadecanoyl-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03010853 | TG(16:0/18:0/22:6(4Z,7Z,10Z,13Z,16Z,19Z)) | 1-hexadecanoyl-2-octadecanoyl-3- | 907.6 | 907.7749 | C₅₉H₁₀₃O₈ | M + H |
| | [iso6] | (4Z,7Z,10Z,13Z,16Z,19Z- | | | | |
| | | docosadienoyl)-sn-glycerol | | | | |
| LMGL03010912 | TG(16:1(9Z)/18:3(9Z,12Z,15Z)/22:2(13Z,16Z)) | 1-(9Z-hexadecenoyl)-2-(9Z,12Z,15Z- | 907.6 | 907.7749 | C₅₉H₁₀₃O₈ | M + H |
| | [iso6] | octadecatrienoyl)-3-(13Z,16Z- | | | | |
| | | docosadienoyl)-sn-glycerol | | | | |
| LMGL03012687 | TG(12:0/22:3(10Z,13Z,16Z)/22:3(10Z,13Z,16Z)) | 1-dodecanoyl)-2,3-di-(10Z,13Z,16Z- | 907.6 | 907.7749 | C₅₉H₁₀₃O₈ | M + H |
| | [iso3] | docosatrienoyl)-sn-glycerol | | | | |
| LMGL03013049 | TG(18:3(6Z,9Z,12Z)/18:3(6Z,9Z,12Z)/20:0)[iso3] | 1,2-di-(6Z,9Z,12Z-octadecatrienoyl)-2-(9Z,12Z,15Z- | 907.6 | 907.7749 | C₅₉H₁₀₃O₈ | M + H |
| | | eicosanoyl)-sn-glycerol | | | | |
| LMGL03013104 | TG(18:4(6Z,9Z,12Z,15Z)/19:1(9Z)/19:1)(9Z)) | 1-(6Z,9Z,12Z,15Z-octadecatetraenoyl)-2,3- | 907.6 | 907.7749 | C₅₉H₁₀₃O₈ | M + H |
| | [iso3] | di9Z-nonadecenoyl-sn-glycerol | | | | |
| LMGL03013656 | TG(12:0/22:0/22:6(4Z,7Z,10Z,13Z,16Z,19Z)) | 1-dodecanoyl-2-docasanoyl-3- | 907.6 | 907.7749 | C₅₉H₁₀₃O₈ | M + H |
| | [iso6] | (4Z,7Z,10Z,13Z,16Z,19Z- | | | | |
| | | docosahexaenoyl)-sn-glycerol | | | | |
| LMGL03013660 | TG(12:0/22:1(11Z)/22:5(7Z,10Z,13Z,16Z,19Z)) | 1-dodecanoyl)-2-11Z-docosenoyl-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03013663 | TG(12:0/22:2(13Z,16Z)/22:4(7Z,10Z,13Z,16Z)) | 1-dodecanoyl-2-(13Z,16Z- | 907.6 | 907.7749 | C₅₉H₁₀₃O₈ | M + H |
| | [iso6] | docosadienoyl)-3-(7Z,10Z,13Z,16Z- | | | | |
| | | docosatetraenoyl)-sn-glycerol | | | | |
| LMGL03014115 | TG(13:0/21:0/22:6(4Z,7Z,10Z,13Z,16Z,19Z)) | 1-tridecanoyl-2-heneicosanoyl-3- | 907.6 | 907.7749 | C₅₉H₁₀₃O₈ | M + H |
| | [iso6] | (4Z,7Z,10Z,13Z,16Z,19Z- | | | | |
| | | docosahexaenoyl)-sn-glycerol | | | | |
| LMGL03014493 | TG(14:0/20:0/22:6(4Z,7Z,10Z,13Z,16Z,19Z)) | 1-tetradecanoyl-2-eicosanoyl-3- | 907.6 | 907.7749 | C₅₉H₁₀₃O₈ | M + H |
| | [iso6] | (4Z,7Z,10Z,13Z,16Z,19Z- | | | | |
| | | docosahexaenoyl)-sn-glycerol | | | | |
| LMGL03014504 | TG(14:0/20:1(11Z)/22:5(7Z,10Z,13Z,16Z,19Z)) | 1-tetradecanoyl)-2-(11Z-eicosenoyl)-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03014514 | TG(14:0/20:2(11Z,14Z)/22:4(7Z,10Z,13Z,16Z)) | 1-tetradecanoyl-2-(11Z,14Z- | 907.6 | 907.7749 | C₅₉H₁₀₃O₈ | M + H |
| | [iso6] | eicosadienoyl-3-(7Z,10Z,13Z,16Z- | | | | |
| | | docosatetraenoyl)-sn-glycerol | | | | |
| LMGL03014523 | TG(14:0/20:3(8Z,11Z,14Z)/22:3(10Z,13Z,16Z)) | 1-tetradecanoyl-2-(8Z,11Z,14Z- | 907.6 | 907.7749 | C₅₉H₁₀₃O₈ | M + H |
| | [iso6] | eicosatrienoyl-3-(10Z,13Z,16Z- | | | | |
| | | docosatrienoyl)-sn-glycerol | | | | |
| LMGL03014531 | TG(14:0/20:4(5Z,8Z,11Z,14Z)/22:2(13Z,16Z)) | 1-tetradecanoyl-2-(5Z,8Z,11Z,14Z- | 907.6 | 907.7749 | C₅₉H₁₀₃O₈ | M + H |
| | [iso6] | eicosatetraenoyl-3-(13Z,16Z- | | | | |
| | | docosadienoyl)-sn-glycerol | | | | |
| LMGL03014538 | TG(14:0/20:5(5Z,8Z,11Z,14Z,17Z)/22:1(11Z)) | 1-tetradecanoyl-2-(5Z,8Z,11Z,14Z,17Z- | 907.6 | 907.7749 | C₅₉H₁₀₃O₈ | M + H |
| | [iso6] | eicosapentaenoyl-3-(11Z-docosenoyl)-sn- | | | | |
| | | glycerol | | | | |
| LMGL03014898 | TG(14:1(9Z)/20:0/22:5(7Z,10Z,13Z,16Z,19Z)) | 1-(9Z-tetradecenoyl)-2-eicosanoyl-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03014909 | TG(14:1(9Z)/20:1(11Z)/22:4(7Z,10Z,13Z,16Z)) | 1-(9Z-tetradecenoyl)-2-(11Z-eicosenoyl-3- | 907.6 | 907.7749 | C₅₉H₁₀₃O₈ | M + H |
| | [iso6] | (7Z,10Z,13Z,16Z-docosatetraenoyl)-sn- | | | | |
| | | glycerol | | | | |
| LMGL03014919 | TG(14:1(9Z)/20:2(11Z,14Z)/22:3(10Z,13Z,16Z)) | 1-(9Z-tetradecenoyl)-2-(11Z,14Z- | 907.6 | 907.7749 | C₅₉H₁₀₃O₈ | M + H |
| | [iso6] | eicosadienoyl)-3-(10Z,13Z,16Z- | | | | |
| | | docosatrienoyl)-sn-glycerol | | | | |
| LMGL03014928 | TG(14:1(9Z)/20:3(8Z,12Z,14Z)/22:2(13Z,16Z)) | 1-(9Z-tetradecenoyl)-2-(8Z,11Z,14Z- | 907.6 | 907.7749 | C₅₉H₁₀₃O₈ | M + H |
| | [iso6] | eicosatrienoyl-3-(13Z,16Z-docosadienoyl)- | | | | |
| | | sn-glycerol | | | | |
| LMGL03014936 | TG(14:1(9Z)/20:4(5Z,8Z,11Z,14Z)/22:1(11Z)) | 1-(9Z-tetradecenoyl-2-(5Z,8Z,11Z,14Z- | 907.6 | 907.7749 | C₅₉H₁₀₃O₈ | M + H |
| | [iso6] | eicosatetraenoyl-3-(11Z-docosenoyl)-sn- | | | | |
| | | glycerol | | | | |
| LMGL03014943 | TG(14:1(9Z)/20:5(5Z,8Z,11Z,14Z,17Z)/22:0) | 1-(9Z-tetradecenoyl)-2- | 907.6 | 907.7749 | C₅₉H₁₀₃O₈ | M + H |
| | [iso6] | (5Z,8Z,11Z,14Z,17Z-eicosapentaenoyl)-3- | | | | |
| | | docosanoyl-sn-glycerol | | | | |
| LMGL03015250 | TG(15:0/19:0/22:6(4Z,7Z,10Z,13Z,16Z,19Z)) | 1-pentadecanoyl-2-nonadecanoyl-3- | 907.6 | 907.7749 | C₅₉H₁₀₃O₈ | M + H |
| | [iso6] | (4Z,7Z,10Z,13Z,16Z,19Z- | | | | |
| | | docosahexaenoyl)-sn-glycerol | | | | |
| LMGL03015263 | TG(15:0/19:1(9Z)/22:5(7Z,10Z,13Z,16Z,19Z)) | 1-pentadecanoyl)-2-9Z-nonadecenoyl)-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03015600 | TG(15:1(9Z)/19:0/22:5(7Z,10Z,13Z,16Z,19Z)) | 1-(9Z-pentadecenoyl)-2-nonadecanoyl)-3- | 907.6 | 907.7749 | C₅₉H₁₀₃O₈ | M + H |
| | [iso6] | (7Z,10Z,13Z,16Z,19Z-docosapentaenoyl)- | | | | |
| | | sn-glycerol | | | | |
| LMGL03015613 | TG(15:1(9Z)/19:1(9Z)/22:4(7Z,10Z,13Z,16Z)) | 1-(9Z-pentadecenoyl)-2-9Z-nonadecenoyl)- | 907.6 | 907.7749 | C₅₉H₁₀₃O₈ | M + H |
| | [iso6] | 3-(7Z,10Z,13Z,16Z-docosatetraenoyl)-sn- | | | | |
| | | glycerol | | | | |
| LMGL03015671 | TG(15:1(9Z)/20:5(5Z,8Z,11Z,14Z,17Z)/21:0) | 1-(9Z-pentadecenoyl)-2- | 907.6 | 907.7749 | C₅₉H₁₀₃O₈ | M + H |
| | [iso6] | (5Z,8Z,11Z,14Z,17Z-eicosapentaenoyl)-3- | | | | |
| | | heneicosanoyl-sn-glycerol | | | | |
| LMGL03015749 | TG(16:0/18:3(6Z,9Z,12Z)/22:3(10Z,13Z,16Z)) | 1-hexadecanoyl-2-(6Z,9Z,12Z- | 907.6 | 907.7749 | C₅₉H₁₀₃O₈ | M + H |
| | [iso6] | octadecatrienoyl)-3-(10Z,13Z,16Z- | | | | |
| | | docosatrienoyl)-sn-glycerol | | | | |
| LMGL03015767 | TG(16:0/18:4(6Z,9Z,12Z,15Z)/22:2(13Z,16Z)) | 1-hexadecanoyl-2-(6Z,9Z,12Z,15Z- | 907.6 | 907.7749 | C₅₉H₁₀₃O₈ | M + H |
| | [iso6] | octadecatetraenoyl-3-(13Z,16Z- | | | | |
| | | docosadienoyl)-sn-glycerol | | | | |
| LMGL03015839 | TG(16:1(9Z)/18:3(6Z,9Z,12Z)/22:2(13Z,16Z)) | 1-(9Z-hexadecenoyl)-2-(6Z,9Z,12Z,15Z- | 907.6 | 907.7749 | C₅₉H₁₀₃O₈ | M + H |
| | [iso6] | octadecatrienoyl-3-(13Z,16Z- | | | | |
| | | docosadienoyl)-sn-glycerol | | | | |
| LMGL03015857 | TG(16:1(9Z)/18:4(6Z,9Z,12Z,15Z)/22:1(11Z)) | 1-(9Z-hexadecenoyl)-2-(6Z,9Z,12Z,15Z- | 907.6 | 907.7749 | C₅₉H₁₀₃O₈ | M + H |
| | [iso6] | octadecatetraenoyl)-3-11Z-docosenoyl-sn- | | | | |
| | | glycerol | | | | |
| LMGL03015957 | TG(17:0/19:1(9Z)/20:5(5Z,8Z,11Z,14Z,17Z)) | 1-heptadecanoyl-2-9Z-nonadecenoyl-3- | 907.6 | 907.7749 | C₅₉H₁₀₃O₈ | M + H |
| | [iso6] | (5Z,8Z,11Z,14Z,17Z-eicosapentaenoyl)-sn- | | | | |
| | | glycerol | | | | |
| LMGL03016039 | TG(17:1(9Z)/19:1(9Z)/20:4(5Z,8Z,11Z,14Z)) | 1-(9Z-heptadecanoyl)-2-9Z-nonadecenoyl- | 907.6 | 907.7749 | C₅₉H₁₀₃O₈ | M + H |
| | [iso6] | 3-(5Z,8Z,11Z,14Z-eicosatetraenoyl)-sn- | | | | |
| | | glycerol | | | | |
| LMGL03016104 | TG(17:2(9Z,12Z)/18:4(6Z,9Z,12Z,15Z))/21:0) | 1-(9Z-12Z-heptadecanoyl)-2- | 907.6 | 907.7749 | C₅₉H₁₀₃O₈ | M + H |
| | [iso6] | (6Z,9Z,12Z,15Z-octadecatetraenoyl)-3- | | | | |
| | | heneicosanoyl-sn-glycerol | | | | |
| LMGL03016117 | TG(17:2(9Z,12Z)/19:1(9Z)/20:3(8Z,11Z,14Z)) | 1-(9Z-12Z-heptadecanoyl)-2-9Z- | 907.6 | 907.7749 | C₅₉H₁₀₃O₈ | M + H |
| | [iso6] | nonadecenoyl-3-(8Z,11Z,14Z- | | | | |
| | | eicosatrienoyl)-sn-glycerol | | | | |
| LMGL03016157 | TG(18:0/18:3(6Z,9Z,12Z)/20:3(8Z,11Z,14Z)) | 1-octadecanoyl-2-(6Z,9Z,12Z- | 907.6 | 907.7749 | C₅₉H₁₀₃O₈ | M + H |
| | [iso6] | octadecatrienoyl)-3-(8Z,11Z,14Z- | | | | |
| | | eicosatrienoyl)-sn-glycerol | | | | |
| LMGL03016175 | TG(18:0/18:4(6Z,9Z,15Z)/20:2(11Z,14Z)) | 1-octadecanoyl-2-(6Z,9Z,12Z,15Z- | 907.6 | 907.7749 | C₅₉H₁₀₃O₈ | M + H |
| | [iso6] | octadecatetraenoyl)-3-(11Z,14Z- | | | | |
| | | eicosadienoyl)-sn-glycerol | | | | |
| LMGL03016227 | TG(18:1(9Z)/18:3(6Z,9Z,12Z)/20:2(11Z,14Z)) | 1-(9Z-octadecanoyl-2-(6Z,9Z,12Z- | 907.6 | 907.7749 | C₅₉H₁₀₃O₈ | M + H |
| | [iso6] | octadecatrienoyl)-3-(11Z,14Z- | | | | |
| | | eicosadienoyl)-sn-glycerol | | | | |
| LMGL03016245 | TG(18:1(9Z)/18:4(6Z,9Z,12Z,15Z)/20:1(11Z)) | 1-(9Z-octadecanoyl-2-(5Z,9Z,12Z,15Z- | 907.6 | 907.7749 | C₅₉H₁₀₃O₈ | M + H |
| | [iso6] | octadecatetraenoyl)-3-(11Z-eicosenoyl)-sn- | | | | |
| | | glycerol | | | | |
| LMGL03016293 | TG(18:2(9Z,12Z)/18:3(6Z,9Z,12Z)/20:1(11Z)) | 1-(9Z,12Z-octadecadienoyl)-2-(6Z,9Z,12Z- | 907.6 | 907.7749 | C₅₉H₁₀₃O₈ | M + H |
| | [iso6] | octadecatetraenoyl)-3-(11Z-eicosenoyl)-sn- | | | | |
| | | glycerol | | | | |
| LMGL03016311 | TG(18:2(9Z,12Z)/18:4(6Z,9Z,12Z,15Z)/20:0) | 1-(9Z,12Z-octadecadienoyl)-2- | 907.6 | 907.7749 | C₅₉H₁₀₃O₈ | M + H |
| | [iso6] | (6Z,9Z,12Z,15Z-octadecatetraenoyl)-3- | | | | |
| | | eicosanoyl)-sn-glycerol | | | | |
| LMGL03016358 | TG(18:3(6Z,9Z,12Z)/18:3(9Z,12Z,15Z)/20:0) | 1-(6Z,9Z,12Z-octadecatrienoyl)-2- | 907.6 | 907.7749 | C₅₉H₁₀₃O₈ | M + H |
| | [iso6] | (9Z,12Z,15Z-octadecatrienoyl)-3- | | | | |
| | | eicosanoyl)-sn-glycerol | | | | |
| LMGP06010213 | PI(17:0/22:1(11Z)) | 1-heptadecanoyl-2-(11Z-docosenoyl)- | 907.6 | 907.6270 | C₄₅H₉₂O₁₃P | M + H |
| | | glycero-3-phospho-(1′-myo-inositol) | | | | |
| LMGP06010241 | PI(17:1(9Z)/22:0) | 1-(9Z-heptadecanoyl-2-docosanoyl)- | 907.6 | 907.6270 | C₄₅H₉₂O₁₃P | M + H |
| | | glycero-3-phospho-(1′-myo-inositol) | | | | |
| LMGP06010304 | PI(18:1(9Z)/21:0) | 1-(9Z-octadecenoyl-2-heneicosanoyl)- | 907.6 | 907.6270 | C₄₅H₉₂O₁₃P | M + H |
| | | glycero-3-phospho-(1′-myo-inositol) | | | | |
| LMGP06010422 | PI(18:4(6Z,9Z,12Z,15Z)/22:4(7Z,10Z,13Z,16Z)) | 1-(6Z,9Z,12Z,15Z-octadecatetraenoyl-2- | 907.6 | 907.5331 | C₄₉H₅₀O₁₃P | M + H |
| | | (7Z,10Z,13Z,16Z-docosatetraenoyl)- | | | | |
| | | glycero-3-phospho-(1′-myo-inositol) | | | | |
| LMGP06010437 | PI(19:0/20:1(11Z)) | 1-nonadecanoyl-2-(11Z-eicosenoyl)- | 907.6 | 907.6270 | C₄₅H₉₂O₁₃P | M + H |
| | | glycero-3-phospho-(1′-myo-inositol) | | | | |
| LMGP06010467 | PI(19:1(9Z)/20:0) | 1-(9Z-nonadecanoyl)-2-eicosanoyl-glycero- | 907.6 | 907.6270 | C₄₅H₉₂O₁₃P | M + H |
| | | 3-phospho-(1′-myo-inositol) | | | | |
| LMGP06010489 | PI(20:0/19:1(9Z)) | 1-eicosanoyl-2-(9Z-nonadecanoyl)-glycero- | 907.6 | 907.6270 | C₄₅H₉₂O₁₃P | M + H |
| | | 3-phospho-(1′-myo-inositol) | | | | |
| LMGP06010513 | PI(20:1(11Z)/19:0) | 1-(11Z-eicosenoyl)-2-nonadecanoyl)- | 907.6 | 907.6270 | C₄₅H₉₂O₁₃P | M + H |
| | | glycero-3-phospho-(1′-myo-inositol) | | | | |
| LMGP06010581 | PI(20:3(8Z,11Z,14Z)/20:5(5Z,8Z,11Z,14Z,17Z)) | 1-(8Z,11Z,14Z-eicosatrienoyl)-2- | 907.6 | 907.5331 | C₄₉H₅₀O₁₃P | M + H |
| | | (5Z,8Z,11Z,14Z,17Z-eicosapentaenoyl)- | | | | |
| | | glycero-3-phospho-(1′-myo-inositol) | | | | |
| LMGP06010638 | PI(20:5(5Z,8Z,11Z,14Z,17Z)/20:3(8Z,11Z,14Z)) | 1-(5Z,8Z,11Z,14Z,17Z- | 907.6 | 907.5331 | C₄₉H₅₀O₁₃P | M + H |
| | | eicosapentaenoyl)-2-(8Z,11Z,14Z- | | | | |
| | | eicosatrienoyl)-glycero-3-phospho-(1′-myo- | | | | |
| | | inositol) | | | | |
| LMGP06010654 | PI(21:0/18:1(9Z)) | 1-heneicosanoyl-2-(9Z-octadecenoyl)- | 907.6 | 907.6270 | C₄₅H₉₂O₁₃P | M + H |
| | | glycero-3-phospho-(1′-myo-inositol) | | | | |
| LMGP06010677 | PI(22:0/17:1(9Z)) | 1-docosanoyl-2-(9Z-heptadecenoyl)- | 907.6 | 907.6270 | C₄₅H₉₂O₁₃P | M + H |
| | | glycero-3-phospho-(1′-myo-inositol) | | | | |
| LMGP06010702 | PI(22:1(11Z)/17:0) | 1-(11Z-docosenoyl)-2-heptadecanoyl- | 907.6 | 907.6270 | C₄₅H₉₂O₁₃P | M + H |
| | | glycero-3-phospho-(1′-myo-inositol) | | | | |
| LMGP06010772 | PI(22:4(7Z,10Z,13Z,16Z)/18:4(6Z,9Z,12Z,15Z)) | 1-(7Z,10Z,13Z,16Z-docosatetraenoyl)-2- | 907.6 | 907.5331 | C₄₉H₅₀O₁₃P | M + H |
| | | (6Z,9Z,12Z,15Z-octadecatetraenoyl)- | | | | |
| | | glycero-3-phospho-(1′-myo-inositol) | | | | |
| LMGP06010798 | PI(22:6(4Z,7Z,10Z,13Z,16Z,19Z)/18:2(9Z,12Z)) | 1-(4Z,7Z,10Z,13Z,16Z,19Z- | 907.6 | 907.5331 | C₄₉H₅₀O₁₃P | M + H |
| | | docosahexaenoyl)-2-(9Z,12Z- | | | | |
| | | octadecadienoyl)-glycero-3-phospho-(1′- | | | | |
| | | myo-inositol) | | | | |
| LMGP06010827 | PI(20:4(5Z,8Z,11Z,14Z)/20:4(5Z,8Z,11Z,14Z)) | 1,2-di-(5Z,8Z,11Z,14Z-eicosatetraenoyl)- | 907.6 | 907.5331 | C₄₉H₅₀O₁₃P | M + H |
| | | sn-glycero-3-phospho-(1′-myo-inositol) | | | | |
| LMGP06010844 | PI(18:2(9Z,12Z)/22:6(4Z,7Z,10Z,13Z,16Z,19Z)) | 1-(9Z,12Z-octadecadienoyl)-2- | 907.6 | 907.5331 | C₄₉H₅₀O₁₃P | M + H |
| | | (4Z,7Z,10Z,13Z,16Z,19Z- | | | | |
| | | docosahexaenoyl)-glycero-3-phospho-(1′- | | | | |
| | | myo-inositol) | | | | |
| LMGP06020039 | PI(O-18:0/22:1(11Z)) | 1-octadecyl-2-(11Z-docosenoyl)-glycero-3- | 907.6 | 907.6634 | C₄₉H₉₅O₁₂P | M + H |
| | | phospho-(1′-myo-inositol) | | | | |
| LMGP06020061 | PI(O-20:0/20:1(11Z)) | 1-eicosyl-2-(11Z-eicosenoyl)-glycero-3- | 907.6 | 907.6634 | C₄₉H₉₅O₁₂P | M + H |
| | | phospho-(1′-myo-inositol) | | | | |
| LMGP06030053 | PI(P-18:0/22:0) | 1-(1Z-octadecenyl)-2-docosanoyl)-glycero-3- | 907.6 | 907.6634 | C₄₉H₉₅O₁₂P | M + H |
| | | phospho-(1′-myo-inositol) | | | | |
| LMGP06030076 | PI(P-20:0/20:0) | 1-(1Z-eicosenyl)-2-eicosanoyl)-glycero-3- | 907.6 | 907.6634 | C₄₉H₉₅O₁₂P | M + H |
| | | phospho-(1′-myo-inositol) | | | | |
| LMST05050019 | Kurilensoside 1 | 3beta-(2-O-methyl-beta-D- | 907.6 | 907.4897 | C₄₄H₇₅O₁₉ | M + H |
| | | xylopyranosyloxy)-24R-[2-O-methyl-beta-D- | | | | |
| | | xylopyranosyl-(1-5)-alpha-L- | | | | |
| | | arabinofuranosyloxy]-5alpha-cholest-22E- | | | | |
| | | en-4beta,6alpha,7alpha,8,15beta-pentol | | | | |
| LMGL03011244 | TG(17:2(9Z,12Z)/20:5(5Z,8Z,11Z,14Z,17Z)/20:5(5Z,8Z,11Z,14Z,17Z)) | 1-(9Z,12Z- | 909.5 | 909.6967 | C₅₉H₂₃O₈ | M + H |
| | [iso3] | heptadecadienoyl)-2,3-di- | | | | |
| | | (5Z,8Z,11Z,14Z,17Z- | | | | |
| | | eicosapentaenoyl)-sn- | | | | |
| | | glycerol | | | | |
| LMGL03012752 | TG(13:0/22:6(4Z,7Z,10Z,13Z,16Z,19Z)/22:6(4Z,7Z,10Z,13Z,16Z,19Z)) | 1-tridecanoyl-2,3-di- | 909.5 | 909.6967 | C₅₉H₂₃O₈ | M + H |
| | [iso3] | (4Z,7Z,10Z,13Z,16Z,19Z- | | | | |
| | | docosahexaenoyl)-sn- | | | | |
| | | glycerol | | | | |
| LMGL03015678 | TG(15:1(9Z)/20:5(5Z,8Z,11Z,14Z,17Z)/22:6(4Z,7Z,10Z,13Z,16Z,19Z)) | 1-(9Z-pentadecenoyl)-2- | 909.5 | 909.6967 | C₅₉H₂₃O₈ | M + H |
| | [iso6] | (5Z,8Z,11Z,14Z,17Z- | | | | |
| | | eicosapentaenoyl)-3- | | | | |
| | | (4Z,7Z,10Z,13Z,16Z,19Z- | | | | |
| | | docosahexaenoyl)-sn- | | | | |
| | | glycerol | | | | |
| LMGL03016111 | TG(17:2(9Z,12Z)/18:4(6Z,9Z,12Z,15Z)/22:6(4Z,7Z,10Z,13Z,16Z,19Z)) | 1-(9Z,12Z- | 909.5 | 909.6967 | C₅₉H₂₃O₈ | M + H |
| | [iso6] | heptadecadienoyl)-2- | | | | |
| | | (6Z,9Z,12Z,15Z- | | | | |
| | | octadecatetraenoyl)-3- | | | | |
| | | (4Z,7Z,10Z,13Z,16Z,19Z- | | | | |
| | | docosahexaenoyl)-sn- | | | | |
| | | glycerol | | | | |
| LMGP06010013 | PI(18:1(9Z)/22:6(4Z,7Z,10Z,13Z,16Z,19Z)) | 1-(9Z-octadecenoyl)-2- | 909.5 | 909.5488 | C₄₉H₅₂O₁₃P | M + H |
| | | (4Z,7Z,10Z,13Z,16Z,19Z- | | | | |
| | | docosahexaenoyl)-sn- | | | | |
| | | glycerol-3-phospho-(1′- | | | | |
| | | myo-inositol | | | | |
| LMGP06010212 | PI(17:0/22:0) | 1-heptadecanoyl-2- | 909.5 | 909.6427 | C₄₅H₂₄O₁₃P | M + H |
| | | docosanoyl-glycerol-3- | | | | |
| | | phospho-(1′-myo-inositol) | | | | |
| LMGP06010287 | PI(18:0/21:0) | 1-octadecanoyl-2- | 909.5 | 909.6427 | C₄₅H₂₄O₁₃P | M + H |
| | | heneicosanoyl-glycerol-3- | | | | |
| | | phospho-(1′-myo-inositol | | | | |
| LMGP06010363 | PI(18:3(6Z,9Z,12Z)/22:4(7Z,10Z,13Z,16Z)) | 1-(6Z,9Z,12Z- | 909.5 | 909.5488 | C₄₉H₈₂O₁₃P | M + H |
| | | octadecatrienoyl)-2- | | | | |
| | | (7Z,10Z,13Z,16Z- | | | | |
| | | docosatetraenoyl)- | | | | |
| | | glycero-3-phospho-(1′- | | | | |
| | | myo-inositol) | | | | |
| LMGP06010391 | PI(16:3(9Z,12Z,15Z)/22:4(7Z,10Z,13Z,16Z)) | 1-(9Z,12Z,15Z- | 909.5 | 909.5488 | C₄₉H₅₂O₁₃P | M + H |
| | | octadecatrienoyl)-2- | | | | |
| | | (7Z,10Z,13Z,16Z- | | | | |
| | | docosatetraenoyl)- | | | | |
| | | glycero-3-phospho-(1′- | | | | |
| | | myo-inositol) | | | | |
| LMGP06010436 | PI(19:0/20:0) | 1-nonadecanoyl-2- | 909.5 | 909.6427 | C₄₉H₉₄O₁₃P | M + H |
| | | eicosanoyl-glycerol-3- | | | | |
| | | phospho-(1′-myo-inositol) | | | | |
| LMGP06010488 | PI(20:0/19:0) | 1-eicosanoyl-2- | 909.5 | 909.6427 | C₄₅H₉₄O₁₃P | M + H |
| | | nonadecanoyl-glycerol-3- | | | | |
| | | phospho-(1′-myo-inositol) | | | | |
| LMGP06010550 | PI(20:2(11Z,14Z)/20:5(5Z,8Z,11Z,14Z,17Z)) | 1-(11Z,14Z- | 909.5 | 909.5488 | C₄₉H₈₂O₁₃P | M + H |
| | | eicosadienoyl)-2- | | | | |
| | | (5Z,8Z,11Z,14Z,17Z- | | | | |
| | | eicosapentaenoyl)- | | | | |
| | | glycero-3-phospho-(1′- | | | | |
| | | myo-inositol) | | | | |
| LMGP06010550 | PI(20:2(11Z,14Z)20:5(5Z,8Z,11Z,14Z,17Z)) | 1-(11Z,14Z- | 909.5 | 909.5488 | C₄₉H₈₂O₁₃P | M + H |
| | | eicosadienoyl)-2- | | | | |
| | | (5Z,8Z,11Z,14Z,17Z- | | | | |
| | | eicosapentaenoyl)- | | | | |
| | | glycero-3-phospho-(1′- | | | | |
| | | myo-inositol) | | | | |
| LMGP06010580 | PI(20:3(8Z,11Z,14Z)/20:4(5Z,8Z,11Z,14Z)) | 1-(8Z,11Z,14Z- | 909.5 | 909.5488 | C₄₉H₈₂O₁₃P | M + H |
| | | eicosatrienoyl)-2- | | | | |
| | | (5Z,8Z,11Z,14Z- | | | | |
| | | eicosatetraenoyl)-glycero- | | | | |
| | | 3-phospho-(1′-myo- | | | | |
| | | inositol) | | | | |
| LMGP06010609 | PI(20:4(5Z,8Z,11Z,14Z)/20:3(8Z,11Z,14Z)) | 1-(5Z,8Z,11Z,14Z- | 909.5 | 909.5488 | C₄₉H₈₂O₁₃P | M + H |
| | | eicosatrienoyl)-2- | | | | |
| | | (8Z,11Z,14Z- | | | | |
| | | eicosatrienoyl)-glycero-3- | | | | |
| | | phospho-(1′-myo-inositol) | | | | |
| LMGP06010637 | PI(20:5(5Z,8Z,11Z,14Z,17Z)/20:2(11Z,14Z)) | 1-(5Z,8Z,11Z,14Z,17Z- | 909.5 | 909.5488 | C₄₉H₈₂O₁₃P | M + H |
| | | eicosapentaenoyl)-2- | | | | |
| | | (11Z,14Z-eicosadienoyl)- | | | | |
| | | glycero-3-phospho-(1′- | | | | |
| | | myo-inositol) | | | | |
| LMGP06010676 | PI(22:0/17:0) | 1-docosanoyl-2- | 909.5 | 909.6427 | C₄₉H₉₄O₁₃P | M + H |
| | | heptadecanoyl-glycero-3- | | | | |
| | | phospho-(1′-myo-inositol) | | | | |
| LMGP06010770 | PI(22:4(7Z,10Z,13Z,16Z)/18:3(6Z,9Z,12Z)) | 1-(7Z,10Z,13Z,16Z- | 909.5 | 909.5488 | C₄₉H₈₂O₁₃P | M + H |
| | | docosatetraenoyl)-2- | | | | |
| | | (6Z,9Z,12Z- | | | | |
| | | octadecatrienoyl)-glycero- | | | | |
| | | 3-phospho-(1′-myo- | | | | |
| | | inositol) | | | | |
| LMGP06010771 | PI(22:4(7Z,10Z,13Z,16Z)/18:3(9Z,12Z,15Z)) | 1-(7Z,10Z,13Z,16Z- | 909.5 | 909.5488 | C₄₉H₈₂O₁₃P | M + H |
| | | docosatetraenoyl)-2- | | | | |
| | | (9Z,12Z,15Z- | | | | |
| | | octadecatrienoyl)-glycero- | | | | |
| | | 3-phospho-(1′-myo- | | | | |
| | | inositol) | | | | |
| LMGP06010797 | PI(22:6(4Z,7Z,10Z,13Z,16Z,19Z)/18:1(9Z)) | 1- | 909.5 | 909.5488 | C₄₉H₈₂O₁₃P | M + H |
| | | (4Z,7Z,10Z,13Z,16Z,19Z- | | | | |
| | | docosahexaenoyl)-2-(9Z- | | | | |
| | | octadecenoyl)-glycero-3- | | | | |
| | | phospho-(1′-myo-inositol) | | | | |
| LMGP06010821 | PI(21:0/18:0) | 1-heneicosanoyl-2- | 909.5 | 909.6427 | C₄₉H₉₄O₁₃P | M + H |
| | | octadecanoyl-glycero-3- | | | | |
| | | phospho-(1′-myo-inositol) | | | | |
| LMGP06020060 | PI(O-20:0/20:0) | 1-eicosyl-2-eicosanoyl- | 909.5 | 909.6790 | C₄₉H₉₈O₁₂P | M + H |
| | | glycero-3-phospho-(1′- | | | | |
| | | myo-inositol) | | | | |
| LMGP06020085 | PI(O-18:0/22:0) | 1-octadecyl-2-docosanoyl- | 909.5 | 909.6790 | C₄₉H₉₈O₁₂P | M + H |
| | | glycero-3-phospho-(1′- | | | | |
| | | myo-inositol) | | | | |
| LMGP06010213 | PI(17:0/22:1(11Z)) | 1-heptadecanoyl-2-(11Z-docosenoyl)- | 907.5 | 907.6270 | C₄₈H₉₂O₁₃P | M + H |
| | | glycero-3-phospho-(1′-myo-inositol) | | | | |
| LMGP06010241 | PI(17:1(9Z)/22:0) | 1-(9Z-heptadecenoyl)-2-docosanoyl-glycero- | 907.5 | 907.6270 | C₄₈H₉₂O₁₃P | M + H |
| | | 3-phospho-(1′-myo-inositol) | | | | |
| LMGP06010304 | PI(18:1(9Z)/21:0) | 1-(9Z-octadecenoyl)-2-heneicosanoyl- | 907.5 | 907.6270 | C₄₈H₉₂O₁₃P | M + H |
| | | glycero-3-phospho-(1′-myo-inositol) | | | | |
| LMGP06010422 | PI(18:4(6Z,9Z,12Z,15Z)/22:4(7Z,10Z,13Z,16Z)) | 1-(6Z,9Z,12Z,15Z-octadecatetraenoyl)-2- | 907.5 | 907.5331 | C₄₉H₈₀O₁₃P | M + H |
| | | (7Z,10Z,13Z,16Z-docosatetraenoyl)-glycero- | | | | |
| | | 3-phospho-(1′-myo-inositol) | | | | |
| LMGP06010437 | PI(19:0/22:1(11Z)) | 1-nonadecanoyl-2-(11Z-eicosenoyl)-glycero- | 907.5 | 907.6270 | C₄₈H₉₂O₁₃P | M + H |
| | | 3-phospho-(1′-myo-inositol) | | | | |
| LMGP06010467 | PI(19:1(9Z)/20:0) | 1-(9Z-nonadecenoyl)-2-eicosanoyl-glycero- | 907.5 | 907.6270 | C₄₈H₉₂O₁₃P | M + H |
| | | 3-phospho-(1′-myo-inositol) | | | | |
| LMGP06010489 | PI(20:0/19:1(9Z)) | 1-eicosanoyl-2-(9Z-nonadecenoyl)-glycero- | 907.5 | 907.6270 | C₄₈H₉₂O₁₃P | M + H |
| | | 3-phospho-(1′-myo-inositol) | | | | |
| LMGP06010913 | PI(20:1(11Z)/19:0) | 1-(11Z-eicosenoyl)-2-nonadecanoyl-glycero- | 907.5 | 907.6270 | C₄₈H₉₂O₁₃P | M + H |
| | | 3-phospho-(1′-myo-inositol) | | | | |
| LMGP06010581 | PI(20:3(8Z,11Z,14Z)/20:5(5Z,8Z,11Z,14Z,17Z)) | 1-(8Z,11Z,14Z-eicosatrienoyl)-2- | 907.5 | 907.5331 | C₄₉H₈₀O₁₃P | M + H |
| | | (5Z,8Z,11Z,14Z,17Z-eicosapentaenoyl)- | | | | |
| | | glycero-3-phospho-(1′-myo-inositol) | | | | |
| LMGP06010638 | PI(20:5(5Z,8Z,11Z,14Z,17Z)/20:3(8Z,11Z,14Z,)) | 1-(5Z,8Z,11Z,14Z,17Z-eicosapentaenoyl)-2- | 907.5 | 907.5331 | C₄₉H₈₀O₁₃P | M + H |
| | | (8Z,11Z,14Z,-eicosatrienoyl)-glycero-3- | | | | |
| | | phospho-(1′-myo-inositol) | | | | |
| LMGP06010654 | PI(21:0/18:1(9Z)) | 1-heneicosanoyl-2-(9Z-octadecenoyl)- | 907.5 | 907.6270 | C₄₈H₉₂O₁₃P | M + H |
| | | glycero-3-phospho-(1′-myo-inositol) | | | | |
| LMGP06010677 | PI(22:0/17:1(9Z)) | 1-docosanoyl-2-(9Z-heptadecenoyl)-glycero- | 907.5 | 907.6270 | C₄₈H₉₂O₁₃P | M + H |
| | | 3-phospho-(1′-myo-inositol) | | | | |
| LMGP06010702 | PI(22:1(11Z)/17:0) | 1-(11Z-docosenoyl)-2-heptadecanoyl- | 907.5 | 907.6270 | C₄₈H₉₂O₁₃P | M + H |
| | | glycero-3-phospho-(1′-myo-inositol) | | | | |
| LMGP06010772 | PI(22:4(7Z,10Z,13Z,16Z)/18:4(6Z,9Z,12Z,15Z)) | 1-(7Z,10Z,13Z,16Z-docosatetraenoyl)-2- | 907.5 | 907.5331 | C₄₉H₈₀O₁₃P | M + H |
| | | (6Z,9Z,12Z,15Z,-octadecatetraenoyl)- | | | | |
| | | glycero-3-phospho-(1′-myo-inositol) | | | | |
| LMGP06010798 | PI(22:6(4Z,7Z,10Z,13Z,16Z,19Z)/18:2(9Z,12Z)) | 1-(4Z,7Z,10Z,13Z,16Z,19Z- | 907.5 | 907.5331 | C₄₉H₈₀O₁₃P | M + H |
| | | docosahexaenoyl)-2-(9Z,12Z- | | | | |
| | | octadecadienoyl)-glycero-3-phospho-(1′- | | | | |
| | | myo-inositol) | | | | |
| LMGP06010827 | PI(20:4(5Z,8Z,11Z,14Z)/20:4(5Z,8Z,11Z,14Z)) | 1,2-di-(5Z,8Z,11Z,14Z-eicosatetraenoyl)-sn- | 907.5 | 907.5331 | C₄₉H₈₀O₁₃P | M + H |
| | | glycero-3-phospho-(1′-myo-inositol) | | | | |
| LMGP06010844 | PI(18:2(9Z,12Z)/22:6(4Z,7Z,10Z,13Z,16Z,19Z)) | 1-(9Z,12Z-octadecadienoyl)-2- | 907.5 | 907.5331 | C₄₉H₈₀O₁₃P | M + H |
| | | (4Z,7Z,10Z,13Z,18Z,19Z- | | | | |
| | | docosahexaenoyl)-glycero-3-phospho-(1′- | | | | |
| | | myo-inositol) | | | | |
| LMGP06020039 | PI(O-18:0/22:1(11Z)) | 1-octadecyl-2-(11Z-docosenoyl)-glycero-3- | 907.5 | 907.6634 | C₄₉H₉₈O₁₂P | M + H |
| | | phospho-(1′-myo-inositol) | | | | |
| LMGP06020061 | PI(O-20:0/20:1(11Z)) | 1-eicosyl-2-(11Z-eicosenoyl)-glycero-3- | 907.5 | 907.6634 | C₄₉H₉₈O₁₂P | M + H |
| | | phospho-(1′-myo-inositol) | | | | |
| LMGP06030053 | PI(P-18:0/22:0) | 1-(1Z-octadecenyl)-2-docosanoyl-glycero-3- | 907.5 | 907.6634 | C₄₉H₉₈O₁₂P | M + H |
| | | phospho-(1′-myo-inositol) | | | | |
| LMGP06030076 | PI(P-20:0/20:0) | 1-(1Z-eicosenyl)-2-eicosanoyl-glycero-3- | 907.5 | 907.6634 | C₄₉H₉₈O₁₂P | M + H |
| | | phospho-(1′-myo-inositol) | | | | |
| LMST05050019 | Kurilensoside I | 3beta-(2-O-methyl-beta-D- | 907.5 | 907.4897 | C₄₄H₇₅O₁₉ | M + H |
| | | xylopyranosyloxy)-24R-[2-O-methyl-beta-D- | | | | |
| | | xylopyranosyl-(1-5)-alpha-L- | | | | |
| | | arabinofuranosyloxy]-5alpha-cholest-22E-en- | | | | |
| | | 4beta,6alpha,7alpha,8,15beta-pentol | | | | |

Note:
Chains containing double bonds and/or functional groups with defined regiochemistry, geometry and stereochemistry are meant to serve as examples for structure-drawing purposes. In many cases there may be alternative isobaric structures.

### REFERENCES

Fleet, G. H. (1990). “Yeasts in dairy products”. Journal of Applied Bacteriology 68 (3): 199-211.
2. Hilary K. Dalton; R. G. Board; & R. R. Davenport (1984). “The yeasts of British fresh sausage and minced beef”. Antonie van Leeuwenhoek 50 (3): 227-248.
3. Kurtzman, C. P. and Robnett, C. J. (1998) Antonie Van Leeuwenhoek 73, 331-71.
4. Leray, C., et al. (1987). “Thin-layer chromatography of human platelet phospholipids with fatty acid analysis”. J Chromatogr. 420(2): p. 411-6.
5. M Turk, L Méjanelle, M Sentjurc, J O Grimalt, N Gunde-Cimerman, A Plemenita\check{s}. (2004). “Salt-induced changes in lipid composition and membrane fluidity of halophilic yeast-like melanized fungi.” Extremophiles, 8, pp. 53-61.
6. Merdinger, E. and E. M. Devine, Jr., Lipids of Debaryomyces Hansenii. (1965). J Bacteriol, 89: p. 1488-93.
Merdinger, E., Frye, R. H. (1966). “Distribution of C14 from glucose-1-C14 in the lipid fractions of Debaryomyces hansenii”. J. Bact. 91, 1831-1833.
8. Szoka F and Papahadjopoulos D. (1980). “Comparative properties and methods of preparation of lipid vesicles (liposomes). Ann Rev Biophys Bioeng 9: 467-508.
9. Uta Breuer; & Hauke Harms (2006). “Debaryomyces hansenii—an extremophilic yeast with biotechnological potential”. Yeast 23 (6): 415-437.

What is claimed is:

1. A medical or cosmetic composition comprising at least 0.1% w/w whole yeast organisms and a medical or cosmetic ingredient.

2. The composition of claim 1, wherein the medical or cosmetic ingredient comprises at least one of absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

3. The composition of claim 1, wherein the medical or cosmetic ingredient comprises a soap.

4. The composition of claim 1, wherein the yeast is Debaryomyces hansenii.

5. A method of making a lipid yeast extract, comprising, a) mixing supernatant from a centrifuged yeast culture with methanol and chloroform; and b) separating, after standing, lipids from the methanol and chloroform portion, to form a lipid yeast extract.

6. The method of claim 5, further comprising, separating)ids in the lipid yeast extract of claim 5 by chromatography.

7. (canceled)

8. A lipid yeast extract composition made by the method of claim 6.

9. A medical or cosmetic composition comprising a lipid yeast extract of claim 8 and a medical or cosmetic ingredient.

10. A medical or cosmetic composition comprising at least 0.1% w/w whole yeast organisms and a lipid yeast extract of claim 8 and at least one other medical or cosmetic ingredient.

11. A method of making a lipid yeast extract comprising a) mixing sonicated cells from a centrifuged yeast culture with methanol and chloroform; and b) separating, after standing, lipids from the methanol and chloroform portion, to form a lipid yeast extract.

12. The method of claim 11, further comprising, separating lipids in the lipid yeast extract of claim 11 by chromatography.

13. (canceled)

14. A lipid yeast extract composition made by the method of claim 12,

15. A medical or cosmetic composition comprising a lipid yeast extract of claim 14 and a medical or cosmetic ingredient.

16. A medical or cosmetic composition comprising at least 0.1% w/w whole yeast organisms and a lipid yeast extract of claim 14 and at least one other medical or cosmetic ingredient.

17. The composition of claim 1 comprising at least 25% w/w whole yeast organisms.

18. The composition of claim 1 comprising at least 50% w/w whole yeast organisms.

19. The composition of claim 1, wherein the yeast is Debaryomyces hansenii and a second yeast that was separately cultured from the Debaryomyces hansenii.

20. A liposome delivery vehicle of one or more medicaments or actives comprising a lipid yeast extract composition of claim 8.

21. A liposome delivery vehicle of one or more medicaments or actives comprising the lipid yeast extract composition of claim 14.

22. A kit comprising the medical or cosmetic composition of claim 16 and instructions for use of the composition.


SEQUENCE LISTING (13 sequences)

SEQ ID NO: 1 - protein, 32 aa, Debaryomyces hansenii
EQGDQPAGAESGGEESAPATFQVHDGLFMTDR

SEQ ID NO: 2 - protein, 19 aa, Debaryomyces hansenii
MAMLTFLHEPAVLYNLKDR

SEQ ID NO: 3 - protein, 32 aa, Debaryomyces hansenii
SDWLGDQDAIHYMTEQAPASVVELENYGMPFS

SEQ ID NO: 4 - protein, 18 aa, Debaryomyces hansenii
SPVKPGIPYKQLTVGVPK

SEQ ID NO: 5 - protein, 29 aa, Debaryomyces hansenii
ELASQPDVDGFLVGGASLKPEFVDIINAK

SEQ ID NO: 6 - protein, 19 aa, Debaryomyces hansenii
YRIPADVDPLTITSSLSSD

SEQ ID NO: 7 - protein, 30 aa, Debaryomyces hansenii
KPVIAAVNGYAFGGGCELAMMCDIIYAGEK

SEQ ID NO: 8 - protein, 19 aa, Debaryomyces hansenii
SSIGTGYDLSASTFSPDGR

SEQ ID NO: 9 - protein, 16 aa, Debaryomyces hansenii
ASSVTTFTGEPNMCPR

SEQ ID NO: 10 - protein, 26 aa, Debaryomyces hansenii
FDCSNFNLTVHEAMGTGDLDLLSAFR

SEQ ID NO: 11 - protein, 15 aa, Debaryomyces hansenii
VEEIPEEWELYYPQK

SEQ ID NO: 12 - protein, 20 aa, Debaryomyces hansenii
EREQIKSLNNQFASFIDKVR

SEQ ID NO: 13 - protein, 12 aa, Debaryomyces hansenii
VTAAPQSVCALR